SEQUENCE LISTING (1123 sequences)

SEQ ID NO: 1 - protein, 234 aa, Artificial Sequence
  Description of Artificial Sequence: Synthetic polypeptide
  MOD_RES (30)..(33): Any amino acid
  MOD_RES (50)..(60): Any amino acid
  MOD_RES (100)..(112): Any amino acid
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 2 - protein, 127 aa, Artificial Sequence
  Description of Artificial Sequence: Synthetic polypeptide
  MOD_RES (30)..(33): Any amino acid
  MOD_RES (50)..(60): Any amino acid
  MOD_RES (100)..(112): Any amino acid
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSAS

SEQ ID NO: 3 - protein, 214 aa, Artificial Sequence
  Description of Artificial Sequence: Synthetic polypeptide
  MOD_RES (91)..(96): Any amino acid
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 109 aa, Artificial Sequence
  Description of Artificial Sequence: Synthetic polypeptide
  MOD_RES (91)..(96): Any amino acid
DIQMTQSPSSLSASVGDRVTITCRASQSVSSAVAWYQQKPGKAPKLLIYSASSLYSGVPSRFSGSRSGTDFTLTISSLQPEDFATYYCQQXXXXXXTFGQGTKVEIKRT

SEQ ID NO: 5 - protein, 455 aa, Homo sapiens
  MOD_RES (30)..(33): Any amino acid
  MOD_RES (50)..(60): Any amino acid
  MOD_RES (100)..(112): Any amino acid
EVQLVESGGGLVQPGGSLRLSCAASGFTFXXXXIHWVRQAPGKGLEWVAXXXXXXXXXXXYADSVKGRFTISADTSKNTAYLQMNSLRAEDTAVYYCARXXXXXXXXXXXXXDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 355 aa, Homo sapiens
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 7-8 - skipped (2 entries)

SEQ ID NO: 9 - protein, 4 aa, Artificial Sequence
  Description of Artificial Sequence: Synthetic peptide
SGYF

SEQ ID NO: 10 - skipped

SEQ ID NO: 11 - protein, 4 aa, Artificial Sequence
  Description of Artificial Sequence: Synthetic peptide
GSYF

SEQ ID NO: 12 - protein, 4 aa, Artificial Sequence
  Description of Artificial Sequence: Synthetic peptide
TSYY

SEQ ID NO: 13-14 - skipped (2 entries)

SEQ ID NO: 15 - protein, 4 aa, Artificial Sequence
  Description of Artificial Sequence: Synthetic peptide
SRYA

SEQ ID NO: 16 - protein, 4 aa, Artificial Sequence
  Description of Artificial Sequence: Synthetic peptide
SSYF

SEQ ID NO: 17 - protein, 4 aa, Artificial Sequence
  Description of Artificial Sequence: Synthetic peptide
DYYV

SEQ ID NO: 18-20 - skipped (3 entries)

SEQ ID NO: 21 - protein, 4 aa, Artificial Sequence
  Description of Artificial Sequence: Synthetic peptide
RYYW

SEQ ID NO: 22 - protein, 4 aa, Artificial Sequence
  Description of Artificial Sequence: Synthetic peptide
SYYW

SEQ ID NO: 23 - protein, 4 aa, Artificial Sequence
  Description of Artificial Sequence: Synthetic peptide
SWYV

SEQ ID NO: 24 - protein, 4 aa, Artificial Sequence
  Description of Artificial Sequence: Synthetic peptide
GYYY

SEQ ID NO: 25 - protein, 4 aa, Artificial Sequence
  Description of Artificial Sequence: Synthetic peptide
RYYY

SEQ ID NO: 26 - protein, 4 aa, Artificial Sequence
  Description of Artificial Sequence: Synthetic peptide
SYYY

SEQ ID NO: 27 - protein, 4 aa, Artificial Sequence
  Description of Artificial Sequence: Synthetic peptide
SSYY

SEQ ID NO: 28-31 - skipped (4 entries)

SEQ ID NO: 32 - protein, 4 aa, Artificial Sequence
  Description of Artificial Sequence: Synthetic peptide
WGYG

SEQ ID NO: 33 - protein, 4 aa, Artificial Sequence
  Description of Artificial Sequence: Synthetic peptide
GFYY

SEQ ID NO: 34-35 - skipped (2 entries)

SEQ ID NO: 36 - protein, 4 aa, Artificial Sequence
  Description of Artificial Sequence: Synthetic peptide
SQYA

SEQ ID NO: 37 - protein, 4 aa, Artificial Sequence
  Description of Artificial Sequence: Synthetic peptide
SAYW

SEQ ID NO: 38 - skipped

SEQ ID NO: 39 - protein, 4 aa, Artificial Sequence
  Description of Artificial Sequence: Synthetic peptide
GTYY

SEQ ID NO: 40-41 - skipped (2 entries)

SEQ ID NO: 42 - protein, 4 aa, Artificial Sequence
  Description of Artificial Sequence: Synthetic peptide
SFYG

SEQ ID NO: 43-49 - skipped (7 entries)

SEQ ID NO: 50 - protein, 4 aa, Artificial Sequence
  Description of Artificial Sequence: Synthetic peptide
SWYE

SEQ ID NO: 51-54 - skipped (4 entries)

SEQ ID NO: 55 - protein, 4 aa, Artificial Sequence
  Description of Artificial Sequence: Synthetic peptide
STYF

SEQ ID NO: 56-57 - skipped (2 entries)

SEQ ID NO: 58 - protein, 4 aa, Artificial Sequence
  Description of Artificial Sequence: Synthetic peptide
SRYY

SEQ ID NO: 59-60 - skipped (2 entries)

SEQ ID NO: 61 - protein, 10 aa, Artificial Sequence
  Description of Artificial Sequence: Synthetic peptide
RISSYGGHTD

SEQ ID NO: 62 - skipped

SEQ ID NO: 63 - protein, 10 aa, Artificial Sequence
  Description of Artificial Sequence: Synthetic peptide
YISPTWGYTY

SEQ ID NO: 64 - protein, 10 aa, Artificial Sequence
  Description of Artificial Sequence: Synthetic peptide
WIWPIGGYTY

SEQ ID NO: 65-66 - skipped (2 entries)

SEQ ID NO: 67 - protein, 10 aa, Artificial Sequence
  Description of Artificial Sequence: Synthetic peptide
AIYSPTGYTD

SEQ ID NO: 68 - protein, 10 aa, Artificial Sequence
  Description of Artificial Sequence: Synthetic peptide
WIYSSGGYTY

SEQ ID NO: 69 - protein, 10 aa, Artificial Sequence
  Description of Artificial Sequence: Synthetic peptide
AIDSYWGDTY

SEQ ID NO: 70-72 - skipped (3 entries)

SEQ ID NO: 73 - protein, 10 aa, Artificial Sequence
  Description of Artificial Sequence: Synthetic peptide
AIFPSGGITT

SEQ ID NO: 74 - protein, 10 aa, Artificial Sequence
  Description of Artificial Sequence: Synthetic peptide
DIYPSSGYTY

SEQ ID NO: 75 - protein, 10 aa, Artificial Sequence
  Description of Artificial Sequence: Synthetic peptide
AIYPYHSKTY

SEQ ID NO: 76 - protein, 10 aa, Artificial Sequence
  Description of Artificial Sequence: Synthetic peptide
WISPSGSVTA

SEQ ID NO: 77 - protein, 10 aa, Artificial Sequence
  Description of Artificial Sequence: Synthetic peptide
GIYPYGGYTS

SEQ ID NO: 78 - protein, 10 aa, Artificial Sequence
  Description of Artificial Sequence: Synthetic peptide
RIHPPSGYTD

SEQ ID NO: 79 - protein, 10 aa, Artificial Sequence
  Description of Artificial Sequence: Synthetic peptide
AIYPFSGGTY

SEQ ID NO: 80-83 - skipped (4 entries)

SEQ ID NO: 84 - protein, 10 aa, Artificial Sequence
  Description of Artificial Sequence: Synthetic peptide
AIYPYGGSTY

SEQ ID NO: 85 - protein, 10 aa, Artificial Sequence
  Description of Artificial Sequence: Synthetic peptide
FIDPHGGSTY

SEQ ID NO: 86-87 - skipped (2 entries)

SEQ ID NO: 88 - protein, 10 aa, Artificial Sequence
  Description of Artificial Sequence: Synthetic peptide
RIYPDSGYTY

SEQ ID NO: 89 - protein, 10 aa, Artificial Sequence
  Description of Artificial Sequence: Synthetic peptide
LIGPDGGYTY

SEQ ID NO: 90 - skipped

SEQ ID NO: 91 - protein, 10 aa, Artificial Sequence
  Description of Artificial Sequence: Synthetic peptide
SILSGGGYTV

SEQ ID NO: 92-93 - skipped (2 entries)

SEQ ID NO: 94 - protein, 10 aa, Artificial Sequence
  Description of Artificial Sequence: Synthetic peptide
WIYPYGGFTD

SEQ ID NO: 95-101 - skipped (7 entries)

SEQ ID NO: 102 - protein, 10 aa, Artificial Sequence
  Description of Artificial Sequence: Synthetic peptide
RIGPYSSYTY

SEQ ID NO: 103-106 - skipped (4 entries)

SEQ ID NO: 107 - protein, 10 aa, Artificial Sequence
  Description of Artificial Sequence: Synthetic peptide
WISPSGSHTG

SEQ ID NO: 108-109 - skipped (2 entries)

SEQ ID NO: 110 - protein, 10 aa, Artificial Sequence
  Description of Artificial Sequence: Synthetic peptide
FISSDSGYTQ

SEQ ID NO: 111-112 - skipped (2 entries)

SEQ ID NO: 113 - protein, 7 aa, Artificial Sequence
  Description of Artificial Sequence: Synthetic peptide
AQYVPGL

SEQ ID NO: 114 - skipped

SEQ ID NO: 115 - protein, 7 aa, Artificial Sequence
  Description of Artificial Sequence: Synthetic peptide
AWFGFAF

SEQ ID NO: 116 - protein, 6 aa, Artificial Sequence
  Description of Artificial Sequence: Synthetic peptide
DAGSGF

SEQ ID NO: 117-118 - skipped (2 entries)

SEQ ID NO: 119 - protein, 6 aa, Artificial Sequence
  Description of Artificial Sequence: Synthetic peptide
EGYIGM

SEQ ID NO: 120 - protein, 10 aa, Artificial Sequence
  Description of Artificial Sequence: Synthetic peptide
FTPSDLGYGL

SEQ ID NO: 121 - protein, 6 aa, Artificial Sequence
  Description of Artificial Sequence: Synthetic peptide
FYFYGF

SEQ ID NO: 122-124 - skipped (3 entries)

SEQ ID NO: 125 - protein, 6 aa, Artificial Sequence
  Description of Artificial Sequence: Synthetic peptide
GWPWGL

SEQ ID NO: 126 - protein, 8 aa, Artificial Sequence
  Description of Artificial Sequence: Synthetic peptide
GWYAAYGM

SEQ ID NO: 127 - protein, 6 aa, Artificial Sequence
  Description of Artificial Sequence: Synthetic peptide
GYWYGM

SEQ ID NO: 128 - protein, 8 aa, Artificial Sequence
  Description of Artificial Sequence: Synthetic peptide
GYYGGWGM

SEQ ID NO: 129 - protein, 8 aa, Artificial Sequence
  Description of Artificial Sequence: Synthetic peptide
GYYVEGVL

SEQ ID NO: 130 - protein, 8 aa, Artificial Sequence
  Description of Artificial Sequence: Synthetic peptide
GYYVFGVM

SEQ ID NO: 131 - protein, 8 aa, Artificial Sequence
  Description of Artificial Sequence: Synthetic peptide
GYYVYVVM

SEQ ID NO: 132-135 - skipped (4 entries)

SEQ ID NO: 136 - protein, 11 aa, Artificial Sequence
  Description of Artificial Sequence: Synthetic peptide
LSDIYHSFSGM

SEQ ID NO: 137 - protein, 7 aa, Artificial Sequence
  Description of Artificial Sequence: Synthetic peptide
LSYPGVL

SEQ ID NO: 138-139 - skipped (2 entries)

SEQ ID NO: 140 - protein, 9 aa, Artificial Sequence
  Description of Artificial Sequence: Synthetic peptide
PYHQYAEGM

SEQ ID NO: 141 - protein, 6 aa, Artificial Sequence
  Description of Artificial Sequence: Synthetic peptide
QASRGL

SEQ ID NO: 142 - skipped

SEQ ID NO: 143 - protein, 6 aa, Artificial Sequence
  Description of Artificial Sequence: Synthetic peptide
RVYPGF

SEQ ID NO: 144-145 - skipped (2 entries)

SEQ ID NO: 146 - protein, 6 aa, Artificial Sequence
  Description of Artificial Sequence: Synthetic peptide
SGFFAF

SEQ ID NO: 147-153 - skipped (7 entries)

SEQ ID NO: 154 - protein, 8 aa, Artificial Sequence
  Description of Artificial Sequence: Synthetic peptide
TYYPSYGM

SEQ ID NO: 155-158 - skipped (4 entries)

SEQ ID NO: 159 - protein, 7 aa, Artificial Sequence
  Description of Artificial Sequence: Synthetic peptide
VRYPGVM

SEQ ID NO: 160-161 - skipped (2 entries)

SEQ ID NO: 162 - protein, 7 aa, Artificial Sequence
  Description of Artificial Sequence: Synthetic peptide
TMSYSAL

SEQ ID NO: 163-164 - skipped (2 entries)

SEQ ID NO: 165 - protein, 9 aa, Artificial Sequence
  Description of Artificial Sequence: Synthetic peptide
RASQSVSSA

SEQ ID NO: 166 - skipped

SEQ ID NO: 167 - protein, 9 aa, Artificial Sequence
  Description of Artificial Sequence: Synthetic peptide
RASQSVSSA

SEQ ID NO: 168 - protein, 9 aa, Artificial Sequence
  Description of Artificial Sequence: Synthetic peptide
RASQSVSSA

SEQ ID NO: 169-170 - skipped (2 entries)

SEQ ID NO: 171 - protein, 9 aa, Artificial Sequence
  Description of Artificial Sequence: Synthetic peptide
RASQSVSSA

SEQ ID NO: 172 - protein, 9 aa, Artificial Sequence
  Description of Artificial Sequence: Synthetic peptide
RASQSVSSA

SEQ ID NO: 173 - protein, 9 aa, Artificial Sequence
  Description of Artificial Sequence: Synthetic peptide
RASQSVSSA

SEQ ID NO: 174-176 - skipped (3 entries)

SEQ ID NO: 177 - protein, 9 aa, Artificial Sequence
  Description of Artificial Sequence: Synthetic peptide
RASQSVSSA

SEQ ID NO: 178 - protein, 9 aa, Artificial Sequence
  Description of Artificial Sequence: Synthetic peptide
RASQSVSSA

SEQ ID NO: 179 - protein, 9 aa, Artificial Sequence
  Description of Artificial Sequence: Synthetic peptide
RASQSVSSA

SEQ ID NO: 180 - protein, 9 aa, Artificial Sequence
  Description of Artificial Sequence: Synthetic peptide
RASQSVSSA

SEQ ID NO: 181 - protein, 9 aa, Artificial Sequence
  Description of Artificial Sequence: Synthetic peptide
RASQSVSSA

SEQ ID NO: 182 - protein, 9 aa, Artificial Sequence
  Description of Artificial Sequence: Synthetic peptide
RASQSVSSA

SEQ ID NO: 183 - protein, 9 aa, Artificial Sequence
  Description of Artificial Sequence: Synthetic peptide
RASQSVSSA

SEQ ID NO: 184-187 - skipped (4 entries)

SEQ ID NO: 188 - protein, 9 aa, Artificial Sequence
  Description of Artificial Sequence: Synthetic peptide
RASQSVSSA

SEQ ID NO: 189 - protein, 9 aa, Artificial Sequence
  Description of Artificial Sequence: Synthetic peptide
RASQSVSSA

SEQ ID NO: 190-191 - skipped (2 entries)

SEQ ID NO: 192 - protein, 9 aa, Artificial Sequence
  Description of Artificial Sequence: Synthetic peptide
RASQSVSSA

SEQ ID NO: 193 - protein, 9 aa, Artificial Sequence
  Description of Artificial Sequence: Synthetic peptide
RASQSVSSA

SEQ ID NO: 194 - skipped

SEQ ID NO: 195 - protein, 9 aa, Artificial Sequence
  Description of Artificial Sequence: Synthetic peptide
RASQSVSSA

SEQ ID NO: 196-197 - skipped (2 entries)

SEQ ID NO: 198 - protein, 9 aa, Artificial Sequence
  Description of Artificial Sequence: Synthetic peptide
RASQSVSSA

SEQ ID NO: 199-205 - skipped (7 entries)

SEQ ID NO: 206 - protein, 9 aa, Artificial Sequence
  Description of Artificial Sequence: Synthetic peptide
RASQSVSSA

SEQ ID NO: 207-210 - skipped (4 entries)

SEQ ID NO: 211 - protein, 9 aa, Artificial Sequence
  Description of Artificial Sequence: Synthetic peptide
RASQSVSSA

SEQ ID NO: 212-213 - skipped (2 entries)

SEQ ID NO: 214 - protein, 9 aa, Artificial Sequence
  Description of Artificial Sequence: Synthetic peptide
RASQSVSSA

SEQ ID NO: 215-216 - skipped (2 entries)

SEQ ID NO: 217 - protein, 7 aa, Artificial Sequence
  Description of Artificial Sequence: Synthetic peptide
SASSLYS

SEQ ID NO: 218 - skipped

SEQ ID NO: 219 - protein, 7 aa, Artificial Sequence
  Description of Artificial Sequence: Synthetic peptide
SASSLYS

SEQ ID NO: 220 - protein, 7 aa, Artificial Sequence
  Description of Artificial Sequence: Synthetic peptide
SASSLYS

SEQ ID NO: 221-222 - skipped (2 entries)

SEQ ID NO: 223 - protein, 7 aa, Artificial Sequence
  Description of Artificial Sequence: Synthetic peptide
SASSLYS

SEQ ID NO: 224 - protein, 7 aa, Artificial Sequence
  Description of Artificial Sequence: Synthetic peptide
SASSLYS

SEQ ID NO: 225 - protein, 7 aa, Artificial Sequence
  Description of Artificial Sequence: Synthetic peptide
SASSLYS

SEQ ID NO: 226-228 - skipped (3 entries)

SEQ ID NO: 229 - protein, 7 aa, Artificial Sequence
  Description of Artificial Sequence: Synthetic peptide
SASSLYS

SEQ ID NO: 230 - protein, 7 aa, Artificial Sequence
  Description of Artificial Sequence: Synthetic peptide
SASSLYS

SEQ ID NO: 231 - protein, 7 aa, Artificial Sequence
  Description of Artificial Sequence: Synthetic peptide
SASSLYS

SEQ ID NO: 232 - protein, 7 aa, Artificial Sequence
  Description of Artificial Sequence: Synthetic peptide
SASSLYS

SEQ ID NO: 233 - protein, 7 aa, Artificial Sequence
  Description of Artificial Sequence: Synthetic peptide
SASSLYS

SEQ ID NO: 234 - protein, 7 aa, Artificial Sequence
  Description of Artificial Sequence: Synthetic peptide
SASSLYS

SEQ ID NO: 235 - protein, 7 aa, Artificial Sequence
  Description of Artificial Sequence: Synthetic peptide
SASSLYS

SEQ ID NO: 236-239 - skipped (4 entries)

SEQ ID NO: 240 - protein, 7 aa, Artificial Sequence
  Description of Artificial Sequence: Synthetic peptide
SASSLYS

SEQ ID NO: 241 - protein, 7 aa, Artificial Sequence
  Description of Artificial Sequence: Synthetic peptide
SASSLYS

SEQ ID NO: 242-243 - skipped (2 entries)

SEQ ID NO: 244 - protein, 7 aa, Artificial Sequence
  Description of Artificial Sequence: Synthetic peptide
SASSLYS

SEQ ID NO: 245 - protein, 7 aa, Artificial Sequence
  Description of Artificial Sequence: Synthetic peptide
SASSLYS

SEQ ID NO: 246 - skipped

SEQ ID NO: 247 - protein, 7 aa, Artificial Sequence
  Description of Artificial Sequence: Synthetic peptide
SASSLYS

SEQ ID NO: 248-249 - skipped (2 entries)

SEQ ID NO: 250 - protein, 7 aa, Artificial Sequence
  Description of Artificial Sequence: Synthetic peptide
SASSLYS

SEQ ID NO: 251-257 - skipped (7 entries)

SEQ ID NO: 258 - protein, 7 aa, Artificial Sequence
  Description of Artificial Sequence: Synthetic peptide
SASSLYS

SEQ ID NO: 259-262 - skipped (4 entries)

SEQ ID NO: 263 - protein, 7 aa, Artificial Sequence
  Description of Artificial Sequence: Synthetic peptide
SASSLYS

SEQ ID NO: 264-265 - skipped (2 entries)

SEQ ID NO: 266 - protein, 7 aa, Artificial Sequence
  Description of Artificial Sequence: Synthetic peptide
SASSLYS

SEQ ID NO: 267-268 - skipped (2 entries)

SEQ ID NO: 269 - protein, 6 aa, Artificial Sequence
  Description of Artificial Sequence: Synthetic peptide
SYPTLG

SEQ ID NO: 270 - skipped

SEQ ID NO: 271 - protein, 6 aa, Artificial Sequence
  Description of Artificial Sequence: Synthetic peptide
VYSSPL

SEQ ID NO: 272 - protein, 6 aa, Artificial Sequence
  Description of Artificial Sequence: Synthetic peptide
YYYSLL

SEQ ID NO: 273-274 - skipped (2 entries)

SEQ ID NO: 275 - protein, 6 aa, Artificial Sequence
  Description of Artificial Sequence: Synthetic peptide
SYSALY

SEQ ID NO: 276 - protein, 6 aa, Artificial Sequence
  Description of Artificial Sequence: Synthetic peptide
ADPSLV

SEQ ID NO: 277 - protein, 6 aa, Artificial Sequence
  Description of Artificial Sequence: Synthetic peptide
YSWSLW

SEQ ID NO: 278-280 - skipped (3 entries)

SEQ ID NO: 281 - protein, 6 aa, Artificial Sequence
  Description of Artificial Sequence: Synthetic peptide
RDRSPY

SEQ ID NO: 282 - protein, 6 aa, Artificial Sequence
  Description of Artificial Sequence: Synthetic peptide
YALRPL

SEQ ID NO: 283 - protein, 6 aa, Artificial Sequence
  Description of Artificial Sequence: Synthetic peptide
YTDAPW

SEQ ID NO: 284 - protein, 6 aa, Artificial Sequence
  Description of Artificial Sequence: Synthetic peptide
YDTSPY

SEQ ID NO: 285 - protein, 6 aa, Artificial Sequence
  Description of Artificial Sequence: Synthetic peptide
YYGSLA

SEQ ID NO: 286 - protein, 6 aa, Artificial Sequence
  Description of Artificial Sequence: Synthetic peptide
SHWYPF

SEQ ID NO: 287 - protein, 6 aa, Artificial Sequence
  Description of Artificial Sequence: Synthetic peptide
YKSSPW

SEQ ID NO: 288-291 - skipped (4 entries)

SEQ ID NO: 292 - protein, 6 aa, Artificial Sequence
  Description of Artificial Sequence: Synthetic peptide
RYSTPV

SEQ ID NO: 293 - protein, 6 aa, Artificial Sequence
  Description of Artificial Sequence: Synthetic peptide
GYSTLV

SEQ ID NO: 294-295 - skipped (2 entries)

SEQ ID NO: 296 - protein, 6 aa, Artificial Sequence
  Description of Artificial Sequence: Synthetic peptide
YYSPLL

SEQ ID NO: 297 - protein, 6 aa, Artificial Sequence
  Description of Artificial Sequence: Synthetic peptide
WSSPLH

SEQ ID NO: 298 - skipped

SEQ ID NO: 299 - protein, 6 aa, Artificial Sequence
  Description of Artificial Sequence: Synthetic peptide
DSWGLW

SEQ ID NO: 300-301 - skipped (2 entries)

SEQ ID NO: 302 - protein, 6 aa, Artificial Sequence
  Description of Artificial Sequence: Synthetic peptide
VQTSLA

SEQ ID NO: 303-309 - skipped (7 entries)

SEQ ID NO: 310 - protein, 6 aa, Artificial Sequence
  Description of Artificial Sequence: Synthetic peptide
SFSSPV

SEQ ID NO: 311-314 - skipped (4 entries)

SEQ ID NO: 315 - protein, 6 aa, Artificial Sequence
  Description of Artificial Sequence: Synthetic peptide
WYPSLI

SEQ ID NO: 316-317 - skipped (2 entries)

SEQ ID NO: 318 - protein, 6 aa, Artificial Sequence
  Description of Artificial Sequence: Synthetic peptide
GFGFLV

SEQ ID NO: 319 - protein, 7 aa, Artificial Sequence
  Description of Artificial Sequence: Synthetic peptide
GFTFSGY

SEQ ID NO: 320 - protein, 10 aa, Artificial Sequence
  Description of Artificial Sequence: Synthetic peptide
GFTFSGYFIH

SEQ ID NO: 321 - protein, 5 aa, Artificial Sequence
  Description of Artificial Sequence: Synthetic peptide
GYFIH

SEQ ID NO: 322 - protein, 6 aa, Artificial Sequence
  Description of Artificial Sequence: Synthetic peptide
SGYFIH

SEQ ID NO: 323 - protein, 8 aa, Artificial Sequence
  Description of Artificial Sequence: Synthetic peptide
GFTFSGYF

SEQ ID NO: 324 - protein, 6 aa, Artificial Sequence
  Description of Artificial Sequence: Synthetic peptide
SSYGGH

SEQ ID NO: 325 - protein, 10 aa, Artificial Sequence
  Description of Artificial Sequence: Synthetic peptide
RISSYGGHTD

SEQ ID NO: 326 - protein, 17 aa, Artificial Sequence
  Description of Artificial Sequence: Synthetic peptide
RISSYGGHTDYADSVKG

SEQ ID NO: 327 - protein, 13 aa, Artificial Sequence
  Description of Artificial Sequence: Synthetic peptide
WVARISSYGGHTD

SEQ ID NO: 328 - protein, 8 aa, Artificial Sequence
  Description of Artificial Sequence: Synthetic peptide
ISSYGGHT

SEQ ID NO: 329 - protein, 9 aa, Artificial Sequence
  Description of Artificial Sequence: Synthetic peptide
AQYVPGLDY

SEQ ID NO: 330 - protein, 9 aa, Artificial Sequence
  Description of Artificial Sequence: Synthetic peptide
AQYVPGLDY

SEQ ID NO: 331 - protein, 9 aa, Artificial Sequence
  Description of Artificial Sequence: Synthetic peptide
AQYVPGLDY

SEQ ID NO: 332 - protein, 10 aa, Artificial Sequence
  Description of Artificial Sequence: Synthetic peptide
ARAQYVPGLD

SEQ ID NO: 333 - protein, 11 aa, Artificial Sequence
  Description of Artificial Sequence: Synthetic peptide
ARAQYVPGLDY

SEQ ID NO: 334 - protein, 11 aa, Artificial Sequence
  Description of Artificial Sequence: Synthetic peptide
RASQSVSSAVA

SEQ ID NO: 335 - protein, 11 aa, Artificial Sequence
  Description of Artificial Sequence: Synthetic peptide
RASQSVSSAVA

SEQ ID NO: 336 - protein, 11 aa, Artificial Sequence
  Description of Artificial Sequence: Synthetic peptide
RASQSVSSAVA

SEQ ID NO: 337 - protein, 7 aa, Artificial Sequence
  Description of Artificial Sequence: Synthetic peptide
SSAVAWY

SEQ ID NO: 338 - protein, 6 aa, Artificial Sequence
  Description of Artificial Sequence: Synthetic peptide
QSVSSA

SEQ ID NO: 339 - protein, 7 aa, Artificial Sequence
  Description of Artificial Sequence: Synthetic peptide
SASSLYS

SEQ ID NO: 340 - protein, 7 aa, Artificial Sequence
  Description of Artificial Sequence: Synthetic peptide
SASSLYS

SEQ ID NO: 341 - protein, 7 aa, Artificial Sequence
  Description of Artificial Sequence: Synthetic peptide
SASSLYS

SEQ ID NO: 342 - protein, 10 aa, Artificial Sequence
  Description of Artificial Sequence: Synthetic peptide
LLIYSASSLY

SEQ ID NO: 343 - protein, 2 aa, Artificial Sequence
  Description of Artificial Sequence: Synthetic peptide
SA

SEQ ID NO: 344 - protein, 9 aa, Artificial Sequence
  Description of Artificial Sequence: Synthetic peptide
QQSYPTLGT

SEQ ID NO: 345 - protein, 9 aa, Artificial Sequence
  Description of Artificial Sequence: Synthetic peptide
QQSYPTLGT

SEQ ID NO: 346 - protein, 9 aa, Artificial Sequence
  Description of Artificial Sequence: Synthetic peptide
QQSYPTLGT

SEQ ID NO: 347 - protein, 8 aa, Artificial Sequence
  Description of Artificial Sequence: Synthetic peptide
QQSYPTLG

SEQ ID NO: 348 - protein, 9 aa, Artificial Sequence
  Description of Artificial Sequence: Synthetic peptide
QQSYPTLGT

SEQ ID NO: 349 - protein, 7 aa, Artificial Sequence
  Description of Artificial Sequence: Synthetic peptide
GFTFGSY

SEQ ID NO: 350 - protein, 10 aa, Artificial Sequence
  Description of Artificial Sequence: Synthetic peptide
GFTFGSYFIH

SEQ ID NO: 351 - protein, 5 aa, Artificial Sequence
  Description of Artificial Sequence: Synthetic peptide
SYFIH

SEQ ID NO: 352 - protein, 6 aa, Artificial Sequence
  Description of Artificial Sequence: Synthetic peptide
GSYFIH

SEQ ID NO: 353 - protein, 8 aa, Artificial Sequence
  Description of Artificial Sequence: Synthetic peptide
GFTFGSYF

SEQ ID NO: 354 - protein, 6 aa, Artificial Sequence
  Description of Artificial Sequence: Synthetic peptide
SPTWGY

SEQ ID NO: 355 - protein, 10 aa, Artificial Sequence
  Description of Artificial Sequence: Synthetic peptide
YISPTWGYTY

SEQ ID NO: 356 - protein, 17 aa, Artificial Sequence
  Description of Artificial Sequence: Synthetic peptide
YISPTWGYTYYADSVKG

SEQ ID NO: 357 - protein, 13 aa, Artificial Sequence
  Description of Artificial Sequence: Synthetic peptide
WVAYISPTWGYTY

SEQ ID NO: 358 - protein, 8 aa, Artificial Sequence
  Description of Artificial Sequence: Synthetic peptide
ISPTWGYT

SEQ ID NO: 359 - protein, 9 aa, Artificial Sequence
  Description of Artificial Sequence: Synthetic peptide
AWFGFAFDY

SEQ ID NO: 360 - protein, 9 aa, Artificial Sequence
  Description of Artificial Sequence: Synthetic peptide
AWFGFAFDY

SEQ ID NO: 361 - protein, 9 aa, Artificial Sequence
  Description of Artificial Sequence: Synthetic peptide
AWFGFAFDY

SEQ ID NO: 362 - protein, 10 aa, Artificial Sequence
  Description of Artificial Sequence: Synthetic peptide
ARAWFGFAFD

SEQ ID NO: 363 - protein, 11 aa, Artificial Sequence
  Description of Artificial Sequence: Synthetic peptide
ARAWFGFAFDY

SEQ ID NO: 364 - protein, 11 aa, Artificial Sequence
  Description of Artificial Sequence: Synthetic peptide
RASQSVSSAVA

SEQ ID NO: 365 - protein, 11 aa, Artificial Sequence
  Description of Artificial Sequence: Synthetic peptide
RASQSVSSAVA

SEQ ID NO: 366 - protein, 11 aa, Artificial Sequence
  Description of Artificial Sequence: Synthetic peptide
RASQSVSSAVA

SEQ ID NO: 367 - protein, 7 aa, Artificial Sequence
  Description of Artificial Sequence: Synthetic peptide
SSAVAWY

SEQ ID NO: 368 - protein, 6 aa, Artificial Sequence
  Description of Artificial Sequence: Synthetic peptide
QSVSSA

SEQ ID NO: 369 - protein, 7 aa, Artificial Sequence
  Description of Artificial Sequence: Synthetic peptide
SASSLYS

SEQ ID NO: 370 - protein, 7 aa, Artificial Sequence
  Description of Artificial Sequence: Synthetic peptide
SASSLYS

SEQ ID NO: 371 - protein, 7 aa, Artificial Sequence
  Description of Artificial Sequence: Synthetic peptide
SASSLYS

SEQ ID NO: 372 - protein, 10 aa, Artificial Sequence
  Description of Artificial Sequence: Synthetic peptide
LLIYSASSLY

SEQ ID NO: 373 - protein, 2 aa, Artificial Sequence
  Description of Artificial Sequence: Synthetic peptide
SA

SEQ ID NO: 374 - protein, 9 aa, Artificial Sequence
  Description of Artificial Sequence: Synthetic peptide
QQVYSSPLT

SEQ ID NO: 375 - protein, 9 aa, Artificial Sequence
  Description of Artificial Sequence: Synthetic peptide
QQVYSSPLT

SEQ ID NO: 376 - protein, 9 aa, Artificial Sequence
  Description of Artificial Sequence: Synthetic peptide
QQVYSSPLT

SEQ ID NO: 377 - protein, 8 aa, Artificial Sequence
  Description of Artificial Sequence: Synthetic peptide
QQVYSSPL

SEQ ID NO: 378 - protein, 9 aa, Artificial Sequence
  Description of Artificial Sequence: Synthetic peptide
QQVYSSPLT

SEQ ID NO: 379 - protein, 7 aa, Artificial Sequence
  Description of Artificial Sequence: Synthetic peptide
GFTFTSY

SEQ ID NO: 380 - protein, 10 aa, Artificial Sequence
  Description of Artificial Sequence: Synthetic peptide
GFTFTSYYIH

SEQ ID NO: 381 - protein, 5 aa, Artificial Sequence
  Description of Artificial Sequence: Synthetic peptide
SYYIH

SEQ ID NO: 382 - protein, 6 aa, Artificial Sequence
  Description of Artificial Sequence: Synthetic peptide
TSYYIH

SEQ ID NO: 383 - protein, 8 aa, Artificial Sequence
  Description of Artificial Sequence: Synthetic peptide
GFTFTSYY

SEQ ID NO: 384 - protein, 6 aa, Artificial Sequence
  Description of Artificial Sequence: Synthetic peptide
WPIGGY

SEQ ID NO: 385 - protein, 10 aa, Artificial Sequence
  Description of Artificial Sequence: Synthetic peptide
WIWPIGGYTY

SEQ ID NO: 386 - protein, 17 aa, Artificial Sequence
  Description of Artificial Sequence: Synthetic peptide
WIWPIGGYTYYADSVKG

SEQ ID NO: 387 - protein, 13 aa, Artificial Sequence
  Description of Artificial Sequence: Synthetic peptide
WVAWIWPIGGYTY

SEQ ID NO: 388 - protein, 8 aa, Artificial Sequence
  Description of Artificial Sequence: Synthetic peptide
IWPIGGYT

SEQ ID NO: 389 - protein, 8 aa, Artificial Sequence
  Description of Artificial Sequence: Synthetic peptide
DAGSGFDY

SEQ ID NO: 390 - protein, 8 aa, Artificial Sequence
  Description of Artificial Sequence: Synthetic peptide
DAGSGFDY

SEQ ID NO: 391 - protein, 8 aa, Artificial Sequence
  Description of Artificial Sequence: Synthetic peptide
DAGSGFDY

SEQ ID NO: 392 - protein, 9 aa, Artificial Sequence
  Description of Artificial Sequence: Synthetic peptide
ARDAGSGFD

SEQ ID NO: 393 - protein, 10 aa, Artificial Sequence
  Description of Artificial Sequence: Synthetic peptide
ARDAGSGFDY

SEQ ID NO: 394 - protein, 11 aa, Artificial Sequence
  Description of Artificial Sequence: Synthetic peptide
RASQSVSSAVA

SEQ ID NO: 395 - protein, 11 aa, Artificial Sequence
  Description of Artificial Sequence: Synthetic peptide
RASQSVSSAVA

SEQ ID NO: 396 - protein, 11 aa, Artificial Sequence
  Description of Artificial Sequence: Synthetic peptide
RASQSVSSAVA

SEQ ID NO: 397 - protein, 7 aa, Artificial Sequence
  Description of Artificial Sequence: Synthetic peptide
SSAVAWY

SEQ ID NO: 398 - protein, 6 aa, Artificial Sequence
  Description of Artificial Sequence: Synthetic peptide
QSVSSA

SEQ ID NO: 399 - protein, 7 aa, Artificial Sequence
  Description of Artificial Sequence: Synthetic peptide
SASSLYS

SEQ ID NO: 400 - protein, 7 aa, Artificial Sequence
  Description of Artificial Sequence: Synthetic peptide
SASSLYS

SEQ ID NO: 401 - protein, 7 aa, Artificial Sequence
  Description of Artificial Sequence: Synthetic peptide
SASSLYS

SEQ ID NO: 402 - protein, 10 aa, Artificial Sequence
  Description of Artificial Sequence: Synthetic peptide
LLIYSASSLY

SEQ ID NO: 403 - protein, 2 aa, Artificial Sequence
  Description of Artificial Sequence: Synthetic peptide
SA

SEQ ID NO: 404 - protein, 9 aa, Artificial Sequence
  Description of Artificial Sequence: Synthetic peptide
QQYYYSLLT

SEQ ID NO: 405 - protein, 9 aa, Artificial Sequence
  Description of Artificial Sequence: Synthetic peptide
QQYYYSLLT

SEQ ID NO: 406 - protein, 9 aa, Artificial Sequence
  Description of Artificial Sequence: Synthetic peptide
QQYYYSLLT

SEQ ID NO: 407 - protein, 8 aa, Artificial Sequence
  Description of Artificial Sequence: Synthetic peptide
QQYYYSLL

SEQ ID NO: 408 - protein, 9 aa, Artificial Sequence
  Description of Artificial Sequence: Synthetic peptide
QQYYYSLLT

SEQ ID NO: 409 - protein, 7 aa, Artificial Sequence
  Description of Artificial Sequence: Synthetic peptide
GFTFSRY

SEQ ID NO: 410 - protein, 10 aa, Artificial Sequence
  Description of Artificial Sequence: Synthetic peptide
GFTFSRYAIH

SEQ ID NO: 411 - protein, 5 aa, Artificial Sequence
  Description of Artificial Sequence: Synthetic peptide
RYAIH

SEQ ID NO: 412 - protein, 6 aa, Artificial Sequence
  Description of Artificial Sequence: Synthetic peptide
SRYAIH

SEQ ID NO: 413 - protein, 8 aa, Artificial Sequence
  Description of Artificial Sequence: Synthetic peptide
GFTFSRYA

SEQ ID NO: 414 - protein, 6 aa, Artificial Sequence
  Description of Artificial Sequence: Synthetic peptide
YSPTGY

SEQ ID NO: 415 - protein, 10 aa, Artificial Sequence
  Description of Artificial Sequence: Synthetic peptide
AIYSPTGYTD

SEQ ID NO: 416 - protein, 17 aa, Artificial Sequence
  Description of Artificial Sequence: Synthetic peptide
AIYSPTGYTDYADSVKG

SEQ ID NO: 417 - protein, 13 aa, Artificial Sequence
  Description of Artificial Sequence: Synthetic peptide
WVAAIYSPTGYTD

SEQ ID NO: 418 - protein, 8 aa, Artificial Sequence
  Description of Artificial Sequence: Synthetic peptide
IYSPTGYT

SEQ ID NO: 419 - protein, 8 aa, Artificial Sequence
  Description of Artificial Sequence: Synthetic peptide
EGYIGMDY

SEQ ID NO: 420 - protein, 8 aa, Artificial Sequence
  Description of Artificial Sequence: Synthetic peptide
EGYIGMDY

SEQ ID NO: 421 - protein, 8 aa, Artificial Sequence
  Description of Artificial Sequence: Synthetic peptide
EGYIGMDY

SEQ ID NO: 422 - protein, 9 aa, Artificial Sequence
  Description of Artificial Sequence: Synthetic peptide
AREGYIGMD

SEQ ID NO: 423 - protein, 10 aa, Artificial Sequence
  Description of Artificial Sequence: Synthetic peptide
AREGYIGMDY

SEQ ID NO: 424 - protein, 11 aa, Artificial Sequence
  Description of Artificial Sequence: Synthetic peptide
RASQSVSSAVA

SEQ ID NO: 425 - protein, 11 aa, Artificial Sequence
  Description of Artificial Sequence: Synthetic peptide
RASQSVSSAVA

SEQ ID NO: 426 - protein, 11 aa, Artificial Sequence
  Description of Artificial Sequence: Synthetic peptide
RASQSVSSAVA

SEQ ID NO: 427 - protein, 7 aa, Artificial Sequence
  Description of Artificial Sequence: Synthetic peptide
SSAVAWY

SEQ ID NO: 428 - protein, 6 aa, Artificial Sequence
  Description of Artificial Sequence: Synthetic peptide
QSVSSA

SEQ ID NO: 429 - protein, 7 aa, Artificial Sequence
  Description of Artificial Sequence: Synthetic peptide
SASSLYS

SEQ ID NO: 430 - protein, 7 aa, Artificial Sequence
  Description of Artificial Sequence: Synthetic peptide
SASSLYS

SEQ ID NO: 431 - protein, 7 aa, Artificial Sequence
  Description of Artificial Sequence: Synthetic peptide
SASSLYS

SEQ ID NO: 432 - protein, 10 aa, Artificial Sequence
  Description of Artificial Sequence: Synthetic peptide
LLIYSASSLY

SEQ ID NO: 433 - protein, 2 aa, Artificial Sequence
  Description of Artificial Sequence: Synthetic peptide
SA

SEQ ID NO: 434 - protein, 9 aa, Artificial Sequence
  Description of Artificial Sequence: Synthetic peptide
QQSYSALYT

SEQ ID NO: 435 - protein, 9 aa, Artificial Sequence
  Description of Artificial Sequence: Synthetic peptide
QQSYSALYT

SEQ ID NO: 436 - protein, 9 aa, Artificial Sequence
  Description of Artificial Sequence: Synthetic peptide
QQSYSALYT

SEQ ID NO: 437 - protein, 8 aa, Artificial Sequence
  Description of Artificial Sequence: Synthetic peptide
QQSYSALY

SEQ ID NO: 438 - protein, 9 aa, Artificial Sequence
  Description of Artificial Sequence: Synthetic peptide
QQSYSALYT

SEQ ID NO: 439 - protein, 7 aa, Artificial Sequence
  Description of Artificial Sequence: Synthetic peptide
GFTFSSY

SEQ ID NO: 440 - protein, 10 aa, Artificial Sequence
  Description of Artificial Sequence: Synthetic peptide
GFTFSSYFIH

SEQ ID NO: 441 - protein, 5 aa, Artificial Sequence
  Description of Artificial Sequence: Synthetic peptide
SYFIH

SEQ ID NO: 442 - protein, 6 aa, Artificial Sequence
  Description of Artificial Sequence: Synthetic peptide
SSYFIH

SEQ ID NO: 443 - protein, 8 aa, Artificial Sequence
  Description of Artificial Sequence: Synthetic peptide
GFTFSSYF

SEQ ID NO: 444 - protein, 6 aa, Artificial Sequence
  Description of Artificial Sequence: Synthetic peptide
YSSGGY

SEQ ID NO: 445 - protein, 10 aa, Artificial Sequence
  Description of Artificial Sequence: Synthetic peptide
WIYSSGGYTY

SEQ ID NO: 446 - protein, 17 aa, Artificial Sequence
  Description of Artificial Sequence: Synthetic peptide
WIYSSGGYTYYADSVKG

SEQ ID NO: 447 - protein, 13 aa, Artificial Sequence
  Description of Artificial Sequence: Synthetic peptide
WVAWIYSSGGYTY

SEQ ID NO: 448 - protein, 8 aa, Artificial Sequence
  Description of Artificial Sequence: Synthetic peptide
IYSSGGYT

SEQ ID NO: 449 - protein, 12 aa, Artificial Sequence
  Description of Artificial Sequence: Synthetic peptide
FTPSDLGYGLDY

SEQ ID NO: 450 - protein, 12 aa, Artificial Sequence
  Description of Artificial Sequence: Synthetic peptide
FTPSDLGYGLDY

SEQ ID NO: 451 - protein, 12 aa, Artificial Sequence
  Description of Artificial Sequence: Synthetic peptide
FTPSDLGYGLDY

SEQ ID NO: 452 - protein, 13 aa, Artificial Sequence
  Description of Artificial Sequence: Synthetic peptide
ARFTPSDLGYGLD

SEQ ID NO: 453 - protein, 14 aa, Artificial Sequence
  Description of Artificial Sequence: Synthetic peptide
ARFTPSDLGYGLDY

SEQ ID NO: 454 - protein, 11 aa, Artificial Sequence
  Description of Artificial Sequence: Synthetic peptide
RASQSVSSAVA

SEQ ID NO: 455 - protein, 11 aa, Artificial Sequence
  Description of Artificial Sequence: Synthetic peptide
RASQSVSSAVA

SEQ ID NO: 456 - protein, 11 aa, Artificial Sequence
  Description of Artificial Sequence: Synthetic peptide
RASQSVSSAVA

SEQ ID NO: 457 - protein, 7 aa, Artificial Sequence
  Description of Artificial Sequence: Synthetic peptide
SSAVAWY

SEQ ID NO: 458 - protein, 6 aa, Artificial Sequence
  Description of Artificial Sequence: Synthetic peptide
QSVSSA

SEQ ID NO: 459 - protein, 7 aa, Artificial Sequence
  Description of Artificial Sequence: Synthetic peptide
SASSLYS

SEQ ID NO: 460 - protein, 7 aa, Artificial Sequence
  Description of Artificial Sequence: Synthetic peptide
SASSLYS

SEQ ID NO: 461 - protein, 7 aa, Artificial Sequence
  Description of Artificial Sequence: Synthetic peptide
SASSLYS

SEQ ID NO: 462 - protein, 10 aa, Artificial Sequence
  Description of Artificial Sequence: Synthetic peptide
LLIYSASSLY

SEQ ID NO: 463 - protein, 2 aa, Artificial Sequence
  Description of Artificial Sequence: Synthetic peptide
SA

SEQ ID NO: 464 - protein, 9 aa, Artificial Sequence
  Description of Artificial Sequence: Synthetic peptide
QQADPSLVT

SEQ ID NO: 465 - protein, 9 aa, Artificial Sequence
  Description of Artificial Sequence: Synthetic peptide
QQADPSLVT

SEQ ID NO: 466 - protein, 9 aa, Artificial Sequence
  Description of Artificial Sequence: Synthetic peptide
QQADPSLVT

SEQ ID NO: 467 - protein, 8 aa, Artificial Sequence
  Description of Artificial Sequence: Synthetic peptide
QQADPSLV

SEQ ID NO: 468 - protein, 9 aa, Artificial Sequence
  Description of Artificial Sequence: Synthetic peptide
QQADPSLVT

SEQ ID NO: 469 - protein, 7 aa, Artificial Sequence
  Description of Artificial Sequence: Synthetic peptide
GFTFDYY

SEQ ID NO: 470 - protein, 10 aa, Artificial Sequence
  Description of Artificial Sequence: Synthetic peptide
GFTFDYYVIH

SEQ ID NO: 471 - protein, 5 aa, Artificial Sequence
  Description of Artificial Sequence: Synthetic peptide
YYVIH

SEQ ID NO: 472 - protein, 6 aa, Artificial Sequence
  Description of Artificial Sequence: Synthetic peptide
DYYVIH

SEQ ID NO: 473 - protein, 8 aa, Artificial Sequence
  Description of Artificial Sequence: Synthetic peptide
GFTFDYYV

SEQ ID NO: 474 - protein, 6 aa, Artificial Sequence
  Description of Artificial Sequence: Synthetic peptide
DSYWGD

SEQ ID NO: 475 - protein, 10 aa, Artificial Sequence
  Description of Artificial Sequence: Synthetic peptide
AIDSYWGDTY

SEQ ID NO: 476 - protein, 17 aa, Artificial Sequence
  Description of Artificial Sequence: Synthetic peptide
AIDSYWGDTYYADSVKG

SEQ ID NO: 477 - protein, 13 aa, Artificial Sequence
  Description of Artificial Sequence: Synthetic peptide
WVAAIDSYWGDTY

SEQ ID NO: 478 - protein, 8 aa, Artificial Sequence
  Description of Artificial Sequence: Synthetic peptide
IDSYWGDT

SEQ ID NO: 479 - protein, 8 aa, Artificial Sequence
  Description of Artificial Sequence: Synthetic peptide
FYFYGFDY

SEQ ID NO: 480 - protein, 8 aa, Artificial Sequence
  Description of Artificial Sequence: Synthetic peptide
FYFYGFDY

SEQ ID NO: 481 - protein, 8 aa, Artificial Sequence
  Description of Artificial Sequence: Synthetic peptide
FYFYGFDY

SEQ ID NO: 482 - protein, 9 aa, Artificial Sequence
  Description of Artificial Sequence: Synthetic peptide
ARFYFYGFD

SEQ ID NO: 483 - protein, 10 aa, Artificial Sequence
  Description of Artificial Sequence: Synthetic peptide
ARFYFYGFDY

SEQ ID NO: 484 - protein, 11 aa, Artificial Sequence
  Description of Artificial Sequence: Synthetic peptide
RASQSVSSAVA

SEQ ID NO: 485 - protein, 11 aa, Artificial Sequence
  Description of Artificial Sequence: Synthetic peptide
RASQSVSSAVA

SEQ ID NO: 486 - protein, 11 aa, Artificial Sequence
  Description of Artificial Sequence: Synthetic peptide
RASQSVSSAVA

SEQ ID NO: 487 - protein, 7 aa, Artificial Sequence
  Description of Artificial Sequence: Synthetic peptide
SSAVAWY

SEQ ID NO: 488 - protein, 6 aa, Artificial Sequence
  Description of Artificial Sequence: Synthetic peptide
QSVSSA

SEQ ID NO: 489 - protein, 7 aa, Artificial Sequence
  Description of Artificial Sequence: Synthetic peptide
SASSLYS

SEQ ID NO: 490 - protein, 7 aa, Artificial Sequence
  Description of Artificial Sequence: Synthetic peptide
SASSLYS

SEQ ID NO: 491 - protein, 7 aa, Artificial Sequence
  Description of Artificial Sequence: Synthetic peptide
SASSLYS

SEQ ID NO: 492 - protein, 10 aa, Artificial Sequence
  Description of Artificial Sequence: Synthetic peptide
LLIYSASSLY

SEQ ID NO: 493 - protein, 2 aa, Artificial Sequence
  Description of Artificial Sequence: Synthetic peptide
SA

SEQ ID NO: 494 - protein, 9 aa, Artificial Sequence
  Description of Artificial Sequence: Synthetic peptide
QQYSWSLWT

SEQ ID NO: 495 - protein, 9 aa, Artificial Sequence
  Description of Artificial Sequence: Synthetic peptide
QQYSWSLWT

SEQ ID NO: 496 - protein, 9 aa, Artificial Sequence
  Description of Artificial Sequence: Synthetic peptide
QQYSWSLWT

SEQ ID NO: 497 - protein, 8 aa, Artificial Sequence
  Description of Artificial Sequence: Synthetic peptide
QQYSWSLW

SEQ ID NO: 498 - protein, 9 aa, Artificial Sequence
  Description of Artificial Sequence: Synthetic peptide
QQYSWSLWT

SEQ ID NO: 499 - protein, 7 aa, Artificial Sequence
  Description of Artificial Sequence: Synthetic peptide
GFTFRYY

SEQ ID NO: 500 - protein, 10 aa, Artificial Sequence
  Description of Artificial Sequence: Synthetic peptide
GFTFRYYWIH

SEQ ID NO: 501 - protein, 5 aa, Artificial Sequence
  Description of Artificial Sequence: Synthetic peptide
YYWIH

SEQ ID NO: 502 - protein, 6 aa, Artificial Sequence
  Description of Artificial Sequence: Synthetic peptide
RYYWIH

SEQ ID NO: 503 - protein, 8 aa, Artificial Sequence
  Description of Artificial Sequence: Synthetic peptide
GFTFRYYW

SEQ ID NO: 504 - protein, 6 aa, Artificial Sequence
  Description of Artificial Sequence: Synthetic peptide
FPSGGI

SEQ ID NO: 505 - protein, 10 aa, Artificial Sequence
  Description of Artificial Sequence: Synthetic peptide
AIFPSGGITT

SEQ ID NO: 506 - protein, 17 aa, Artificial Sequence
  Description of Artificial Sequence: Synthetic peptide
AIFPSGGITTYADSVKG

SEQ ID NO: 507 - protein, 13 aa, Artificial Sequence
  Description of Artificial Sequence: Synthetic peptide
WVAAIFPSGGITT

SEQ ID NO: 508 - protein, 8 aa, Artificial Sequence
  Description of Artificial Sequence: Synthetic peptide
IFPSGGIT

SEQ ID NO: 509 - protein, 8 aa, Artificial Sequence
  Description of Artificial Sequence: Synthetic peptide
GWPWGLDY

SEQ ID NO: 510 - protein, 8 aa, Artificial Sequence
  Description of Artificial Sequence: Synthetic peptide
GWPWGLDY

SEQ ID NO: 511 - protein, 8 aa, Artificial Sequence
  Description of Artificial Sequence: Synthetic peptide
GWPWGLDY

SEQ ID NO: 512 - protein, 9 aa, Artificial Sequence
  Description of Artificial Sequence: Synthetic peptide
ARGWPWGLD

SEQ ID NO: 513 - protein, 10 aa, Artificial Sequence
  Description of Artificial Sequence: Synthetic peptide
ARGWPWGLDY

SEQ ID NO: 514 - protein, 11 aa, Artificial Sequence
  Description of Artificial Sequence: Synthetic peptide
RASQSVSSAVA

SEQ ID NO: 515 - protein, 11 aa, Artificial Sequence
  Description of Artificial Sequence: Synthetic peptide
RASQSVSSAVA

SEQ ID NO: 516 - protein, 11 aa, Artificial Sequence
  Description of Artificial Sequence: Synthetic peptide
RASQSVSSAVA

SEQ ID NO: 517 - protein, 7 aa, Artificial Sequence
  Description of Artificial Sequence: Synthetic peptide
SSAVAWY

SEQ ID NO: 518 - protein, 6 aa, Artificial Sequence
  Description of Artificial Sequence: Synthetic peptide
QSVSSA

SEQ ID NO: 519 - protein, 7 aa, Artificial Sequence
  Description of Artificial Sequence: Synthetic peptide
SASSLYS

SEQ ID NO: 520 - protein, 7 aa, Artificial Sequence
  Description of Artificial Sequence: Synthetic peptide
SASSLYS

SEQ ID NO: 521 - protein, 7 aa, Artificial Sequence
  Description of Artificial Sequence: Synthetic peptide
SASSLYS

SEQ ID NO: 522 - protein, 10 aa, Artificial Sequence
  Description of Artificial Sequence: Synthetic peptide
LLIYSASSLY

SEQ ID NO: 523 - protein, 2 aa, Artificial Sequence
  Description of Artificial Sequence: Synthetic peptide
SA

SEQ ID NO: 524 - protein, 9 aa, Artificial Sequence
  Description of Artificial Sequence: Synthetic peptide
QQRDRSPYT

SEQ ID NO: 525 - protein, 9 aa, Artificial Sequence
  Description of Artificial Sequence: Synthetic peptide
QQRDRSPYT

SEQ ID NO: 526 - protein, 9 aa, Artificial Sequence
  Description of Artificial Sequence: Synthetic peptide
QQRDRSPYT

SEQ ID NO: 527 - protein, 8 aa, Artificial Sequence
  Description of Artificial Sequence: Synthetic peptide
QQRDRSPY

SEQ ID NO: 528 - protein, 9 aa, Artificial Sequence
  Description of Artificial Sequence: Synthetic peptide
QQRDRSPYT

SEQ ID NO: 529 - protein, 7 aa, Artificial Sequence
  Description of Artificial Sequence: Synthetic peptide
GFTFSYY

SEQ ID NO: 530 - protein, 10 aa, Artificial Sequence
  Description of Artificial Sequence: Synthetic peptide
GFTFSYYWIH

SEQ ID NO: 531 - protein, 5 aa, Artificial Sequence
  Description of Artificial Sequence: Synthetic peptide
YYWIH

SEQ ID NO: 532 - protein, 6 aa, Artificial Sequence
  Description of Artificial Sequence: Synthetic peptide
SYYWIH

SEQ ID NO: 533 - protein, 8 aa, Artificial Sequence
  Description of Artificial Sequence: Synthetic peptide
GFTFSYYW

SEQ ID NO: 534 - protein, 6 aa, Artificial Sequence
  Description of Artificial Sequence: Synthetic peptide
YPSSGY

SEQ ID NO: 535 - protein, 10 aa, Artificial Sequence
  Description of Artificial Sequence: Synthetic peptide
DIYPSSGYTY

SEQ ID NO: 536 - protein, 17 aa, Artificial Sequence
  Description of Artificial Sequence: Synthetic peptide
DIYPSSGYTYYADSVKG

SEQ ID NO: 537 - protein, 13 aa, Artificial Sequence
  Description of Artificial Sequence: Synthetic peptide
WVADIYPSSGYTY

SEQ ID NO: 538 - protein, 8 aa, Artificial Sequence
  Description of Artificial Sequence: Synthetic peptide
IYPSSGYT

SEQ ID NO: 539 - protein, 10 aa, Artificial Sequence
  Description of Artificial Sequence: Synthetic peptide
GWYAAYGMDY

SEQ ID NO: 540 - protein, 10 aa, Artificial Sequence
  Description of Artificial Sequence: Synthetic peptide
GWYAAYGMDY

SEQ ID NO: 541 - protein, 10 aa, Artificial Sequence
  Description of Artificial Sequence: Synthetic peptide
GWYAAYGMDY

SEQ ID NO: 542 - protein, 11 aa, Artificial Sequence
  Description of Artificial Sequence: Synthetic peptide
ARGWYAAYGMD

SEQ ID NO: 543 - protein, 12 aa, Artificial Sequence
  Description of Artificial Sequence: Synthetic peptide
ARGWYAAYGMDY

SEQ ID NO: 544 - protein, 11 aa, Artificial Sequence
  Description of Artificial Sequence: Synthetic peptide
RASQSVSSAVA

SEQ ID NO: 545 - protein, 11 aa, Artificial Sequence
  Description of Artificial Sequence: Synthetic peptide
RASQSVSSAVA

SEQ ID NO: 546 - protein, 11 aa, Artificial Sequence
  Description of Artificial Sequence: Synthetic peptide
RASQSVSSAVA

SEQ ID NO: 547 - protein, 7 aa, Artificial Sequence
  Description of Artificial Sequence: Synthetic peptide
SSAVAWY

SEQ ID NO: 548 - protein, 6 aa, Artificial Sequence
  Description of Artificial Sequence: Synthetic peptide
QSVSSA

SEQ ID NO: 549 - protein, 7 aa, Artificial Sequence
  Description of Artificial Sequence: Synthetic peptide
SASSLYS

SEQ ID NO: 550 - protein, 7 aa, Artificial Sequence
  Description of Artificial Sequence: Synthetic peptide
SASSLYS

SEQ ID NO: 551 - protein, 7 aa, Artificial Sequence
  Description of Artificial Sequence: Synthetic peptide
SASSLYS

SEQ ID NO: 552 - protein, 10 aa, Artificial Sequence
  Description of Artificial Sequence: Synthetic peptide
LLIYSASSLY

SEQ ID NO: 553 - protein, 2 aa, Artificial Sequence
  Description of Artificial Sequence: Synthetic peptide
SA

SEQ ID NO: 554 - protein, 9 aa, Artificial Sequence
  Description of Artificial Sequence: Synthetic peptide
QQYALRPLT

SEQ ID NO: 555 - protein, 9 aa, Artificial Sequence
  Description of Artificial Sequence: Synthetic peptide
QQYALRPLT

SEQ ID NO: 556 - protein, 9 aa, Artificial Sequence
  Description of Artificial Sequence: Synthetic peptide
QQYALRPLT

SEQ ID NO: 557 - protein, 8 aa, Artificial Sequence
  Description of Artificial Sequence: Synthetic peptide
QQYALRPL

SEQ ID NO: 558 - protein, 9 aa, Artificial Sequence
  Description of Artificial Sequence: Synthetic peptide
QQYALRPLT

SEQ ID NO: 559 - protein, 7 aa, Artificial Sequence
  Description of Artificial Sequence: Synthetic peptide
GFTFSWY

SEQ ID NO: 560 - protein, 10 aa, Artificial Sequence
  Description of Artificial Sequence: Synthetic peptide
GFTFSWYVIH

SEQ ID NO: 561 - protein, 5 aa, Artificial Sequence
  Description of Artificial Sequence: Synthetic peptide
WYVIH

SEQ ID NO: 562 - protein, 6 aa, Artificial Sequence
  Description of Artificial Sequence: Synthetic peptide
SWYVIH

SEQ ID NO: 563 - protein, 8 aa, Artificial Sequence
  Description of Artificial Sequence: Synthetic peptide
GFTFSWYV

SEQ ID NO: 564 - protein, 6 aa, Artificial Sequence
  Description of Artificial Sequence: Synthetic peptide
YPYHSK

SEQ ID NO: 565 - protein, 10 aa, Artificial Sequence
  Description of Artificial Sequence: Synthetic peptide
AIYPYHSKTY

SEQ ID NO: 566 - protein, 17 aa, Artificial Sequence
  Description of Artificial Sequence: Synthetic peptide
AIYPYHSKTYYADSVKG

SEQ ID NO: 567 - protein, 13 aa, Artificial Sequence
  Description of Artificial Sequence: Synthetic peptide
WVAAIYPYHSKTY

SEQ ID NO: 568 - protein, 8 aa, Artificial Sequence
  Description of Artificial Sequence: Synthetic peptide
IYPYHSKT

SEQ ID NO: 569 - protein, 8 aa, Artificial Sequence
  Description of Artificial Sequence: Synthetic peptide
GYWYGMDY

SEQ ID NO: 570 - protein, 8 aa, Artificial Sequence
  Description of Artificial Sequence: Synthetic peptide
GYWYGMDY

SEQ ID NO: 571 - protein, 8 aa, Artificial Sequence
  Description of Artificial Sequence: Synthetic peptide
GYWYGMDY

SEQ ID NO: 572 - protein, 9 aa, Artificial Sequence
  Description of Artificial Sequence: Synthetic peptide
ARGYWYGMD

SEQ ID NO: 573 - protein, 10 aa, Artificial Sequence
  Description of Artificial Sequence: Synthetic peptide
ARGYWYGMDY

SEQ ID NO: 574 - protein, 11 aa, Artificial Sequence
  Description of Artificial Sequence: Synthetic peptide
RASQSVSSAVA

SEQ ID NO: 575 - protein, 11 aa, Artificial Sequence
  Description of Artificial Sequence: Synthetic peptide
RASQSVSSAVA

SEQ ID NO: 576 - protein, 11 aa, Artificial Sequence
  Description of Artificial Sequence: Synthetic peptide
RASQSVSSAVA

SEQ ID NO: 577 - protein, 7 aa, Artificial Sequence
  Description of Artificial Sequence: Synthetic peptide
SSAVAWY

SEQ ID NO: 578 - protein, 6 aa, Artificial Sequence
  Description of Artificial Sequence: Synthetic peptide
QSVSSA

SEQ ID NO: 579 - protein, 7 aa, Artificial Sequence
  Description of Artificial Sequence: Synthetic peptide
SASSLYS

SEQ ID NO: 580 - protein, 7 aa, Artificial Sequence
  Description of Artificial Sequence: Synthetic peptide
SASSLYS

SEQ ID NO: 581 - protein, 7 aa, Artificial Sequence
  Description of Artificial Sequence: Synthetic peptide
SASSLYS

SEQ ID NO: 582 - protein, 10 aa, Artificial Sequence
  Description of Artificial Sequence: Synthetic peptide
LLIYSASSLY

SEQ ID NO: 583 - protein, 2 aa, Artificial Sequence
  Description of Artificial Sequence: Synthetic peptide
SA

SEQ ID NO: 584 - protein, 9 aa, Artificial Sequence
  Description of Artificial Sequence: Synthetic peptide
QQYTDAPWT

SEQ ID NO: 585 - protein, 9 aa, Artificial Sequence
  Description of Artificial Sequence: Synthetic peptide
QQYTDAPWT

SEQ ID NO: 586 - protein, 9 aa, Artificial Sequence
  Description of Artificial Sequence: Synthetic peptide
QQYTDAPWT

SEQ ID NO: 587 - protein, 8 aa, Artificial Sequence
  Description of Artificial Sequence: Synthetic peptide
QQYTDAPW

SEQ ID NO: 588 - protein, 9 aa, Artificial Sequence
  Description of Artificial Sequence: Synthetic peptide
QQYTDAPWT

SEQ ID NO: 589 - protein, 7 aa, Artificial Sequence
  Description of Artificial Sequence: Synthetic peptide
GFTFGYY

SEQ ID NO: 590 - protein, 10 aa, Artificial Sequence
  Description of Artificial Sequence: Synthetic peptide
GFTFGYYYIH

SEQ ID NO: 591 - protein, 5 aa, Artificial Sequence
  Description of Artificial Sequence: Synthetic peptide
YYYIH

SEQ ID NO: 592 - protein, 6 aa, Artificial Sequence
  Description of Artificial Sequence: Synthetic peptide
GYYYIH

SEQ ID NO: 593 - protein, 8 aa, Artificial Sequence
  Description of Artificial Sequence: Synthetic peptide
GFTFGYYY

SEQ ID NO: 594 - protein, 6 aa, Artificial Sequence
  Description of Artificial Sequence: Synthetic peptide
SPSGSV

SEQ ID NO: 595 - protein, 10 aa, Artificial Sequence
  Description of Artificial Sequence: Synthetic peptide
WISPSGSVTA

SEQ ID NO: 596 - protein, 17 aa, Artificial Sequence
  Description of Artificial Sequence: Synthetic peptide
WISPSGSVTAYADSVKG

SEQ ID NO: 597 - protein, 13 aa, Artificial Sequence
  Description of Artificial Sequence: Synthetic peptide
WVAWISPSGSVTA

SEQ ID NO: 598 - protein, 8 aa, Artificial Sequence
  Description of Artificial Sequence: Synthetic peptide
ISPSGSVT

SEQ ID NO: 599 - protein, 10 aa, Artificial Sequence
  Description of Artificial Sequence: Synthetic peptide
GYYGGWGMDY

SEQ ID NO: 600 - protein, 10 aa, Artificial Sequence
  Description of Artificial Sequence: Synthetic peptide
GYYGGWGMDY

SEQ ID NO: 601 - protein, 10 aa, Artificial Sequence
  Description of Artificial Sequence: Synthetic peptide
GYYGGWGMDY

SEQ ID NO: 602 - protein, 11 aa, Artificial Sequence
  Description of Artificial Sequence: Synthetic peptide
ARGYYGGWGMD

SEQ ID NO: 603 - protein, 12 aa, Artificial Sequence
  Description of Artificial Sequence: Synthetic peptide
ARGYYGGWGMDY

SEQ ID NO: 604 - protein, 11 aa, Artificial Sequence
  Description of Artificial Sequence: Synthetic peptide
RASQSVSSAVA

SEQ ID NO: 605 - protein, 11 aa, Artificial Sequence
  Description of Artificial Sequence: Synthetic peptide
RASQSVSSAVA

SEQ ID NO: 606 - protein, 11 aa, Artificial Sequence
  Description of Artificial Sequence: Synthetic peptide
RASQSVSSAVA

SEQ ID NO: 607 - protein, 7 aa, Artificial Sequence
  Description of Artificial Sequence: Synthetic peptide
SSAVAWY

SEQ ID NO: 608 - protein, 6 aa, Artificial Sequence
  Description of Artificial Sequence: Synthetic peptide
QSVSSA

SEQ ID NO: 609 - protein, 7 aa, Artificial Sequence
  Description of Artificial Sequence: Synthetic peptide
SASSLYS

SEQ ID NO: 610 - protein, 7 aa, Artificial Sequence
  Description of Artificial Sequence: Synthetic peptide
SASSLYS

SEQ ID NO: 611 - protein, 7 aa, Artificial Sequence
  Description of Artificial Sequence: Synthetic peptide
SASSLYS

SEQ ID NO: 612 - protein, 10 aa, Artificial Sequence
  Description of Artificial Sequence: Synthetic peptide
LLIYSASSLY

SEQ ID NO: 613 - protein, 2 aa, Artificial Sequence
  Description of Artificial Sequence: Synthetic peptide
SA

SEQ ID NO: 614 - protein, 9 aa, Artificial Sequence
  Description of Artificial Sequence: Synthetic peptide
QQYDTSPYT

SEQ ID NO: 615 - protein, 9 aa, Artificial Sequence
  Description of Artificial Sequence: Synthetic peptide
QQYDTSPYT

SEQ ID NO: 616 - protein, 9 aa, Artificial Sequence
  Description of Artificial Sequence: Synthetic peptide
QQYDTSPYT

SEQ ID NO: 617 - protein, 8 aa, Artificial Sequence
  Description of Artificial Sequence: Synthetic peptide
QQYDTSPY

SEQ ID NO: 618 - protein, 9 aa, Artificial Sequence
  Description of Artificial Sequence: Synthetic peptide
QQYDTSPYT

SEQ ID NO: 619 - protein, 7 aa, Artificial Sequence
  Description of Artificial Sequence: Synthetic peptide
GFTFRYY

SEQ ID NO: 620 - protein, 10 aa, Artificial Sequence
  Description of Artificial Sequence: Synthetic peptide
GFTFRYYYIH

SEQ ID NO: 621 - protein, 5 aa, Artificial Sequence
  Description of Artificial Sequence: Synthetic peptide
YYYIH

SEQ ID NO: 622 - protein, 6 aa, Artificial Sequence
  Description of Artificial Sequence: Synthetic peptide
RYYYIH

SEQ ID NO: 623 - protein, 8 aa, Artificial Sequence
  Description of Artificial Sequence: Synthetic peptide
GFTFRYYY

SEQ ID NO: 624 - protein, 6 aa, Artificial Sequence
  Description of Artificial Sequence: Synthetic peptide
YPYGGY

SEQ ID NO: 625 - protein, 10 aa, Artificial Sequence
  Description of Artificial Sequence: Synthetic peptide
GIYPYGGYTS

SEQ ID NO: 626 - protein, 17 aa, Artificial Sequence
  Description of Artificial Sequence: Synthetic peptide
GIYPYGGYTSYADSVKG

SEQ ID NO: 627 - protein, 13 aa, Artificial Sequence
  Description of Artificial Sequence: Synthetic peptide
WVAGIYPYGGYTS

SEQ ID NO: 628 - protein, 8 aa, Artificial Sequence
  Description of Artificial Sequence: Synthetic peptide
IYPYGGYT

SEQ ID NO: 629 - protein, 10 aa, Artificial Sequence
  Description of Artificial Sequence: Synthetic peptide
GYYVEGVLDY

SEQ ID NO: 630 - protein, 10 aa, Artificial Sequence
  Description of Artificial Sequence: Synthetic peptide
GYYVEGVLDY

SEQ ID NO: 631 - protein, 10 aa, Artificial Sequence
  Description of Artificial Sequence: Synthetic peptide
GYYVEGVLDY

SEQ ID NO: 632 - protein, 11 aa, Artificial Sequence
  Description of Artificial Sequence: Synthetic peptide
ARGYYVEGVLD

SEQ ID NO: 633 - protein, 12 aa, Artificial Sequence
  Description of Artificial Sequence: Synthetic peptide
ARGYYVEGVLDY

SEQ ID NO: 634 - protein, 11 aa, Artificial Sequence
  Description of Artificial Sequence: Synthetic peptide
RASQSVSSAVA

SEQ ID NO: 635 - protein, 11 aa, Artificial Sequence
  Description of Artificial Sequence: Synthetic peptide
RASQSVSSAVA

SEQ ID NO: 636 - protein, 11 aa, Artificial Sequence
  Description of Artificial Sequence: Synthetic peptide
RASQSVSSAVA

SEQ ID NO: 637 - protein, 7 aa, Artificial Sequence
  Description of Artificial Sequence: Synthetic peptide
SSAVAWY

SEQ ID NO: 638 - protein, 6 aa, Artificial Sequence
  Description of Artificial Sequence: Synthetic peptide
QSVSSA

SEQ ID NO: 639 - protein, 7 aa, Artificial Sequence
  Description of Artificial Sequence: Synthetic peptide
SASSLYS

SEQ ID NO: 640 - protein, 7 aa, Artificial Sequence
  Description of Artificial Sequence: Synthetic peptide
SASSLYS

SEQ ID NO: 641 - protein, 7 aa, Artificial Sequence
  Description of Artificial Sequence: Synthetic peptide
SASSLYS

SEQ ID NO: 642 - protein, 10 aa, Artificial Sequence
  Description of Artificial Sequence: Synthetic peptide
LLIYSASSLY

SEQ ID NO: 643 - protein, 2 aa, Artificial Sequence
  Description of Artificial Sequence: Synthetic peptide
SA

SEQ ID NO: 644 - protein, 9 aa, Artificial Sequence
  Description of Artificial Sequence: Synthetic peptide
QQYYGSLAT

SEQ ID NO: 645 - protein, 9 aa, Artificial Sequence
  Description of Artificial Sequence: Synthetic peptide
QQYYGSLAT

SEQ ID NO: 646 - protein, 9 aa, Artificial Sequence
  Description of Artificial Sequence: Synthetic peptide
QQYYGSLAT

SEQ ID NO: 647 - protein, 8 aa, Artificial Sequence
  Description of Artificial Sequence: Synthetic peptide
QQYYGSLA

SEQ ID NO: 648 - protein, 9 aa, Artificial Sequence
  Description of Artificial Sequence: Synthetic peptide
QQYYGSLAT

SEQ ID NO: 649 - protein, 7 aa, Artificial Sequence
  Description of Artificial Sequence: Synthetic peptide
GFTFSYY

SEQ ID NO: 650 - protein, 10 aa, Artificial Sequence
  Description of Artificial Sequence: Synthetic peptide
GFTFSYYYIH

SEQ ID NO: 651 - protein, 5 aa, Artificial Sequence
  Description of Artificial Sequence: Synthetic peptide
YYYIH

SEQ ID NO: 652 - protein, 6 aa, Artificial Sequence
  Description of Artificial Sequence: Synthetic peptide
SYYYIH

SEQ ID NO: 653 - protein, 8 aa, Artificial Sequence
  Description of Artificial Sequence: Synthetic peptide
GFTFSYYY

SEQ ID NO: 654 - protein, 6 aa, Artificial Sequence
  Description of Artificial Sequence: Synthetic peptide
HPPSGY

SEQ ID NO: 655 - protein, 10 aa, Artificial Sequence
  Description of Artificial Sequence: Synthetic peptide
RIHPPSGYTD

SEQ ID NO: 656 - protein, 17 aa, Artificial Sequence
  Description of Artificial Sequence: Synthetic peptide
RIHPPSGYTDYADSVKG

SEQ ID NO: 657 - protein, 13 aa, Artificial Sequence
  Description of Artificial Sequence: Synthetic peptide
WVARIHPPSGYTD

SEQ ID NO: 658 - protein, 8 aa, Artificial Sequence
  Description of Artificial Sequence: Synthetic peptide
IHPPSGYT

SEQ ID NO: 659 - protein, 10 aa, Artificial Sequence
  Description of Artificial Sequence: Synthetic peptide
GYYVFGVMDY

SEQ ID NO: 660 - protein, 10 aa, Artificial Sequence
  Description of Artificial Sequence: Synthetic peptide
GYYVFGVMDY

SEQ ID NO: 661 - protein, 10 aa, Artificial Sequence
  Description of Artificial Sequence: Synthetic peptide
GYYVFGVMDY

SEQ ID NO: 662 - protein, 11 aa, Artificial Sequence
  Description of Artificial Sequence: Synthetic peptide
ARGYYVFGVMD

SEQ ID NO: 663 - protein, 12 aa, Artificial Sequence
  Description of Artificial Sequence: Synthetic peptide
ARGYYVFGVMDY

SEQ ID NO: 664 - protein, 11 aa, Artificial Sequence
  Description of Artificial Sequence: Synthetic peptide
RASQSVSSAVA

SEQ ID NO: 665 - protein, 11 aa, Artificial Sequence
  Description of Artificial Sequence: Synthetic peptide
RASQSVSSAVA

SEQ ID NO: 666 - protein, 11 aa, Artificial Sequence
  Description of Artificial Sequence: Synthetic peptide
RASQSVSSAVA

SEQ ID NO: 667 - protein, 7 aa, Artificial Sequence
  Description of Artificial Sequence: Synthetic peptide
SSAVAWY

SEQ ID NO: 668 - protein, 6 aa, Artificial Sequence
  Description of Artificial Sequence: Synthetic peptide
QSVSSA

SEQ ID NO: 669 - protein, 7 aa, Artificial Sequence
  Description of Artificial Sequence: Synthetic peptide
SASSLYS

SEQ ID NO: 670 - protein, 7 aa, Artificial Sequence
  Description of Artificial Sequence: Synthetic peptide
SASSLYS

SEQ ID NO: 671 - protein, 7 aa, Artificial Sequence
  Description of Artificial Sequence: Synthetic peptide
SASSLYS

SEQ ID NO: 672 - protein, 10 aa, Artificial Sequence
  Description of Artificial Sequence: Synthetic peptide
LLIYSASSLY

SEQ ID NO: 673 - protein, 2 aa, Artificial Sequence
  Description of Artificial Sequence: Synthetic peptide
SA

SEQ ID NO: 674 - protein, 9 aa, Artificial Sequence
  Description of Artificial Sequence: Synthetic peptide
QQSHWYPFT

SEQ ID NO: 675 - protein, 9 aa, Artificial Sequence
  Description of Artificial Sequence: Synthetic peptide
QQSHWYPFT

SEQ ID NO: 676 - protein, 9 aa, Artificial Sequence
  Description of Artificial Sequence: Synthetic peptide
QQSHWYPFT

SEQ ID NO: 677 - protein, 8 aa, Artificial Sequence
  Description of Artificial Sequence: Synthetic peptide
QQSHWYPF

SEQ ID NO: 678 - protein, 9 aa, Artificial Sequence
  Description of Artificial Sequence: Synthetic peptide
QQSHWYPFT

SEQ ID NO: 679 - protein, 7 aa, Artificial Sequence
  Description of Artificial Sequence: Synthetic peptide
GFTFSSY

SEQ ID NO: 680 - protein, 10 aa, Artificial Sequence
  Description of Artificial Sequence: Synthetic peptide
GFTFSSYYIH

SEQ ID NO: 681 - protein, 5 aa, Artificial Sequence
  Description of Artificial Sequence: Synthetic peptide
SYYIH

SEQ ID NO: 682 - protein, 6 aa, Artificial Sequence
  Description of Artificial Sequence: Synthetic peptide
SSYYIH

SEQ ID NO: 683 - protein, 8 aa, Artificial Sequence
  Description of Artificial Sequence: Synthetic peptide
GFTFSSYY

SEQ ID NO: 684 - protein, 6 aa, Artificial Sequence
  Description of Artificial Sequence: Synthetic peptide
YPFSGG

SEQ ID NO: 685 - protein, 10 aa, Artificial Sequence
  Description of Artificial Sequence: Synthetic peptide
AIYPFSGGTY

SEQ ID NO: 686 - protein, 17 aa, Artificial Sequence
  Description of Artificial Sequence: Synthetic peptide
AIYPFSGGTYYADSVKG

SEQ ID NO: 687 - protein, 13 aa, Artificial Sequence
  Description of Artificial Sequence: Synthetic peptide
WVAAIYPFSGGTY

SEQ ID NO: 688 - protein, 8 aa, Artificial Sequence
  Description of Artificial Sequence: Synthetic peptide
IYPFSGGT

SEQ ID NO: 689 - protein, 10 aa, Artificial Sequence
  Description of Artificial Sequence: Synthetic peptide
GYYVYVVMDY

SEQ ID NO: 690 - protein, 10 aa, Artificial Sequence
  Description of Artificial Sequence: Synthetic peptide
GYYVYVVMDY

SEQ ID NO: 691 - protein, 10 aa, Artificial Sequence
  Description of Artificial Sequence: Synthetic peptide
GYYVYVVMDY

SEQ ID NO: 692 - protein, 11 aa, Artificial Sequence
  Description of Artificial Sequence: Synthetic peptide
ARGYYVYVVMD

SEQ ID NO: 693 - protein, 12 aa, Artificial Sequence
  Description of Artificial Sequence: Synthetic peptide
ARGYYVYVVMDY

SEQ ID NO: 694 - protein, 11 aa, Artificial Sequence
  Description of Artificial Sequence: Synthetic peptide
RASQSVSSAVA

SEQ ID NO: 695 - protein, 11 aa, Artificial Sequence
  Description of Artificial Sequence: Synthetic peptide
RASQSVSSAVA

SEQ ID NO: 696 - protein, 11 aa, Artificial Sequence
  Description of Artificial Sequence: Synthetic peptide
RASQSVSSAVA

SEQ ID NO: 697 - protein, 7 aa, Artificial Sequence
  Description of Artificial Sequence: Synthetic peptide
SSAVAWY

SEQ ID NO: 698 - protein, 6 aa, Artificial Sequence
  Description of Artificial Sequence: Synthetic peptide
QSVSSA

SEQ ID NO: 699 - protein, 7 aa, Artificial Sequence
  Description of Artificial Sequence: Synthetic peptide
SASSLYS

SEQ ID NO: 700 - protein, 7 aa, Artificial Sequence
  Description of Artificial Sequence: Synthetic peptide
SASSLYS

SEQ ID NO: 701 - protein, 7 aa, Artificial Sequence
  Description of Artificial Sequence: Synthetic peptide
SASSLYS

SEQ ID NO: 702 - protein, 10 aa, Artificial Sequence
  Description of Artificial Sequence: Synthetic peptide
LLIYSASSLY

SEQ ID NO: 703 - protein, 2 aa, Artificial Sequence
  Description of Artificial Sequence: Synthetic peptide
SA

SEQ ID NO: 704 - protein, 9 aa, Artificial Sequence
  Description of Artificial Sequence: Synthetic peptide
QQYKSSPWT

SEQ ID NO: 705 - protein, 9 aa, Artificial Sequence
  Description of Artificial Sequence: Synthetic peptide
QQYKSSPWT

SEQ ID NO: 706 - protein, 9 aa, Artificial Sequence
  Description of Artificial Sequence: Synthetic peptide
QQYKSSPWT

SEQ ID NO: 707 - protein, 8 aa, Artificial Sequence
  Description of Artificial Sequence: Synthetic peptide
QQYKSSPW

SEQ ID NO: 708 - protein, 9 aa, Artificial Sequence
  Description of Artificial Sequence: Synthetic peptide
QQYKSSPWT

SEQ ID NO: 709 - protein, 7 aa, Artificial Sequence
  Description of Artificial Sequence: Synthetic peptide
GFTFWGY

SEQ ID NO: 710 - protein, 10 aa, Artificial Sequence
  Description of Artificial Sequence: Synthetic peptide
GFTFWGYGIH

SEQ ID NO: 711 - protein, 5 aa, Artificial Sequence
  Description of Artificial Sequence: Synthetic peptide
GYGIH

SEQ ID NO: 712 - protein, 6 aa, Artificial Sequence
  Description of Artificial Sequence: Synthetic peptide
WGYGIH

SEQ ID NO: 713 - protein, 8 aa, Artificial Sequence
  Description of Artificial Sequence: Synthetic peptide
GFTFWGYG

SEQ ID NO: 714 - protein, 6 aa, Artificial Sequence
  Description of Artificial Sequence: Synthetic peptide
YPYGGS

SEQ ID NO: 715 - protein, 10 aa, Artificial Sequence
  Description of Artificial Sequence: Synthetic peptide
AIYPYGGSTY

SEQ ID NO: 716 - protein, 17 aa, Artificial Sequence
  Description of Artificial Sequence: Synthetic peptide
AIYPYGGSTYYADSVKG

SEQ ID NO: 717 - protein, 13 aa, Artificial Sequence
  Description of Artificial Sequence: Synthetic peptide
WVAAIYPYGGSTY

SEQ ID NO: 718 - protein, 8 aa, Artificial Sequence
  Description of Artificial Sequence: Synthetic peptide
IYPYGGST

SEQ ID NO: 719 - protein, 13 aa, Artificial Sequence
  Description of Artificial Sequence: Synthetic peptide
LSDIYHSFSGMDY

SEQ ID NO: 720 - protein, 13 aa, Artificial Sequence
  Description of Artificial Sequence: Synthetic peptide
LSDIYHSFSGMDY

SEQ ID NO: 721 - protein, 13 aa, Artificial Sequence
  Description of Artificial Sequence: Synthetic peptide
LSDIYHSFSGMDY

SEQ ID NO: 722 - protein, 14 aa, Artificial Sequence
  Description of Artificial Sequence: Synthetic peptide
ARLSDIYHSFSGMD

SEQ ID NO: 723 - protein, 15 aa, Artificial Sequence
  Description of Artificial Sequence: Synthetic peptide
ARLSDIYHSFSGMDY

SEQ ID NO: 724 - protein, 11 aa, Artificial Sequence
  Description of Artificial Sequence: Synthetic peptide
RASQSVSSAVA

SEQ ID NO: 725 - protein, 11 aa, Artificial Sequence
  Description of Artificial Sequence: Synthetic peptide
RASQSVSSAVA

SEQ ID NO: 726 - protein, 11 aa, Artificial Sequence
  Description of Artificial Sequence: Synthetic peptide
RASQSVSSAVA

SEQ ID NO: 727 - protein, 7 aa, Artificial Sequence
  Description of Artificial Sequence: Synthetic peptide
SSAVAWY

SEQ ID NO: 728 - protein, 6 aa, Artificial Sequence
  Description of Artificial Sequence: Synthetic peptide
QSVSSA

SEQ ID NO: 729 - protein, 7 aa, Artificial Sequence
  Description of Artificial Sequence: Synthetic peptide
SASSLYS

SEQ ID NO: 730 - protein, 7 aa, Artificial Sequence
  Description of Artificial Sequence: Synthetic peptide
SASSLYS

SEQ ID NO: 731 - protein, 7 aa, Artificial Sequence
  Description of Artificial Sequence: Synthetic peptide
SASSLYS

SEQ ID NO: 732 - protein, 10 aa, Artificial Sequence
  Description of Artificial Sequence: Synthetic peptide
LLIYSASSLY

SEQ ID NO: 733 - protein, 2 aa, Artificial Sequence
  Description of Artificial Sequence: Synthetic peptide
SA

SEQ ID NO: 734 - protein, 9 aa, Artificial Sequence
  Description of Artificial Sequence: Synthetic peptide
QQRYSTPVT

SEQ ID NO: 735 - protein, 9 aa, Artificial Sequence
  Description of Artificial Sequence: Synthetic peptide
QQRYSTPVT

SEQ ID NO: 736 - protein, 9 aa, Artificial Sequence
  Description of Artificial Sequence: Synthetic peptide
QQRYSTPVT

SEQ ID NO: 737 - protein, 8 aa, Artificial Sequence
  Description of Artificial Sequence: Synthetic peptide
QQRYSTPV

SEQ ID NO: 738 - protein, 9 aa, Artificial Sequence
  Description of Artificial Sequence: Synthetic peptide
QQRYSTPVT

SEQ ID NO: 739 - protein, 7 aa, Artificial Sequence
  Description of Artificial Sequence: Synthetic peptide
GFTFGFY

SEQ ID NO: 740 - protein, 10 aa, Artificial Sequence
  Description of Artificial Sequence: Synthetic peptide
GFTFGFYYIH

SEQ ID NO: 741 - protein, 5 aa, Artificial Sequence
  Description of Artificial Sequence: Synthetic peptide
FYYIH

SEQ ID NO: 742 - protein, 6 aa, Artificial Sequence
  Description of Artificial Sequence: Synthetic peptide
GFYYIH

SEQ ID NO: 743 - protein, 8 aa, Artificial Sequence
  Description of Artificial Sequence: Synthetic peptide
GFTFGFYY

SEQ ID NO: 744 - protein, 6 aa, Artificial Sequence
  Description of Artificial Sequence: Synthetic peptide
DPHGGS

SEQ ID NO: 745 - protein, 10 aa, Artificial Sequence
  Description of Artificial Sequence: Synthetic peptide
FIDPHGGSTY

SEQ ID NO: 746 - protein, 17 aa, Artificial Sequence
  Description of Artificial Sequence: Synthetic peptide
FIDPHGGSTYYADSVKG

SEQ ID NO: 747 - protein, 13 aa, Artificial Sequence
  Description of Artificial Sequence: Synthetic peptide
WVAFIDPHGGSTY

SEQ ID NO: 748 - protein, 8 aa, Artificial Sequence
  Description of Artificial Sequence: Synthetic peptide
IDPHGGST

SEQ ID NO: 749 - protein, 9 aa, Artificial Sequence
  Description of Artificial Sequence: Synthetic peptide
LSYPGVLDY

SEQ ID NO: 750 - protein, 9 aa, Artificial Sequence
  Description of Artificial Sequence: Synthetic peptide
LSYPGVLDY

SEQ ID NO: 751 - protein, 9 aa, Artificial Sequence
  Description of Artificial Sequence: Synthetic peptide
LSYPGVLDY

SEQ ID NO: 752 - protein, 10 aa, Artificial Sequence
  Description of Artificial Sequence: Synthetic peptide
ARLSYPGVLD

SEQ ID NO: 753 - protein, 11 aa, Artificial Sequence
  Description of Artificial Sequence: Synthetic peptide
ARLSYPGVLDY

SEQ ID NO: 754 - protein, 11 aa, Artificial Sequence
  Description of Artificial Sequence: Synthetic peptide
RASQSVSSAVA

SEQ ID NO: 755 - protein, 11 aa, Artificial Sequence
  Description of Artificial Sequence: Synthetic peptide
RASQSVSSAVA

SEQ ID NO: 756 - protein, 11 aa, Artificial Sequence
  Description of Artificial Sequence: Synthetic peptide
RASQSVSSAVA

SEQ ID NO: 757 - protein, 7 aa, Artificial Sequence
  Description of Artificial Sequence: Synthetic peptide
SSAVAWY

SEQ ID NO: 758 - protein, 6 aa, Artificial Sequence
  Description of Artificial Sequence: Synthetic peptide
QSVSSA

SEQ ID NO: 759 - protein, 7 aa, Artificial Sequence
  Description of Artificial Sequence: Synthetic peptide
SASSLYS

SEQ ID NO: 760 - protein, 7 aa, Artificial Sequence
  Description of Artificial Sequence: Synthetic peptide
SASSLYS

SEQ ID NO: 761 - protein, 7 aa, Artificial Sequence
  Description of Artificial Sequence: Synthetic peptide
SASSLYS

SEQ ID NO: 762 - protein, 10 aa, Artificial Sequence
  Description of Artificial Sequence: Synthetic peptide
LLIYSASSLY

SEQ ID NO: 763 - protein, 2 aa, Artificial Sequence
  Description of Artificial Sequence: Synthetic peptide
SA

SEQ ID NO: 764 - protein, 9 aa, Artificial Sequence
  Description of Artificial Sequence: Synthetic peptide
QQGYSTLVT

SEQ ID NO: 765 - protein, 9 aa, Artificial Sequence
  Description of Artificial Sequence: Synthetic peptide
QQGYSTLVT

SEQ ID NO: 766 - protein, 9 aa, Artificial Sequence
  Description of Artificial Sequence: Synthetic peptide
QQGYSTLVT

SEQ ID NO: 767 - protein, 8 aa, Artificial Sequence
  Description of Artificial Sequence: Synthetic peptide
QQGYSTLV

SEQ ID NO: 768 - protein, 9 aa, Artificial Sequence
  Description of Artificial Sequence: Synthetic peptide
QQGYSTLVT

SEQ ID NO: 769 - protein, 7 aa, Artificial Sequence
  Description of Artificial Sequence: Synthetic peptide
GFTFSQY

SEQ ID NO: 770 - protein, 10 aa, Artificial Sequence
  Description of Artificial Sequence: Synthetic peptide
GFTFSQYAIH

SEQ ID NO: 771 - protein, 5 aa, Artificial Sequence
  Description of Artificial Sequence: Synthetic peptide
QYAIH

SEQ ID NO: 772 - protein, 6 aa, Artificial Sequence
  Description of Artificial Sequence: Synthetic peptide
SQYAIH

SEQ ID NO: 773 - protein, 8 aa, Artificial Sequence
  Description of Artificial Sequence: Synthetic peptide
GFTFSQYA

SEQ ID NO: 774 - protein, 6 aa, Artificial Sequence
  Description of Artificial Sequence: Synthetic peptide
YPDSGY

SEQ ID NO: 775 - protein, 10 aa, Artificial Sequence
  Description of Artificial Sequence: Synthetic peptide
RIYPDSGYTY

SEQ ID NO: 776 - protein, 17 aa, Artificial Sequence
  Description of Artificial Sequence: Synthetic peptide
RIYPDSGYTYYADSVKG

SEQ ID NO: 777 - protein, 13 aa, Artificial Sequence
  Description of Artificial Sequence: Synthetic peptide
WVARIYPDSGYTY

SEQ ID NO: 778 - protein, 8 aa, Artificial Sequence
  Description of Artificial Sequence: Synthetic peptide
IYPDSGYT

SEQ ID NO: 779 - protein, 11 aa, Artificial Sequence
  Description of Artificial Sequence: Synthetic peptide
PYHQYAEGMDY

SEQ ID NO: 780-799 - skipped (20 entries)

SEQ ID NO: 800 - protein, 11 aa, Artificial Sequence
  Description of Artificial Sequence: Synthetic peptide
PYHQYAEGMDY

SEQ ID NO: 801 - protein, 11 aa, Artificial Sequence
  Description of Artificial Sequence: Synthetic peptide
PYHQYAEGMDY

SEQ ID NO: 802 - protein, 12 aa, Artificial Sequence
  Description of Artificial Sequence: Synthetic peptide
ARPYHQYAEGMD

SEQ ID NO: 803 - protein, 13 aa, Artificial Sequence
  Description of Artificial Sequence: Synthetic peptide
ARPYHQYAEGMDY

SEQ ID NO: 804 - protein, 11 aa, Artificial Sequence
  Description of Artificial Sequence: Synthetic peptide
RASQSVSRAVA

SEQ ID NO: 805 - protein, 11 aa, Artificial Sequence
  Description of Artificial Sequence: Synthetic peptide
RASQSVSRAVA

SEQ ID NO: 806 - protein, 11 aa, Artificial Sequence
  Description of Artificial Sequence: Synthetic peptide
RASQSVSRAVA

SEQ ID NO: 807 - protein, 7 aa, Artificial Sequence
  Description of Artificial Sequence: Synthetic peptide
SRAVAWY

SEQ ID NO: 808 - protein, 6 aa, Artificial Sequence
  Description of Artificial Sequence: Synthetic peptide
QSVSRA

SEQ ID NO: 809 - protein, 7 aa, Artificial Sequence
  Description of Artificial Sequence: Synthetic peptide
SASSLYS

SEQ ID NO: 810 - protein, 7 aa, Artificial Sequence
  Description of Artificial Sequence: Synthetic peptide
SASSLYS

SEQ ID NO: 811 - protein, 7 aa, Artificial Sequence
  Description of Artificial Sequence: Synthetic peptide
SASSLYS

SEQ ID NO: 812 - protein, 10 aa, Artificial Sequence
  Description of Artificial Sequence: Synthetic peptide
LLIYSASSLY

SEQ ID NO: 813 - protein, 2 aa, Artificial Sequence
  Description of Artificial Sequence: Synthetic peptide
SA

SEQ ID NO: 814 - protein, 9 aa, Artificial Sequence
  Description of Artificial Sequence: Synthetic peptide
QQYYSPLLT

SEQ ID NO: 815 - protein, 9 aa, Artificial Sequence
  Description of Artificial Sequence: Synthetic peptide
QQYYSPLLT

SEQ ID NO: 816 - protein, 9 aa, Artificial Sequence
  Description of Artificial Sequence: Synthetic peptide
QQYYSPLLT

SEQ ID NO: 817 - protein, 8 aa, Artificial Sequence
  Description of Artificial Sequence: Synthetic peptide
QQYYSPLL

SEQ ID NO: 818 - protein, 9 aa, Artificial Sequence
  Description of Artificial Sequence: Synthetic peptide
QQYYSPLLT

SEQ ID NO: 819 - protein, 7 aa, Artificial Sequence
  Description of Artificial Sequence: Synthetic peptide
GFTFSAY

SEQ ID NO: 820 - protein, 10 aa, Artificial Sequence
  Description of Artificial Sequence: Synthetic peptide
GFTFSAYWIH

SEQ ID NO: 821 - protein, 5 aa, Artificial Sequence
  Description of Artificial Sequence: Synthetic peptide
AYWIH

SEQ ID NO: 822 - protein, 6 aa, Artificial Sequence
  Description of Artificial Sequence: Synthetic peptide
SAYWIH

SEQ ID NO: 823 - protein, 8 aa, Artificial Sequence
  Description of Artificial Sequence: Synthetic peptide
GFTFSAYW

SEQ ID NO: 824 - protein, 6 aa, Artificial Sequence
  Description of Artificial Sequence: Synthetic peptide
GPDGGY

SEQ ID NO: 825 - protein, 10 aa, Artificial Sequence
  Description of Artificial Sequence: Synthetic peptide
LIGPDGGYTY

SEQ ID NO: 826 - protein, 17 aa, Artificial Sequence
  Description of Artificial Sequence: Synthetic peptide
LIGPDGGYTYYADSVKG

SEQ ID NO: 827 - protein, 13 aa, Artificial Sequence
  Description of Artificial Sequence: Synthetic peptide
WVALIGPDGGYTY

SEQ ID NO: 828 - protein, 8 aa, Artificial Sequence
  Description of Artificial Sequence: Synthetic peptide
IGPDGGYT

SEQ ID NO: 829 - protein, 8 aa, Artificial Sequence
  Description of Artificial Sequence: Synthetic peptide
QASRGLDY

SEQ ID NO: 830 - protein, 8 aa, Artificial Sequence
  Description of Artificial Sequence: Synthetic peptide
QASRGLDY

SEQ ID NO: 831 - protein, 8 aa, Artificial Sequence
  Description of Artificial Sequence: Synthetic peptide
QASRGLDY

SEQ ID NO: 832 - protein, 9 aa, Artificial Sequence
  Description of Artificial Sequence: Synthetic peptide
ARQASRGLD

SEQ ID NO: 833 - protein, 10 aa, Artificial Sequence
  Description of Artificial Sequence: Synthetic peptide
ARQASRGLDY

SEQ ID NO: 834 - protein, 11 aa, Artificial Sequence
  Description of Artificial Sequence: Synthetic peptide
RASQSVSSAVA

SEQ ID NO: 835 - protein, 11 aa, Artificial Sequence
  Description of Artificial Sequence: Synthetic peptide
RASQSVSSAVA

SEQ ID NO: 836 - protein, 11 aa, Artificial Sequence
  Description of Artificial Sequence: Synthetic peptide
RASQSVSSAVA

SEQ ID NO: 837 - protein, 7 aa, Artificial Sequence
  Description of Artificial Sequence: Synthetic peptide
SSAVAWY

SEQ ID NO: 838 - protein, 6 aa, Artificial Sequence
  Description of Artificial Sequence: Synthetic peptide
QSVSSA

SEQ ID NO: 839 - protein, 7 aa, Artificial Sequence
  Description of Artificial Sequence: Synthetic peptide
SASSLYS

SEQ ID NO: 840 - protein, 7 aa, Artificial Sequence
  Description of Artificial Sequence: Synthetic peptide
SASSLYS

SEQ ID NO: 841 - protein, 7 aa, Artificial Sequence
  Description of Artificial Sequence: Synthetic peptide
SASSLYS

SEQ ID NO: 842 - protein, 10 aa, Artificial Sequence
  Description of Artificial Sequence: Synthetic peptide
LLIYSASSLY

SEQ ID NO: 843 - protein, 2 aa, Artificial Sequence
  Description of Artificial Sequence: Synthetic peptide
SA

SEQ ID NO: 844 - protein, 9 aa, Artificial Sequence
  Description of Artificial Sequence: Synthetic peptide
QQWSSPLHT

SEQ ID NO: 845 - protein, 9 aa, Artificial Sequence
  Description of Artificial Sequence: Synthetic peptide
QQWSSPLHT

SEQ ID NO: 846 - protein, 9 aa, Artificial Sequence
  Description of Artificial Sequence: Synthetic peptide
QQWSSPLHT

SEQ ID NO: 847 - protein, 8 aa, Artificial Sequence
  Description of Artificial Sequence: Synthetic peptide
QQWSSPLH

SEQ ID NO: 848 - protein, 9 aa, Artificial Sequence
  Description of Artificial Sequence: Synthetic peptide
QQWSSPLHT

SEQ ID NO: 849 - protein, 7 aa, Artificial Sequence
  Description of Artificial Sequence: Synthetic peptide
GFTFGTY

SEQ ID NO: 850 - protein, 10 aa, Artificial Sequence
  Description of Artificial Sequence: Synthetic peptide
GFTFGTYYIH

SEQ ID NO: 851 - protein, 5 aa, Artificial Sequence
  Description of Artificial Sequence: Synthetic peptide
TYYIH

SEQ ID NO: 852 - protein, 6 aa, Artificial Sequence
  Description of Artificial Sequence: Synthetic peptide
GTYYIH

SEQ ID NO: 853 - protein, 8 aa, Artificial Sequence
  Description of Artificial Sequence: Synthetic peptide
GFTFGTYY

SEQ ID NO: 854 - protein, 6 aa, Artificial Sequence
  Description of Artificial Sequence: Synthetic peptide
LSGGGY

SEQ ID NO: 855 - protein, 10 aa, Artificial Sequence
  Description of Artificial Sequence: Synthetic peptide
SILSGGGYTV

SEQ ID NO: 856 - protein, 17 aa, Artificial Sequence
  Description of Artificial Sequence: Synthetic peptide
SILSGGGYTVYADSVKG

SEQ ID NO: 857 - protein, 13 aa, Artificial Sequence
  Description of Artificial Sequence: Synthetic peptide
WVASILSGGGYTV

SEQ ID NO: 858 - protein, 8 aa, Artificial Sequence
  Description of Artificial Sequence: Synthetic peptide
ILSGGGYT

SEQ ID NO: 859 - protein, 8 aa, Artificial Sequence
  Description of Artificial Sequence: Synthetic peptide
RVYPGFDY

SEQ ID NO: 860 - protein, 8 aa, Artificial Sequence
  Description of Artificial Sequence: Synthetic peptide
RVYPGFDY

SEQ ID NO: 861 - protein, 8 aa, Artificial Sequence
  Description of Artificial Sequence: Synthetic peptide
RVYPGFDY

SEQ ID NO: 862 - protein, 9 aa, Artificial Sequence
  Description of Artificial Sequence: Synthetic peptide
ARRVYPGFD

SEQ ID NO: 863 - protein, 10 aa, Artificial Sequence
  Description of Artificial Sequence: Synthetic peptide
ARRVYPGFDY

SEQ ID NO: 864 - protein, 11 aa, Artificial Sequence
  Description of Artificial Sequence: Synthetic peptide
RASQSVSSAVA

SEQ ID NO: 865 - protein, 11 aa, Artificial Sequence
  Description of Artificial Sequence: Synthetic peptide
RASQSVSSAVA

SEQ ID NO: 866 - protein, 11 aa, Artificial Sequence
  Description of Artificial Sequence: Synthetic peptide
RASQSVSSAVA

SEQ ID NO: 867 - protein, 7 aa, Artificial Sequence
  Description of Artificial Sequence: Synthetic peptide
SSAVAWY

SEQ ID NO: 868 - protein, 6 aa, Artificial Sequence
  Description of Artificial Sequence: Synthetic peptide
QSVSSA

SEQ ID NO: 869 - protein, 7 aa, Artificial Sequence
  Description of Artificial Sequence: Synthetic peptide
SASSLYS

SEQ ID NO: 870 - protein, 7 aa, Artificial Sequence
  Description of Artificial Sequence: Synthetic peptide
SASSLYS

SEQ ID NO: 871 - protein, 7 aa, Artificial Sequence
  Description of Artificial Sequence: Synthetic peptide
SASSLYS

SEQ ID NO: 872 - protein, 10 aa, Artificial Sequence
  Description of Artificial Sequence: Synthetic peptide
LLIYSASSLY

SEQ ID NO: 873 - protein, 2 aa, Artificial Sequence
  Description of Artificial Sequence: Synthetic peptide
SA

SEQ ID NO: 874 - protein, 9 aa, Artificial Sequence
  Description of Artificial Sequence: Synthetic peptide
QQDSWGLWT

SEQ ID NO: 875 - protein, 9 aa, Artificial Sequence
  Description of Artificial Sequence: Synthetic peptide
QQDSWGLWT

SEQ ID NO: 876 - protein, 9 aa, Artificial Sequence
  Description of Artificial Sequence: Synthetic peptide
QQDSWGLWT

SEQ ID NO: 877 - protein, 8 aa, Artificial Sequence
  Description of Artificial Sequence: Synthetic peptide
QQDSWGLW

SEQ ID NO: 878 - protein, 9 aa, Artificial Sequence
  Description of Artificial Sequence: Synthetic peptide
QQDSWGLWT

SEQ ID NO: 879 - protein, 7 aa, Artificial Sequence
  Description of Artificial Sequence: Synthetic peptide
GFTFSFY

SEQ ID NO: 880 - protein, 10 aa, Artificial Sequence
  Description of Artificial Sequence: Synthetic peptide
GFTFSFYGIH

SEQ ID NO: 881 - protein, 5 aa, Artificial Sequence
  Description of Artificial Sequence: Synthetic peptide
FYGIH

SEQ ID NO: 882 - protein, 6 aa, Artificial Sequence
  Description of Artificial Sequence: Synthetic peptide
SFYGIH

SEQ ID NO: 883 - protein, 8 aa, Artificial Sequence
  Description of Artificial Sequence: Synthetic peptide
GFTFSFYG

SEQ ID NO: 884 - protein, 6 aa, Artificial Sequence
  Description of Artificial Sequence: Synthetic peptide
YPYGGF

SEQ ID NO: 885 - protein, 10 aa, Artificial Sequence
  Description of Artificial Sequence: Synthetic peptide
WIYPYGGFTD

SEQ ID NO: 886 - protein, 17 aa, Artificial Sequence
  Description of Artificial Sequence: Synthetic peptide
WIYPYGGFTDYADSVKG

SEQ ID NO: 887 - protein, 13 aa, Artificial Sequence
  Description of Artificial Sequence: Synthetic peptide
WVAWIYPYGGFTD

SEQ ID NO: 888 - protein, 8 aa, Artificial Sequence
  Description of Artificial Sequence: Synthetic peptide
IYPYGGFT

SEQ ID NO: 889 - protein, 8 aa, Artificial Sequence
  Description of Artificial Sequence: Synthetic peptide
SGFFAFDY

SEQ ID NO: 890 - protein, 8 aa, Artificial Sequence
  Description of Artificial Sequence: Synthetic peptide
SGFFAFDY

SEQ ID NO: 891 - protein, 8 aa, Artificial Sequence
  Description of Artificial Sequence: Synthetic peptide
SGFFAFDY

SEQ ID NO: 892 - protein, 9 aa, Artificial Sequence
  Description of Artificial Sequence: Synthetic peptide
ARSGFFAFD

SEQ ID NO: 893 - protein, 10 aa, Artificial Sequence
  Description of Artificial Sequence: Synthetic peptide
ARSGFFAFDY

SEQ ID NO: 894 - protein, 11 aa, Artificial Sequence
  Description of Artificial Sequence: Synthetic peptide
RASQSVSSAVA

SEQ ID NO: 895 - protein, 11 aa, Artificial Sequence
  Description of Artificial Sequence: Synthetic peptide
RASQSVSSAVA

SEQ ID NO: 896 - protein, 11 aa, Artificial Sequence
  Description of Artificial Sequence: Synthetic peptide
RASQSVSSAVA

SEQ ID NO: 897 - protein, 7 aa, Artificial Sequence
  Description of Artificial Sequence: Synthetic peptide
SSAVAWY

SEQ ID NO: 898 - protein, 6 aa, Artificial Sequence
  Description of Artificial Sequence: Synthetic peptide
QSVSSA

SEQ ID NO: 899 - protein, 7 aa, Artificial Sequence
  Description of Artificial Sequence: Synthetic peptide
SASSLYS

SEQ ID NO: 900 - protein, 7 aa, Artificial Sequence
  Description of Artificial Sequence: Synthetic peptide
SASSLYS

SEQ ID NO: 901 - protein, 7 aa, Artificial Sequence
  Description of Artificial Sequence: Synthetic peptide
SASSLYS

SEQ ID NO: 902 - protein, 10 aa, Artificial Sequence
  Description of Artificial Sequence: Synthetic peptide
LLIYSASSLY

SEQ ID NO: 903 - protein, 2 aa, Artificial Sequence
  Description of Artificial Sequence: Synthetic peptide
SA

SEQ ID NO: 904 - protein, 9 aa, Artificial Sequence
  Description of Artificial Sequence: Synthetic peptide
QQVQTSLAT

SEQ ID NO: 905 - protein, 9 aa, Artificial Sequence
  Description of Artificial Sequence: Synthetic peptide
QQVQTSLAT

SEQ ID NO: 906 - protein, 9 aa, Artificial Sequence
  Description of Artificial Sequence: Synthetic peptide
QQVQTSLAT

SEQ ID NO: 907 - protein, 8 aa, Artificial Sequence
  Description of Artificial Sequence: Synthetic peptide
QQVQTSLA

SEQ ID NO: 908 - protein, 9 aa, Artificial Sequence
  Description of Artificial Sequence: Synthetic peptide
QQVQTSLAT

SEQ ID NO: 909-938 - skipped (30 entries)

SEQ ID NO: 939 - protein, 7 aa, Artificial Sequence
  Description of Artificial Sequence: Synthetic peptide
GFTFSWY

SEQ ID NO: 940 - protein, 10 aa, Artificial Sequence
  Description of Artificial Sequence: Synthetic peptide
GFTFSWYEIH

SEQ ID NO: 941 - protein, 5 aa, Artificial Sequence
  Description of Artificial Sequence: Synthetic peptide
WYEIH

SEQ ID NO: 942 - protein, 6 aa, Artificial Sequence
  Description of Artificial Sequence: Synthetic peptide
SWYEIH

SEQ ID NO: 943 - protein, 8 aa, Artificial Sequence
  Description of Artificial Sequence: Synthetic peptide
GFTFSWYE

SEQ ID NO: 944 - protein, 6 aa, Artificial Sequence
  Description of Artificial Sequence: Synthetic peptide
GPYSSY

SEQ ID NO: 945 - protein, 10 aa, Artificial Sequence
  Description of Artificial Sequence: Synthetic peptide
RIGPYSSYTY

SEQ ID NO: 946 - protein, 17 aa, Artificial Sequence
  Description of Artificial Sequence: Synthetic peptide
RIGPYSSYTYYADSVKG

SEQ ID NO: 947 - protein, 13 aa, Artificial Sequence
  Description of Artificial Sequence: Synthetic peptide
WVARIGPYSSYTY

SEQ ID NO: 948 - protein, 8 aa, Artificial Sequence
  Description of Artificial Sequence: Synthetic peptide
IGPYSSYT

SEQ ID NO: 949 - protein, 10 aa, Artificial Sequence
  Description of Artificial Sequence: Synthetic peptide
TYYPSYGMDY

SEQ ID NO: 950 - protein, 10 aa, Artificial Sequence
  Description of Artificial Sequence: Synthetic peptide
TYYPSYGMDY

SEQ ID NO: 951 - protein, 10 aa, Artificial Sequence
  Description of Artificial Sequence: Synthetic peptide
TYYPSYGMDY

SEQ ID NO: 952 - protein, 11 aa, Artificial Sequence
  Description of Artificial Sequence: Synthetic peptide
ARTYYPSYGMD

SEQ ID NO: 953 - protein, 12 aa, Artificial Sequence
  Description of Artificial Sequence: Synthetic peptide
ARTYYPSYGMDY

SEQ ID NO: 954 - protein, 11 aa, Artificial Sequence
  Description of Artificial Sequence: Synthetic peptide
RASQSVSSAVA

SEQ ID NO: 955 - protein, 11 aa, Artificial Sequence
  Description of Artificial Sequence: Synthetic peptide
RASQSVSSAVA

SEQ ID NO: 956 - protein, 11 aa, Artificial Sequence
  Description of Artificial Sequence: Synthetic peptide
RASQSVSSAVA

SEQ ID NO: 957 - protein, 7 aa, Artificial Sequence
  Description of Artificial Sequence: Synthetic peptide
SSAVAWY

SEQ ID NO: 958 - protein, 6 aa, Artificial Sequence
  Description of Artificial Sequence: Synthetic peptide
QSVSSA

SEQ ID NO: 959 - protein, 7 aa, Artificial Sequence
  Description of Artificial Sequence: Synthetic peptide
SASSLYS

SEQ ID NO: 960 - protein, 7 aa, Artificial Sequence
  Description of Artificial Sequence: Synthetic peptide
SASSLYS

SEQ ID NO: 961 - protein, 7 aa, Artificial Sequence
  Description of Artificial Sequence: Synthetic peptide
SASSLYS

SEQ ID NO: 962 - protein, 10 aa, Artificial Sequence
  Description of Artificial Sequence: Synthetic peptide
LLIYSASSLY

SEQ ID NO: 963 - protein, 2 aa, Artificial Sequence
  Description of Artificial Sequence: Synthetic peptide
SA

SEQ ID NO: 964 - protein, 9 aa, Artificial Sequence
  Description of Artificial Sequence: Synthetic peptide
QQSFSSPVT

SEQ ID NO: 965 - protein, 9 aa, Artificial Sequence
  Description of Artificial Sequence: Synthetic peptide
QQSFSSPVT

SEQ ID NO: 966 - protein, 9 aa, Artificial Sequence
  Description of Artificial Sequence: Synthetic peptide
QQSFSSPVT

SEQ ID NO: 967 - protein, 8 aa, Artificial Sequence
  Description of Artificial Sequence: Synthetic peptide
QQSFSSPV

SEQ ID NO: 968 - protein, 9 aa, Artificial Sequence
  Description of Artificial Sequence: Synthetic peptide
QQSFSSPVT

SEQ ID NO: 969 - protein, 7 aa, Artificial Sequence
  Description of Artificial Sequence: Synthetic peptide
GFTFSTY

SEQ ID NO: 970 - protein, 10 aa, Artificial Sequence
  Description of Artificial Sequence: Synthetic peptide
GFTFSTYFIH

SEQ ID NO: 971 - protein, 5 aa, Artificial Sequence
  Description of Artificial Sequence: Synthetic peptide
TYFIH

SEQ ID NO: 972 - protein, 6 aa, Artificial Sequence
  Description of Artificial Sequence: Synthetic peptide
STYFIH

SEQ ID NO: 973 - protein, 8 aa, Artificial Sequence
  Description of Artificial Sequence: Synthetic peptide
GFTFSTYF

SEQ ID NO: 974 - protein, 6 aa, Artificial Sequence
  Description of Artificial Sequence: Synthetic peptide
SPSGSH

SEQ ID NO: 975 - protein, 10 aa, Artificial Sequence
  Description of Artificial Sequence: Synthetic peptide
WISPSGSHTG

SEQ ID NO: 976 - protein, 17 aa, Artificial Sequence
  Description of Artificial Sequence: Synthetic peptide
WISPSGSHTGYADSVKG

SEQ ID NO: 977 - protein, 13 aa, Artificial Sequence
  Description of Artificial Sequence: Synthetic peptide
WVAWISPSGSHTG

SEQ ID NO: 978 - protein, 8 aa, Artificial Sequence
  Description of Artificial Sequence: Synthetic peptide
ISPSGSHT

SEQ ID NO: 979 - protein, 9 aa, Artificial Sequence
  Description of Artificial Sequence: Synthetic peptide
VRYPGVMDY

SEQ ID NO: 980 - protein, 9 aa, Artificial Sequence
  Description of Artificial Sequence: Synthetic peptide
VRYPGVMDY

SEQ ID NO: 981 - protein, 9 aa, Artificial Sequence
  Description of Artificial Sequence: Synthetic peptide
VRYPGVMDY

SEQ ID NO: 982 - protein, 10 aa, Artificial Sequence
  Description of Artificial Sequence: Synthetic peptide
ARVRYPGVMD

SEQ ID NO: 983 - protein, 11 aa, Artificial Sequence
  Description of Artificial Sequence: Synthetic peptide
ARVRYPGVMDY

SEQ ID NO: 984 - protein, 11 aa, Artificial Sequence
  Description of Artificial Sequence: Synthetic peptide
RASQSVSSAVA

SEQ ID NO: 985 - protein, 11 aa, Artificial Sequence
  Description of Artificial Sequence: Synthetic peptide
RASQSVSSAVA

SEQ ID NO: 986 - protein, 11 aa, Artificial Sequence
  Description of Artificial Sequence: Synthetic peptide
RASQSVSSAVA

SEQ ID NO: 987 - protein, 7 aa, Artificial Sequence
  Description of Artificial Sequence: Synthetic peptide
SSAVAWY

SEQ ID NO: 988 - protein, 6 aa, Artificial Sequence
  Description of Artificial Sequence: Synthetic peptide
QSVSSA

SEQ ID NO: 989 - protein, 7 aa, Artificial Sequence
  Description of Artificial Sequence: Synthetic peptide
SASSLYS

SEQ ID NO: 990 - protein, 7 aa, Artificial Sequence
  Description of Artificial Sequence: Synthetic peptide
SASSLYS

SEQ ID NO: 991 - protein, 7 aa, Artificial Sequence
  Description of Artificial Sequence: Synthetic peptide
SASSLYS

SEQ ID NO: 992 - protein, 10 aa, Artificial Sequence
  Description of Artificial Sequence: Synthetic peptide
LLIYSASSLY

SEQ ID NO: 993 - protein, 2 aa, Artificial Sequence
  Description of Artificial Sequence: Synthetic peptide
SA

SEQ ID NO: 994 - protein, 9 aa, Artificial Sequence
  Description of Artificial Sequence: Synthetic peptide
QQWYPSLIT

SEQ ID NO: 995 - protein, 9 aa, Artificial Sequence
  Description of Artificial Sequence: Synthetic peptide
QQWYPSLIT

SEQ ID NO: 996 - protein, 9 aa, Artificial Sequence
  Description of Artificial Sequence: Synthetic peptide
QQWYPSLIT

SEQ ID NO: 997 - protein, 8 aa, Artificial Sequence
  Description of Artificial Sequence: Synthetic peptide
QQWYPSLI

SEQ ID NO: 998 - protein, 9 aa, Artificial Sequence
  Description of Artificial Sequence: Synthetic peptide
QQWYPSLIT

SEQ ID NO: 999 - protein, 7 aa, Artificial Sequence
  Description of Artificial Sequence: Synthetic peptide
GFTFSRY

SEQ ID NO: 1000 - protein, 10 aa, Artificial Sequence
  Description of Artificial Sequence: Synthetic peptide
GFTFSRYYIH

SEQ ID NO: 1001 - protein, 5 aa, Artificial Sequence
  Description of Artificial Sequence: Synthetic peptide
RYYIH

SEQ ID NO: 1002 - protein, 6 aa, Artificial Sequence
  Description of Artificial Sequence: Synthetic peptide
SRYYIH

SEQ ID NO: 1003 - protein, 8 aa, Artificial Sequence
  Description of Artificial Sequence: Synthetic peptide
GFTFSRYY

SEQ ID NO: 1004 - protein, 6 aa, Artificial Sequence
  Description of Artificial Sequence: Synthetic peptide
SSDSGY

SEQ ID NO: 1005 - protein, 10 aa, Artificial Sequence
  Description of Artificial Sequence: Synthetic peptide
FISSDSGYTQ

SEQ ID NO: 1006 - protein, 17 aa, Artificial Sequence
  Description of Artificial Sequence: Synthetic peptide
FISSDSGYTQYADSVKG

SEQ ID NO: 1007 - protein, 13 aa, Artificial Sequence
  Description of Artificial Sequence: Synthetic peptide
WVAFISSDSGYTQ

SEQ ID NO: 1008 - protein, 8 aa, Artificial Sequence
  Description of Artificial Sequence: Synthetic peptide
ISSDSGYT

SEQ ID NO: 1009 - protein, 9 aa, Artificial Sequence
  Description of Artificial Sequence: Synthetic peptide
TMSYSALDY

SEQ ID NO: 1010 - protein, 9 aa, Artificial Sequence
  Description of Artificial Sequence: Synthetic peptide
TMSYSALDY

SEQ ID NO: 1011 - protein, 9 aa, Artificial Sequence
  Description of Artificial Sequence: Synthetic peptide
TMSYSALDY

SEQ ID NO: 1012 - protein, 10 aa, Artificial Sequence
  Description of Artificial Sequence: Synthetic peptide
ARTMSYSALD

SEQ ID NO: 1013 - protein, 11 aa, Artificial Sequence
  Description of Artificial Sequence: Synthetic peptide
ARTMSYSALDY

SEQ ID NO: 1014 - protein, 11 aa, Artificial Sequence
  Description of Artificial Sequence: Synthetic peptide
RASQSVSSAVA

SEQ ID NO: 1015 - protein, 11 aa, Artificial Sequence
  Description of Artificial Sequence: Synthetic peptide
RASQSVSSAVA

SEQ ID NO: 1016 - protein, 11 aa, Artificial Sequence
  Description of Artificial Sequence: Synthetic peptide
RASQSVSSAVA

SEQ ID NO: 1017 - protein, 7 aa, Artificial Sequence
  Description of Artificial Sequence: Synthetic peptide
SSAVAWY

SEQ ID NO: 1018 - protein, 6 aa, Artificial Sequence
  Description of Artificial Sequence: Synthetic peptide
QSVSSA

SEQ ID NO: 1019 - protein, 7 aa, Artificial Sequence
  Description of Artificial Sequence: Synthetic peptide
SASSLYS

SEQ ID NO: 1020 - protein, 7 aa, Artificial Sequence
  Description of Artificial Sequence: Synthetic peptide
SASSLYS

SEQ ID NO: 1021 - protein, 7 aa, Artificial Sequence
  Description of Artificial Sequence: Synthetic peptide
SASSLYS

SEQ ID NO: 1022 - protein, 10 aa, Artificial Sequence
  Description of Artificial Sequence: Synthetic peptide
LLIYSASSLY

SEQ ID NO: 1023 - protein, 2 aa, Artificial Sequence
  Description of Artificial Sequence: Synthetic peptide
SA

SEQ ID NO: 1024 - protein, 9 aa, Artificial Sequence
  Description of Artificial Sequence: Synthetic peptide
QQGFGFLVT

SEQ ID NO: 1025 - protein, 9 aa, Artificial Sequence
  Description of Artificial Sequence: Synthetic peptide
QQGFGFLVT

SEQ ID NO: 1026 - protein, 9 aa, Artificial Sequence
  Description of Artificial Sequence: Synthetic peptide
QQGFGFLVT

SEQ ID NO: 1027 - protein, 8 aa, Artificial Sequence
  Description of Artificial Sequence: Synthetic peptide
QQGFGFLV

SEQ ID NO: 1028 - protein, 9 aa, Artificial Sequence
  Description of Artificial Sequence: Synthetic peptide
QQGFGFLVT

SEQ ID NO: 1029 - protein, 448 aa, Artificial Sequence
  Description of Artificial Sequence: Synthetic polypeptide
EVQLVESGGGLVQPGGSLRLSCAASGFTFSGYFIHWVRQAPGKGLEWVARISSYGGHTDYADSVKGRFTISADTSKNTAYLQMNSLRAEDTAVYYCARAQYVPGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1030 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSYPTLGTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1031 - protein, 118 aa, Artificial Sequence
  Description of Artificial Sequence: Synthetic polypeptide
EVQLVESGGGLVQPGGSLRLSCAASGFTFSGYFIHWVRQAPGKGLEWVARISSYGGHTDYADSVKGRFTISADTSKNTAYLQMNSLRAEDTAVYYCARAQYVPGLDYWGQGTLVTVSS

SEQ ID NO: 1032 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSYPTLGTFGQGTKVEIKRTV

SEQ ID NO: 1033 - protein, 448 aa, Artificial Sequence
  Description of Artificial Sequence: Synthetic polypeptide
EVQLVESGGGLVQPGGSLRLSCAASGFTFGSYFIHWVRQAPGKGLEWVAYISPTWGYTYYADSVKGRFTISADTSKNTAYLQMNSLRAEDTAVYYCARAWFGFAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1034 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQVYSSPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1035 - protein, 118 aa, Artificial Sequence
  Description of Artificial Sequence: Synthetic polypeptide
EVQLVESGGGLVQPGGSLRLSCAASGFTFGSYFIHWVRQAPGKGLEWVAYISPTWGYTYYADSVKGRFTISADTSKNTAYLQMNSLRAEDTAVYYCARAWFGFAFDYWGQGTLVTVSS

SEQ ID NO: 1036 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQVYSSPLTFGQGTKVEIKRTV

SEQ ID NO: 1037 - protein, 447 aa, Artificial Sequence
  Description of Artificial Sequence: Synthetic polypeptide
EVQLVESGGGLVQPGGSLRLSCAASGFTFTSYYIHWVRQAPGKGLEWVAWIWPIGGYTYYADSVKGRFTISADTSKNTAYLQMNSLRAEDTAVYYCARDAGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1038 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYYYSLLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1039 - protein, 117 aa, Artificial Sequence
  Description of Artificial Sequence: Synthetic polypeptide
EVQLVESGGGLVQPGGSLRLSCAASGFTFTSYYIHWVRQAPGKGLEWVAWIWPIGGYTYYADSVKGRFTISADTSKNTAYLQMNSLRAEDTAVYYCARDAGSGFDYWGQGTLVTVSS

SEQ ID NO: 1040 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYYYSLLTFGQGTKVEIKRTV

SEQ ID NO: 1041 - protein, 447 aa, Artificial Sequence
  Description of Artificial Sequence: Synthetic polypeptide
EVQLVESGGGLVQPGGSLRLSCAASGFTFSRYAIHWVRQAPGKGLEWVAAIYSPTGYTDYADSVKGRFTISADTSKNTAYLQMNSLRAEDTAVYYCAREGYIGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1042 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSYSALYTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1043 - protein, 117 aa, Artificial Sequence
  Description of Artificial Sequence: Synthetic polypeptide
EVQLVESGGGLVQPGGSLRLSCAASGFTFSRYAIHWVRQAPGKGLEWVAAIYSPTGYTDYADSVKGRFTISADTSKNTAYLQMNSLRAEDTAVYYCAREGYIGMDYWGQGTLVTVSS

SEQ ID NO: 1044 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSYSALYTFGQGTKVEIKRTV

SEQ ID NO: 1045 - protein, 451 aa, Artificial Sequence
  Description of Artificial Sequence: Synthetic polypeptide
EVQLVESGGGLVQPGGSLRLSCAASGFTFSSYFIHWVRQAPGKGLEWVAWIYSSGGYTYYADSVKGRFTISADTSKNTAYLQMNSLRAEDTAVYYCARFTPSDLGYGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1046 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQADPSLVTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1047 - protein, 121 aa, Artificial Sequence
  Description of Artificial Sequence: Synthetic polypeptide
EVQLVESGGGLVQPGGSLRLSCAASGFTFSSYFIHWVRQAPGKGLEWVAWIYSSGGYTYYADSVKGRFTISADTSKNTAYLQMNSLRAEDTAVYYCARFTPSDLGYGLDYWGQGTLVTVSS

SEQ ID NO: 1048 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQADPSLVTFGQGTKVEIKRTV

SEQ ID NO: 1049 - protein, 447 aa, Artificial Sequence
  Description of Artificial Sequence: Synthetic polypeptide
EVQLVESGGGLVQPGGSLRLSCAASGFTFDYYVIHWVRQAPGKGLEWVAAIDSYWGDTYYADSVKGRFTISADTSKNTAYLQMNSLRAEDTAVYYCARFYFYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1050 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYSWSLWTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1051 - protein, 117 aa, Artificial Sequence
  Description of Artificial Sequence: Synthetic polypeptide
EVQLVESGGGLVQPGGSLRLSCAASGFTFDYYVIHWVRQAPGKGLEWVAAIDSYWGDTYYADSVKGRFTISADTSKNTAYLQMNSLRAEDTAVYYCARFYFYGFDYWGQGTLVTVSS

SEQ ID NO: 1052 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYSWSLWTFGQGTKVEIKRTV

SEQ ID NO: 1053 - protein, 447 aa, Artificial Sequence
  Description of Artificial Sequence: Synthetic polypeptide
EVQLVESGGGLVQPGGSLRLSCAASGFTFRYYWIHWVRQAPGKGLEWVAAIFPSGGITTYADSVKGRFTISADTSKNTAYLQMNSLRAEDTAVYYCARGWPWGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1054 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQRDRSPYTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1055 - protein, 117 aa, Artificial Sequence
  Description of Artificial Sequence: Synthetic polypeptide
EVQLVESGGGLVQPGGSLRLSCAASGFTFRYYWIHWVRQAPGKGLEWVAAIFPSGGITTYADSVKGRFTISADTSKNTAYLQMNSLRAEDTAVYYCARGWPWGLDYWGQGTLVTVSS

SEQ ID NO: 1056 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQRDRSPYTFGQGTKVEIKRTV

SEQ ID NO: 1057 - protein, 449 aa, Artificial Sequence
  Description of Artificial Sequence: Synthetic polypeptide
EVQLVESGGGLVQPGGSLRLSCAASGFTFSYYWIHWVRQAPGKGLEWVADIYPSSGYTYYADSVKGRFTISADTSKNTAYLQMNSLRAEDTAVYYCARGWYAAYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1058 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYALRPLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1059 - protein, 119 aa, Artificial Sequence
  Description of Artificial Sequence: Synthetic polypeptide
EVQLVESGGGLVQPGGSLRLSCAASGFTFSYYWIHWVRQAPGKGLEWVADIYPSSGYTYYADSVKGRFTISADTSKNTAYLQMNSLRAEDTAVYYCARGWYAAYGMDYWGQGTLVTVSS

SEQ ID NO: 1060 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYALRPLTFGQGTKVEIKRTV

SEQ ID NO: 1061 - protein, 447 aa, Artificial Sequence
  Description of Artificial Sequence: Synthetic polypeptide
EVQLVESGGGLVQPGGSLRLSCAASGFTFSWYVIHWVRQAPGKGLEWVAAIYPYHSKTYYADSVKGRFTISADTSKNTAYLQMNSLRAEDTAVYYCARGYWYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1062 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYTDAPWTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1063 - protein, 117 aa, Artificial Sequence
  Description of Artificial Sequence: Synthetic polypeptide
EVQLVESGGGLVQPGGSLRLSCAASGFTFSWYVIHWVRQAPGKGLEWVAAIYPYHSKTYYADSVKGRFTISADTSKNTAYLQMNSLRAEDTAVYYCARGYWYGMDYWGQGTLVTVSS

SEQ ID NO: 1064 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYTDAPWTFGQGTKVEIKRTV

SEQ ID NO: 1065 - protein, 449 aa, Artificial Sequence
  Description of Artificial Sequence: Synthetic polypeptide
EVQLVESGGGLVQPGGSLRLSCAASGFTFGYYYIHWVRQAPGKGLEWVAWISPSGSVTAYADSVKGRFTISADTSKNTAYLQMNSLRAEDTAVYYCARGYYGGWGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1066 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYDTSPYTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1067 - protein, 119 aa, Artificial Sequence
  Description of Artificial Sequence: Synthetic polypeptide
EVQLVESGGGLVQPGGSLRLSCAASGFTFGYYYIHWVRQAPGKGLEWVAWISPSGSVTAYADSVKGRFTISADTSKNTAYLQMNSLRAEDTAVYYCARGYYGGWGMDYWGQGTLVTVSS

SEQ ID NO: 1068 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYDTSPYTFGQGTKVEIKRTV

SEQ ID NO: 1069 - protein, 449 aa, Artificial Sequence
  Description of Artificial Sequence: Synthetic polypeptide
  MOD_RES (247)..(247): Any amino acid
EVQLVESGGGLVQPGGSLRLSCAASGFTFRYYYIHWVRQAPGKGLEWVAGIYPYGGYTSYADSVKGRFTISADTSKNTAYLQMNSLRAEDTAVYYCARGYYVEGVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPXKPKDTLMISRTPEVTCFVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1070 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYYGSLATFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1071 - protein, 119 aa, Artificial Sequence
  Description of Artificial Sequence: Synthetic polypeptide
EVQLVESGGGLVQPGGSLRLSCAASGFTFRYYYIHWVRQAPGKGLEWVAGIYPYGGYTSYADSVKGRFTISADTSKNTAYLQMNSLRAEDTAVYYCARGYYVEGVLDYWGQGTLVTVSS

SEQ ID NO: 1072 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYYGSLATFGQGTKVEIKRTV

SEQ ID NO: 1073 - protein, 449 aa, Artificial Sequence
  Description of Artificial Sequence: Synthetic polypeptide
EVQLVESGGGLVQPGGSLRLSCAASGFTFSYYYIHWVRQAPGKGLEWVARIHPPSGYTDYADSVKGRFTISADTSKNTAYLQMNSLRAEDTAVYYCARGYYVFGVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1074 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSHWYPFTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1075 - protein, 119 aa, Artificial Sequence
  Description of Artificial Sequence: Synthetic polypeptide
EVQLVESGGGLVQPGGSLRLSCAASGFTFSYYYIHWVRQAPGKGLEWVARIHPPSGYTDYADSVKGRFTISADTSKNTAYLQMNSLRAEDTAVYYCARGYYVFGVMDYWGQGTLVTVSS

SEQ ID NO: 1076 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSHWYPFTFGQGTKVEIKRTV

SEQ ID NO: 1077 - protein, 449 aa, Artificial Sequence
  Description of Artificial Sequence: Synthetic polypeptide
EVQLVESGGGLVQPGGSLRLSCAASGFTFSSYYIHWVRQAPGKGLEWVAAIYPFSGGTYYADSVKGRFTISADTSKNTAYLQMNSLRAEDTAVYYCARGYYVYVVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1078 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYKSSPWTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1079 - protein, 119 aa, Artificial Sequence
  Description of Artificial Sequence: Synthetic polypeptide
EVQLVESGGGLVQPGGSLRLSCAASGFTFSSYYIHWVRQAPGKGLEWVAAIYPFSGGTYYADSVKGRFTISADTSKNTAYLQMNSLRAEDTAVYYCARGYYVYVVMDYWGQGTLVTVSS

SEQ ID NO: 1080 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQYKSSPWTFGQGTKVEIKRTV

SEQ ID NO: 1081 - protein, 452 aa, Artificial Sequence
  Description of Artificial Sequence: Synthetic polypeptide
EVQLVESGGGLVQPGGSLRLSCAASGFTFWGYGIHWVRQAPGKGLEWVAAIYPYGGSTYYADSVKGRFTISADTSKNTAYLQMNSLRAEDTAVYYCARLSDIYHSFSGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1082 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQRYSTPVTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1083 - protein, 122 aa, Artificial Sequence
  Description of Artificial Sequence: Synthetic polypeptide
EVQLVESGGGLVQPGGSLRLSCAASGFTFWGYGIHWVRQAPGKGLEWVAAIYPYGGSTYYADSVKGRFTISADTSKNTAYLQMNSLRAEDTAVYYCARLSDIYHSFSGMDYWGQGTLVTVSS

SEQ ID NO: 1084 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQRYSTPVTFGQGTKVEIKRTV

SEQ ID NO: 1085 - protein, 447 aa, Artificial Sequence
  Description of Artificial Sequence: Synthetic polypeptide
EVQLVESGGGLVQPGGSLRLSCAASGFTFGFYYIHWVRQAPGKGLEWVAFIDPHGGSTYYADSVKGRFTISADTSKNTAYLQMNSLRAEDTAVYYCARLSYPGVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1086 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQGYSTLVTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1087 - protein, 118 aa, Artificial Sequence
  Description of Artificial Sequence: Synthetic polypeptide
EVQLVESGGGLVQPGGSLRLSCAASGFTFGFYYIHWVRQAPGKGLEWVAFIDPHGGSTYYADSVKGRFTISADTSKNTAYLQMNSLRAEDTAVYYCARLSYPGVLDYWGQGTLVTVSS

SEQ ID NO: 1088 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQGYSTLVTFGQGTKVEIKRTV

SEQ ID NO: 1089 - protein, 450 aa, Artificial Sequence
  Description of Artificial Sequence: Synthetic polypeptide
EVQLVESGGGLVQPGGSLRLSCAASGFTFSQYAIHWVRQAPGKGLEWVARIYPDSGYTYYADSVKGRFTISADTSKNTAYLQMNSLRAEDTAVYYCARPYHQYAEGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1090 - protein, 214 aa, Artificial Sequence
  Description of Artificial Sequence: Synthetic polypeptide
DIQMTQSPSSLSASVGDRVTITCRASQSVSRAVAWYQQKPGKAPKLLIYSASSLYSGVPSRFSGSRSGTDFTLTISSLQPEDFATYYCQQYYSPLLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1091 - protein, 120 aa, Artificial Sequence
  Description of Artificial Sequence: Synthetic polypeptide
EVQLVESGGGLVQPGGSLRLSCAASGFTFSQYAIHWVRQAPGKGLEWVARIYPDSGYTYYADSVKGRFTISADTSKNTAYLQMNSLRAEDTAVYYCARPYHQYAEGMDYWGQGTLVTVSS

SEQ ID NO: 1092 - protein, 110 aa, Artificial Sequence
  Description of Artificial Sequence: Synthetic polypeptide
DIQMTQSPSSLSASVGDRVTITCRASQSVSRAVAWYQQKPGKAPKLLIYSASSLYSGVPSRFSGSRSGTDFTLTISSLQPEDFATYYCQQYYSPLLTFGQGTKVEIKRTV

SEQ ID NO: 1093 - protein, 447 aa, Artificial Sequence
  Description of Artificial Sequence: Synthetic polypeptide
EVQLVESGGGLVQPGGSLRLSCAASGFTFSAYWIHWVRQAPGKGLEWVALIGPDGGYTYYADSVKGRFTISADTSKNTAYLQMNSLRAEDTAVYYCARQASRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1094 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQWSSPLHTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1095 - protein, 117 aa, Artificial Sequence
  Description of Artificial Sequence: Synthetic polypeptide
EVQLVESGGGLVQPGGSLRLSCAASGFTFSAYWIHWVRQAPGKGLEWVALIGPDGGYTYYADSVKGRFTISADTSKNTAYLQMNSLRAEDTAVYYCARQASRGLDYWGQGTLVTVSS

SEQ ID NO: 1096 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQWSSPLHTFGQGTKVEIKRTV

SEQ ID NO: 1097 - protein, 447 aa, Artificial Sequence
  Description of Artificial Sequence: Synthetic polypeptide
EVQLVESGGGLVQPGGSLRLSCAASGFTFGTYYIHWVRQAPGKGLEWVASILSGGGYTVYADSVKGRFTISADTSKNTAYLQMNSLRAEDTAVYYCARRVYPGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1098 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQDSWGLWTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1099 - protein, 117 aa, Artificial Sequence
  Description of Artificial Sequence: Synthetic polypeptide
EVQLVESGGGLVQPGGSLRLSCAASGFTFGTYYIHWVRQAPGKGLEWVASILSGGGYTVYADSVKGRFTISADTSKNTAYLQMNSLRAEDTAVYYCARRVYPGFDYWGQGTLVTVSS

SEQ ID NO: 1100 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQDSWGLWTFGQGTKVEIKRTV

SEQ ID NO: 1101 - protein, 447 aa, Artificial Sequence
  Description of Artificial Sequence: Synthetic polypeptide
EVQLVESGGGLVQPGGSLRLSCAASGFTFSFYGIHWVRQAPGKGLEWVAWIYPYGGFTDYADSVKGRFTISADTSKNTAYLQMNSLRAEDTAVYYCARSGFFAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1102 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQVQTSLATFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1103 - protein, 117 aa, Artificial Sequence
  Description of Artificial Sequence: Synthetic polypeptide
EVQLVESGGGLVQPGGSLRLSCAASGFTFSFYGIHWVRQAPGKGLEWVAWIYPYGGFTDYADSVKGRFTISADTSKNTAYLQMNSLRAEDTAVYYCARSGFFAFDYWGQGTLVTVSS

SEQ ID NO: 1104 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQVQTSLATFGQGTKVEIKRTV

SEQ ID NO: 1105 - protein, 449 aa, Artificial Sequence
  Description of Artificial Sequence: Synthetic polypeptide
EVQLVESGGGLVQPGGSLRLSCAASGFTFSWYEIHWVRQAPGKGLEWVARIGPYSSYTYYADSVKGRFTISADTSKNTAYLQMNSLRAEDTAVYYCARTYYPSYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1106 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSFSSPVTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1107 - protein, 119 aa, Artificial Sequence
  Description of Artificial Sequence: Synthetic polypeptide
EVQLVESGGGLVQPGGSLRLSCAASGFTFSWYEIHWVRQAPGKGLEWVARIGPYSSYTYYADSVKGRFTISADTSKNTAYLQMNSLRAEDTAVYYCARTYYPSYGMDYWGQGTLVTVSS

SEQ ID NO: 1108 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQSFSSPVTFGQGTKVEIKRTV

SEQ ID NO: 1109 - protein, 448 aa, Artificial Sequence
  Description of Artificial Sequence: Synthetic polypeptide
EVQLVESGGGLVQPGGSLRLSCAASGFTFSTYFIHWVRQAPGKGLEWVAWISPSGSHTGYADSVKGRFTISADTSKNTAYLQMNSLRAEDTAVYYCARVRYPGVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1110 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQWYPSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1111 - protein, 118 aa, Artificial Sequence
  Description of Artificial Sequence: Synthetic polypeptide
EVQLVESGGGLVQPGGSLRLSCAASGFTFSTYFIHWVRQAPGKGLEWVAWISPSGSHTGYADSVKGRFTISADTSKNTAYLQMNSLRAEDTAVYYCARVRYPGVMDYWGQGTLVTVSS

SEQ ID NO: 1112 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQWYPSLITFGQGTKVEIKRTV

SEQ ID NO: 1113 - protein, 452 aa, Artificial Sequence
  Description of Artificial Sequence: Synthetic polypeptide
EVQLVESGGGLVQPGGSLRLSCAASGFTFATYYIHWVRQAPGKGLEWVAYIDSESGYTYYADSVKGRFTISADTSKNTAYLQMNSLRAEDTAVYYCARVSRGSSGTHVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1114 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQRYSSLLTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1115 - protein, 122 aa, Artificial Sequence
  Description of Artificial Sequence: Synthetic polypeptide
EVQLVESGGGLVQPGGSLRLSCAASGFTFATYYIHWVRQAPGKGLEWVAYIDSESGYTYYADSVKGRFTISADTSKNTAYLQMNSLRAEDTAVYYCARVSRGSSGTHVMDYWGQGTLVTVSS

SEQ ID NO: 1116 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQRYSSLLTFGQGTKVEIKRTV

SEQ ID NO: 1117 - protein, 448 aa, Artificial Sequence
  Description of Artificial Sequence: Synthetic polypeptide
EVQLVESGGGLVQPGGSLRLSCAASGFTFSRYYIHWVRQAPGKGLEWVAFISSDSGYTQYADSVKGRFTISADTSKNTAYLQMNSLRAEDTAVYYCARTMSYSALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1118 - protein, 214 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQGFGFLVTFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1119 - protein, 118 aa, Artificial Sequence
  Description of Artificial Sequence: Synthetic polypeptide
EVQLVESGGGLVQPGGSLRLSCAASGFTFSRYYIHWVRQAPGKGLEWVAFISSDSGYTQYADSVKGRFTISADTSKNTAYLQMNSLRAEDTAVYYCARTMSYSALDYWGQGTLVTVSS

SEQ ID NO: 1120 - protein, 110 aa, Artificial Sequence
  Description of Artificial Sequence: Synthetic polypeptide
DIQMTQSPSSLSASVGDRVTITCRASQSVSSAVAWYQQKPGKAPKLLIYSASSLYSGVPSRFSGSRSGTDFTLTISSLQPEDFATYYCQQGFGFLVTFGQGTKVEIKRTV

SEQ ID NO: 1121 - protein, 4 aa, Artificial Sequence
  Description of Artificial Sequence: Synthetic peptide
LLQG

SEQ ID NO: 1122 - protein, 6 aa, Artificial Sequence
  Description of Artificial Sequence: Synthetic peptide
FHENSP

SEQ ID NO: 1123 - protein, 5 aa, Artificial Sequence
  Description of Artificial Sequence: Synthetic peptide
LPRWG

(19) United States
(12) Patent Application Publication
(10) Pub. No.: US 2022/0235135 A1
(43) Pub. Date: Jul. 28, 2022

(54) ACTIVATING ANTI-GAL9 BINDING MOLECULES

(71) Applicant: THE COUNCIL OF THE QUEENSLAND INSTITUTE OF MEDICAL RESEARCH, Brisbane QLD (AU)

(72) Inventors: Dileep K. PULUKKUNAT, Middleton, WI (US); Michelle WYKES, Brisbane QLD (AU)

(73) Assignee: THE COUNCIL OF THE QUEENSLAND INSTITUTE OF MEDICAL RESEARCH, Brisbane QLD (AU)

(21) Appl. No.: 17/614,704

(22) PCT Filed: May 29, 2020
(86) PCT No.: PCT/US2020/035399

§ 371 Date: Nov. 29, 2021

Related U.S. Application Data
(60) Provisional application No. 62/855,590, filed on May 31, 2019.
(60) Provisional application No. 62/900,105, filed on Sep. 13, 2019.
(60) Provisional application No. 62/964,487, filed on Jan. 22, 2020.

(51) Int. Cl. C07K 16/28; A61P 35/00

(52) U.S. Cl. CPC C07K 16/2851; C07K 2317/75; C07K 2317/565; A61P 35/00

(57) ABSTRACT

Anti-GAL9 antibody constructs, pharmaceutical compositions comprising the constructs, and methods of use thereof are presented.

### 1. CROSS-REFERENCE TO RELATED APPLICATIONS

This application claims the benefit under 35 U.S.C. § 119(e) of prior co-pending U.S. Provisional Patent Application No. 62/964,487, filed on Jan. 22, 2020, U.S. Provisional Patent Application No. 62/900,105, filed on Sep. 13, 2019, and U.S. Provisional Patent Application No. 62/855,590, filed on May 31, 2019.

### 2. SEQUENCE LISTING

The instant application contains a Sequence Listing which has been submitted via EFS-Web and is hereby incorporated herein by reference in its entirety. Said ASCII copy, created on Apr. 9, 2020, is named 42700WO_CRF_sequencelisting.txt, and is 389,339 bytes in size.

### 3. BACKGROUND

Immune therapy has great potential for the treatment of cancer. However, tumors can become resistant to immune therapy, for example by recruiting immunosuppressive cells or signaling molecules to the tumor microenvironment or by co-opting immune checkpoint signaling pathways.

Galectin-9 (GAL9) is an S-type lectin beta-galactoside-binding protein with N- and C-terminal carbohydrate-binding domains connected by a linker peptide. GAL9 has been implicated in modulating cell-cell and cell-matrix interactions. GAL9 has been shown to bind soluble PD-L2, and at least some of the immunological effects of PD-L2 have been suggested to be mediated through binding of multimeric PD-L2 to GAL9, rather than through PD-1 (WO 2016/008005, which is incorporated herein by reference in its entirety). However, mechanisms by which GAL9 and PD-L2 impact immune effector function are not yet fully characterized.

There remains a need for therapeutic agents that can enhance immune effector function and reduce immunosuppressive or T cell exhaustion pathways. Such therapeutic agents may be useful for improving cancer immune therapy.

### 4. SUMMARY

In a first aspect the disclosure provides a Galectin-9 (GAL9) antigen binding molecule comprising a first antigen binding site specific (ABS) for a first epitope of a first GAL9 antigen, wherein the first antigen binding site comprises all three VH CDRs from any one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58.

In a second aspect, the disclosure provides a Galectin-9 (GAL9) antigen binding molecule, comprising a first antigen binding site specific for a first epitope of a first GAL9 antigen, wherein the first antigen binding site comprises all three VL CDRs from any one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58.

In a third aspect, the disclosure provides a Galectin-9 (GAL9) antigen binding molecule, comprising a first antigen binding site specific for a first epitope of a first GAL9 antigen, wherein the first antigen binding site comprises all three VH CDRs and all three VL CDRs from any one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58.

In a fourth aspect, the disclosure provides a Galectin-9 (GAL9) antigen binding molecule, comprising a first antigen binding site specific for a first epitope of a first GAL9 antigen, comprising the VL sequence and the VH sequence from any one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58.

In some embodiments, the GAL9 antigen binding molecule comprises a full immunoglobulin heavy chain “IgG1” sequence comprising the VH sequence and a full immunoglobulin light chain sequence comprising the VL sequence, wherein the VH sequence and the VL sequence are from any one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58.

In some embodiments, the GAL9 antigen binding molecule comprises a full immunoglobulin heavy chain “IgG4” sequence comprising the VH sequence and a full immunoglobulin light chain sequence comprising the VL sequence, wherein the VH sequence and the VL sequence are from any one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58.

In some embodiments, the GAL9 antigen binding molecule comprises a full immunoglobulin heavy chain “IgG3” sequence comprising the VH sequence and a full immunoglobulin light chain sequence comprising the VL sequence, wherein the VH sequence and the VL sequence are from any one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58.

In some embodiments, the GAL9 antigen binding molecule can comprise a GAL9 antigen that is a human GAL9 antigen.

In some embodiments, the GAL9 antigen binding molecule can further comprises a second antigen binding site.

In certain embodiments, the second antigen binding site is specific for the GAL9 antigen. In other embodiments, the second antigen binding site is identical to the first antigen binding site.

In other embodiments, the second antigen binding site is specific for a second epitope of the first GAL9 antigen.

In some embodiments, the second antigen binding site comprises all three VH CDRs, all three VL CDRs, or all three VH CDRs and all three VL CDRs from another ABS clone selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58.

In some embodiments, the second antigen binding site comprises the VL sequence and the VH sequence from the other ABS clone.

In some embodiments, the second antigen binding site comprises a full immunoglobulin heavy chain sequence comprising the VH sequence and a full immunoglobulin light chain sequence comprising the VL sequence from the other ABS clone.

In some embodiments, the second antigen binding site is specific for an antigen other than the first GAL9 antigen.

In some embodiments, the first antigen binding site comprises all three VH CDRs, all three VL CDRs, or all three VH CDRs and all three VL CDRs from any one of the ABS clones selected from: P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9- 58.

In some embodiments, the first antigen binding site comprises all three VH CDRs, all three VL CDRs, or all three VH CDRs and all three VL CDRs from any one of the ABS clones selected from: P9-18, P9-15, P9-21, and P9-28.

In some embodiments the first antigen binding site comprises all three VH CDRs, all three VL CDRs, or all three VH CDRs and all three VL CDRs from ABS clone P9-15.

In some embodiments, the first antigen binding site comprises all three VH CDRs, all three VL CDRs, or all three VH CDRs and all three VL CDRs from ABS clone P9-18.

In some embodiments, the first antigen binding site comprises all three VH CDRs, all three VL CDRs, or all three VH CDRs and all three VL CDRs from ABS clone P9-21.

In some embodiments, the first antigen binding site comprises all three VH CDRs, all three VL CDRs, or all three VH CDRs and all three VL CDRs from ABS clone P9-22.

In some embodiments, the first antigen binding site comprises all three VH CDRs, all three VL CDRs, or all three VH CDRs and all three VL CDRs from ABS clone P9-28.

In some embodiments, the GAL9 antigen binding molecule comprises an antibody format selected from the group consisting of: full-length antibodies, Fab fragments, F(ab)′2 fragments, Fvs, scFvs, tandcFvs, diabodies, scDiabodies, DARTs, single chain VHH camelid antibodies, tandAbs, minibodies, and B-bodies. B-bodies are described in US pre-grant publication number US 2018/0118811, which is incorporated herein by reference in its entirety.

In some embodiments, the GAL9 antigen binding molecule increases TNF-α secretion by activated immune cells, wherein the increase is greater than an 20, 30, 40, 50, 60, 70, or 80-fold increase relative to activated immune cells treated with a control agent.

In some embodiments, the GAL9 antigen binding molecule increases IFN-γ secretion by activated immune cells, wherein the increase is greater than an 1.2-fold increase relative to activated immune cells treated with a control agent.

In some embodiments, the GAL9 antigen binding molecule increases CD40L surface expression of activated CD8+ T-cells, wherein the increase is greater than a 2-fold increase relative to activated CD8+ T-cells treated with a control agent.

In some embodiments, the GAL9 antigen binding molecule increases OX40 surface expression of Activated CD8+ T-cells, wherein the increase is greater than a 2-fold increase relative to activated CD8+ T-cells treated with a control agent.

In some embodiments, the GAL9 antigen binding molecule increases IL-12 production of activated dendritic cells (DCs), wherein the increase is greater than an 20-fold increase relative to activated DCs treated with a control agent.

In some embodiments, GAL9 antigen binding molecule increases PD-L2 surface expression on activated dendritic cells (DCs), wherein the increase is greater than an 4-fold increase relative to activated DCs treated with a control agent.

In some embodiments, the control agent is a negative control agent or positive control agent.

In some embodiments, the control agent is a control antibody.

In some embodiments, the control antibody is selected from the group consisting of: an ECA42 clone anti-GAL9 antibody, an RG9.1 clone anti-GAL9 antibody, an RG9.35 clone anti-GAL9 antibody, an anti-PD1 antibody, and a non-GAL9 binding isotype control antibody.

In some embodiments, the activated immune cells, activated CD8+ T-cells, or activated DCs were activated by peptide stimulation, e.g., by a peptide or plurality of peptides known to induce an immune response.

In a fifth aspect, the disclosure provides a GAL9 antigen binding molecule increases TNF-α secretion by activated immune cells, wherein the increase is greater than an 80-fold increase relative to activated immune cells treated with a control agent.

In a sixth aspect, the disclosure provides a GAL9 antigen binding molecule increases IFN-γ secretion by activated immune cells, wherein the increase is greater than an 1.2-fold increase relative to activated immune cells treated with a control agent.

In a seventh aspect, the disclosure provides a GAL9 antigen binding molecule increases CD40L surface expression of Activated CD8+ T-cells, wherein the increase is greater than a 2-fold increase relative to activated CD8+ T-cells treated with a control agent.

In an eighth aspect, the disclosure provides a GAL9 antigen binding molecule increases OX40 surface expression of Activated CD8+ T-cells, wherein the increase is greater than a 2-fold increase relative to activated CD8+ T-cells treated with a control agent.

In a ninth aspect, the disclosure provides a GAL9 antigen binding molecule increases IL-12 production of activated dendritic cells (DCs), wherein the increase is greater than an 20-fold increase relative to activated DCs treated with a control agent.

In a tenth aspect, the disclosure provides a GAL9 antigen binding molecule increases PD-L2 surface expression on activated dendritic cells (DCs), wherein the increase is greater than an 4-fold increase relative to activated DCs treated with a control agent.

In an eleventh aspect, the disclosure provides a GAL9 antigen binding molecule demonstrates one or more of the following properties: A) increases TNF-α secretion by activated immune cells, wherein the increase is greater than an 80-fold increase relative to activated immune cells treated with a control agent; B) increases IFN-γ secretion by activated immune cells, wherein the increase is greater than an 1.2-fold increase relative to activated immune cells treated with a control agent; C) increases CD40L surface expression of activated CD8+ T-cells, wherein the increase is greater than a 2-fold increase relative to activated CD8+ T-cells treated with a control agent; D) increases OX40 surface expression of activated CD8+ T-cells, wherein the increase is greater than a 2-fold increase relative to activated CD8+ T-cells treated with a control agent; E) increases IL-12 production of activated dendritic cells (DCs), wherein the increase is greater than an 20-fold increase relative to activated DCs treated with a control agent; F) increases PD-L2 surface expression on activated dendritic cells (DCs), wherein the increase is greater than an 4-fold increase relative to activated DCs treated with a control agent.

In some embodiments, the control agent is a negative control agent or positive control agent.

In some embodiments, the control agent is a control antibody.

In some embodiments, the control antibody is selected from the group consisting of: an ECA42 clone anti-GAL9 antibody, an RG9.1 clone anti-GAL9 antibody, an RG9.35 clone anti-GAL9 antibody, an anti-PD1 antibody, and a non-GAL9 binding isotype control antibody.

In some embodiments, the activated immune cells, activated CD8+ T-cells, or activated DCs were activated by peptide stimulation, e.g., by a peptide or plurality of peptides known to induce an immune response.

In some embodiments, the GAL9 antigen binding molecule of the fifth-eleventh aspects provided herein comprise a first antigen binding site specific for a first epitope of a first GAL9 antigen, wherein the first antigen binding site comprises all three VH CDRs and all three VL CDRs from any one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58.

In some embodiments, the VL sequence and the VH sequence from any one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58.

In some certain embodiments, the GAL9 antigen binding molecule comprises a full immunoglobulin heavy chain sequence comprising the VH sequence and a full immunoglobulin light chain sequence comprising the VL sequence, wherein the VH sequence and the VL sequence are from any one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58.

In some embodiments, the GAL9 antigen is a human GAL9 antigen.

In some embodiments, the GAL9 antigen binding molecule further comprises a second antigen binding site.

In some embodiments, the second antigen binding site is specific for the GAL9 antigen.

The GAL9 antigen binding molecule of claim 48, wherein the second antigen binding site is identical to the first antigen binding site.

In some embodiments, the second antigen binding site is specific for a second epitope of the first GAL9 antigen.

In some embodiments, the second antigen binding site comprises all three VH CDRs and all three VL CDRs from another ABS clone selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58.

In some embodiments, the second antigen binding site comprises the VL sequence and the VH sequence from the other ABS clone.

In some embodiments, the second antigen binding site comprises a full immunoglobulin heavy chain sequence comprising the VH sequence and a full immunoglobulin light chain sequence comprising the VL sequence from the other ABS clone.

In some embodiments, the second antigen binding site is specific for an antigen other than the first GAL9 antigen.

In some embodiments, the first antigen binding site comprises all three VH CDRs and all three VL CDRs from any one of the ABS clones selected from: P9-10, P9-15, P9-18, P9-21, P9-22, and P9-28.

In some embodiments, the first antigen binding site comprises all three VH CDRs and all three VL CDRs from any one of the ABS clones selected from: P9-10, P9-15, P9-18, P9-21, P9-22, and P9-28.

In some embodiments, the first antigen binding site comprises all three VH CDRs and all three VL CDRs from ABS clone P9-15.

In some embodiments, the first antigen binding site comprises all three VH CDRs and all three VL CDRs from ABS clone P9-18.

In some embodiments, the first antigen binding site comprises all three VH CDRs and all three VL CDRs from ABS clone P9-21.

In some embodiments, the first antigen binding site comprises all three VH CDRs and all three VL CDRs from ABS clone P9-22.

In some embodiments, the first antigen binding site comprises all three VH CDRs and all three VL CDRs from ABS clone P9-28.

In some embodiments, the GAL9 antigen binding molecule comprises an antibody format selected from the group consisting of: full-length antibodies, Fab fragments, Fvs, scFvs, tandem scFvs, Diabodies, scDiabodies, DARTs, tandAbs, minibodies, and B-bodies.

In a twelfth aspect, the disclosure provides a GAL9 antigen binding molecule which binds to the same epitope as a GAL9 antigen binding molecule of any one of the preceding claims.

In a thirteenth aspect, the disclosure provides a GAL9 antigen binding molecule which competes for binding with a GAL9 antigen binding molecule of any one of the preceding claims.

In some embodiments, the GAL9 antigen binding molecule is purified.

In a fourteenth aspect, the disclosure provides a pharmaceutical composition comprising the GAL9 antigen binding molecule of any one of the preceding claims and a pharmaceutically acceptable diluent.

In a fifteenth aspect, the disclosure provides a method for treating a subject with cancer, the method comprising administering a therapeutically effective amount of the pharmaceutical composition as provided herein to the subject.

In some embodiments, the cancer is selected from the group consisting of: pancreatic cancer, ovarian cancer, breast cancer, lung cancer, gastric cancer, melanoma, Ewing sarcoma, chronic lymphocytic leukemia, mantle cell lymphoma, B-ALL, hematological cancer, head and neck squamous cell carcinoma, prostate cancer, colon cancer, renal cancer, and uterine cancer.

In some embodiments, cancer is selected from the group consisting of: the breast cancer, colon cancer, lung cancer and prostate cancer, cancers of the blood and lymphatic systems (including Hodgkin's disease, leukemias, lymphomas, multiple myeloma, and Waldenstrom's disease), skin cancers (including malignant melanoma), cancers of the digestive tract (including head and neck cancers, esophageal cancer, stomach cancer, cancer of the pancreas, liver cancer, colon and rectal cancer, anal cancer), cancers of the genital and urinary systems (including kidney cancer, bladder cancer, testis cancer, prostate cancer), cancers in women (including breast cancer, ovarian cancer, gynecological cancers and choriocarcinoma) as well as in brain, bone carcinoid, nasopharyngeal, retroperitoneal, thyroid and soft tissue tumors.

In some embodiments, the cancer is a viral induced tumor caused by a cancer virus. In some embodiments, the cancer virus is a Epstein-Barr virus (EBV), Hepatitis B virus, Hepatitis C virus, Human papilloma virus, Human T-lymphotropic virus 1 (HTLV-1), Kaposi sarcoma associated-herpesvirus (KHSV), Merkel cell polyomavirus, or Cytomegalovirus.

### 5. BRIEF DESCRIPTION OF THE DRAWINGS

FIG. 1 shows results of administering immune-activating anti-GAL9 (α-GAL9) antibodies in a colon cancer tumor model. BALB/c mice were implanted subcutaneously with CT26 tumor line cells and treated with control rat IgG, or anti-GAL9 antibodies P9-18 or P9-21. All treatments were intraperitoneal (I.P.), 200 μg, on days 7, 11, 15, and 19. n=10/group. Tumor growth was assessed by measuring tumor volume. Mice treated with P9-18 and P9-21 demonstrated reduced growth of the implanted CT26 tumors as compared to treatment with control IgG.

FIG. 2 shows results of administering immune-activating anti-GAL9 antibodies in a melanoma tumor model. C57BL/6 mice were implanted subcutaneously with B16.F0 tumor line cells and treated with control IgG or α-GAL9 antibodies P9-18 or P9-21. All treatments were intraperitoneal (I.P.), 200 μg, on days 7, 11, 15, and 19. n=10/group. Tumor growth was assessed by measuring tumor volume. Mice treated with P9-18 and P9-21 demonstrated reduced growth of the implanted B16.F0 tumors as compared to treatment with control IgG.

FIGS. 3A and 3B show INF-γ (3A) and TNF-α (3B) secretion from activated PBMCs stimulated in vitro with various GAL9 antibody candidates, a known comparator Tool antibody (Tool mAb), an anti-PD-1 antibody, a control antibody (IgG Ctrl), and a vehicle control (PBS Ctrl). Black diamond shapes show secretion from activated PBMCs stimulated by comparator Tool mAb and anti-PD-1 antibody, positive controls.

FIG. 4 shows levels of immune stimulatory markers CD27, CD40L, ICOS, 4-1BB, and OX40 on the surface of activated CD8+ T cells stimulated in vitro with various GAL9 antibody candidates or an IgG control antibody.

FIG. 5 shows representative flow cytometry plots quantifying IL-12 production by DCs stimulated in vitro with control IgG or α-GAL9 candidate P9-18, along with a staining control.

FIGS. 6A and 6B show representative flow cytometry plots of TNF-α secretion by CD56⁺ NK cells following 72 hours' stimulation with control antibody P9-55 (Clone 55), anti-GAL9 candidate antibody P9-15 (Clone 15), or α-GAL9 candidate antibody P9-18 (Clone 18) at dosages 5 μg (FIG. 6A) or 20 μg (FIG. 6B).

FIGS. 7A-7E show illustrative examples of Martin numbering scheme with various CDR definitions—Chothia, AbM, Kabat, Contact, IMGT—as applied to the P9-28 anti-GAL9 candidate antibody provided herein. FIGS. 7A-7E each disclose SEQ ID NOS 187 and 188, respectively, in order of appearance.

FIGS. 8A-8C show representative confocal microscopy images demonstrating co-localization and clustering of GAL9 and PD-L2 on DCs after treatment with IgG control (FIG. 8A), P9-18 (FIG. 8B), and P9-21 (FIG. 8C). The blue staining shows DNA (DAPI), red staining shows PD-L2, green staining shows CD11c, and yellow staining shows GAL9. Non-labeled microscopy images are bright field; rendered in gray scale in the attached figures.

FIGS. 9A and 9B show representative confocal images demonstrating retention of PD-L2 and PD-L1 on the surface of CT26 tumor cells after treatment with anti-GAL9 P9-18 (FIG. 9B) compared to IgG control (FIG. 9A). The speckles in the images highlight increased expression of PD-L2 and PD-L1 ligands. The blue staining shows DNA (DAPI), the red staining shows PD-L2, and the green staining shows PD-L1; rendered in gray scale in the attached figures.

FIGS. 10A-E show representative data from an EBV-infected humanized mouse model treated with anti-GAL9 P9-15. FIG. 10A shows a schematic of the protocol with treatment timeline. FIG. 10B shows images of spleens from mice treated with IgG control and P9-15. Arrows point to uncontrolled tumor growth in IgG control mice. FIG. 10C shows bar graphs of the weights of spleens. FIG. 10D shows bar graphs of the number of cells per spleen. FIG. 10E shows bar graphs of spleen viral load.

FIG. 11 shows representative data from an EBV-infected humanized mouse model treated with anti-GAL9 P9-28. FIG. 10A shows a schematic of the protocol with treatment timeline. FIG. 11 shows images of spleens from IgG control and anti-GAL9 P9-28 treated mice. Arrows point to uncontrolled tumor growth in IgG control treated mice.

FIG. 12A shows in vivo evaluation of tumor growth in a CT26 tumor model with P9-18-IgG1 (diamond [symbol] ), sFc-P9-18-IgG2a (upside-down triangle [symbol] ), P9-18-IgG2a (circle [symbol] ), and IgG (IgG2a) control #1 (black squares [symbol] ).

FIG. 12B shows an in vivo evaluation of immune memory in previously treated CT26 tumors with sFc-P9-18 IgG2a (upside-down triangle [symbol] ), P9-18 IgG2a (circle [symbol] ), and IgG (IgG2a) control #2 (black diamond [symbol] ).

FIG. 13 shows a bar graph of the mean percentage of PD-L1+ or PD-L2+ tumor-associated dendritic cells (CD11c⁺) and the mean cell surface expression (GMI) of PD-L1 or PD-L2 on tumor-associated dendritic cells (CD11c⁺) after treatment with anti-GAL9 P9-18 or control.

FIG. 14 shows bar graphs of the mean percentage of PD-L1⁺or PD-L2⁺ tumor cells and the mean cell surface expression level of PD-L1 or PD-L2 (GMI) on tumor cells after treatment with anti-GAL9 P9-18 or IgG control.

### 6. DETAILED DESCRIPTION

### 6.1. Definitions

Unless defined otherwise, all technical and scientific terms used herein have the meaning commonly understood by a person skilled in the art to which this invention belongs. As used herein, the following terms have the meanings ascribed to them below.

By “antigen binding site” or “ABS” is meant a region of a GAL9 binding molecule that specifically recognizes or binds to a given antigen or epitope.

As used herein, the terms “treat” or “treatment” are used in their broadest accepted clinical sense. The terms include, without limitation, lessening a sign or symptom of disease; improving a sign or symptom of disease; alleviation of symptoms; diminishment of extent of disease; stabilized (i.e., not worsening) state of disease; delay or slowing of disease progression; amelioration or palliation of the disease state; remission (whether partial or total), whether detectable or undetectable; cure; prolonging survival as compared to expected survival if not receiving treatment. Unless explicitly stated otherwise, “treat” or “treatment” do not intend prophylaxis or prevention of disease.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on. Unless otherwise stated, “patient” intends a human “subject.”

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease.

The term “prophylactically effective amount” is an amount that is effective to prevent a symptom of a disease.

### 6.2. Other Interpretational Conventions

Unless otherwise specified, all references to sequences herein are to amino acid sequences.

Unless otherwise specified, antibody constant region residue numbering is according to the Eu index as described at www.imgt.org/IMGTScientificChart/Numbering/Hu_IGHGnber.html#refs (accessed Aug. 22, 2017), which is hereby incorporated by reference in its entirety, and residue numbers identify the residue according to its location in an endogenous constant region sequence regardless of the residue's physical location within a chain of the GAL9 binding molecules described herein.

Unless otherwise specified as “Kabat CDR”, “Chothia CDR”, “Contact CDR”, or “IMGT CDR”, all references to “CDRs” are to CDRs defined using the Martin (AbM) definition.

By “endogenous sequence” or “native sequence” is meant any sequence, including both nucleic acid and amino acid sequences, which originates from an organism, tissue, or cell and has not been artificially modified or mutated.

Polypeptide chain numbers (e.g., a “first” polypeptide chains, a “second” polypeptide chain. etc. or polypeptide “chain 1,” “chain 2,” etc.) are used herein as a unique identifier for specific polypeptide chains that form a binding molecule and is not intended to connote order or quantity of the different polypeptide chains within the binding molecule.

In this disclosure, “comprises,” “comprising,” “containing,” “having,” “includes,” “including,” and linguistic variants thereof have the meaning ascribed to them in U.S. Patent law, permitting the presence of additional components beyond those explicitly recited.

As used herein, the singular forms “a,” “an,” and “the” include the plural referents unless the context clearly indicates otherwise. The terms “include,” “such as,” and the like are intended to convey inclusion without limitation, unless otherwise specifically indicated.

Ranges provided herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

### 6.3. General Overview

The present disclosure provides Galectin-9 (GAL9) antigen binding molecules, such as anti-GAL9 antibodies and antigen-binding fragments thereof, compositions comprising the GAL9-binding molecules; and pharmaceutical compositions comprising the GAL9-binding molecules. The disclosure particularly provides various GAL9 antigen binding molecules that are stimulatory, acting as activators of the immune system, increasing secretion and production of various cytokines in various immune cells and increasing surface expression of stimulatory molecules.

Also provided by the disclosure are methods of treating a disease or condition in a subject by administering an immune-stimulatory Galectin-9 antibody binding molecule. The methods provided by the disclosure are particularly useful for the treatment of a proliferative disease or cancer. In some embodiments, the cancer is a viral-induced cancer, for example, a cancer caused by an infection by an oncovirus or tumor virus. In some embodiments, the compositions and methods provided by the disclosure can be used for the treatment of a disease or condition that is immunosuppressive, such as malaria, HIV or AIDs, or the like.

### 6.4. GAL9 Antigen Binding Molecules

In a first aspect, antigen binding molecules are provided. In every embodiment, the antigen binding molecule includes at least a first antigen binding site specific for a GAL9 antigen; the binding molecules are therefore termed GAL9 antigen binding molecules or GAL9 binding molecules.

The GAL9 antigen binding molecules described herein bind specifically to GAL9 antigens.

As used herein, “GAL9 antigens” refer to Galectin-9 family members and homologs. GAL9 is also referred to as LGALS9, HUAT, LGALS9A, tumor antigen HOM-HD-21, and ecalectin. In particular embodiments, the GAL9 binding molecule has antigen binding sites that specifically bind to at least a portion of more than one GAL9 domain, such as the junction between a first and a second GAL9 domain.

In specific embodiments, the GAL9 antigen is human. GenBank Accession #NP_033665.1 describes a canonical human GAL9 protein, including its sequences and domain features, and is hereby incorporated by reference in its entirety. SEQ ID NO:6 provides the full-length GAL9 protein sequence.

[SEQ ID NO: 6]
MAFSGSQAPYLSPAVPFSGTIQGGLQDGLQITVNGTVLSSSGTRFAVNFQ
TGFSGNDIAFHFNPRFEDGGYVVCNTRQNGSWGPEERKTHMPFQKGMPFD
LCFLVQSSDFKVMVNGILFVQYFHRVPFHRVDTISVNGSVQLSYISFQNP
RTVPVQPAFSTVPFSQPVCFPPRPRGRRQKPPGVWPANPAPITQTVIHTV
QSAPGQMFSTPAIPPMMYPHPAYPMPFITTILGGLYPSKSILLSGTVLPS
AQRFHINLCSGNHIAFHLNPRFDENAVVRNTQIDNSWGSEERSLPRKMPF
VRGQSFSVWILCEAHCLKVAVDGQHLFEYYHRLRNLPTINRLEVGGDIQL
THVQT

In various embodiments, the GAL9 binding molecule additionally binds specifically to at least one antigen additional to a GAL9 antigen.

### 6.4.1. Functional Characteristics of the GAL9 Antigen Binding Molecules

In some embodiments, upon contact therewith, the GAL9 antigen binding molecule increases cytokine secretion by activated immune cells, e.g., activated human immune cells. In some embodiments, the immune cells are peripheral blood mononuclear cells (PBMCs). In some embodiments, the immune cells are T cells. In some embodiments, the T cells are effector T cells. In some embodiments, the T cells are CD8+ T cells. In some embodiments, the T cells are CD4⁺ T cells. In some embodiments, the immune cells are natural killer (NK) cells. In some embodiments, the immune cells are dendritic cells (DC).

The impact of the GAL9 antigen binding molecule on immune cell cytokine secretion may be determined by any suitable means. For instance, the impact of the GAL9 antigen binding molecule on immune cell cytokine secretion may be determined in vivo, ex vivo, or in vitro. In some embodiments, cytokine secretion is determined in activated immune cells contacted with a GAL9 antigen binding molecule, as compared to activated immune cells contacted with a control agent, e.g., a control antigen binding molecule or vehicle control. The immune cells may be activated by peptide stimulation. For example, the immune cells may be activated by a peptide or plurality of peptides known to induce an immune response. The control agent can be a negative control or a positive control. In some embodiments, the GAL9 antigen binding molecule increases cytokine secretion in immune cells, relative to a negative control agent or negative control antigen binding molecule. In some embodiments, the negative control antigen binding molecule is an isotype control binding molecule that does not bind GAL9. In some embodiments, the positive control antibody is an anti-PD1 antibody, such as nivolumab. In some embodiments, the positive control antibody is a GAL9 control antibody. The GAL9 control antibody can be Gal9 antibody clone RG9.1 (Cat. No. BE0218, InVivoMab Antibodies) or RG9.35. RG9.1 and RG9.35 are both described in Fukushima A, Sumi T, Fukuda K, Kumagai N, Nishida T, et al. (2008), “Roles of galectin-9 in the development of experimental allergic conjunctivitis in mice,” Int Arch Allergy Immunol 146: 36-43, which is hereby incorporated by reference in its entirety. The GAL9 control antibody can be Gal9 antibody clone ECA42 (Cat. No. LS-C179449, LifeSpan BioScience). In some embodiments, the GAL9 antigen binding molecule increases cytokine secretion in immune cells, relative to the positive control antibody.

Cytokine secretion by the immune cells can be assessed by any suitable means. By way of example only, cytokine secretion by in vitro or ex vivo immune cell culture models may be assessed by analyzing cytokine content of the cultured cell supernatants, e.g., by cytokine bead array.

In some embodiments, the cytokine is IFN-γ. In some embodiments, the GAL9 antigen binding molecule increases IFN-γ secretion in activated immune cells by at least 10%, 15%, 20%, 25%, 30%, 35%, 40%, 45%, 50%, 55%, 60%, 65%. 70%, 75%, 80%, 85%, 90%, 95%, 100%. 105%, 110%. 115%, or 120%. In some embodiments, the GAL9 antigen binding molecule increases IFN-γ secretion in activated immune cells by at least 10-15%, 15-20%, 20-25%, 25-30%, 30-35%, 35%-40%, 40%-45%, 45%-50%, 50%-55%, 55%-60%, 60%-65%, 70%-75%, 75%-80%, 80%-85%, 85%-90%, 90%-95%, 95%-100%, 100%-105%, 105%-110%, 110%-115%, or 115%-120%.

In some embodiments, the cytokine is TNF-α. In some embodiments, the GAL9 antigen binding molecule increases TNF-α secretion in activated immune cells by at least 100%, 150%, 200%, 250%, 300%, 350%, 400%, 450%, 500%, 550%, 600%, 650%, 700%, 750%, 800%, 850%, 900%, 950%, 10,00%, 10,500%, 11,000%, 11,500%, 12,000%, 12,500%, 13,000%, 13,500%, 14,000%, 14,500%, 15,000%, 15,500%, 16,000%, 16,500%, 17,000%, 17,500%, 18,000%, 18,500%, 19,000%, 19,500%, 20,000%, 20,500%, 30,000%, 30,500%, 40,000%, 40,500%, 50,000%, 50,500%, 60,000%, 60,500%, 70,000%, 70,500%, 80,000%, 80,500%, 90,000%, or 90,500% as compared to a negative control agent described herein. In some embodiments, the GAL9 antigen binding molecule increases TNF-α secretion in activated immune cells by at least 100%-150%, 150%-200%, 200%-250%, 250%-300%, 300%-350%, 350%-400%, 400%-450%, 500%-550%, 550%-600%, 600%-650%, 650%-700%, 700%-750%, 750%-800%, 800%-850%, 850%-900%, 900%-950%, 950%-10,000%, 10,000%-10,500%, 10,500%-11,000%, 11,000-11,500%, 11,500-12,000%, 12,000%-12,500%, 13,000%-13,500%, 13,500%-14,000%, 14,000%-14,500%, 14,500%-15,000%, 15,000-15,500%, 15,550%-16,000%, 16,000%-16,500%, 17,000%-17,500%, 17,500%-18,000%, 17,500%-18,500%, 18,500%-19,000%, 19,000%-19,500%, 19,500%-20,000%, 20,000%-20,500%, 20,500%-30,000%, 30,000%-30,500%, 30,500%-40,000%, 40,000%-40,500%, 45,500%-50,000%, 50,000%-50,500%, 55,500%-60,000%, 60,000%-60,500%, 70,000%-70,500%, 70,500%-80,000%, 80,000%-80,500%, 85,000%-90,000%, or 90,000%-90,500% as compared to a negative control agent described herein.

In various embodiments, the activated immune cells are T-cells, CD8⁺ T cells, NK cells, CD4⁺ T cells, or Dendritic Cells (DC).

In some embodiments, the GAL9 antigen binding molecule increases surface expression of one or more costimulatory molecules on immune cells, e.g., human immune cells. In certain embodiments, the GAL9 antigen binding molecule increases surface expression of the one or more costimulatory molecules in activated immune cells. In particular embodiments, the immune cells are T cells. In specific embodiments, the activated immune cells are CD8+ T cells. In certain embodiments, the activated immune cell is an NK cell. In certain embodiments, the activated immune cell is a dendritic cell.

In some embodiments, the one or more costimulatory molecules is selected from 4-1BB, CD27, CD40L, ICOS, and OX40. In some embodiments, the one or more costimulatory molecules is selected from 4-1BB, CD27, CD40L, and OX40. In some embodiments, the one or more costimulatory molecules is selected from 4-1BB, CD40L, and OX40.

The impact of the GAL9 antigen binding molecule on surface expression of the one or more costimulatory molecules may be determined by any suitable means. For instance, the impact of the GAL9 antigen binding molecule on surface expression of the one or more costimulatory molecules may be determined in vivo, ex vivo, or in vitro.

In some embodiments, the GAL9 antigen binding molecule increases surface expression of the one or more costimulatory molecules in activated immune cells as compared to activated immune cells treated with a control agent. Exemplary control agents are described herein. In particular embodiments, the control agent is an isotype control binding molecule that does not bind GAL9.

In some embodiments, the GAL9 antigen binding molecule increases CD40L surface expression of activated CD8+ T-cells, relative to activated CD8+ T-cells treated with the control agent. In some embodiments, activated CD8+ T-cells treated with the GAL9 antigen binding molecule exhibits at least about a 0.1× increase, 0.2× increase, 0.3× increase, 0.4× increase, 0.5× increase, 0.6× increase, 0.7× increase, 0.8× increase, 0.9× increase, 1× increase, 2× increase, 3× increase, 4× increase, 5× increase, 6× increase, 7× increase, 8× increase, 9× increase, 10× increase, or greater than 10× increase in CD40L surface expression relative to activated CD8+ T-cells treated with the control agent. In some embodiments, activated CD8+ T-cells treated with the GAL9 antigen binding molecule exhibits about a 0.1×-10× increase, a 0.5×-5× increase, a 1×-4× increase, or about a 1.5×-2.5× increase in CD40L surface expression, relative to activated CD8+ T-cells treated with the control agent.

In some embodiments, the GAL9 antigen binding molecule increases OX40 surface expression of activated CD8+ T-cells, relative to activated CD8+ T-cells treated with the control agent. In some embodiments, activated CD8+ T-cells treated with the GAL9 antigen binding molecule exhibits about at least a 0.1× increase, 0.2× increase, 0.3× increase, 0.4× increase, 0.5× increase, 0.6× increase, 0.7× increase, 0.8× increase, 0.9× increase, 1× increase, 2× increase, 3× increase, 4× increase, 5× increase, 6× increase, 7× increase, 8× increase, 9× increase, 10× increase, or greater than 10× increase in OX40 surface expression relative to activated CD8+ T-cells treated with the control agent. In some embodiments, activated CD8+ T-cells treated with the GAL9 antigen binding molecule exhibits about a 0.1×-10× increase, a 0.5×-5× increase, or about a 1.0×-2.0× increase in OX40 surface expression, relative to activated CD8+ T-cells treated with the control agent.

In some embodiments, the GAL9 antigen binding molecule increases 4-1BB surface expression of activated CD8+ T-cells, relative to activated CD8+ T-cells treated with the control agent. In some embodiments, activated CD8+ T-cells treated with the GAL9 antigen binding molecule exhibits about at least a 0.1× increase, 0.2× increase, 0.3× increase, 0.4× increase, 0.5× increase, 0.6× increase, 0.7× increase, 0.8× increase, 0.9× increase, 1× increase, 2× increase, 3× increase, 4× increase, 5× increase, 6× increase, 7× increase, 8× increase, 9× increase, 10× increase, or greater than 10× increase in 4-1BB surface expression relative to activated CD8+ T-cells treated with the control agent. In some embodiments, activated CD8+ T-cells treated with the GAL9 antigen binding molecule exhibits about a 0.1×-10× increase, a 0.2×-2× increase, or about a 0.5×-1× increase in 4-1BB surface expression, relative to activated CD8+ T-cells treated with the control agent.

In some embodiments, the GAL9 antigen binding molecule increases CD27 surface expression of activated CD8+ T-cells, relative to activated CD8+ T-cells treated with the control agent. In some embodiments, activated CD8+ T-cells treated with the GAL9 antigen binding molecule exhibits about at least a 1% increase, 2% increase, 3% increase, 4% increase, 5% increase, 6% increase, 7% increase, 8% increase, 9% increase, 10% increase, 11% increase, 12% increase, 13% increase, 14% increase, 15% increase, 16% increase, 17% increase, 18% increase, 19% increase, 20% increase, 21% increase, 22% increase, 23% increase, 24% increase, 25% increase, 26% increase, 27% increase, 28% increase, 29% increase, 30% increase, 31% increase, 32% increase, 33% increase, 34% increase, 35% increase, 36% increase, 37% increase, 38% increase, 39% increase, 40% increase, 41% increase, 42% increase, 43% increase, 44% increase, 45% increase, 46% increase, 47% increase, 48% increase, 49% increase, 50% increase, 51% increase, 52% increase, 53% increase, 54% increase, 55% increase, 56% increase, 57% increase, 58% increase, 59% increase, 60% increase, 61% increase, 62% increase, 63% increase, 64% increase, 65% increase, 66% increase, 67% increase, 68% increase, 69% increase, 70% increase, 71% increase, 72% increase, 73% increase, 74% increase, 75% increase, 76% increase, 77% increase, 78% increase, 79% increase, 80% increase, 81% increase, 82% increase, 83% increase, 84% increase, 85% increase, 86% increase, 87% increase, 88% increase, 89% increase, 90% increase, 91% increase, 92% increase, 93% increase, 94% increase, 95% increase, 96% increase, 97% increase, 98% increase, 99% increase, or 100% increase in CD27 surface expression, relative to activated CD8+ T-cells treated with the control agent. In some embodiments, activated CD8+ T-cells treated with the GAL9 antigen binding molecule exhibits about at least a 1%-100% increase, a 5%-50% increase, a 10%-40% increase, or about a 20%-30% increase in CD27 surface expression, relative to activated CD8+ T-cells treated with the control agent.

In some embodiments, the GAL9 antigen binding molecule increases ICOS surface expression of activated CD8+ T-cells, relative to activated CD8+ T-cells treated with the control agent. In some embodiments, activated CD8+ T-cells treated with the GAL9 antigen binding molecule exhibits about at least a 1% increase, 2% increase, 3% increase, 4% increase, 5% increase, 6% increase, 7% increase, 8% increase, 9% increase, 10% increase, 11% increase, 12% increase, 13% increase, 14% increase, 15% increase, 16% increase, 17% increase, 18% increase, 19% increase, 20% increase, 21% increase, 22% increase, 23% increase, 24% increase, 25% increase, 26% increase, 27% increase, 28% increase, 29% increase, 30% increase, 31% increase, 32% increase, 33% increase, 34% increase, 35% increase, 36% increase, 37% increase, 38% increase, 39% increase, 40% increase, 41% increase, 42% increase, 43% increase, 44% increase, 45% increase, 46% increase, 47% increase, 48% increase, 49% increase, 50% increase, 51% increase, 52% increase, 53% increase, 54% increase, 55% increase, 56% increase, 57% increase, 58% increase, 59% increase, 60% increase, 61% increase, 62% increase, 63% increase, 64% increase, 65% increase, 66% increase, 67% increase, 68% increase, 69% increase, 70% increase, 71% increase, 72% increase, 73% increase, 74% increase, 75% increase, 76% increase, 77% increase, 78% increase, 79% increase, 80% increase, 81% increase, 82% increase, 83% increase, 84% increase, 85% increase, 86% increase, 87% increase, 88% increase, 89% increase, 90% increase, 91% increase, 92% increase, 93% increase, 94% increase, 95% increase, 96% increase, 97% increase, 98% increase, 99% increase, or 100% increase in ICOS surface expression, relative to activated CD8+ T-cells treated with the control agent. In some embodiments, activated CD8+ T-cells treated with the GAL9 antigen binding molecule exhibits about at least a 1%-100% increase, a 5%-50% increase, a 10%-40% increase, or about a 20%-30% increase in ICOS surface expression, relative to activated CD8+ T-cells treated with the control agent.

In some embodiments, the GAL9 antigen binding molecule increases retention of PD-L1, PD-L2, or both PD-L1 and PD-L2 on the surface of tumor cells. In some embodiments, the increased retention of PD-L1, PD-L2, or both PD-L1 and PD-L2 on the surface of tumor cells is demonstrated by microscopy techniques, e.g., confocal microscopy.

In some embodiments, the GAL9 antigen binding molecule increases PD-L2 expression on the surface of dendritic cells (DCs). In some embodiments, the GAL9 antigen binding molecule decreases PD-L1 expression on the surface of dendritic cells (DCs). In some embodiments, the DCs are activated DCs. Activation of immune cells, including DCs is described herein. Surface expression of proteins, including PD-L1 and PD-L2 on DCs can be assessed by any suitable means. For example, the percentage of DCs that exhibit detectable surface PD-L1 and/or PD-L2 may be measured by, e.g., flow cytometry. In some embodiments, a population of dendritic cells treated with the GAL9 antigen binding molecule exhibits a greater percentage of cells positive for surface PD-L2 as compared to a control population of dendritic cells treated with a control agent. Exemplary control agents are described herein. In some embodiments, the control agent is an isotype antigen binding molecule that does not bind GAL9. In some embodiments, the population of dendritic cells treated with the GAL9 antigen binding molecule exhibits about a 0.1×-100×, a 0.5×-20×, a 1×-10×, or about a 5×-6× increase in the percentage of DCs exhibiting detectable surface PD-L2 expression, relative to a control population of dendritic cells treated with the control agent, e.g., the isotype control antigen binding molecule. In some embodiments, the population of dendritic cells treated with the GAL9 antigen binding molecule exhibits about a 1%-50% decrease, a 5%-30% decrease, or about a 10%-20% decrease in the percentage of DCs exhibiting detectable surface PD-L1 expression, relative to a control population of dendritic cells treated with the control agent, e.g., the isotype control antigen binding molecule.

In some embodiments, the GAL9 antigen binding molecule increases cell surface aggregation of PD-L2 in dendritic cells (DCs). In some embodiments, the DCs are activated DCs. Activation of immune cells, including DCs is described herein. In some embodiments, the increase in cell surface aggregation of PD-L2 is relative to DCs treated with a control agent. Control agents are described herein. In some embodiments, the control agent is an isotype antigen binding molecule that does not bind GAL9. Cell surface aggregation of PD-L2 in DCs may be assessed by any suitable means, e.g., confocal microscopy.

In some embodiments, the GAL9 antigen binding molecule increases IL-12 production by DCs. The DCs may be activated DCs. In some embodiments, the GAL9 antigen binding molecule increases IL-12 production in DCs, relative to DCs treated with a control agent. Exemplary control agents are described herein. In some embodiments, the control agent is an isotype antigen binding molecule that does not bind GAL9. In some embodiments, a population of DCs treated with the GAL9 antigen binding molecule exhibits about a 0.1×-100× increase, a 10×-75× increase, a 20×-40× increase, a 25×-35× increase, or about a 28× increase in the percentage of DCs that are IL-12 positive, as compared to a population of DCs treated with the control agent.

In some embodiments, the GAL9 antigen binding molecule induces clustering of GAL9 and PD-L2 on the surface of the immune cell. In some embodiments, the immune cells can be DCs. In some embodiments, the immune cells can be NK cells.

In some embodiments, the GAL9 antigen binding molecule reduces tumor burden in a subject. The subject can be a mammal. The mammal can be a mouse. In some embodiments, the mammal is a human. In some embodiments, the GAL9 antigen binding molecule prevents growth of a tumor in the subject. The tumor can be, e.g., a colon tumor. In some embodiments, the GAL9 antigen binding molecule reduces tumor growth. In some embodiments, the GAL9 antigen binding molecule reduces tumor growth by about 25%, 50%, or more than 50%. In some embodiments, the tumor is a melanoma tumor. In some embodiments, the reduction in tumor growth is relative to a subject treated with a control agent. Exemplary control agents are described herein. In some embodiments, the control agent is an isotype antigen binding molecule that does not bind GAL9.

### 6.4.2. Variable Regions

The GAL9 binding molecules described herein have variable region domain amino acid sequences of an antibody, including VH and VL antibody domain sequences. VH and VL sequences are described in greater detail below in Sections 6.4.2.1 and 6.4.2.2, respectively.

### 6.4.2.1. VH Regions

The VH amino acid sequences in the GAL9 binding molecules described herein are antibody heavy chain variable domain sequences. In a typical antibody arrangement in both nature and in the GAL9 binding molecules described herein, a specific VH amino acid sequence associates with a specific VL amino acid sequence to form an antigen-binding site. In various embodiments, VH amino acid sequences are mammalian sequences, including human sequences, synthesized sequences, or combinations of non-human mammalian, mammalian, and/or synthesized sequences, as described in further detail above in Sections 6.4.2.3 and 6.4.2.4. In various embodiments, VH amino acid sequences are mutated sequences of naturally occurring sequences.

### 6.4.2.2. VL Regions

The VL amino acid sequences useful in the GAL9 binding molecules described herein are antibody light chain variable domain sequences. In a typical arrangement in both natural antibodies and the antibody constructs described herein, a specific VL amino acid sequence associates with a specific VH amino acid sequence to form an antigen-binding site. In various embodiments, the VL amino acid sequences are mammalian sequences, including human sequences, synthesized sequences, or combinations of human, non-human mammalian, mammalian, and/or synthesized sequences, as described in further detail below in Sections 6.4.2.3 and 6.4.2.4.

In various embodiments, VL amino acid sequences are mutated sequences of naturally occurring sequences. In certain embodiments, the VL amino acid sequences are lambda (λ) light chain variable domain sequences. In certain embodiments, the VL amino acid sequences are kappa (κ) light chain variable domain sequences. In a preferred embodiment, the VL amino acid sequences are kappa (κ) light chain variable domain sequences.

### 6.4.2.3. Complementarity Determining Regions

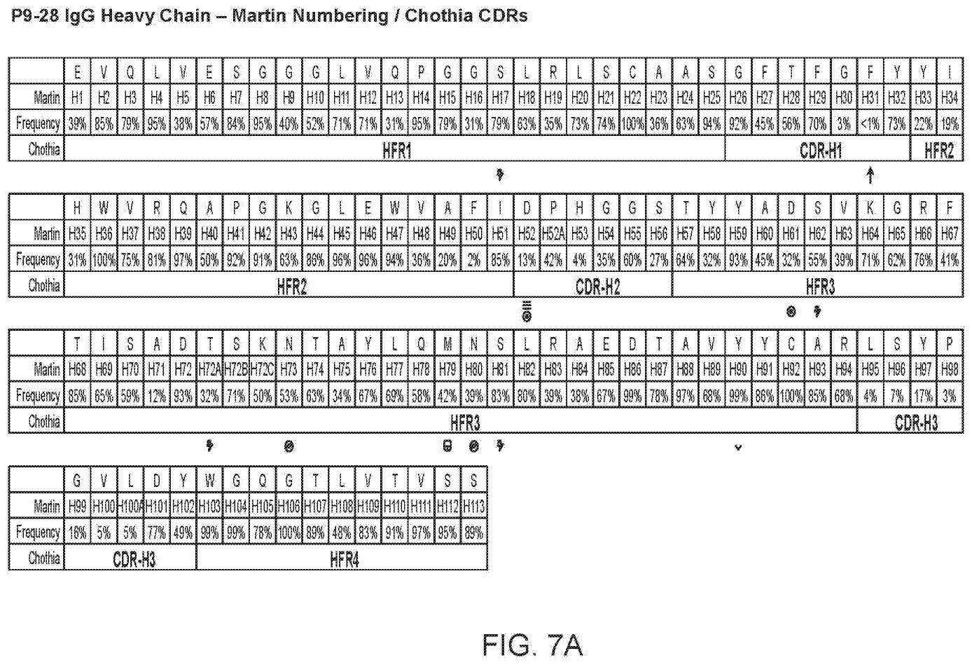

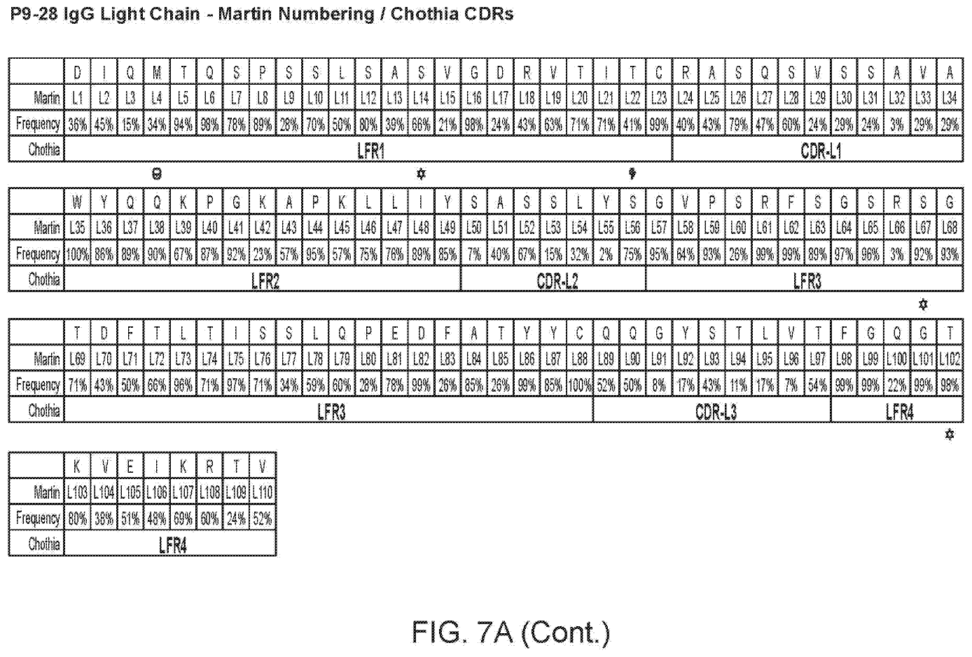

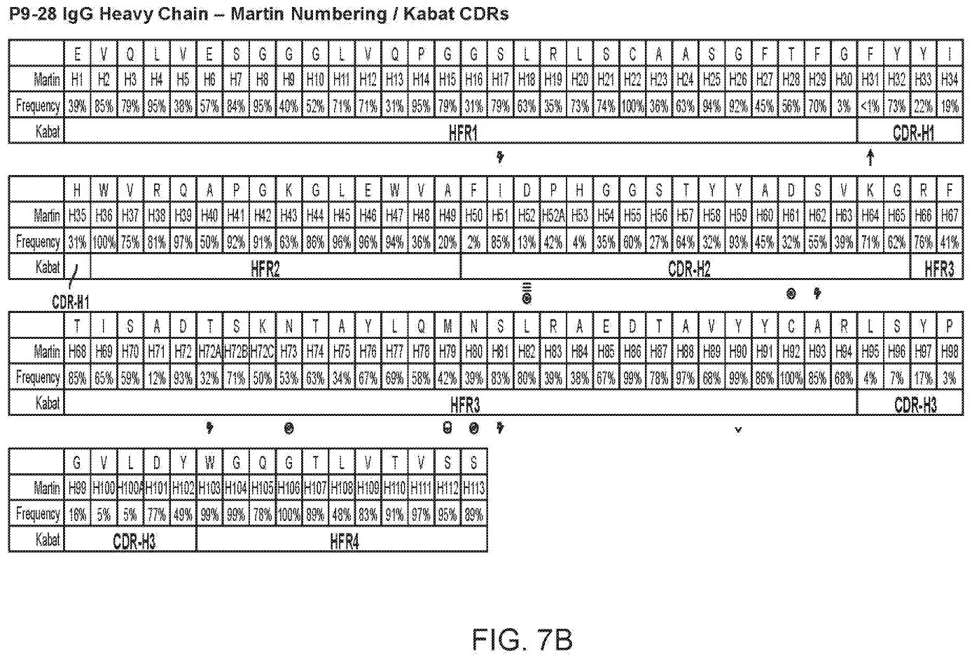

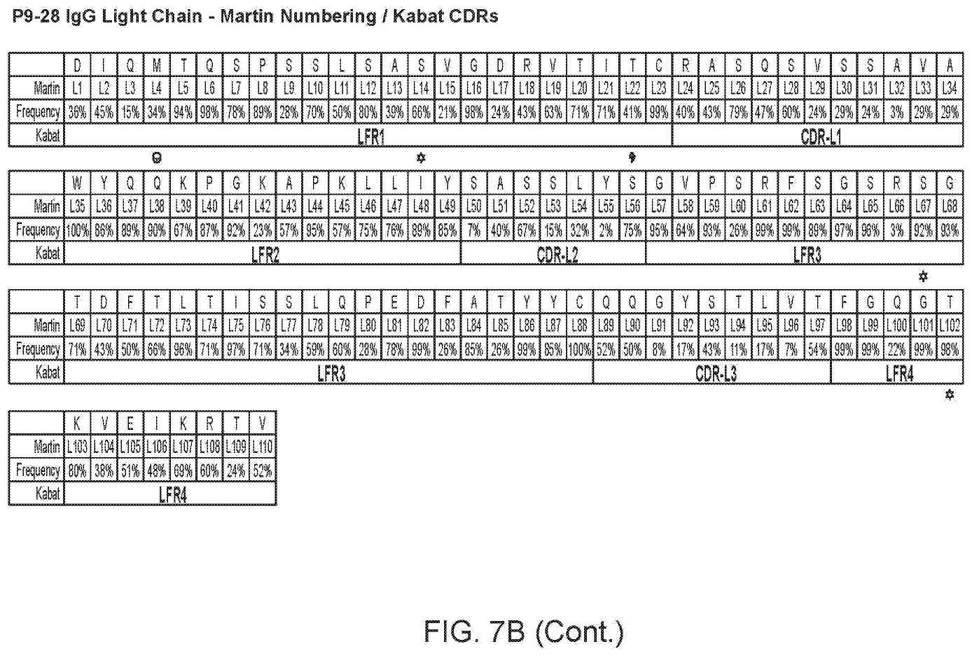

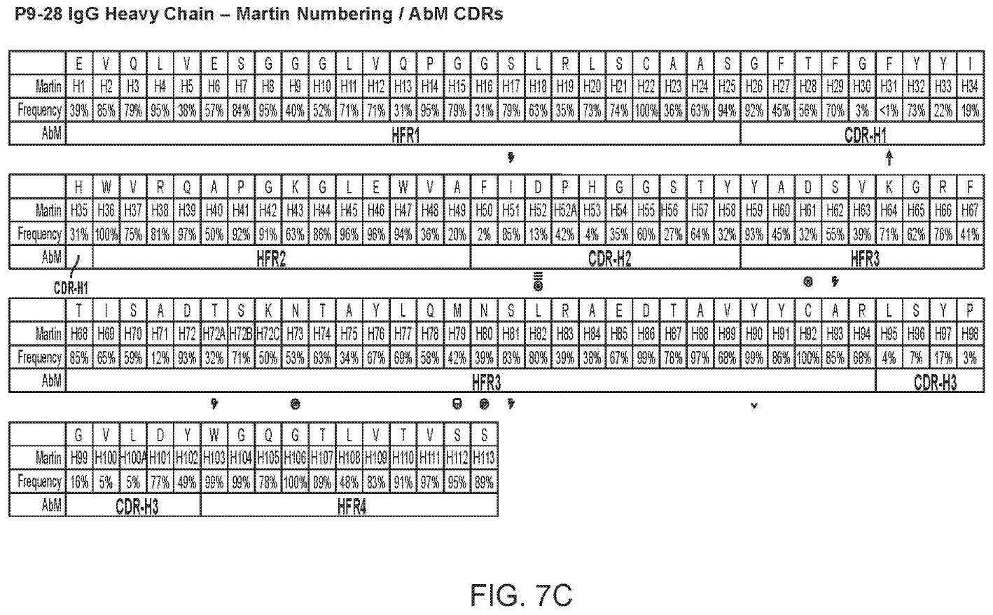

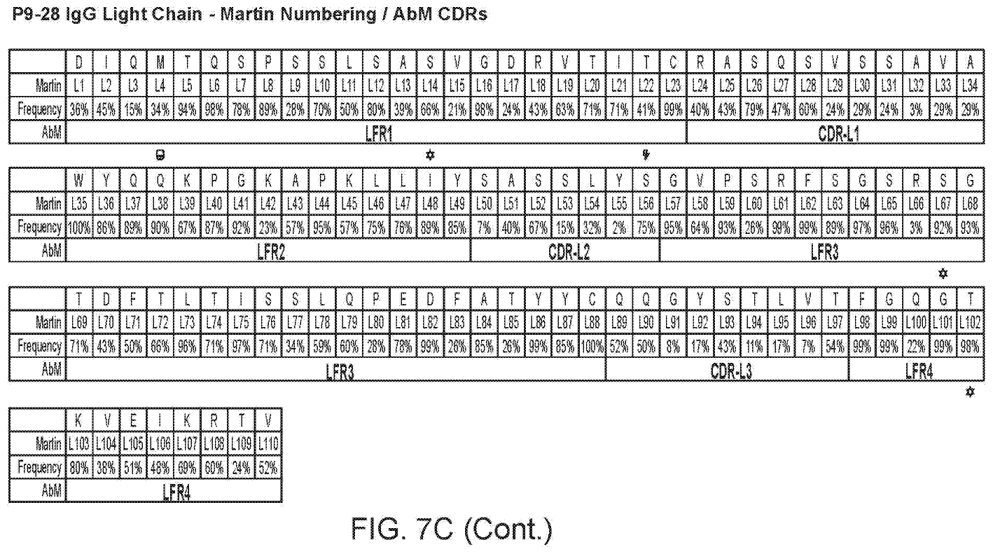

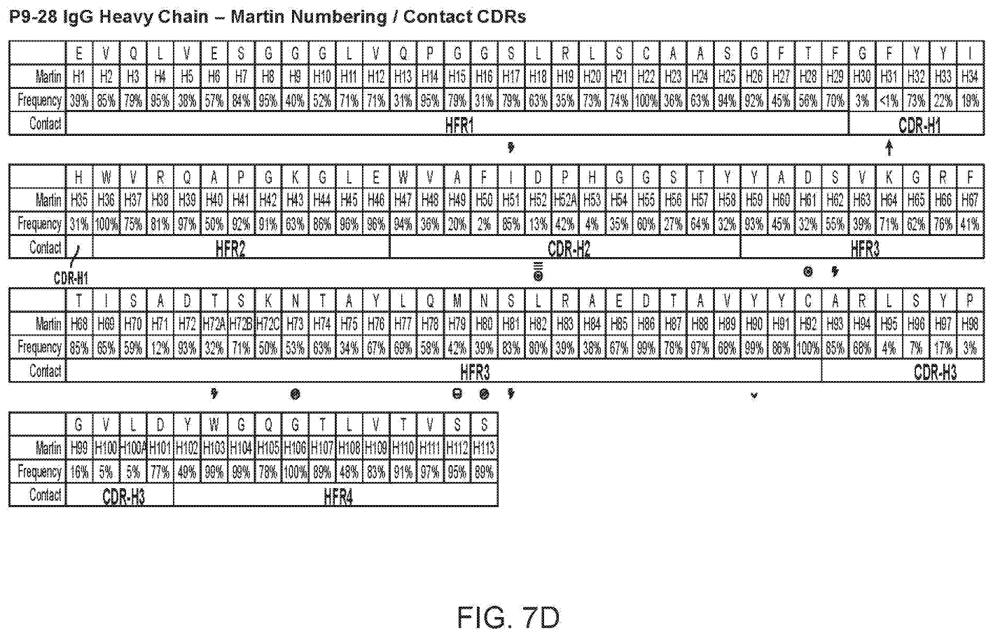

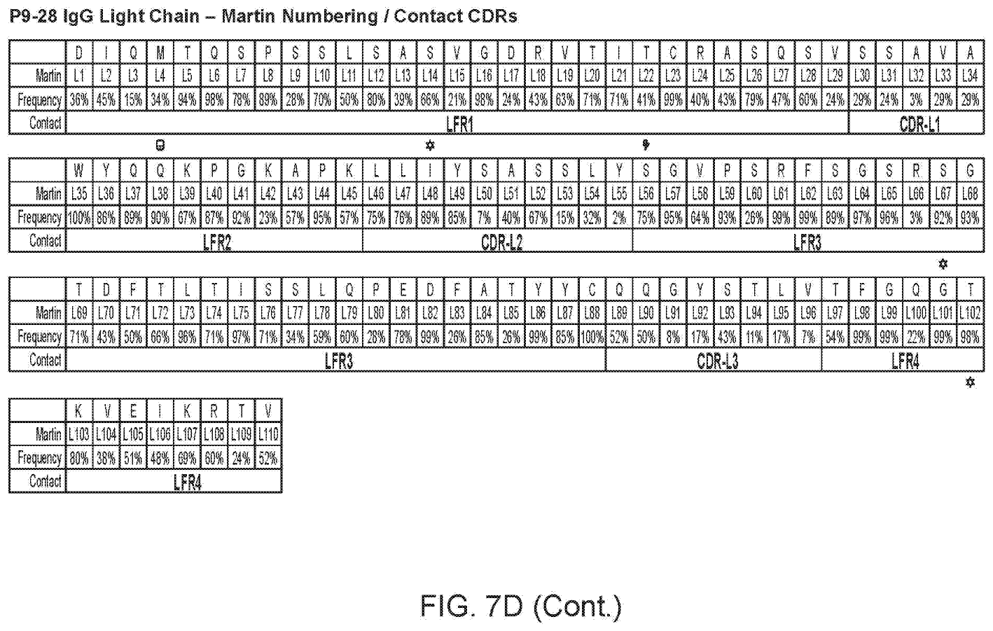

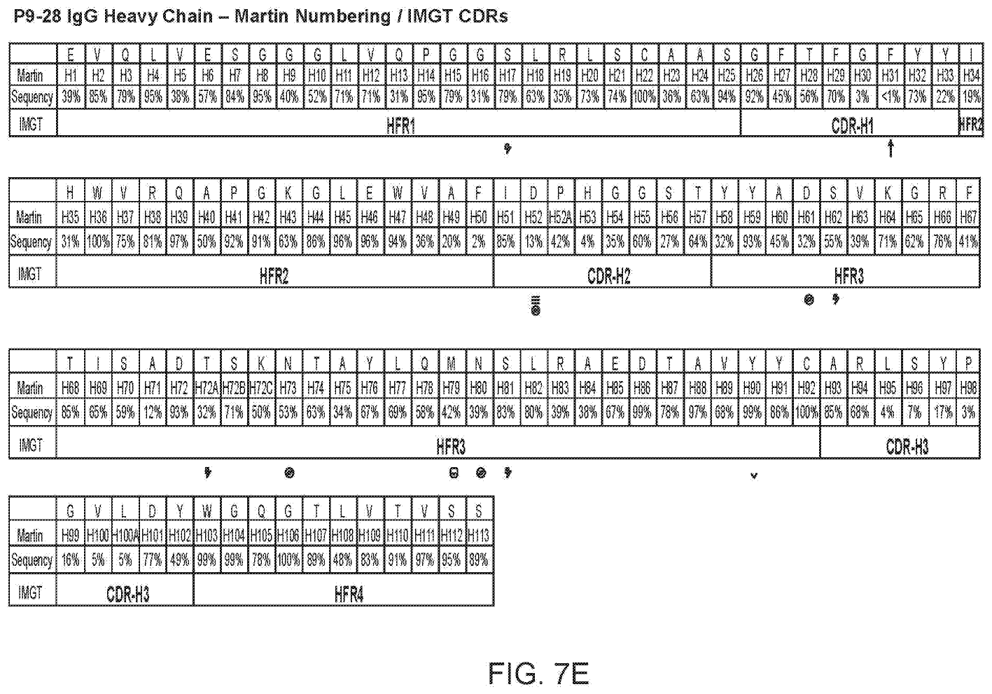

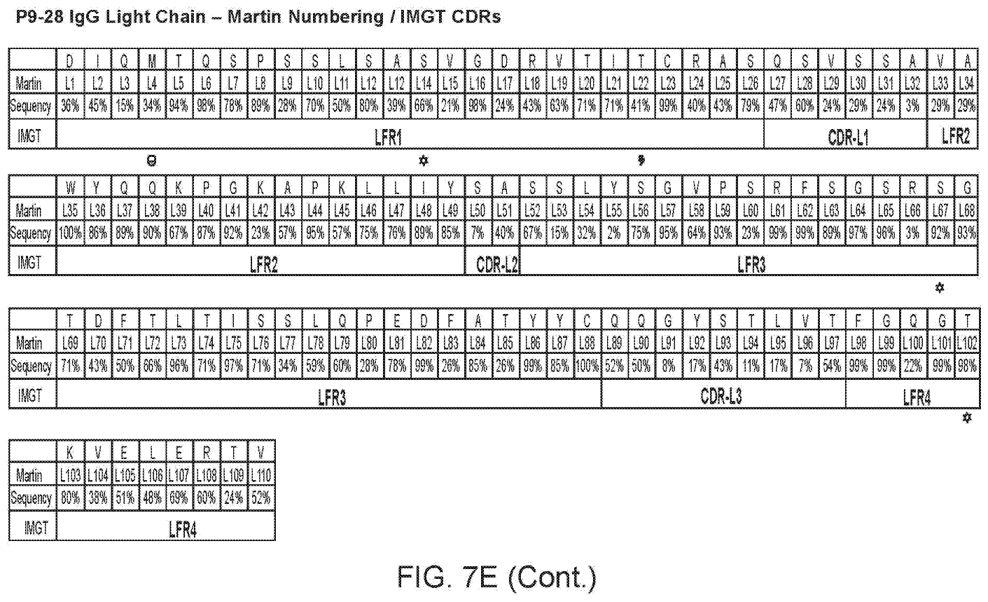

The VH and VL amino acid sequences comprise highly variable sequences termed “complementarity determining regions” (CDRs), typically three CDRs (CDR1, CDR2, and CDR3). In a variety of embodiments, the CDRs are mammalian sequences, including, but not limited to, mouse, rat, hamster, rabbit, camel, donkey, goat, and human sequences. In a preferred embodiment, the CDRs are human sequences. In various embodiments, the CDRs are naturally occurring sequences. In various embodiments, the CDRs are naturally occurring sequences that have been mutated to alter the binding affinity of the antigen-binding site for a particular antigen or epitope. In certain embodiments, the naturally occurring CDRs have been mutated in an in vivo host through affinity maturation and somatic hypermutation. In certain embodiments, the CDRs have been mutated in vitro through methods including, but not limited to, PCR-mutagenesis and chemical mutagenesis. In various embodiments, the CDRs are synthesized sequences including, but not limited to, CDRs obtained from random sequence CDR libraries and rationally designed CDR libraries. Martin numbering scheme was used to determine the CDR boundaries. See FIGS. 7A-7E.

In various embodiments, CDRs identified as binding an antigen of interest are further mutated (i.e., “affinity matured”) to achieve a desired binding characteristic, such as an increased affinity for the antigen of interest relative to the original CDR. For example, targeted introduction of diversity into the CDRs, including those CDRs identified to bind an antigen of interest, can be introduced using degenerate oligonucleotides. Various randomization schemes can be employed. For example, “soft-randomization” can be used that provides a high bias towards the identity of wild-type sequence at a given amino acid position, such as allowing a given position in CDRs to vary among all twenty amino acids while biasing towards the wild-type sequence by doping the four bases at each codon position at non-equivalent level. As an illustrative example of soft-randomization, if achieving approximately 50% of the wild-type sequence is desired, each base of each codon is kept 70% wild-type and 10% each of other nucleotides and the degenerate oligonucleotides are used to make a focused phage library around the selected CDRs with the resulting phage particles used for phage panning under various stringent selection conditions depending on the need.

### 6.4.2.4. Framework Regions and CDR Grafting

The VH and VL amino acid sequences comprise “framework region” (FR) sequences. FRs are generally conserved sequence regions that act as a scaffold for interspersed CDRs (see Section 6.4.2.3), typically in a FR1-CDR1-FR2-CDR2-FR3-CDR3-FR4 arrangement (from N-terminus to C-terminus). In a variety of embodiments, the FRs are mammalian sequences, including, but not limited to mouse, rat, hamster, rabbit, camel, donkey, goat, and human sequences. In a preferred embodiment, the FRs are human sequences. In various embodiments, the FRs are naturally occurring sequences. In various embodiments, the FRs are synthesized sequences including, but not limited, rationally designed sequences.

In a variety of embodiments, the FRs and the CDRs are both from the same naturally occurring variable domain sequence. In a variety of embodiments, the FRs and the CDRs are from different variable domain sequences, wherein the CDRs are grafted onto the FR scaffold with the CDRs providing specificity for a particular antigen. In certain embodiments, the grafted CDRs are all derived from the same naturally occurring variable domain sequence. In certain embodiments, the grafted CDRs are derived from different variable domain sequences. In certain embodiments, the grafted CDRs are synthesized sequences including, but not limited to, CDRs obtained from random sequence CDR libraries and rationally designed CDR libraries. In certain embodiments, the grafted CDRs and the FRs are from the same species. In certain embodiments, the grafted CDRs and the FRs are from different species. In a preferred grafted CDR embodiment, an antibody is “humanized”, wherein the grafted CDRs are non-human mammalian sequences including, but not limited to, mouse, rat, hamster, rabbit, camel, donkey, and goat sequences, and the FRs are human sequences. Humanized antibodies are discussed in more detail in U.S. Pat. No. 6,407,213, the entirety of which is hereby incorporated by reference for all it teaches. In various embodiments, portions or specific sequences of FRs from one species are used to replace portions or specific sequences of another species' FRs.

### 6.4.3. Exemplary Amino Acid Sequences of the GAL9 Binding Molecules

In various embodiment, the GAL9 binding molecule comprises a particular VH CDR3 (CDR-H3) sequence and a particular VL CDR3 (CDR-L3) sequence.

In some embodiments, the GAL9 binding molecule comprises the CDR-H3 and the CDR-L3 from any one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58. VH CDR amino acid sequences of the ABS clones are disclosed in Table 3. VL CDR amino acid sequences of the ABS clones are disclosed in Table 4. For clarity, each GAL9 ABS clone is assigned a unique ABS clone number which is used throughout this disclosure.

In one currently preferred embodiment, the GAL9 binding molecule comprises the CDR-H3 and CDR-L3 of ABS clone P9-28.

In some embodiments, the GAL9 binding molecule comprises all three VH CDRs from one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58. In one currently preferred embodiment, the GAL9 binding molecule comprises all three VH CDRs from ABS clone P9-28.

In some embodiments, the GAL9 binding molecule comprises all three VL CDRs from one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58. In one currently preferred embodiment, the GAL9 binding molecule comprises all three VL CDRs from ABS clone P9-28.

In some embodiments, the GAL9 binding molecule comprises all six CDRs from any one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58. In one currently preferred embodiment, the GAL9 binding molecule comprises all six CDRs from ABS clone P9-28.

In some embodiments, the GAL9 binding molecule comprises a VH amino acid sequence, a VL amino acid sequence, or a VH and VL amino acid sequence from any one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58. Full immunoglobulin heavy chain and immunoglobulin light chain sequences, as well as VH and VL amino acid sequences, are provided in Table 6. In certain currently preferred embodiments, the GAL9 binding molecule comprises a VH amino acid sequence, a VL amino acid sequence, or a VH and VL amino acid sequence from ABS clone P9-28.

In some embodiments, the GAL9 binding molecule comprises the full IgG heavy chain sequence and the full IgG light chain sequence from any one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58. In one currently preferred embodiment, the GAL9 binding molecule comprises the full IgG heavy chain sequence and the full IgG light chain sequence from ABS clone P9-28.

### 6.4.4. Constant Regions

In the GAL9 binding molecules, the GAL9 binding molecule can have a constant region domain sequence. Constant region domain amino acid sequences, as described herein, are sequences of a constant region domain of an antibody. Constant regions can refer to CH1, CH2, CH3, CH4, or CL constant domain.

In a variety of embodiments, the constant region sequences are mammalian sequences, including, but not limited to, mouse, rat, hamster, rabbit, camel, donkey, goat, and human sequences. In a preferred embodiment, the constant region sequences are human sequences. In certain embodiments, the constant region sequences are from an antibody light chain. In particular embodiments, the constant region sequences are from a lambda or kappa light chain. In certain embodiments, the constant region sequences are from an antibody heavy chain. In particular embodiments, the constant region sequences are an antibody heavy chain sequence that is an IgA1, IgA2, IgD, IgE, IgG1, IgG2, IgG3, IgG4, or IgM isotype. In a specific embodiment, the constant region sequences are from an IgG isotype. In a preferred embodiment, the constant region sequences are from an IgG1 isotype.

Exemplary constant regions and modifications thereof are described in WO2018075692, which is hereby incorporated by reference in its entirety.

### 6.4.4.1. CH1 and CL Regions

CH1 amino acid sequences, as described herein, are sequences of the second domain of an antibody heavy chain, with reference from the N-terminus to C-terminus of a native antibody heavy chain architecture. In certain embodiments, the CH1 sequences are endogenous sequences. In a variety of embodiments, the CH1 sequences are mammalian sequences, including, but not limited to mouse, rat, hamster, rabbit, camel, donkey, goat, and human sequences. In a preferred embodiment, the CH1 sequences are human sequences. In certain embodiments, the CH1 sequences are from an IgA1, IgA2, IgD, IgE, IgG1, IgG2, IgG3, IgG4, or IgM isotype. In a preferred embodiment, the CH1 sequences are from an IgG1 isotype. In preferred embodiments, the CH1 sequence is UniProt accession number P01857 amino acids 1-98.

The CL amino acid sequences useful in the GAL9 binding molecules described herein are antibody light chain constant domain sequences, with reference to a native antibody light chain architecture. In certain embodiments, the CL sequences are endogenous sequences. In a variety of embodiments, the CL sequences are mammalian sequences, including, but not limited to mouse, rat, hamster, rabbit, camel, donkey, goat, and human sequences. In a preferred embodiment, CL sequences are human sequences.

In certain embodiments, the CL amino acid sequences are lambda (λ) light chain constant domain sequences. In particular embodiments, the CL amino acid sequences are human lambda light chain constant domain sequences. In preferred embodiments, the lambda (λ) light chain sequence is UniProt accession number P0CG04.

In certain embodiments, the CL amino acid sequences are kappa (κ) light chain constant domain sequences. In a preferred embodiment, the CL amino acid sequences are human kappa (κ) light chain constant domain sequences. In a preferred embodiment, the kappa light chain sequence is UniProt accession number P01834.

In certain embodiments, the CH1 sequence and the CL sequences are both endogenous sequences. In certain embodiments, the CH1 sequence and the CL sequences separately comprise respectively orthogonal modifications in endogenous CH1 and CL sequences, as discussed below in greater detail in Section 6.4.4.1. CH1 and CL sequences can also be portions thereof, either of an endogenous or modified sequence, such that a domain having the CH1 sequence, or portion thereof, can associate with a domain having the CL sequence, or portion thereof.

### 6.4.4.2. CH1 and CL Orthogonal Modifications

In certain embodiments, the CH1 sequence and the CL sequences separately comprise respectively orthogonal modifications in endogenous CH1 and CL sequences. Orthogonal mutations, in general, are described in more detail below in Sections 6.4.6.1-6.4.6.3.

In particular embodiments, the orthogonal modifications in endogenous CH1 and CL sequences are an engineered disulfide bridge selected from engineered cysteines at position 138 of the CH1 sequence and position 116 of the CL sequence, at position 128 of the CH1 sequence and position 119 of the CL sequence, or at position 129 of the CH1 sequence and position 210 of the CL sequence, as numbered and discussed in more detail in U.S. Pat. Nos. 8,053,562 and 9,527,927, each incorporated herein by reference in its entirety. In a preferred embodiment, the engineered cysteines are at position 128 of the CH1 sequence and position 118 of the CL Kappa sequence, as numbered by the Eu index.

In a series of preferred embodiments, the mutations that provide non-endogenous cysteine amino acids are a F118C mutation in the CL sequence with a corresponding A141C in the CH1 sequence, or a F118C mutation in the CL sequence with a corresponding L128C in the CH1 sequence, or a S162C mutations in the CL sequence with a corresponding P171C mutation in the CH1 sequence, as numbered by the Eu index.

In a variety of embodiments, the orthogonal mutations in the CL sequence and the CH1 sequence are charge-pair mutations. In specific embodiments the charge-pair mutations are a F118S, F118A or F118V mutation in the CL sequence with a corresponding A141L in the CH1 sequence, or a T129R mutation in the CL sequence with a corresponding K147D in the CH1 sequence, as numbered by the Eu index and described in greater detail in Bonisch et al. (Protein Engineering, Design & Selection, 2017, pp. 1-12), herein incorporated by reference for all that it teaches. In a series of preferred embodiments, the charge-pair mutations are a N138K mutation in the CL sequence with a corresponding G166D in the CH1 sequence, or a N138D mutation in the CL sequence with a corresponding G166K in the CH1 sequence, as numbered by the Eu index.

### 6.4.4.3. CH2 Regions

In the GAL9 binding molecules described herein, the GAL9 binding molecules can have a CH2 amino acid sequence. CH2 amino acid sequences, as described herein, are CH2 amino acid sequences of the third domain of an antibody heavy chain, with reference from the N-terminus to C-terminus of a native antibody heavy chain architecture. In a variety of embodiments, the CH2 sequences are mammalian sequences, including but not limited to mouse, rat, hamster, rabbit, camel, donkey, goat, and human sequences. In a preferred embodiment, the CH2 sequences are human sequences. In certain embodiments, the CH2 sequences are from an IgA1, IgA2, IgD, IgE, IgG1, IgG2, IgG3, IgG4, or IgM isotype. In a preferred embodiment, the CH2 sequences are from an IgG1 isotype.

In certain embodiments, the CH2 sequences are endogenous sequences. In particular embodiments, the sequence is UniProt accession number P01857 amino acids 111-223.

In a series of embodiments, a GAL9 binding molecule has more than one paired set of CH2 domains that have CH2 sequences, wherein a first set has CH2 amino acid sequences from a first isotype and one or more orthologous sets of CH2 amino acid sequences from another isotype. The orthologous CH2 amino acid sequences, as described herein, are able to interact with CH2 amino acid sequences from a shared isotype, but not significantly interact with the CH2 amino acid sequences from another isotype present in the GAL9 binding molecule. In particular embodiments, all sets of CH2 amino acid sequences are from the same species. In preferred embodiments, all sets of CH2 amino acid sequences are human CH2 amino acid sequences. In other embodiments, the sets of CH2 amino acid sequences are from different species. In particular embodiments, the first set of CH2 amino acid sequences is from the same isotype as the other non-CH2 domains in the GAL9 binding molecule. In a specific embodiment, the first set has CH2 amino acid sequences from an IgG isotype and the one or more orthologous sets have CH2 amino acid sequences from an IgM or IgE isotype. In certain embodiments, one or more of the sets of CH2 amino acid sequences are endogenous CH2 sequences. In other embodiments, one or more of the sets of CH2 amino acid sequences are endogenous CH2 sequences that have one or more mutations. In particular embodiments, the one or more mutations are orthogonal knob-hole mutations, orthogonal charge-pair mutations, or orthogonal hydrophobic mutations. Orthologous CH2 amino acid sequences useful for the GAL9 binding molecules are described in more detail in international PCT applications WO2017/011342 and WO2017/106462, herein incorporated by reference in their entirety.

### 6.4.4.4. CH3 Regions

CH3 amino acid sequences, as described herein, are sequences of the C-terminal domain of an antibody heavy chain, with reference from the N-terminus to C-terminus of a native antibody heavy chain architecture.

In a variety of embodiments, the CH3 sequences are mammalian sequences, including, but not limited to, mouse, rat, hamster, rabbit, camel, donkey, goat, and human sequences. In a preferred embodiment, the CH3 sequences are human sequences. In certain embodiments, the CH3 sequences are from an IgA1, IgA2, IgD, IgE, IgM, IgG1, IgG2, IgG3, IgG4 isotype or CH4 sequences from an IgE or IgM isotype. In a specific embodiment, the CH3 sequences are from an IgG isotype. In a preferred embodiment, the CH3 sequences are from an IgG1 isotype.

In certain embodiments, the CH3 sequences are endogenous sequences. In particular embodiments, the CH3 sequence is UniProt accession number P01857 amino acids 224-330. In various embodiments, a CH3 sequence is a segment of an endogenous CH3 sequence. In particular embodiments, a CH3 sequence has an endogenous CH3 sequence that lacks the N-terminal amino acids G224 and Q225. In particular embodiments, a CH3 sequence has an endogenous CH3 sequence that lacks the C-terminal amino acids P328, G329, and K330. In particular embodiments, a CH3 sequence has an endogenous CH3 sequence that lacks both the N-terminal amino acids G224 and Q225 and the C-terminal amino acids P328, G329, and K330. In preferred embodiments, a GAL9 binding molecule has multiple domains that have CH3 sequences, wherein a CH3 sequence can refer to both a full endogenous CH3 sequence as well as a CH3 sequence that lacks N-terminal amino acids, C-terminal amino acids, or both.

In certain embodiments, the CH3 sequences are endogenous sequences that have one or more mutations. In particular embodiments, the mutations are one or more orthogonal mutations that are introduced into an endogenous CH3 sequence to guide specific pairing of specific CH3 sequences, as described in more detail below in Sections 6.4.6.1-6.4.6.3.

In certain embodiments, the CH3 sequences are engineered to reduce immunogenicity of the antibody by replacing specific amino acids of one allotype with those of another allotype and referred to herein as isoallotype mutations, as described in more detail in Stickler et al. (Genes Immun. 2011 April; 12(3): 213-221), which is herein incorporated by reference for all that it teaches. In particular embodiments, specific amino acids of the G1 ml allotype are replaced. In a preferred embodiment, isoallotype mutations D356E and L358M are made in the CH3 sequence.

In some embodiments, an IgG1 CH3 amino acid sequence comprises the following mutational changes: P343V; Y349C; and a tripeptide insertion, 445P, 446G, 447K. In other preferred embodiments, domain B has a human IgG1 CH3 sequence with the following mutational changes: T366K; and a tripeptide insertion, 445K, 446S, 447C. In still other preferred embodiments, domain B has a human IgG1 CH3 sequence with the following mutational changes: Y349C and a tripeptide insertion, 445P, 446G, 447K.

In some embodiments, an IgG1 CH3 amino acid sequence comprises a 447C mutation incorporated into an otherwise endogenous CH3 sequence.

### 6.4.5. Antigen Binding Sites

In some embodiments, a VL or VH amino acid sequence and a cognate VL or VH amino acid sequence are associated and form a first antigen binding site (ABS). The antigen binding site (ABS) is capable of specifically binding an epitope of an antigen. Antigen binding by an ABS is described in greater detail below in Section 6.4.5.1.

In alternative embodiments, e.g., wherein the GAL9 binding molecule is a single domain antibody, a VH or VL amino acid sequence forms the first ABS.

In some embodiments, the GAL9 antigen binding molecule comprises a second ABS. In some embodiments, the second ABS is specific for the same GAL9 antigen as the first ABS. In some embodiments, the second ABS specifically binds the same epitope of the same GAL9 antigen as the first ABS. In some embodiments, the second ABS is identical to the first ABS.

In some embodiments, the second ABS is specific for a different epitope of the first GAL9 antigen. For example if the first ABS comprises CDRs or variable domains from any one of the ABS clones selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58, the second ABS may comprise CDRs or variable domains from another ABS clone selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58.

In some embodiments, the GAL9 antigen binding molecule is multispecific, e.g., the second ABS of the GAL9 antigen binding molecule specifically binds an antigen that is different than the GAL9 antigen specifically bound by the first ABS.

### 6.4.5.1. Binding of Antigen by ABS

An ABS, and the GAL9 binding molecule comprising such ABS, is said to “recognize” the epitope (or more generally, the antigen) to which the ABS specifically binds, and the epitope (or more generally, the antigen) is said to be the “recognition specificity” or “binding specificity” of the ABS.

The ABS is said to bind to its specific antigen or epitope with a particular affinity. As described herein, “affinity” refers to the strength of interaction of non-covalent intermolecular forces between one molecule and another. The affinity, i.e. the strength of the interaction, can be expressed as a dissociation equilibrium constant (K_{D}), wherein a lower K_{D} value refers to a stronger interaction between molecules. K_{D} values of antibody constructs are measured by methods well known in the art including, but not limited to, bio-layer interferometry (e.g., Octet/FORTEBIO®), surface plasmon resonance (SPR) technology (e.g., Biacore®), and cell binding assays. For purposes herein, affinities are dissociation equilibrium constants measured by bio-layer interferometry using Octet/FORTEBIO®.

“Specific binding,” as used herein, refers to an affinity between an ABS and its cognate antigen or epitope in which the K_{D} value is below 10⁻⁶M, 10⁻⁷M, 10⁻⁸M, 10⁻⁹M, or 10⁻¹⁰M.

The number of ABSs in a GAL9 binding molecule as described herein defines the “valency” of the GAL9 binding molecule. A GAL9 binding molecule having a single ABS is “monovalent”. A GAL9 binding molecule having a plurality of ABSs is said to be “multivalent”. A multivalent GAL9 binding molecule having two ABSs is “bivalent.” A multivalent GAL9 binding molecule having three ABSs is “trivalent.” A multivalent GAL9 binding molecule having four ABSs is “tetravalent.”

In various multivalent embodiments, all of the plurality of ABSs have the same recognition specificity. Such a GAL9 binding molecule is a “monospecific” “multivalent” binding construct. In other multivalent embodiments, at least two of the plurality of ABSs have different recognition specificities. Such GAL9 binding molecules are multivalent and “multispecific”. In multivalent embodiments in which the ABSs collectively have two recognition specificities, the GAL9 binding molecule is “bispecific.” In multivalent embodiments in which the ABSs collectively have three recognition specificities, the GAL9 binding molecule is “trispecific.”

In multivalent embodiments in which the ABSs collectively have a plurality of recognition specificities for different epitopes present on the same antigen, the GAL9 binding molecule is “multiparatopic.” Multivalent embodiments in which the ABSs collectively recognize two epitopes on the same antigen are “biparatopic.”

In various multivalent embodiments, multivalency of the GAL9 binding molecule improves the avidity of the GAL9 binding molecule for a specific target. As described herein, “avidity” refers to the overall strength of interaction between two or more molecules, e.g. a multivalent GAL9 binding molecule for a specific target, wherein the avidity is the cumulative strength of interaction provided by the affinities of multiple ABSs. Avidity can be measured by the same methods as those used to determine affinity, as described above. In certain embodiments, the avidity of a GAL9 binding molecule for a specific target is such that the interaction is a specific binding interaction, wherein the avidity between two molecules has a K_{D} value below 10⁻⁶M, 10⁻⁷M, 10⁻⁸M, 10⁻⁹M, or 10⁻¹⁰M. In certain embodiments, the avidity of a GAL9 binding molecule for a specific target has a K_{D} value such that the interaction is a specific binding interaction, wherein the one or more affinities of individual ABSs do not have has a K_{D} value that qualifies as specifically binding their respective antigens or epitopes on their own. In certain embodiments, the avidity is the cumulative strength of interaction provided by the affinities of multiple ABSs for separate antigens on a shared specific target or complex, such as separate antigens found on an individual cell. In certain embodiments, the avidity is the cumulative strength of interaction provided by the affinities of multiple ABSs for separate epitopes on a shared individual antigen.

### 6.4.6. Orthogonal Modifications

In the GAL9 binding molecules described herein, a GAL9 binding molecule can have constant region domains comprising orthogonal modifications. Constant region domain amino acid sequences are described in greater detail above in Section 6.4.4.

“Orthogonal modifications” or synonymously “orthogonal mutations” as described herein are one or more engineered mutations in an amino acid sequence of an antibody domain that increase the affinity of binding of a first domain having orthogonal modification for a second domain having a complementary orthogonal modification. In certain embodiments, the orthogonal modifications decrease the affinity of a domain having the orthogonal modifications for a domain lacking the complementary orthogonal modifications. In certain embodiments, orthogonal modifications are mutations in an endogenous antibody domain sequence. In a variety of embodiments, orthogonal modifications are modifications of the N-terminus or C-terminus of an endogenous antibody domain sequence including, but not limited to, amino acid additions or deletions. In particular embodiments, orthogonal modifications include, but are not limited to, engineered disulfide bridges, knob-in-hole mutations, and charge-pair mutations, as described in greater detail below in Sections 6.4.6.1-6.4.6.3. In particular embodiments, orthogonal modifications include a combination of orthogonal modifications selected from, but not limited to, engineered disulfide bridges, knob-in-hole mutations, and charge-pair mutations. In particular embodiments, the orthogonal modifications can be combined with amino acid substitutions that reduce immunogenicity, such as isoallotype mutations, as described in greater detail above in Section 6.4.4.4.

### 6.4.6.1. Orthogonal Engineered Disulfide Bridges

In a variety of embodiments, the orthogonal modifications comprise mutations that generate engineered disulfide bridges between a first and a second domain. As described herein, “engineered disulfide bridges” are mutations that provide non-endogenous cysteine amino acids in two or more domains such that a non-native disulfide bond forms when the two or more domains associate. Engineered disulfide bridges are described in greater detail in Merchant et al. (Nature Biotech (1998) 16:677-681), the entirety of which is hereby incorporated by reference for all it teaches. In certain embodiments, engineered disulfide bridges improve orthogonal association between specific domains. In a particular embodiment, the mutations that generate engineered disulfide bridges are a K392C mutation in one of a first or second CH3 domains, and a D399C in the other CH3 domain. In a preferred embodiment, the mutations that generate engineered disulfide bridges are a S354C mutation in one of a first or second CH3 domains, and a Y349C in the other CH3 domain. In another preferred embodiment, the mutations that generate engineered disulfide bridges are a 447C mutation in both the first and second CH3 domains that are provided by extension of the C-terminus of a CH3 domain incorporating a KSC tripeptide sequence.

### 6.4.6.2. Orthogonal Knob-Hole Mutations

In a variety of embodiments, orthogonal modifications comprise knob-hole (synonymously, knob-in-hole) mutations. As described herein, knob-hole mutations are mutations that change the steric features of a first domain's surface such that the first domain will preferentially associate with a second domain having complementary steric mutations relative to association with domains without the complementary steric mutations. Knob-hole mutations are described in greater detail in U.S. Pat. Nos. 5,821,333 and 8,216,805, each of which is incorporated herein in its entirety. In various embodiments, knob-hole mutations are combined with engineered disulfide bridges, as described in greater detail in Merchant et al. (Nature Biotech (1998) 16:677-681), incorporated herein by reference in its entirety. In various embodiments, knob-hole mutations, isoallotype mutations, and engineered disulfide mutations are combined.

In certain embodiments, the knob-in-hole mutations are a T366Y mutation in a first domain, and a Y407T mutation in a second domain. In certain embodiments, the knob-in-hole mutations are a F405A in a first domain, and a T394W in a second domain. In certain embodiments, the knob-in-hole mutations are a T366Y mutation and a F405A in a first domain, and a T394W and a Y407T in a second domain. In certain embodiments, the knob-in-hole mutations are a T366W mutation in a first domain, and a Y407A in a second domain. In certain embodiments, the combined knob-in-hole mutations and engineered disulfide mutations are a S354C and T366W mutations in a first domain, and a Y349C, T366S, L368A, and a Y407V mutation in a second domain. In a preferred embodiment, the combined knob-in-hole mutations, isoallotype mutations, and engineered disulfide mutations are a S354C and T366W mutations in a first domain, and a Y349C, D356E, L358M, T366S, L368A, and a Y407V mutation in a second domain.

### 6.4.6.3. Orthogonal Charge-Pair Mutations

In a variety of embodiments, orthogonal modifications are charge-pair mutations. As used herein, charge-pair mutations are mutations that affect the charge of an amino acid in a domain's surface such that the domain will preferentially associate with a second domain having complementary charge-pair mutations relative to association with domains without the complementary charge-pair mutations. In certain embodiments, charge-pair mutations improve orthogonal association between specific domains. Charge-pair mutations are described in greater detail in U.S. Pat. Nos. 8,592,562, 9,248,182, and 9,358,286, each of which is incorporated by reference herein for all they teach. In certain embodiments, charge-pair mutations improve stability between specific domains. In a preferred embodiment, the charge-pair mutations are a T366K mutation in a first domain, and a L351D mutation in the other domain.

In specific embodiments, the orthogonal mutations are charge-pair mutations at the VH/VL interface. In preferred embodiments, the charge-pair mutations at the VH/VL interface are a Q39E in VH with a corresponding Q38K in VL, or a Q39K in VH with a corresponding Q38E in VL, as described in greater detail in Igawa et al. (Protein Eng. Des. Sel., 2010, vol. 23, 667-677), herein incorporated by reference for all it teaches.

### 6.4.7. Trivalent and Tetravalent GAL9 Binding Molecules

In another series of embodiments, the GAL9 binding molecules have three antigen binding sites and are therefore termed “trivalent.” In a variety of embodiments, the GAL9 binding molecules have 4 antigen binding sites and are therefore termed “tetravalent.”

### 6.5. GAL9 Binding Molecule Architecture

The antigen binding sites described herein, including specific CDR subsets, can be formatted into any binding molecule architecture including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches. The antigen binding sites described herein, including specific CDR subsets, can also be formatted into a “B-body” format, as described in more detail in US pre-grant publication no. US 2018/0118811 and International Application Pub. No. WO 2018/075692, each of which is herein incorporated by reference in their entireties.

### 6.6. Further Modification

In a further series of embodiments, the GAL9 binding molecule has additional modifications.

### 6.6.1. Antibody-Drug Conjugates

In various embodiments, the GAL9 binding molecule is conjugated to a therapeutic agent (i.e. drug) to form a GAL9 binding molecule-drug conjugate. Therapeutic agents include, but are not limited to, chemotherapeutic agents, imaging agents (e.g. radioisotopes), immune modulators (e.g. cytokines, chemokines, or checkpoint inhibitors), and toxins (e.g. cytotoxic agents). In certain embodiments, the therapeutic agents are attached to the GAL9 binding molecule through a linker peptide, as discussed in more detail below in Section 6.6.3.

Methods of preparing antibody-drug conjugates (ADCs) that can be adapted to conjugate drugs to the GAL9 binding molecules disclosed herein are described, e.g., in U.S. Pat. No. 8,624,003 (pot method), U.S. Pat. No. 8,163,888 (one-step), U.S. Pat. No. 5,208,020 (two-step method), U.S. Pat. Nos. 8,337,856, 5,773,001, 7,829,531, 5,208,020, 7,745,394, WO 2017/136623, WO 2017/015502, WO 2017/015496, WO 2017/015495, WO 2004/010957, WO 2005/077090, WO 2005/082023, WO 2006/065533, WO 2007/030642, WO 2007/103288, WO 2013/173337, WO 2015/057699, WO 2015/095755, WO 2015/123679, WO 2015/157286, WO 2017/165851, WO 2009/073445, WO 2010/068759, WO 2010/138719, WO 2012/171020, WO 2014/008375, WO 2014/093394, WO 2014/093640, WO 2014/160360, WO 2015/054659, WO 2015/195925, WO 2017/160754, Storz (MAbs. 2015 November-December; 7(6): 989-1009), Lambert et al. (Adv Ther, 2017 34: 1015), Diamantis et al. (British Journal of Cancer, 2016, 114, 362-367), Carrico et al. (Nat Chem Biol, 2007. 3: 321-2), We et al. (Proc Natl Acad Sci USA, 2009. 106: 3000-5), Rabuka et al. (Curr Opin Chem Biol., 2011 14: 790-6), Hudak et al. (Angew Chem Int Ed Engl., 2012: 4161-5), Rabuka et al. (Nat Protoc., 2012 7:1052-67), Agarwal et al. (Proc Natl Acad Sci USA., 2013, 110: 46-51), Agarwal et al. (Bioconjugate Chem., 2013, 24: 846-851), Barfield et al. (Drug Dev. and D., 2014, 14:34-41), Drake et al. (Bioconjugate Chem., 2014, 25:1331-41), Liang et al. (J Am Chem Soc., 2014, 136:10850-3), Drake et al. (Curr Opin Chem Biol., 2015, 28:174-80), and York et al. (BMC Biotechnology, 2016, 16(1):23), each of which is hereby incorporated by reference in its entirety for all that it teaches.

### 6.6.2. Additional Binding Moieties

In various embodiments, the GAL9 binding molecule has modifications that comprise one or more additional binding moieties. In certain embodiments the binding moieties are antibody fragments or antibody formats including, but not limited to, full-length antibodies, Fab fragments, Fvs, scFvs, tandem scFvs, Diabodies, scDiabodies, DARTs, tandAbs, minibodies, camelid VHH, and other antibody fragments or formats known to those skilled in the art. Exemplary antibody and antibody fragment formats are described in detail in Brinkmann et al. (MABS, 2017, Vol. 9, No. 2, 182-212), herein incorporated by reference for all that it teaches.

In particular embodiments, the one or more additional binding moieties are attached to the C-terminus of the first or third polypeptide chain. In particular embodiments, the one or more additional binding moieties are attached to the C-terminus of both the first and third polypeptide chain. In particular embodiments, the one or more additional binding moieties are attached to the C-terminus of both the first and third polypeptide chains. In certain embodiments, individual portions of the one or more additional binding moieties are separately attached to the C-terminus of the first and third polypeptide chains such that the portions form the functional binding moiety.

In particular embodiments, the one or more additional binding moieties are attached to the N-terminus of any of the polypeptide chains (e.g. the first, second, third, fourth, fifth, or sixth polypeptide chains). In certain embodiments, individual portions of the additional binding moieties are separately attached to the N-terminus of different polypeptide chains such that the portions form the functional binding moiety.

In certain embodiments, the one or more additional binding moieties are specific for a different antigen or epitope of the ABSs within the GAL9 binding molecule. In certain embodiments, the one or more additional binding moieties are specific for the same antigen or epitope of the ABSs within the GAL9 binding molecule. In certain embodiments, wherein the modification is two or more additional binding moieties, the additional binding moieties are specific for the same antigen or epitope. In certain embodiments, wherein the modification is two or more additional binding moieties, the additional binding moieties are specific for different antigens or epitopes.

In certain embodiments, the one or more additional binding moieties are attached to the GAL9 binding molecule using in vitro methods including, but not limited to, reactive chemistry and affinity tagging systems, as discussed in more detail below in Section 6.6.3. In certain embodiments, the one or more additional binding moieties are attached to the GAL9 binding molecule through Fc-mediated binding (e.g. Protein A/G). In certain embodiments, the one or more additional binding moieties are attached to the GAL9 binding molecule using recombinant DNA techniques, such as encoding the nucleotide sequence of the fusion product between the GAL9 binding molecule and the additional binding moieties on the same expression vector (e.g., plasmid).

### 6.6.3. Functional/Reactive Groups

In various embodiments, the GAL9 binding molecule has modifications that comprise functional groups or chemically reactive groups that can be used in downstream processes, such as linking to additional moieties (e.g., drug conjugates and additional binding moieties, as discussed in more detail above in Sections 6.6.1. and 6.6.2.) and downstream purification processes.

In certain embodiments, the modifications are chemically reactive groups including, but not limited to, reactive thiols (e.g. maleimide based reactive groups), reactive amines (e.g., N-hydroxysuccinimide based reactive groups), “click chemistry” groups (e.g. reactive alkyne groups), and aldehydes bearing formylglycine (FGly). In certain embodiments, the modifications are functional groups including, but not limited to, affinity peptide sequences (e.g., HA, HIS, FLAG, GST, MBP, and Strep systems etc.). In certain embodiments, the functional groups or chemically reactive groups have a cleavable peptide sequence. In particular embodiments, the cleavable peptide is cleaved by means including, but not limited to, photocleavage, chemical cleavage, protease cleavage, reducing conditions, and pH conditions. In particular embodiments, protease cleavage is carried out by intracellular proteases. In particular embodiments, protease cleavage is carried out by extracellular or membrane associated proteases. ADC therapies adopting protease cleavage are described in more detail in Choi et al. (Theranostics, 2012; 2(2): 156-178), the entirety of which is hereby incorporated by reference for all it teaches.

### 6.6.4. Reduced Effector Function

In certain embodiments, the GAL9 binding molecule has one or more engineered mutations in an amino acid sequence of an antibody domain that reduce the effector functions naturally associated with antibody binding. Effector functions include, but are not limited to, cellular functions that result from an Fc receptor binding to an Fc portion of an antibody, such as antibody-dependent cellular cytotoxicity (ADCC, also referred to as antibody-dependent cell-mediated cytotoxicity), complement fixation (e.g. C1q binding), antibody dependent cellular-mediated phagocytosis (ADCP), and opsonization. Exemplary engineered mutations that reduce the effector functions are described in more detail in U.S. Pub. No. 2017/0137530, Armour, et al. (Eur. J. Immunol. 29(8) (1999) 2613-2624), Shields, et al. (J. Biol. Chem. 276(9) (2001) 6591-6604), and Oganesyan, et al. (Acta Cristallographica D64 (2008) 700-704), each of which is herein incorporated by reference in its entirety.

### 6.7. Methods of Purification

A method of purifying a GAL9 binding molecule is provided herein. Purification steps include, but are not limited to, purifying the GAL9 binding molecules based on protein characteristics, such as size (e.g., size exclusion chromatography), charge (e.g., ion exchange chromatography), or hydrophobicity (e.g., hydrophobicity interaction chromatography). In one embodiment, cation exchange chromatograph is performed. Other purification methods known to those skilled in the art can be performed including, but not limited to, use of Protein A, Protein G, or Protein A/G reagents. Multiple iterations of a single purification method can be performed. A combination of purification methods can be performed.

### 6.7.1. Assembly and Purity of Complexes

In the embodiments of the present invention, at least four distinct polypeptide chains associate together to form a complete complex, i.e., the GAL9 binding molecule. However, incomplete complexes can also form that do not contain the at least four distinct polypeptide chains. For example, incomplete complexes may form that only have one, two, or three of the polypeptide chains. In other examples, an incomplete complex may contain more than three polypeptide chains, but does not contain the at least four distinct polypeptide chains, e.g., the incomplete complex inappropriately associates with more than one copy of a distinct polypeptide chain. The method of the invention purifies the complex, i.e., the completely assembled GAL9 binding molecule, from incomplete complexes.

Methods to assess the efficacy and efficiency of the purification steps are well known to those skilled in the art and include, but are not limited to, SDS-PAGE analysis, ion exchange chromatography, size exclusion chromatography, and mass spectrometry. Purity can also be assessed according to a variety of criteria. Examples of criterion include, but are not limited to: 1) assessing the percentage of the total protein in an eluate that is provided by the completely assembled GAL9 binding molecule, 2) assessing the fold enrichment or percent increase of the method for purifying the desired products, e.g., comparing the total protein provided by the completely assembled GAL9 binding molecule in the eluate to that in a starting sample, 3) assessing the percentage of the total protein or the percent decrease of undesired products, e.g., the incomplete complexes described above, including determining the percent or the percent decrease of specific undesired products (e.g., unassociated single polypeptide chains, dimers of any combination of the polypeptide chains, or trimers of any combination of the polypeptide chains). Purity can be assessed after any combination of methods described herein.

### 6.8. Methods of Manufacturing

The GAL9 binding molecules described herein can readily be manufactured by expression using standard cell free translation, transient transfection, and stable transfection approaches currently used for antibody manufacture. In specific embodiments, Expi293 cells (ThermoFisher) can be used for production of the GAL9 binding molecules using protocols and reagents from ThermoFisher, such as ExpiFectamine, or other reagents known to those skilled in the art, such as polyethylenimine as described in detail in Fang et al. (Biological Procedures Online, 2017, 19:11), herein incorporated by reference for all it teaches.

The expressed proteins can be readily separated from undesired proteins and protein complexes using various purification strategies including, but not limited to, use of Protein A, Protein G, or Protein A/G reagents. Further purification can be affected using ion exchange chromatography as is routinely used in the art.

### 6.9. Pharmaceutical Compositions

In another aspect, pharmaceutical compositions are provided that comprise a GAL9 binding molecule as described herein and a pharmaceutically acceptable carrier or diluent. In typical embodiments, the pharmaceutical composition is sterile.

In various embodiments, the pharmaceutical composition comprises the GAL9 binding molecule at a concentration of 0.1 mg/ml-100 mg/ml. In specific embodiments, the pharmaceutical composition comprises the GAL9 binding molecule at a concentration of 0.5 mg/ml, 1 mg/ml, 1.5 mg/ml, 2 mg/ml, 2.5 mg/ml, 5 mg/ml, 7.5 mg/ml, or 10 mg/ml. In some embodiments, the pharmaceutical composition comprises the GAL9 binding molecule at a concentration of more than 10 mg/ml. In certain embodiments, the GAL9 binding molecule is present at a concentration of 20 mg/ml, 25 mg/ml, 30 mg/ml, 35 mg/ml, 40 mg/ml, 45 mg/ml, or even 50 mg/ml or higher. In particular embodiments, the GAL9 binding molecule is present at a concentration of more than 50 mg/ml.

In various embodiments, the pharmaceutical compositions are described in more detail in U.S. Pat. Nos. 8,961,964, 8,945,865, 8,420,081, 6,685,940, 6,171,586, 8,821,865, 9,216,219, U.S. application Ser. No. 10/813,483, WO 2014/066468, WO 2011/104381, and WO 2016/180941, each of which is incorporated herein in its entirety.

### 6.10. Methods of Treatment

In another aspect, methods of treatment are provided, the methods comprising administering a GAL9 binding molecule as described herein to a patient with a disease or condition in an amount effective to treat the patient.

### 6.10.1. Subjects

In some embodiments, the subject can be a mammal. In some embodiments, the mammal is a mouse. In a preferred embodiment, the mammal is a human.

### 6.10.2. Combination Therapy

The GAL9 binding molecule can be used alone or in combination with other therapeutic agents or procedures to treat or prevent a disease or condition. The GAL9 binding molecule can be administered either simultaneously or sequentially with a second therapeutic agent, dependent upon the disease to be treated.

In some embodiments, the anti-GAL9 binding molecules is used in combination with an agent or procedure that is used in the clinic or is within the current standard of care to treat or prevent a disease or condition, such as proliferative disease or cancer. In some embodiments, the GAL9 binding molecule is administered in combination with an immune checkpoint inhibitor, such as an anti-PD-L1 antibody, anti-PD-1 antibody, anti-CTLA4 antibody, anti-LAB3 antibody, anti-TIM1 antibody, anti-TIGIT antibody, anti-PVRIG antibody.

### 6.10.3. Proliferative Diseases

In some embodiments, the treatment comprises administration one or more GAL9 binding molecule as described herein to a subject with a proliferative disease in an amount effective to treat the subject.

In some embodiments, the treatment comprises administration of an effective amount of one or more GAL9 binding molecules as described herein for the treatment of cancer and/or precancer. In some embodiments, the treatment comprises administration of an effective amount of one or more GAL9 binding molecules as described herein, in combination with another cancer therapeutic and/or treatment regimen (radiation, surgery, or the like, etc.).

In various embodiments, the cancer is a cancer of the bladder, blood, bone, bone marrow, brain, breast, colon, esophagus, gastrointestinal, gum, head, kidney, liver, lung, nasopharynx, neck, head and neck, ovary, prostate, pancreas, skin, stomach, testis, tongue, or uterus.

In some embodiments, the cancerous or pre-cancerous tumor is a neoplasm, malignant tumor, carcinoma, undifferentiated tumor, giant and spindle cell carcinoma, small cell carcinoma, papillary carcinoma, squamous cell carcinoma, head and neck squamous cell carcinoma, lymphoepithelial carcinoma, basal cell carcinoma, pilomatrix carcinoma, transitional cell carcinoma, papillary transitional cell carcinoma, adenocarcinoma, gastrinoma, malignant, cholangiocarcinoma, hepatocellular carcinoma, combined hepatocellular carcinoma and cholangiocarcinoma, trabecular adenocarcinoma, adenoid cystic carcinoma, adenocarcinoma in adenomatous polyp, adenocarcinoma, familial polyposis coli, solid carcinoma, carcinoid tumor, malignant, branchiolo-alveolar adenocarcinoma, papillary adenocarcinoma, chromophobe carcinoma, acidophil carcinoma, oxyphilic adenocarcinoma, basophil carcinoma, clear cell adenocarcinoma, granular cell carcinoma, follicular adenocarcinoma, papillary and follicular adenocarcinoma, nonencapsulating sclerosing carcinoma, adrenal cortical carcinoma, endometroid carcinoma, skin appendage carcinoma, apocrine adenocarcinoma, sebaceous adenocarcinoma, ceruminous adenocarcinoma, mucoepidermoid carcinoma, cystadenocarcinoma, pancreatic adenocarcinoma, pancreatic ductal adenocarcinoma, cystadenocarcinomas, pancreatic neuroendocrine tumors (PanNETs), adenosquamous carcinomas of the pancreas, signet ring cell carcinomas of the pancreas, hepatoid carcinomas of the pancreas, colloid carcinomas of the pancreas, undifferentiated carcinomas of the pancreas, and undifferentiated carcinomas with osteoclast-like giant cells of the pancreas, acinar cell carcinomas of the pancreas, solid pseudopapillary neoplasms of the pancreas, pancreatoblastoma, rare exocrine cancers of the pancreas, pancreatic serous cystadenomas, pancreatic mucinous cystic neoplasms, papillary cystadenocarcinoma, papillary serous cystadenocarcinoma, mucinous cystadenocarcinoma, mucinous adenocarcinoma, signet ring cell carcinoma, infiltrating duct carcinoma, medullary carcinoma, lobular carcinoma, inflammatory carcinoma, Paget's disease, mammary, acinar cell carcinoma, adenosquamous carcinoma, adenocarcinoma w/squamous metaplasia, thymoma, malignant, ovarian stromal tumor, malignant thecoma, malignant granulosa cell tumor, malignant androblastoma, malignant sertoli cell carcinoma, leydig cell tumor, malignant lipid cell tumor, malignant paraganglioma, malignant extra-mammary paraganglioma, malignant pheochromocytoma, glomangiosarcoma, malignant melanoma, amelanotic melanoma, superficial spreading melanoma, melanoma in giant pigmented nevus, epithelioid cell melanoma, blue nevus, malignant sarcoma, fibrosarcoma, fibrous histiocytoma, malignant myxosarcoma, liposarcoma, leiomyosarcoma, rhabdomyosarcoma, embryonal rhabdomyosarcoma, alveolar rhabdomyosarcoma, stromal sarcoma, mixed tumor, malignant mullerian mixed tumor, nephroblastoma, hepatoblastoma, carcinosarcoma, mesenchymoma, malignant brenner tumor, malignant phyllodes tumor, malignant synovial sarcoma, mesothelioma, malignant dysgerminoma, embryonal carcinoma, teratoma, malignant struma ovarii, malignant choriocarcinoma, mesonephroma, malignant hemangiosarcoma, hemangioendothelioma, malignant Kaposi's sarcoma, hemangiopericytoma, malignant, lymphangiosarcoma, osteosarcoma, juxtacortical osteosarcoma, chondrosarcoma, chondroblastoma, malignant mesenchymal chondrosarcoma, giant cell tumor of bone, Ewing's sarcoma, odontogenic tumor, malignant ameloblastic odontosarcoma, ameloblastoma, malignant ameloblastic fibrosarcoma, pinealoma, malignant chordoma, glioma, malignant ependymoma, astrocytoma, protoplasmic astrocytoma, fibrillary astrocytoma, astroblastoma, glioblastoma, oligodendroglioma, oligodendroblastoma, primitive neuroectodermal, cerebellar sarcoma, ganglioneuroblastoma, neuroblastoma, retinoblastoma, olfactory neurogenic tumor, meningioma, malignant, neurofibrosarcoma, neurilemmoma, malignant granular cell tumor, malignant lymphoma, Hodgkin's disease, Hodgkin's paragranuloma, malignant lymphoma, small lymphocytic, malignant lymphoma, large cell, diffuse, malignant lymphoma, follicular, mycosis fungoides, other specified Non-Hodgkin's lymphomas, malignant histiocytosis, multiple myeloma, mast cell sarcoma, immunoproliferative small intestinal disease, leukemia, lymphoid leukemia, plasma cell leukemia, erythroleukemia, lymphosarcoma cell leukemia, myeloid leukemia, basophilic leukemia, eosinophilic leukemia, monocytic leukemia, mast cell leukemia, megakaryoblastic leukemia, myeloid sarcoma, or hairy cell leukemia.

In some embodiments, the cancer is a viral-induced cancer, for example, a cancer caused by an infection from a oncovirus or a tumor virus (which are also known as a “cancer virus”). In some embodiments, the cancer virus is a DNA virus. In some embodiments, the cancer virus is an RNA virus.

In some embodiments, the cancerous or pre-cancerous tumor is associated or caused by a cancer virus. Non-limiting examples of a cancer virus include: a Epstein-Barr virus (EBV), a Hepatitis B virus, a Hepatitis C virus, a Human papilloma virus, a Human T-lymphotropic virus 1 (HTLV-1), a Kaposi sarcoma associated-herpesvirus (KHSV), a Merkel cell polyomavirus, or a Cytomegalovirus.

In some embodiments, the cancerous or pre-cancerous tumor is associated or caused by a cancer virus that directly induces transformation of the infected host cell, thereby regulating the host cell's growth and survival or alternatively initiating a DNA damage response which in turn increases genetic instability and accelerates the acquisition of the cancer causing mutations in the genome of the host cell.

In some embodiments, the cancerous or pre-cancerous tumor is associated or caused by a cancer virus that induces chronic inflammation in a host. For example, infections with HBV and HCV can induce chronic liver inflammation associated with oxidative DNA damage followed by cirrhosis resulting in some cases in the development of hepatocellular carcinoma.

In some embodiments, the cancerous or pre-cancerous tumor is associated or caused by a cancer virus that is not oncogenic but inhibits the host's immune system, disrupting immunosurveillance and thereby allowing for the emergence of mutated malignant cells, for example HIV-infected patients.

In some embodiments, the treatment comprises administration one or more GAL9 binding molecules as described herein to a subject with an infectious disease(s), such as infection with HIV, HCV, HBV, EBV, or HPV.

In some embodiments, the treatment comprises administration one or more GAL9 binding molecules as described herein to a subject with HIV or AIDs in an amount effective to treat the subject.

### 6.10.4. Administration

The GAL9 binding molecule may be administered to a subject by any route known in the art. For example, the GAL9 binding molecule is administered to a human subject via, e.g., intravenous, subcutaneous, intramuscular, intradermal, intraarterial, intraperitoneal, intranasal, parenteral, pulmonary, topical, oral, sublingual, intratumoral, peritumoral, intralesional, intrasynovial, intrathecal, intra-cerebrospinal, or perilesional administration. The GAL9 binding molecule may be administered to a subject per se or as a pharmaceutical composition. Exemplary pharmaceutical compositions are described herein.

### 6.11. Examples

The following examples are provided by way of illustration, not limitation. In particular, the methods for the expression and purification of the various antigen-binding proteins and their use in various assays as described in more detail below are non-limiting and illustrative.

### 6.11.1. Methods

### 6.11.1.1. Expi293 Expression

Various antigen-binding proteins tested were expressed using the Expi293 transient transfection system according to manufacturer's instructions (Thermo Fisher Scientific). Briefly, plasmids coding for individual chains were mixed at 1:1 mass ratio, unless otherwise stated, and transfected into Expi 293 cells with ExpiFectamine 293 transfection kit. Cells were cultured at 37° C. with 8% CO₂, 100% humidity and shaking at 125 rpm. Transfected cells were fed once after 16-18 hours of transfections. The cells were harvested at day 5 by centrifugation at 2000 g for 10 minutes. The supernatant was collected for affinity chromatography purification.

### 6.11.1.2. ExpiCHO Expression

Various GAL9 antigen-binding proteins are tested and expressed using the ExpiCHO transient transfection system according to manufacturer's instructions. Briefly, plasmids coding for individual chains are mixed at, for example, a 1:1 mass ratio, and transfected with ExpiFectamine CHO transfection kit into ExpiCHO.

Cells are cultured at 37° C. with 8% CO₂, 100% humidity and shaking at 125 rpm. Transfected cells are generally be fed once after 16-18 hours of transfections. The cells are harvested at day 5 by centrifugation at 2000 g for 10 munities. The supernatant is then collected for affinity chromatography purification.

### 6.11.1.3. Protein A Purification

Cleared supernatants containing the various antigen-binding proteins were separated using either a Protein A (ProtA) resin or an anti-CH1 resin on a Gravity flow purifier. In examples where a head-to-head comparison was performed, supernatants containing the various antigen-binding proteins were split into two equal samples. For ProtA purification, a 1 mL Protein A column (GE Healthcare) was equilibrated with PBS (5 mM sodium potassium phosphate pH 7.4, 150 mM sodium chloride). The sample was loaded onto the column at 5 ml/min. The sample was eluted using 0.1M sodium acetate pH 3.5. The elution was monitored by absorbance at 280 nm and the elution peaks were pooled for analysis. The elution was monitored by absorbance at 280 nm and the elution peaks were pooled for analysis.

### 6.11.1.4. SDS-Page Analysis

Samples containing the various separated antigen-binding proteins were analyzed by reducing and non-reducing SDS-PAGE for the presence of complete product, incomplete product, and overall purity. 2 μg of each sample was added to 15 μL SDS loading buffer. Reducing samples were incubated in the presence of 10 mM reducing agent at 75° C. for 10 minutes. Non-reducing samples were incubated at 70° C. for 5 minutes without reducing agent. The reducing and non-reducing samples were loaded into a 4-15% gradient TGX gel (BioRad) with running buffer and run for 30 minutes at 220 volts. Upon completion of the run, the gel was washed with deionized (DI) water and stained using GelCode Blue Safe Protein Stain (ThermoFisher). The gels were destained with DI water prior to analysis. Densitometry analysis of scanned images of the destained gels was performed using standard image analysis software to calculate the relative abundance of bands in each sample.

### 6.11.1.5. IEX Chromatography

Samples containing the various separated antigen-binding proteins were analyzed by cation exchange chromatography for the ratio of complete product to incomplete product and impurities. Cleared supernatants were analyzed with a 5-ml MonoS (GE Lifesciences) on an AKTA Purifier FPLC. The MonoS column was equilibrated with buffer A (10 mM MES pH 6.0). The samples were loaded onto the column at 2 ml/min. The sample was eluted using a 0-30% gradient with buffer B (10 mM MES pH 6.0, 1 M sodium chloride) over 6 column bed volumes (CV). The elution was monitored by absorbance at 280 nm and the purity of the samples were calculated by peak integration to identify the abundance of the monomer peak and contaminants peaks. The monomer peak and contaminant peaks were separately pooled for analysis by SDS-PAGE as described above.

Analytical SEC Chromatography of each sample at 1 mg/mL was loaded onto the column at 1 ml/min. The sample was eluted using an isocratic flow of PBS for 1.5 CV. The elution was monitored by absorbance at 280 nm and the elution peaks were analyzed by peak integration.

### 6.11.1.6. Mass Spectrometry

Samples containing the various separated antigen-binding proteins were analyzed by mass spectrometry to confirm the correct species by molecular weight. All analysis was performed by a third-party research organization. Briefly, samples were treated with a cocktail of enzymes to remove glycosylation. Samples were tested in the reduced format to specifically identify each chain by molecular weight and under non-reducing conditions to identify the molecular weights of all complexes in the samples. Mass spec analysis was used to identify the number of unique products based on molecular weight.

### 6.11.1.7. Antibody Discovery by Phage Display

Phage display of human Fab libraries was carried out using standard protocols. Human GAL9 protein was purchased from Acro Biosystems (Human Gal9 His-tag Cat #LG9-H5244) and biotinylated using EZ-Link NHS-PEG₁₂-Biotin (ThermoScientific Cat #21312) using standard protocols. Phage clones were screened for the ability to bind the GAL9 protein by phage ELISA using standard protocols.

Briefly, Fab-formatted phage libraries were constructed using expression vectors capable of replication and expression in phage (also referred to as a phagemid). Both the heavy chain and the light chain were encoded for in the same expression vector, where the heavy chain was fused to a truncated variant of the phage coat protein pIII. The light chain and heavy chain-pIII fusion were expressed as separate polypeptides and assembled in the bacterial periplasm, where the redox potential enables disulfide bond formation, to form the phage display antibody containing the candidate ABS.

The library was created using sequences derived from a specific human heavy chain variable domain (VH3-23) and a specific human light chain variable domain (Vκ-1). For the screened library, all three CDRs of the VH domain were diversified to match the positional amino acid frequency by CDR length found in the human antibody repertoire. Light chain variable domains within the screened library were generated with diversity introduced solely into the VL CDR3 (L3); the light chain VL CDR1 (L1) and CDR2 (L2) retained the human germline sequence.

The heavy chain scaffold (SEQ ID NO:2), light chain scaffold (SEQ ID NO:4), full heavy chain Fab polypeptide (SEQ ID NO:1), and full light chain Fab polypeptide (SEQ ID NO:3) used in the phage display library are shown below, where a lower case “x” represents CDR amino acids that were varied to create the library.

Phage display VH scaffold
[SEQ ID NO: 2]:
EVQLVESGGGLVQPGGSLRLSCAASGFTFxxxxIHWVRQAPGKGLEWVAx
xxxxxxxxxxYADSVKGRFTISADTSKNTAYLQMNSLRAEDTAVYYCARx
xxxxxxxxxxxxDYWGQGTLVTVSSAS
Phage display VL scaffold
[SEQ ID NO: 4]:
DIQMTQSPSSLSASVGDRVTITCRASQSVSSAVAWYQQKPGKAPKLLIYS
ASSLYSGVPSRFSGSRSGTDFTLTISSLQPEDFATYYCQQxxxxxxTFGQ
GTKVEIKRT
Phage display heavy chain Fab polypeptide
[SEQ ID NO: 1]:
EVQLVESGGGLVQPGGSLRLSCAASGFTFxxxxIHWVRQAPGKGLEWVAx
xxxxxxxxxxYADSVKGRFTISADTSKNTAYLQMNSLRAEDTAVYYCARx
xxxxxxxxxxxxDYWGQGTLVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTHTC
Phage display light chain Fab polypeptide
[SEQ ID NO: 3]:
DIQMTQSPSSLSASVGDRVTITCRASQSVSSAVAWYQQKPGKAPKLLIYS
ASSLYSGVPSRFSGSRSGTDFTLTISSLQPEDFATYYCQQxxxxxxTFGQ
GTKVEIKRTVAAPSVFIFPPSDSQLKSGTASVVCLLNNFYPREAKVQWKV
DNALQSGNSQESVTEQDSKDSTYSLSSTLTLSKADYEKHKVYACEVTHQG
LSSPVTKSFNRGEC

Diversity was created through Kunkel mutagenesis using primers to introduce diversity into VH CDR1 (H1), VH CDR2 (H2), VH CDR3 (H3) and VL CDR3 (L3) to mimic the diversity found in the natural antibody repertoire, as described in more detail in Kunkel, TA (PNAS Jan. 1, 1985. 82 (2) 488-492), incorporated herein by reference in its entirety. Briefly, single-stranded DNA was prepared from isolated phage using standard procedures and Kunkel mutagenesis was carried out. Chemically synthesized DNA was then electroporated into MC1061F-cells. Phagemid obtained from overnight culture was digested with restriction enzymes (Bam HI and Xba I) to remove the wild-type sequence. The digested sample was electroporated into TG1 cells, followed by recovery. Recovered cells were sub-cultured and infected with M13K07 helper phage to produce the phage library.

Phage panning was performed using standard procedures. Briefly, the first round of phage panning was performed with target immobilized on streptavidin magnetic beads which were subjected to ˜5×10¹² phages from the prepared library in a volume of 1 mL in PBST-2% BSA. After a one-hour incubation, the bead-bound phage were separated from the supernatant using a magnetic stand. Beads were washed three times to remove non-specifically bound phage and were then added to ER2738 cells (5 mL) at OD₆₀₀˜0.6. After 20 minutes, infected cells were sub-cultured in 25 mL 2×YT+ Ampicillin and M13K07 helper phage (final concentration, ˜10¹⁰ pfu/ml) and allowed to grow overnight at 37° C. with vigorous shaking. The next day, phage were prepared using standard procedures by PEG precipitation. Pre-clearance of phage specific to SAV-coated beads was performed prior to panning. The second round of panning was performed using the KingFisher magnetic bead handler with 100 nM bead-immobilized antigen using standard procedures. In total, 3-4 rounds of phage panning were performed to enrich in phage displaying Fabs specific for the target antigen. Target-specific enrichment was confirmed using polyclonal and monoclonal phage ELISA. DNA sequencing was used to determine isolated Fab clones containing a candidate ABS.

The VL and VH domains identified in the phage screen described above were reformatted into a bivalent monospecific native human full-length IgG1 architecture.

Native human full-length IgG1 heavy chain
architecture [SEQ ID NO: 5]:
[SEQ ID NO: 5]
EVQLVESGGGLVQPGGSLRLSCAASGFTFxxxxIHWVRQAPGKGLEWVAx
xxxxxxxxxxYADSVKGRFTISADTSKNTAYLQMNSLRAEDTAVYYCARx
xxxxxxxxxxxxDYWGQGTLVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLS
LSPGK

Native Human Full-Length IgG1 Light Chain Architecture:

Equivalent to phage display light chain Fab, see [SEQ ID NO:3].

### 6.11.1.8. Octet Determination of Binding Kinetics

To measure qualitative binding affinity in the GAL9 binder discovery campaigns, IgG1 reformatted binders were immobilized to a biosensor on an Octet (Pall ForteBio) biolayer interferometer.

Soluble GAL9 antigen was then added to the system and binding measured. Qualitative binding affinity was assessed by visualizing the slope of the dissociation phase of the octet sensogram from weakest (+) to strongest (+++). A slow off rate represented by a negligible drop in the dissociation phase of the sensogram and indicated a tight binding antibody (+++). To obtain accurate kinetic constants for monovalent affinities, a dilution series involving of at least five concentrations of the GAL9 analyte (ranging from approximately 10 to 20× K_{D} to 0.1× K_{D} value, 2-fold dilutions) were measured in the association step. In the dissociation step, the sensor was dipped into buffer solution that did not contain the GAL9 analyte, permitting the bound complex on the surface of the sensor to dissociate. Octet kinetic analysis software was used to calculate the kinetic and equilibrium binding constants based on the rate of association and dissociation curves. Analysis was performed globally (global fit) where kinetic constants were derived simultaneously from all analyte concentration included in the experiment.

### 6.11.1.9. Epitope Binning

Anti-GAL9 candidates formatted into a bivalent monospecific native human full-length IgG1, as described above, were tested for GAL9 binding in a pair-wise manner using an octet-based ‘tandem’ assay. Briefly, biotinylated GAL9 was immobilized on a streptavidin sensor and two anti-GAL9 candidates were bound in tandem. A competitive blocking profile was generated determining whether a given anti-GAL9 candidate blocked binding of a panel of other anti-GAL9 candidates to GAL9. Anti-GAL9 candidates that competed for the same or non-overlapping binding regions were grouped together and referred to as belonging to the same bin.

### 6.11.1.10. PBMC Activation and Galectin 9 Antibody Treatment

Individual aliquots of PepMix HCMVA (pp65) (>90%) Protein ID: P06725 (Cat. No. PM-PP65-2, JPT Peptide Technologies) were prepared according to manufacturer's instructions. PepMix™ HCMVA (pp65) is a complete protein-spanning mixture of overlapping 15mer peptides of the 65 kDa phosphoprotein (pp65) (Swiss-Prot ID: P06725) of human cytomegalovirus (HHV-5). Aliquots of PepMix were used for immunostimulation of PBMCs to assess immune cell responses.

Frozen human peripheral blood mononuclear cells (PBMCs) were thawed according to standard conditions, then resuspended in growth media (10% FBS in RPMI).

Resuspended PBMCs were seeded at 5×10⁵ cells in 96-well plates. Cells were incubated with 2 μg/mL PepMix™ HCMVA (pp65) plus 40 μg/mL of candidate GAL9 antibodies or control antibodies in growth media for 24 hours at 37° C., 5% CO₂.

### 6.11.1.11. LEGENDplex Human Th Cytokine Assay

Cytokine secretion by PBMCs and by specific immune cell subpopulations was assessed by cytokine bead array at 24 hours and 72 hours after PBMC activation by PepMix HCMVA (pp65) and Galectin 9 antibody treatment as follows.

200 μl cell culture supernatant was collected and centrifuged to pellet cell debris. The resulting supernatants were analyzed using the LEGENDplex™ Human Th1 Panel (5-plex) (Cat. No. 740009, Biolegend). The LEGENDplex™ Human Th1 Panel is a bead-based assay to allows for simultaneous quantification of human cytokines IL-2, IL-6, IL-10, IFN-γ and TNF-α using flow cytometry.

Briefly, cytokine standards and capture bead mixtures were prepared according to manufacturer's instructions. Assay master mixes of 1:1:1 capture bead mixture: biotinylated detection antibodies, and assay buffer were prepared.

12.5 μl of supernatant samples or cytokine standards were incubated with 37.5 μl assay master mix. Plates were sealed, covered with foil, and shaken at 600 rpm for 2 hours at room temperature. Wells were then incubated, with shaking at 600 rpm, with streptavidin-phycoerythrin (SA-PE) for 30 minutes at room temperature. Beads were then washed twice and resuspended before proceeding to flow cytometry analysis according to manufacturer's instructions.

### 6.11.1.12. PBMC Staining with Marker Antibodies

Following PBMC activation and Galectin 9 antibody treatment as described above, PBMC immune cells were stained with marker antibodies according to the following procedures.

Cells were resuspended at 5×10⁶ cells/mL in growth media (10% FBS in RPMI). 200 μL of resuspended cells were aliquoted to 96 well plates, then incubated with Fixable Viability Dye eFluor® 780 for 30 minutes at 2-8° C. to irreversibly label dead cells. Cells were then washed and then incubated with human Fc Block solution (Cat. No. 14-9161-73, eBiosciences) for 10 minutes at room temperature.

An antibody cocktail working solution was prepared according to the following table.

TABLE 1
Antibody Staining Working Solutions

| | Antibody | Dilution |
|---|---|---|
| T cell surface | BV510 anti-human CD3 | 1 in 20 |
| markers | (Cat. No. 563109, BD Biosciences) | |
| | PerCP/Cy5.5 anti-human CD56 | 1 in 20 |
| | (Cat. No. 362505, BD Biosciences) | |
| Monocyte surface | FITC anti-human CD14 | 1 in 20 |
| markers | (Cat. No. 367115, BD Biosciences) | |
| | Alexa Fluor ® 700 anti-human | 1 in 20 |
| | CD16 (Cat. No. 302025, Biolegend) | |
| Dendritic cell | Brilliant Violet 421 ™ anti-human | 1 in 20 |
| surface markers | CD11c (Cat. No. 301627, Biolegend) | |
| | Alexa Fluor 647 anti-human CD123 | 1 in 40 |
| | (Cat. No. 306023, Biolegend) | |
| | BV510 anti-human Lineage Cocktail | 1 in 10 |
| | (CD3, CD14, CD16, CD19, CD20, | |
| | CD56) (Cat. No. 348807, Biolegend) | |
| | FITC anti-human HLA-DR | 1 in 20 |
| | (Cat. No. 307603, Biolegend) | |
| B cell surface | PerCP/Cy5.5 anti-human CD19 | 1 in 20 |
| markers | (Cat. No. 363015, Biolegend) | |
| Galectin-9 | PE anti-human galectin 9 | 1 in 10 |
| | (Cat. No. 348905, Biolegend) | |

Wells were incubated with 10 μL of diluted antibody cocktail for 30 minutes at 2-8° C. Cells were then washed and resuspended and analyzed by flow cytometry analysis.

To analyze immune stimulatory markers CD27, CD40L, ICOS, 4-1BB, and OX40 the same protocol provided above was followed but cells were incubated with the alternative antibody cocktail as detailed in Table 2 below:

TABLE 2
Antibody Staining Working Solutions

| Antibody | Dilution |
|---|---|
| FITC anti-human CD134 (OX40) (Cat. No. | 1 in 50 |
| 350006, BioLegend) | |
| PerCP/Cy5.5 anti-human CD3 (Cat. No. | 1 in 100 |
| 560835, BD Biosciences) | |
| AF700 anti-human CD4 (Cat. No. 344622, | 1 in 100 |
| BioLegend) | |
| eFluor ™ Fixable Viability Dye (Cat. No. | 1 in 2000 |
| 65-0865-14, eBioscienceTM) | |
| BV421 anti-human CD8 (Cat. No. 344748, | 1 in 100 |
| BioLegend) | |
| BV650 anti-human CD137 (4-1BB) (Cat. | 1 in 50 |
| No. 309828, BioLegend) | |
| BV711 anti-human ICOS (Cat. No. 563833, | 1 in 100 |
| BD Biosciences) | |
| PE anti-human CD154 (CD40L) (Cat. No. | 1 in 50 |
| 310806, BioLegend) | |
| PE/Cy7 anti- mouse/rat/human CD27 (Cat. | 1 in 100 |
| No. 124216, BioLegend) | |

### 6.11.2. Example 1: GAL9 Binding Arm Discovery Campaign

A chemically synthetic Fab phage library with diversity introduced into the Fab CDRs was screened against GAL9 antigens using a monoclonal phage ELISA format as described above. Phage clones expressing Fabs that recognized GAL9 were sequenced.

The campaign initially identified 52 GAL9 binding candidates (antigen binding site clones). Functional assays conducted after the variable regions of these clones had been reformatted into a bivalent monospecific human IgG1 format identified 22 antibodies having immune activating properties.

Table 3 lists the VH CDR1/2/3 sequences from the 22 activating ABS clones, showing only the residues of the CDRs that had been varied in constructing the library. Table 4 lists the VL CDR1/2/3 sequences from the identified ABS clones; the light chain CDR1 and CDR2 sequences are invariant, and only the residues of CDR3 that were varied in constructing the library are shown.

TABLE 3
Candidate hGAL9 VH Antigen Binding Sites

| | CDR1 | SEQ | CDR2 | SEQ | CDR3 | SEQ |
|---|---|---|---|---|---|---|
| ABS | (variant | ID | (valiant | ID | (variant | ID |
| clone | residues) | NO | residues) | NO | residues) | NO |
| P9-02B | SGYF | 9 | RISSYGGHTD | 61 | AQYVPGL | 113 |
| P9-04 | GSYF | 11 | YISPTWGYTY | 63 | AWFGFAF | 115 |
| P9-05 | TSYY | 12 | WIWPIGGYTY | 64 | DAGSGF | 116 |
| P9-08 | SRYA | 15 | AIYSPTGYTD | 67 | EGYIGM | 119 |
| P9-09 | SSYF | 16 | WIYSSGGYTY | 68 | FTPSDLGYGL | 120 |
| P9-10 | DYYV | 17 | AIDSYWGDTY | 69 | FYFYGF | 121 |
| P9-15 | RYYW | 21 | AIFPSGGITT | 73 | GWPWGL | 125 |
| P9-16 | SYYW | 22 | DIYPSSGYTY | 74 | GWYAAYGM | 126 |
| P9-18 | SWYV | 23 | AIYPYHSKTY | 75 | GYWYGM | 127 |
| P9-19 | GYYY | 24 | WISPSGSVTA | 76 | GYYGGWGM | 128 |
| P9-20 | RYYY | 25 | GIYPYGGYTS | 77 | GYYVEGVL | 129 |
| P9-21 | SYYY | 26 | RIHPPSGYTD | 78 | GYYVFGVM | 130 |
| P9-22 | SSYY | 27 | AIYPFSGGTY | 79 | GYYVYVVM | 131 |
| P9-27 | WGYG | 32 | AIYPYGGSTY | 84 | LSDIYHSFSGM | 136 |
| P9-28 | GFYY | 33 | FIDPHGGSTY | 85 | LSYPGVL | 137 |
| P9-31 | SQYA | 36 | RIYPDSGYTY | 88 | PYHQYAEGM | 140 |
| P9-32 | SAYW | 37 | LIGPDGGYTY | 89 | QASRGL | 141 |
| P9-36 | GTYY | 39 | SILSGGGYTV | 91 | RVYPGF | 143 |
| P9-39 | SFYG | 42 | WIYPYGGFTD | 94 | SGFFAF | 146 |
| P9-49 | SWYE | 50 | RIGPYSSYTY | 102 | TYYPSYGM | 154 |
| P9-54 | STYF | 55 | WISPSGSHTG | 107 | VRYPGVM | 159 |
| P9-58 | SRYY | 58 | FISSDSGYTQ | 110 | TMSYSAL | 162 |

TABLE 4
Candidate hGAL9 VL Antigen Binding Sites

| | | SEQ | | SEQ | CDR3 | SEQ |
|---|---|---|---|---|---|---|
| ABS | CDR1 | ID | CDR2 | ID | (variant | ID |
| clone | (invariant) | NO | (invariant) | NO | residues) | NO |
| P9-02B | RASQSVSSA | 165 | SASSLYS | 217 | SYPTLG | 269 |
| P9-04 | RASQSVSSA | 167 | SASSLYS | 219 | VYSSPL | 271 |
| P9-05 | RASQSVSSA | 168 | SASSLYS | 220 | YYYSLL | 272 |
| P9-08 | RASQSVSSA | 171 | SASSLYS | 223 | SYSALY | 275 |
| P9-09 | RASQSVSSA | 172 | SASSLYS | 224 | ADPSLV | 276 |
| P9-10 | RASQSVSSA | 173 | SASSLYS | 225 | YSWSLW | 277 |
| P9-15 | RASQSVSSA | 177 | SASSLYS | 229 | RDRSPY | 281 |
| P9-16 | RASQSVSSA | 178 | SASSLYS | 230 | YALRPL | 282 |
| P9-18 | RASQSVSSA | 179 | SASSLYS | 231 | YTDAPW | 283 |
| P9-19 | RASQSVSSA | 180 | SASSLYS | 232 | YDTSPY | 284 |
| P9-20 | RASQSVSSA | 181 | SASSLYS | 233 | YYGSLA | 285 |
| P9-21 | RASQSVSSA | 182 | SASSLYS | 234 | SHWYPF | 286 |
| P9-22 | RASQSVSSA | 183 | SASSLYS | 235 | YKSSPW | 287 |
| P9-27 | RASQSVSSA | 188 | SASSLYS | 240 | RYSTPV | 292 |
| P9-28 | RASQSVSSA | 189 | SASSLYS | 241 | GYSTLV | 293 |
| P9-31 | RASQSVSSA | 192 | SASSLYS | 244 | YYSPLL | 296 |
| P9-32 | RASQSVSSA | 193 | SASSLYS | 245 | WSSPLH | 297 |
| P9-36 | RASQSVSSA | 195 | SASSLYS | 247 | DSWGLW | 299 |
| P9-39 | RASQSVSSA | 198 | SASSLYS | 250 | VQTSLA | 302 |
| P9-49 | RASQSVSSA | 206 | SASSLYS | 258 | SFSSPV | 310 |
| P9-54 | RASQSVSSA | 211 | SASSLYS | 263 | WYPSLI | 315 |
| P9-58 | RASQSVSSA | 214 | SASSLYS | 266 | GFGFLV | 318 |

Table 5 presents the full CDR sequences, according to multiple art-accepted definitions, for the 22 candidate anti-GAL9 immune-activating antibodies.

TABLE 5
CDR definitions

| | | | | | |
|---|---|---|---|---|---|
| | | | | | SEQ |
| Re- | Defini- | | Resi- | | ID |
| gion | tion | Sequence | dues | Length | NO: |

P9-02B

| | | | | | |
|---|---|---|---|---|---|
| CDR- | Chothia | GFTFSGY--- | 26-32 | 7 | 319 |
| H1 | | | | | |
| | AbM | GFTFSGYFIH | 26-35 | 10 | 320 |
| | Kabat | -----GYFIH | 31-35 | 5 | 321 |
| | Contact | ----SGYFIH | 30-35 | 6 | 322 |
| | IMGT | GFTFSGYF-- | 26-33 | 8 | 323 |
| CDR- | Chothia | -----SSYGGH------ | 52-57 | 6 | 324 |
| H2 | | --- | | | |
| | AbM | ---RISSYGGHTD---- | 50-59 | 10 | 325 |
| | | --- | | | |
| | Kabat | --- | 50-66 | 17 | 326 |
| | | RISSYGGHTDYADSVKG | | | |
| | Contact | WVARISSYGGHTD---- | 47-59 | 13 | 327 |
| | | --- | | | |
| | IMGT | ----ISSYGGHT----- | 51-58 | 8 | 328 |
| | | --- | | | |
| CDR- | Chothia | --AQYVPGLDY | 99-107 | 9 | 329 |
| H3 | | | | | |
| | AbM | --AQYVPGLDY | 99-107 | 9 | 330 |
| | Kabat | --AQYVPGLDY | 99-107 | 9 | 331 |
| | Contact | ARAQYVPGLD- | 97-106 | 10 | 332 |
| | IMGT | ARAQYVPGLDY | 97-107 | 11 | 333 |
| CDR- | Chothia | RASQSVSSAVA-- | 24-34 | 11 | 334 |
| L1 | | | | | |
| | AbM | RASQSVSSAVA-- | 24-34 | 11 | 335 |
| | Kabat | RASQSVSSAVA-- | 24-34 | 11 | 336 |
| | Contact | ------SSAVAWY | 30-36 | 7 | 337 |
| | IMGT | ---QSVSSA---- | 27-32 | 6 | 338 |
| CDR- | Chothia | ----SASSLYS | 50-56 | 7 | 339 |
| L2 | | | | | |
| | AbM | ----SASSLYS | 50-56 | 7 | 340 |
| | Kabat | ----SASSLYS | 50-56 | 7 | 341 |
| | Contact | LLIYSASSLY- | 46-55 | 10 | 342 |
| | IMGT | ----SA----- | 50-51 | 2 | 343 |
| CDR- | Chothia | QQSYPTLGT | 89-97 | 9 | 344 |
| L3 | | | | | |
| | AbM | QQSYPTLGT | 89-97 | 9 | 345 |
| | Kabat | QQSYPTLGT | 89-97 | 9 | 346 |
| | Contact | QQSYPTLG- | 89-96 | 8 | 347 |
| | IMGT | QQSYPTLGT | 89-97 | 9 | 348 |

P9-04

| | | | | | |
|---|---|---|---|---|---|
| CDR- | Chothia | GFTFGSY--- | 26-32 | 7 | 349 |
| H1 | | | | | |
| | AbM | GFTFGSYFIH | 26-35 | 10 | 350 |
| | Kabat | -----SYFIH | 31-35 | 5 | 351 |
| | Contact | ----GSYFIH | 30-35 | 6 | 352 |
| | IMGT | GFTFGSYF-- | 26-33 | 8 | 353 |
| CDR- | Chothia | -----SPTWGY------ | 52-57 | 6 | 354 |
| H2 | | --- | | | |
| | AbM | ---YISPTWGYTY---- | 50-59 | 10 | 355 |
| | | --- | | | |
| | Kabat | --- | 50-66 | 17 | 356 |
| | | YISPTWGYTYYADSVKG | | | |
| | Contact | WVAYISPTWGYTY---- | 47-59 | 13 | 357 |
| | | --- | | | |
| | IMGT | ----ISPTWGYT----- | 51-58 | 8 | 358 |
| | | --- | | | |
| CDR- | Chothia | --AWFGFAFDY | 99-107 | 9 | 359 |
| H3 | | | | | |
| | AbM | --AWFGFAFDY | 99-107 | 9 | 360 |
| | Kabat | --AWFGFAFDY | 99-107 | 9 | 361 |
| | Contact | ARAWFGFAFD- | 97-106 | 10 | 362 |
| | IMGT | ARAWFGFAFDY | 97-107 | 11 | 363 |
| CDR- | Chothia | RASQSVSSAVA-- | 24-34 | 11 | 364 |
| L1 | | | | | |
| | AbM | RASQSVSSAVA-- | 24-34 | 11 | 365 |
| | Kabat | RASQSVSSAVA-- | 24-34 | 11 | 366 |
| | Contact | ------SSAVAWY | 30-36 | 7 | 367 |
| | IMGT | ---QSVSSA---- | 27-32 | 6 | 368 |
| CDR- | Chothia | ----SASSLYS | 50-56 | 7 | 369 |
| L2 | | | | | |
| | AbM | ----SASSLYS | 50-56 | 7 | 370 |
| | Kabat | ----SASSLYS | 50-56 | 7 | 371 |
| | Contact | LLIYSASSLY- | 46-55 | 10 | 372 |
| | IMGT | ----SA----- | 50-51 | 2 | 373 |
| CDR- | Chothia | QQVYSSPLT | 89-97 | 9 | 374 |
| L3 | | | | | |
| | AbM | QQVYSSPLT | 89-97 | 9 | 375 |
| | Kabat | QQVYSSPLT | 89-97 | 9 | 376 |
| | Contact | QQVYSSPL- | 89-96 | 8 | 377 |
| | IMGT | QQVYSSPLT | 89-97 | 9 | 378 |

P9-05

| | | | | | |
|---|---|---|---|---|---|
| CDR- | Chothia | GFTFTSY--- | 26-32 | 7 | 379 |
| H1 | | | | | |
| | AbM | GFTFTSYYIH | 26-35 | 10 | 380 |
| | Kabat | -----SYYIH | 31-35 | 5 | 381 |
| | Contact | ----TSYYIH | 30-35 | 6 | 382 |
| | IMGT | GFTFTSYY-- | 26-33 | 8 | 383 |
| CDR- | Chothia | -----WPIGGY------ | 52-57 | 6 | 384 |
| H2 | | --- | | | |
| | AbM | ---WIWPIGGYTY---- | 50-59 | 10 | 385 |
| | | --- | | | |
| | Kabat | --- | 50-66 | 17 | 386 |
| | | WIWPIGGYTYYADSVKG | | | |
| | Contact | WVAWIWPIGGYTY---- | 47-59 | 13 | 387 |
| | | --- | | | |
| | IMGT | ----IWPIGGYT----- | 51-58 | 8 | 388 |
| | | --- | | | |
| CDR- | Chothia | --DAGSGFDY | 99-106 | 8 | 389 |
| H3 | | | | | |
| | AbM | --DAGSGFDY | 99-106 | 8 | 390 |
| | Kabat | --DAGSGFDY | 99-106 | 8 | 391 |
| | Contact | ARDAGSGFD- | 97-105 | 9 | 392 |
| | IMGT | ARDAGSGFDY | 97-106 | 10 | 393 |
| CDR- | Chothia | RASQSVSSAVA-- | 24-34 | 11 | 394 |
| L1 | | | | | |
| | AbM | RASQSVSSAVA-- | 24-34 | 11 | 395 |
| | Kabat | RASQSVSSAVA-- | 24-34 | 11 | 396 |
| | Contact | ------SSAVAWY | 30-36 | 7 | 397 |
| | IMGT | ---QSVSSA---- | 27-32 | 6 | 398 |
| CDR- | Chothia | ----SASSLYS | 50-56 | 7 | 399 |
| L2 | | | | | |
| | AbM | ----SASSLYS | 50-56 | 7 | 400 |
| | Kabat | ----SASSLYS | 50-56 | 7 | 401 |
| | Contact | LLIYSASSLY- | 46-55 | 10 | 402 |
| | IMGT | ----SA----- | 50-51 | 2 | 403 |
| CDR- | Chothia | QQYYYSLLT | 89-97 | 9 | 404 |
| L3 | | | | | |
| | AbM | QQYYYSLLT | 89-97 | 9 | 405 |
| | Kabat | QQYYYSLLT | 89-97 | 9 | 406 |
| | Contact | QQYYYSLL- | 89-96 | 8 | 407 |
| | IMGT | QQYYYSLLT | 89-97 | 9 | 408 |

P9-08

| | | | | | |
|---|---|---|---|---|---|
| CDR- | Chothia | GFTFSRY--- | 26-32 | 7 | 409 |
| H1 | | | | | |
| | AbM | GFTFSRYAIH | 26-35 | 10 | 410 |
| | Kabat | -----RYAIH | 31-35 | 5 | 411 |
| | Contact | ----SRYAIH | 30-35 | 6 | 412 |
| | IMGT | GFTFSRYA-- | 26-33 | 8 | 413 |
| CDR- | Chothia | -----YSPTGY------ | 52-57 | 6 | 414 |
| H2 | | --- | | | |
| | AbM | ---AIYSPTGYTD---- | 50-59 | 10 | 415 |
| | | --- | | | |
| | Kabat | --- | 50-66 | 17 | 416 |
| | | AIYSPTGYTDYADSVKG | | | |
| | Contact | WVAAIYSPTGYTD---- | 47-59 | 13 | 417 |
| | | --- | | | |
| | IMGT | ----IYSPTGYT----- | 51-58 | 8 | 418 |
| | | --- | | | |
| CDR- | Chothia | --EGYIGMDY | 99-106 | 8 | 419 |
| H3 | | | | | |
| | AbM | --EGYIGMDY | 99-106 | 8 | 420 |
| | Kabat | --EGYIGMDY | 99-106 | 8 | 421 |
| | Contact | AREGYIGMD- | 97-105 | 9 | 422 |
| | IMGT | AREGYIGMDY | 97-106 | 10 | 423 |
| CDR- | Chothia | RASQSVSSAVA-- | 24-34 | 11 | 424 |
| L1 | | | | | |
| | AbM | RASQSVSSAVA-- | 24-34 | 11 | 425 |
| | Kabat | RASQSVSSAVA-- | 24-34 | 11 | 426 |
| | Contact | ------SSAVAWY | 30-36 | 7 | 427 |
| | IMGT | ---QSVSSA---- | 27-32 | 6 | 428 |
| CDR- | Chothia | ----SASSLYS | 50-56 | 7 | 429 |
| L2 | | | | | |
| | AbM | ----SASSLYS | 50-56 | 7 | 430 |
| | Kabat | ----SASSLYS | 50-56 | 7 | 431 |
| | Contact | LLIYSASSLY- | 46-55 | 10 | 432 |
| | IMGT | ----SA----- | 50-51 | 2 | 433 |
| CDR- | Chothia | QQSYSALYT | 89-97 | 9 | 434 |
| L3 | | | | | |
| | AbM | QQSYSALYT | 89-97 | 9 | 435 |
| | Kabat | QQSYSALYT | 89-97 | 9 | 436 |
| | Contact | QQSYSALY- | 89-96 | 8 | 437 |
| | IMGT | QQSYSALYT | 89-97 | 9 | 438 |

P9-09

| | | | | | |
|---|---|---|---|---|---|
| CDR- | Chothia | GFTFSSY--- | 26-32 | 7 | 439 |
| H1 | | | | | |
| | AbM | GFTFSSYFIH | 26-35 | 10 | 440 |
| | Kabat | -----SYFIH | 31-35 | 5 | 441 |
| | Contact | ----SSYFIH | 30-35 | 6 | 442 |
| | IMGT | GFTFSSYF-- | 26-33 | 8 | 443 |
| CDR- | Chothia | -----YSSGGY------ | 52-57 | 6 | 444 |
| H2 | | --- | | | |
| | AbM | ---WIYSSGGYTY---- | 50-59 | 10 | 445 |
| | | --- | | | |
| | Kabat | --- | 50-66 | 17 | 446 |
| | | WIYSSGGYTYYADSVKG | | | |
| | Contact | WVAWIYSSGGYTY---- | 47-59 | 13 | 447 |
| | | --- | | | |
| | IMGT | ----IYSSGGYT----- | 51-58 | 8 | 448 |
| | | --- | | | |
| CDR- | Chothia | --FTPSDLGYGLDY | 99-110 | 12 | 449 |
| H3 | | | | | |
| | AbM | --FTPSDLGYGLDY | 99-110 | 12 | 450 |
| | Kabat | --FTPSDLGYGLDY | 99-110 | 12 | 451 |
| | Contact | ARFTPSDLGYGLD- | 97-109 | 13 | 452 |
| | IMGT | ARFTPSDLGYGLDY | 97-110 | 14 | 453 |
| CDR- | Chothia | RASQSVSSAVA-- | 24-34 | 11 | 454 |
| L1 | | | | | |
| | AbM | RASQSVSSAVA-- | 24-34 | 11 | 455 |
| | Kabat | RASQSVSSAVA-- | 24-34 | 11 | 456 |
| | Contact | ------SSAVAWY | 30-36 | 7 | 457 |
| | IMGT | ---QSVSSA---- | 27-32 | 6 | 458 |
| CDR- | Chothia | ----SASSLYS | 50-56 | 7 | 459 |
| L2 | | | | | |
| | AbM | ----SASSLYS | 50-56 | 7 | 460 |
| | Kabat | ----SASSLYS | 50-56 | 7 | 461 |
| | Contact | LLIYSASSLY- | 46-55 | 10 | 462 |
| | IMGT | ----SA----- | 50-51 | 2 | 463 |
| CDR- | Chothia | QQADPSLVT | 89-97 | 9 | 464 |
| L3 | | | | | |
| | AbM | QQADPSLVT | 89-97 | 9 | 465 |
| | Kabat | QQADPSLVT | 89-97 | 9 | 466 |
| | Contact | QQADPSLV- | 89-96 | 8 | 467 |
| | IMGT | QQADPSLVT | 89-97 | 9 | 468 |

P9-10

| | | | | | |
|---|---|---|---|---|---|
| CDR- | Chothia | GFTFDYY--- | 26-32 | 7 | 469 |
| H1 | | | | | |
| | AbM | GFTFDYYVIH | 26-35 | 10 | 470 |
| | Kabat | -----YYVIH | 31-35 | 5 | 471 |
| | Contact | ----DYYVIH | 30-35 | 6 | 472 |
| | IMGT | GFTFDYYV-- | 26-33 | 8 | 473 |
| CDR- | Chothia | -----DSYWGD------ | 52-57 | 6 | 474 |
| H2 | | --- | | | |
| | AbM | ---AIDSYWGDTY---- | 50-59 | 10 | 475 |
| | | --- | | | |
| | Kabat | --- | 50-66 | 17 | 476 |
| | | AIDSYWGDTYYADSVKG | | | |
| | Contact | WVAAIDSYWGDTY---- | 47-59 | 13 | 477 |
| | | --- | | | |
| | IMGT | ----IDSYWGDT----- | 51-58 | 8 | 478 |
| | | --- | | | |
| CDR- | Chothia | --FYFYGFDY | 99-106 | 8 | 479 |
| H3 | | | | | |
| | AbM | --FYFYGFDY | 99-106 | 8 | 480 |
| | Kabat | --FYFYGFDY | 99-106 | 8 | 481 |
| | Contact | ARFYFYGFD- | 97-105 | 9 | 482 |
| | IMGT | ARFYFYGFDY | 97-106 | 10 | 483 |
| CDR- | Chothia | RASQSVSSAVA-- | 24-34 | 11 | 484 |
| L1 | | | | | |
| | AbM | RASQSVSSAVA-- | 24-34 | 11 | 485 |
| | Kabat | RASQSVSSAVA-- | 24-34 | 11 | 486 |
| | Contact | ------SSAVAWY | 30-36 | 7 | 487 |
| | IMGT | ---QSVSSA---- | 27-32 | 6 | 488 |
| CDR- | Chothia | ----SASSLYS | 50-56 | 7 | 489 |
| L2 | | | | | |
| | AbM | ----SASSLYS | 50-56 | 7 | 490 |
| | Kabat | ----SASSLYS | 50-56 | 7 | 491 |
| | Contact | LLIYSASSLY- | 46-55 | 10 | 492 |
| | IMGT | ----SA----- | 50-51 | 2 | 493 |
| CDR- | Chothia | QQYSWSLWT | 89-97 | 9 | 494 |
| L3 | | | | | |
| | AbM | QQYSWSLWT | 89-97 | 9 | 495 |
| | Kabat | QQYSWSLWT | 89-97 | 9 | 496 |
| | Contact | QQYSWSLW- | 89-96 | 8 | 497 |
| | IMGT | QQYSWSLWT | 89-97 | 9 | 498 |

P9-15

| | | | | | |
|---|---|---|---|---|---|
| CDR- | Chothia | GFTFRYY--- | 26-32 | 7 | 499 |
| H1 | | | | | |
| | AbM | GFTFRYYWIH | 26-35 | 10 | 500 |
| | Kabat | -----YYWIH | 31-35 | 5 | 501 |
| | Contact | ----RYYWIH | 30-35 | 6 | 502 |
| | IMGT | GFTFRYYW-- | 26-33 | 8 | 503 |
| CDR- | Chothia | -----FPSGGI------ | 52-57 | 6 | 504 |
| H2 | | --- | | | |
| | AbM | ---AIFPSGGITT---- | 50-59 | 10 | 505 |
| | | --- | | | |
| | Kabat | --- | 50-66 | 17 | 506 |
| | | AIFPSGGITTYADSVKG | | | |
| | Contact | WVAAIFFSGGITT---- | 47-59 | 13 | 507 |
| | | --- | | | |
| | IMGT | ----IFPSGGIT----- | 51-58 | 8 | 508 |
| | | --- | | | |
| CDR- | Chothia | --GWPWGLDY | 99-106 | 8 | 509 |
| H3 | | | | | |
| | AbM | --GWPWGLDY | 99-106 | 8 | 510 |
| | Kabat | --GWPWGLDY | 99-106 | 8 | 511 |
| | Contact | ARGWPWGLD- | 97-105 | 9 | 512 |
| | IMGT | ARGWPWGLDY | 97-106 | 10 | 513 |
| CDR- | Chothia | RASQSVSSAVA-- | 24-34 | 11 | 514 |
| L1 | | | | | |
| | AbM | RASQSVSSAVA-- | 24-34 | 11 | 515 |
| | Kabat | RASQSVSSAVA-- | 24-34 | 11 | 516 |
| | Contact | ------SSAVAWY | 30-36 | 7 | 517 |
| | IMGT | ---QSVSSA---- | 27-32 | 6 | 518 |
| CDR- | Chothia | ----SASSLYS | 50-56 | 7 | 519 |
| L2 | | | | | |
| | AbM | ----SASSLYS | 50-56 | 7 | 520 |
| | Kabat | ----SASSLYS | 50-56 | 7 | 521 |
| | Contact | LLIYSASSLY- | 46-55 | 10 | 522 |
| | IMGT | ----SA----- | 50-51 | 2 | 523 |
| CDR- | Chothia | QQRDRSPYT | 89-97 | 9 | 524 |
| L3 | | | | | |
| | AbM | QQRDRSPYT | 89-97 | 9 | 525 |
| | Kabat | QQRDRSPYT | 89-97 | 9 | 526 |
| | Contact | QQRDRSPY- | 89-96 | 8 | 527 |
| | IMGT | QQRDRSPYT | 89-97 | 9 | 528 |

P9-16

| | | | | | |
|---|---|---|---|---|---|
| CDR- | Chothia | GFTFSYY--- | 26-32 | 7 | 529 |
| H1 | | | | | |
| | AbM | GFTFSYYWIH | 26-35 | 10 | 530 |
| | Kabat | -----YYWIH | 31-35 | 5 | 531 |
| | Contact | ----SYYWIH | 30-35 | 6 | 532 |
| | IMGT | GFTFSYYW-- | 26-33 | 8 | 533 |
| CDR- | Chothia | -----YPSSGY------ | 52-57 | 6 | 534 |
| H2 | | --- | | | |
| | AbM | ---DIYPSSGYTY---- | 50-59 | 10 | 535 |
| | | --- | | | |
| | Kabat | --- | 50-66 | 17 | 536 |
| | | DIYPSSGYTYYADSVKG | | | |
| | Contact | WVADIYPSSGYTY---- | 47-59 | 13 | 537 |
| | | --- | | | |
| | IMGT | ----IYPSSGYT----- | 51-58 | 8 | 538 |
| | | --- | | | |
| CDR- | Chothia | --GWYAAYGMDY | 99-108 | 10 | 539 |
| H3 | | | | | |
| | AbM | --GWYAAYGMDY | 99-108 | 10 | 540 |
| | Kabat | --GWYAAYGMDY | 99-108 | 10 | 541 |
| | Contact | ARGWYAAYGMD- | 97-107 | 11 | 542 |
| | IMGT | ARGWYAAYGMDY | 97-108 | 12 | 543 |
| CDR- | Chothia | RASQSVSSAVA-- | 24-34 | 11 | 544 |
| L1 | | | | | |
| | AbM | RASQSVSSAVA-- | 24-34 | 11 | 545 |
| | Kabat | RASQSVSSAVA-- | 24-34 | 11 | 546 |
| | Contact | ------SSAVAWY | 30-36 | 7 | 547 |
| | IMGT | ---QSVSSA---- | 27-32 | 6 | 548 |
| CDR- | Chothia | ----SASSLYS | 50-56 | 7 | 549 |
| L2 | | | | | |
| | AbM | ----SASSLYS | 50-56 | 7 | 550 |
| | Kabat | ----SASSLYS | 50-56 | 7 | 551 |
| | Contact | LLIYSASSLY- | 46-55 | 10 | 552 |
| | IMGT | ----SA----- | 50-51 | 2 | 553 |
| CDR- | Chothia | QQYALRPLT | 89-97 | 9 | 554 |
| L3 | | | | | |
| | AbM | QQYALRPLT | 89-97 | 9 | 555 |
| | Kabat | QQYALRPLT | 89-97 | 9 | 556 |
| | Contact | QQYALRPL- | 89-96 | 8 | 557 |
| | IMGT | QQYALRPLT | 89-97 | 9 | 558 |

P9-18

| | | | | | |
|---|---|---|---|---|---|
| CDR- | Chothia | GFTFSWY--- | 26-32 | 7 | 559 |
| H1 | | | | | |
| | AbM | GFTFSWYVIH | 26-35 | 10 | 560 |
| | Kabat | -----WYVIH | 31-35 | 5 | 561 |
| | Contact | ----SWYVIH | 30-35 | 6 | 562 |
| | IMGT | GFTFSWYV-- | 26-33 | 8 | 563 |
| CDR- | Chothia | -----YPYHSK------ | 52-57 | 6 | 564 |
| H2 | | --- | | | |
| | AbM | ---AIYPYHSKTY---- | 50-59 | 10 | 565 |
| | | --- | | | |
| | Kabat | --- | 50-66 | 17 | 566 |
| | | AIYPYHSKTYYADSVKG | | | |
| | Contact | WVAAIYPYHSKTY---- | 47-59 | 13 | 567 |
| | | --- | | | |
| | IMGT | ----IYPYHSKT----- | 51-58 | 8 | 568 |
| | | --- | | | |
| CDR- | Chothia | --GYWYGMDY | 99-106 | 8 | 569 |
| H3 | | | | | |
| | AbM | --GYWYGMDY | 99-106 | 8 | 570 |
| | Kabat | --GYWYGMDY | 99-106 | 8 | 571 |
| | Contact | ARGYWYGMD- | 97-105 | 9 | 572 |
| | IMGT | ARGYWYGMDY | 97-106 | 10 | 573 |
| CDR- | Chothia | RASQSVSSAVA-- | 24-34 | 11 | 574 |
| L1 | | | | | |
| | AbM | RASQSVSSAVA-- | 24-34 | 11 | 575 |
| | Kabat | RASQSVSSAVA-- | 24-34 | 11 | 576 |
| | Contact | ------SSAVAWY | 30-36 | 7 | 577 |
| | IMGT | ---QSVSSA---- | 27-32 | 6 | 578 |
| CDR- | Chothia | ----SASSLYS | 50-56 | 7 | 579 |
| L2 | | | | | |
| | AbM | ----SASSLYS | 50-56 | 7 | 580 |
| | Kabat | ----SASSLYS | 50-56 | 7 | 581 |
| | Contact | LLIYSASSLY- | 46-55 | 10 | 582 |
| | IMGT | ----SA----- | 50-51 | 2 | 583 |
| CDR- | Chothia | QQYTDAPWT | 89-97 | 9 | 584 |
| L3 | | | | | |
| | AbM | QQYTDAPWT | 89-97 | 9 | 585 |
| | Kabat | QQYTDAPWT | 89-97 | 9 | 586 |
| | Contact | QQYTDAPW- | 89-96 | 8 | 587 |
| | IMGT | QQYTDAPWT | 89-97 | 9 | 588 |

P9-19

| | | | | | |
|---|---|---|---|---|---|
| CDR- | Chothia | GFTFGYY--- | 26-32 | 7 | 589 |
| H1 | | | | | |
| | AbM | GFTFGYYYIH | 26-35 | 10 | 590 |
| | Kabat | -----YYYIH | 31-35 | 5 | 591 |
| | Contact | ----GYYYIH | 30-35 | 6 | 592 |
| | IMGT | GFTFGYYY-- | 26-33 | 8 | 593 |
| CDR- | Chothia | -----SPSGSV------ | 52-57 | 6 | 594 |
| H2 | | --- | | | |
| | AbM | ---WISPSGSVTA---- | 50-59 | 10 | 595 |
| | | --- | | | |
| | Kabat | --- | 50-66 | 17 | 596 |
| | | WISPSGSVTAYADSVKG | | | |
| | Contact | WVAWISPSGSVTA---- | 47-59 | 13 | 597 |
| | | --- | | | |
| | IMGT | ----ISPSGSVT----- | 51-58 | 8 | 598 |
| | | --- | | | |
| CDR- | Chothia | --GYYGGWGMDY | 99-108 | 10 | 599 |
| H3 | | | | | |
| | AbM | --GYYGGWGMDY | 99-108 | 10 | 600 |
| | Kabat | --GYYGGWGMDY | 99-108 | 10 | 601 |
| | Contact | ARGYYGGWGMD- | 97-107 | 11 | 602 |
| | IMGT | ARGYYGGWGMDY | 97-108 | 12 | 603 |
| CDR- | Chothia | RASQSVSSAVA-- | 24-34 | 11 | 604 |
| L1 | | | | | |
| | AbM | RASQSVSSAVA-- | 24-34 | 11 | 605 |
| | Kabat | RASQSVSSAVA-- | 24-34 | 11 | 606 |
| | Contact | ------SSAVAWY | 30-36 | 7 | 607 |
| | IMGT | ---QSVSSA---- | 27-32 | 6 | 608 |
| CDR- | Chothia | ----SASSLYS | 50-56 | 7 | 609 |
| L2 | | | | | |
| | AbM | ----SASSLYS | 50-56 | 7 | 610 |
| | Kabat | ----SASSLYS | 50-56 | 7 | 611 |
| | Contact | LLIYSASSLY- | 46-55 | 10 | 612 |
| | IMGT | ----SA----- | 50-51 | 2 | 613 |
| CDR- | Chothia | QQYDTSPYT | 89-97 | 9 | 614 |
| L3 | | | | | |
| | AbM | QQYDTSPYT | 89-97 | 9 | 615 |
| | Kabat | QQYDTSPYT | 89-97 | 9 | 616 |
| | Contact | QQYDTSPY- | 89-96 | 8 | 617 |
| | IMGT | QQYDTSPYT | 89-97 | 9 | 618 |

P9-20

| | | | | | |
|---|---|---|---|---|---|
| CDR- | Chothia | GFTFRYY--- | 26-32 | 7 | 619 |
| H1 | | | | | |
| | AbM | GFTFRYYYIH | 26-35 | 10 | 620 |
| | Kabat | -----YYYIH | 31-35 | 5 | 621 |
| | Contact | ----RYYYIH | 30-35 | 6 | 622 |
| | IMGT | GFTFRYYY-- | 26-33 | 8 | 623 |
| CDR- | Chothia | -----YPYGGY------ | 52-57 | 6 | 624 |
| H2 | | --- | | | |
| | AbM | ---GIYPYGGYTS---- | 50-59 | 10 | 625 |
| | | --- | | | |
| | Kabat | --- | 50-66 | 17 | 626 |
| | | GIYPYGGYTSYADSVKG | | | |
| | Contact | WVAGIYPYGGYTS---- | 47-59 | 13 | 627 |
| | | --- | | | |
| | IMGT | ----IYPYGGYT----- | 51-58 | 8 | 628 |
| | | --- | | | |
| CDR- | Chothia | --GYYVEGVLDY | 99-108 | 10 | 629 |
| H3 | | | | | |
| | AbM | --GYYVEGVLDY | 99-108 | 10 | 630 |
| | Kabat | --GYYVEGVLDY | 99-108 | 10 | 631 |
| | Contact | ARGYYVEGVLD- | 97-107 | 11 | 632 |
| | IMGT | ARGYYVEGVLDY | 97-108 | 12 | 633 |
| CDR- | Chothia | RASQSVSSAVA-- | 24-34 | 11 | 634 |
| L1 | | | | | |
| | AbM | RASQSVSSAVA-- | 24-34 | 11 | 635 |
| | Kabat | RASQSVSSAVA-- | 24-34 | 11 | 636 |
| | Contact | ------SSAVAWY | 30-36 | 7 | 637 |
| | IMGT | ---QSVSSA---- | 27-32 | 6 | 638 |
| CDR- | Chothia | ----SASSLYS | 50-56 | 7 | 639 |
| L2 | | | | | |
| | AbM | ----SASSLYS | 50-56 | 7 | 640 |
| | Kabat | ----SASSLYS | 50-56 | 7 | 641 |
| | Contact | LLIYSASSLY- | 46-55 | 10 | 642 |
| | IMGT | ----SA----- | 50-51 | 2 | 643 |
| CDR- | Chothia | QQYYGSLAT | 89-97 | 9 | 644 |
| L3 | | | | | |
| | AbM | QQYYGSLAT | 89-97 | 9 | 645 |
| | Kabat | QQYYGSLAT | 89-97 | 9 | 646 |
| | Contact | QQYYGSLA- | 89-96 | 8 | 647 |
| | IMGT | QQYYGSLAT | 89-97 | 9 | 648 |

P9-21

| | | | | | |
|---|---|---|---|---|---|
| CDR- | Chothia | GFTFSYY--- | 26-32 | 7 | 649 |
| H1 | | | | | |
| | AbM | GFTFSYYYIH | 26-35 | 10 | 650 |
| | Kabat | -----YYYIH | 31-35 | 5 | 651 |
| | Contact | ----SYYYIH | 30-35 | 6 | 652 |
| | IMGT | GFTFSYYY-- | 26-33 | 8 | 653 |
| CDR- | Chothia | -----HPPSGY------ | 52-57 | 6 | 654 |
| H2 | | --- | | | |
| | AbM | ---RIHPPSGYTD---- | 50-59 | 10 | 655 |
| | | --- | | | |
| | Kabat | --- | 50-66 | 17 | 656 |
| | | RIHPPSGYTDYADSVKG | | | |
| | Contact | WVARIHPFSGYTD---- | 47-59 | 13 | 657 |
| | | --- | | | |
| | IMGT | ----IHPPSGYT----- | 51-58 | 8 | 658 |
| | | --- | | | |
| CDR- | Chothia | --GYYVFGVMDY | 99-108 | 10 | 659 |
| H3 | | | | | |
| | AbM | --GYYVFGVMDY | 99-108 | 10 | 660 |
| | Kabat | --GYYVFGVMDY | 99-108 | 10 | 661 |
| | Contact | ARGYYVFGVMD- | 97-107 | 11 | 662 |
| | IMGT | ARGYYVFGVMDY | 97-108 | 12 | 663 |
| CDR- | Chothia | RASQSVSSAVA-- | 24-34 | 11 | 664 |
| L1 | | | | | |
| | AbM | RASQSVSSAVA-- | 24-34 | 11 | 665 |
| | Kabat | RASQSVSSAVA-- | 24-34 | 11 | 666 |
| | Contact | ------SSAVAWY | 30-36 | 7 | 667 |
| | IMGT | ---QSVSSA---- | 27-2 | 6 | 668 |
| CDR- | Chothia | ----SASSLYS | 50-56 | 7 | 669 |
| L2 | | | | | |
| | AbM | ----SASSLYS | 50-56 | 7 | 670 |
| | Kabat | ----SASSLYS | 50-56 | 7 | 671 |
| | Contact | LLIYSASSLY- | 46-55 | 10 | 672 |
| | IMGT | ----SA----- | 50-51 | 2 | 673 |
| CDR- | Chothia | QQSHWYPFT | 89-97 | 9 | 674 |
| L3 | | | | | |
| | AbM | QQSHWYPFT | 89-97 | 9 | 675 |
| | Kabat | QQSHWYPFT | 89-97 | 9 | 676 |
| | Contact | QQSHWYPF- | 89-96 | 8 | 677 |
| | IMGT | QQSHWYPFT | 89-97 | 9 | 678 |

P9-22

| | | | | | |
|---|---|---|---|---|---|
| CDR- | Chothia | GFTFSSY--- | 26-32 | 7 | 679 |
| H1 | | | | | |
| | AbM | GFTFSSYYIH | 26-35 | 10 | 680 |
| | Kabat | -----SYYIH | 31-35 | 5 | 681 |
| | Contact | ----SSYYIH | 30-35 | 6 | 682 |
| | IMGT | GFTFSSYY-- | 26-33 | 8 | 683 |
| CDR- | Chothia | -----YPFSGG------ | 52-57 | 6 | 684 |
| H2 | | --- | | | |
| | AbM | ---AIYPFSGGTY---- | 50-59 | 10 | 685 |
| | | --- | | | |
| | Kabat | --- | 50-66 | 17 | 686 |
| | | AIYPFSGGTYYADSVKG | | | |
| | Contact | WVAAIYPFSGGTY---- | 47-59 | 13 | 687 |
| | | --- | | | |
| | IMGT | ----IYPFSGGT----- | 51-58 | 8 | 688 |
| | | --- | | | |
| CDR- | Chothia | --GYYVYVVMDY | 99-108 | 10 | 689 |
| H3 | | | | | |
| | AbM | --GYYVYVVMDY | 99-108 | 10 | 690 |
| | Kabat | --GYYVYVVMDY | 99-108 | 10 | 691 |
| | Contact | ARGYYVYVVMD- | 97-107 | 11 | 692 |
| | IMGT | ARGYYVYVVMDY | 97-108 | 12 | 693 |
| CDR- | Chothia | RASQSVSSAVA-- | 24-34 | 11 | 694 |
| L1 | | | | | |
| | AbM | RASQSVSSAVA-- | 24-34 | 11 | 695 |
| | Kabat | RASQSVSSAVA-- | 24-34 | 11 | 696 |
| | Contact | ------SSAVAWY | 30-36 | 7 | 697 |
| | IMGT | ---QSVSSA---- | 27-32 | 6 | 698 |
| CDR- | Chothia | ----SASSLYS | 50-56 | 7 | 699 |
| L2 | | | | | |
| | AbM | ----SASSLYS | 50-56 | 7 | 700 |
| | Kabat | ----SASSLYS | 50-56 | 7 | 701 |
| | Contact | LLIYSASSLY- | 46-55 | 10 | 702 |
| | IMGT | ----SA----- | 50-51 | 2 | 703 |
| CDR- | Chothia | QQYKSSPWT | 89-97 | 9 | 704 |
| L3 | | | | | |
| | AbM | QQYKSSPWT | 89-97 | 9 | 705 |
| | Kabat | QQYKSSPWT | 89-97 | 9 | 706 |
| | Contact | QQYKSSPW- | 89-96 | 8 | 707 |
| | IMGT | QQYKSSPWT | 89-97 | 9 | 708 |

P9-27

| | | | | | |
|---|---|---|---|---|---|
| CDR- | Chothia | GFTFWGY--- | 26-32 | 7 | 709 |
| H1 | | | | | |
| | AbM | GFTFWGYGIH | 26-35 | 10 | 710 |
| | Kabat | -----GYGIH | 31-35 | 5 | 711 |
| | Contact | ----WGYGIH | 30-35 | 6 | 712 |
| | IMGT | GFTFWGYG-- | 26-33 | 8 | 713 |
| CDR- | Chothia | -----YPYGGS------ | 52-57 | 6 | 714 |
| H2 | | --- | | | |
| | AbM | AIYPYGGSTY---- | 50-59 | 10 | 715 |
| | | --- | | | |
| | Kabat | --- | 50-66 | 17 | 716 |
| | | AIYPYGGSTYYADSVKG | | | |
| | Contact | WVAAIYPYGGSTY---- | 47-59 | 13 | 717 |
| | | --- | | | |
| | IMGT | ----IYPYGGST----- | 51-58 | 8 | 718 |
| | | --- | | | |
| CDR- | Chothia | --LSDIYHSFSGMDY | 99-111 | 13 | 719 |
| H3 | | | | | |
| | AbM | --LSDIYHSFSGMDY | 99-111 | 13 | 720 |
| | Kabat | --LSDIYHSFSGMDY | 99-111 | 13 | 721 |
| | Contact | ARLSDIYHSFSGMD- | 97-110 | 14 | 722 |
| | IMGT | ARLSDIYHSFSGMDY | 97-111 | 15 | 723 |
| CDR- | Chothia | RASQSVSSAVA-- | 24-34 | 11 | 724 |
| L1 | | | | | |
| | AbM | RASQSVSSAVA-- | 24-34 | 11 | 725 |
| | Kabat | RASQSVSSAVA-- | 24-34 | 11 | 726 |
| | Contact | ------SSAVAWY | 30-36 | 7 | 727 |
| | IMGT | ---QSVSSA---- | 27-32 | 6 | 728 |
| CDR- | Chothia | ----SASSLYS | 50-56 | 7 | 729 |
| L2 | | | | | |
| | AbM | ----SASSLYS | 50-56 | 7 | 730 |
| | Kabat | ----SASSLYS | 50-56 | 7 | 731 |
| | Contact | LLIYSASSLY- | 46-55 | 10 | 732 |
| | IMGT | ----SA----- | 50-51 | 2 | 733 |
| CDR- | Chothia | QQRYSTPVT | 89-97 | 9 | 734 |
| L3 | | | | | |
| | AbM | QQRYSTPVT | 89-97 | 9 | 735 |
| | Kabat | QQRYSTPVT | 89-97 | 9 | 736 |
| | Contact | QQRYSTPV- | 89-96 | 8 | 737 |
| | IMGT | QQRYSTPVT | 89-97 | 9 | 738 |

P9-28

| | | | | | |
|---|---|---|---|---|---|
| CDR- | Chothia | GFTFGFY--- | 26-32 | 7 | 739 |
| H1 | | | | | |
| | AbM | GFTFGFYYIH | 26-35 | 10 | 740 |
| | Kabat | -----FYYIH | 31-35 | 5 | 741 |
| | Contact | ----GFYYIH | 30-35 | 6 | 742 |
| | IMGT | GFTFGFYY-- | 26-33 | 8 | 743 |
| CDR- | Chothia | -----DPHGGS------ | 52-57 | 6 | 744 |
| H2 | | --- | | | |
| | AbM | ---FIDPHGGSTY---- | 50-59 | 10 | 745 |
| | | --- | | | |
| | Kabat | --- | 50-66 | 17 | 746 |
| | | FIDPHGGSTYYADSVKG | | | |
| | Contact | WVAFIDPHGGSTY---- | 47-59 | 13 | 747 |
| | | --- | | | |
| | IMGT | ----IDPHGGST----- | 51-58 | 8 | 748 |
| | | --- | | | |
| CDR- | Chothia | --LSYPGVLDY | 99-107 | 9 | 749 |
| H3 | | | | | |
| | AbM | --LSYPGVLDY | 99-107 | 9 | 750 |
| | Kabat | --LSYPGVLDY | 99-107 | 9 | 751 |
| | Contact | ARLSYPGVLD- | 97-106 | 10 | 752 |
| | IMGT | ARLSYPGVLDY | 97-107 | 11 | 753 |
| CDR- | Chothia | RASQSVSSAVA-- | 24-34 | 11 | 754 |
| L1 | | | | | |
| | AbM | RASQSVSSAVA-- | 24-34 | 11 | 755 |
| | Kabat | RASQSVSSAVA-- | 24-34 | 11 | 756 |
| | Contact | ------SSAVAWY | 30-36 | 7 | 757 |
| | IMGT | ---QSVSSA---- | 27-32 | 6 | 758 |
| CDR- | Chothia | ----SASSLYS | 50-56 | 7 | 759 |
| L2 | | | | | |
| | AbM | ----SASSLYS | 50-56 | 7 | 760 |
| | Kabat | ----SASSLYS | 50-56 | 7 | 761 |
| | Contact | LLIYSASSLY- | 46-55 | 10 | 762 |
| | IMGT | ----SA----- | 50-51 | 2 | 763 |
| CDR- | Chothia | QQGYSTLVT | 89-97 | 9 | 764 |
| L3 | | | | | |
| | AbM | QQGYSTLVT | 89-97 | 9 | 765 |
| | Kabat | QQGYSTLVT | 89-97 | 9 | 766 |
| | Contact | QQGYSTLV- | 89-96 | 8 | 767 |
| | IMGT | QQGYSTLVT | 89-97 | 9 | 768 |

P9-31

| | | | | | |
|---|---|---|---|---|---|
| ‘CDR- | Chothia | GFTFSQY--- | 26-32 | 7 | 769 |
| H1 | | | | | |
| | AbM | GFTFSQYAIH | 26-35 | 10 | 770 |
| | Kabat | -----QYAIH | 31-35 | 5 | 771 |
| | Contact | ----SQYAIH | 30-35 | 6 | 772 |
| | IMGT | GFTFSQYA-- | 26-33 | 8 | 773 |
| CDR- | Chothia | -----YPDSGY------ | 52-57 | 6 | 774 |
| H2 | | --- | | | |
| | AbM | ---RIYPDSGYTY---- | 50-59 | 10 | 775 |
| | | --- | | | |
| | Kabat | --- | 50-66 | 17 | 776 |
| | | RIYPDSGYTYYADSVKG | | | |
| | Contact | WVARIYPDSGYTY---- | 47-59 | 13 | 777 |
| | | --- | | | |
| | IMGT | ----IYPDSGYT----- | 51-58 | 8 | 778 |
| | | --- | | | |
| CDR- | Chothia | --PYHQYAEGMDY | 99-109 | 11 | 779 |
| H3 | | | | | |
| | AbM | --PYHQYAEGMDY | 99-109 | 11 | 800 |
| | Kabat | --PYHQYAEGMDY | 99-109 | 11 | 801 |
| | Contact | ARPYHQYAEGMD- | 97-108 | 12 | 802 |
| | IMGT | ARPYHQYAEGMDY | 97-109 | 13 | 803 |
| CDR- | Chothia | RASQSVSRAVA-- | 24-34 | 11 | 804 |
| L1 | | | | | |
| | AbM | RASQSVSRAVA-- | 24-34 | 11 | 805 |
| | Kabat | RASQSVSRAVA-- | 24-34 | 11 | 806 |
| | Contact | ------SRAVAWY | 30-36 | 7 | 807 |
| | IMGT | ---QSVSRA---- | 27-32 | 6 | 808 |
| CDR- | Chothia | ----SASSLYS | 50-56 | 7 | 809 |
| L2 | | | | | |
| | AbM | ----SASSLYS | 50-56 | 7 | 810 |
| | Kabat | ----SASSLYS | 50-56 | 7 | 811 |
| | Contact | LLIYSASSLY- | 46-55 | 10 | 812 |
| | IMGT | ----SA----- | 50-51 | 2 | 813 |
| CDR- | Chothia | QQYYSPLLT | 89-97 | 9 | 814 |
| L3 | | | | | |
| | AbM | QQYYSPLLT | 89-97 | 9 | 815 |
| | Kabat | QQYYSPLLT | 89-97 | 9 | 816 |
| | Contact | QQYYSPLL- | 89-96 | 8 | 817 |
| | IMGT | QQYYSPLLT | 89-97 | 9 | 818 |

P9-32

| | | | | | |
|---|---|---|---|---|---|
| CDR- | Chothia | GFTFSAY--- | 26-32 | 7 | 819 |
| H1 | | | | | |
| | AbM | GFTFSAYWIH | 26-35 | 10 | 820 |
| | Kabat | -----AYWIH | 31-35 | 5 | 821 |
| | Contact | ----SAYWIH | 30-35 | 6 | 822 |
| | IMGT | GFTFSAYW-- | 26-33 | 8 | 823 |
| CDR- | Chothia | -----GPDGGY------ | 52-57 | 6 | 824 |
| H2 | | --- | | | |
| | AbM | ---LIGPDGGYTY---- | 50-59 | 10 | 825 |
| | | --- | | | |
| | Kabat | --- | 50-66 | 17 | 826 |
| | | LIGPDGGYTYYADSVKG | | | |
| | Contact | WVALIGPDGGYTY---- | 47-59 | 13 | 827 |
| | | --- | | | |
| | IMGT | ----IGPDGGYT----- | 51-58 | 8 | 828 |
| | | --- | | | |
| CDR- | Chothia | --QASRGLDY | 99-106 | 8 | 829 |
| H3 | | | | | |
| | AbM | --QASRGLDY | 99-106 | 8 | 830 |
| | Kabat | --QASRGLDY | 99-106 | 8 | 831 |
| | Contact | ARQASRGLD- | 97-105 | 9 | 832 |
| | IMGT | ARQASRGLDY | 97-106 | 10 | 833 |
| CDR- | Chothia | RASQSVSSAVA-- | 24-34 | 11 | 834 |
| L1 | | | | | |
| | AbM | RASQSVSSAVA-- | 24-34 | 11 | 835 |
| | Kabat | RASQSVSSAVA-- | 24-34 | 11 | 836 |
| | Contact | ------SSAVAWY | 30-36 | 7 | 837 |
| | IMGT | ---QSVSSA---- | 27-32 | 6 | 838 |
| CDR- | Chothia | ----SASSLYS | 50-56 | 7 | 839 |
| L2 | | | | | |
| | AbM | ----SASSLYS | 50-56 | 7 | 840 |
| | Kabat | ----SASSLYS | 50-56 | 7 | 841 |
| | Contact | LLIYSASSLY- | 46-55 | 10 | 842 |
| | IMGT | ----SA----- | 50-51 | 2 | 843 |
| CDR- | Chothia | QQWSSPLHT | 89-97 | 9 | 844 |
| L3 | | | | | |
| | AbM | QQWSSPLHT | 89-97 | 9 | 845 |
| | Kabat | QQWSSPLHT | 89-97 | 9 | 846 |
| | Contact | QQWSSPLH- | 89-96 | 8 | 847 |
| | IMGT | QQWSSPLHT | 89-97 | 9 | 848 |

P9-36

| | | | | | |
|---|---|---|---|---|---|
| CDR- | Chothia | GFTFGTY--- | 26-32 | 7 | 849 |
| H1 | | | | | |
| | AbM | GFTFGTYYIH | 26-35 | 10 | 850 |
| | Kabat | -----TYYIH | 31-35 | 5 | 851 |
| | Contact | ----GTYYIH | 30-35 | 6 | 852 |
| | IMGT | GFTFGTYY-- | 26-33 | 8 | 853 |
| CDR- | Chothia | -----LSGGGY------ | 52-57 | 6 | 854 |
| H2 | | --- | | | |
| | AbM | ---SILSGGGYTV---- | 50-59 | 10 | 855 |
| | | --- | | | |
| | Kabat | --- | 50-66 | 17 | 856 |
| | | SILSGGGYTVYADSVKG | | | |
| | Contact | WVASILSGGGYTV---- | 47-59 | 13 | 857 |
| | | --- | | | |
| | IMGT | ----ILSGGGYT----- | 51-58 | 8 | 858 |
| | | --- | | | |
| CDR- | Chothia | --RVYPGFDY | 99-106 | 8 | 859 |
| H3 | | | | | |
| | AbM | --RVYPGFDY | 99-106 | 8 | 860 |
| | Kabat | --RVYPGFDY | 99-106 | 8 | 861 |
| | Contact | ARRVYPGFD- | 97-105 | 9 | 862 |
| | IMGT | ARRVYPGFDY | 97-106 | 10 | 863 |
| CDR- | Chothia | RASQSVSSAVA-- | 24-34 | 11 | 864 |
| L1 | | | | | |
| | AbM | RASQSVSSAVA-- | 24-34 | 11 | 865 |
| | Kabat | RASQSVSSAVA-- | 24-34 | 11 | 866 |
| | Contact | ------SSAVAWY | 30-36 | 7 | 867 |
| | IMGT | ---QSVSSA---- | 27-32 | 6 | 868 |
| CDR- | Chothia | ----SASSLYS | 50-56 | 7 | 869 |
| L2 | | | | | |
| | AbM | ----SASSLYS | 50-56 | 7 | 870 |
| | Kabat | ----SASSLYS | 50-56 | 7 | 871 |
| | Contact | LLIYSASSLY- | 46-55 | 10 | 872 |
| | IMGT | ----SA----- | 50-51 | 2 | 873 |
| CDR- | Chothia | QQDSWGLWT | 89-97 | 9 | 874 |
| L3 | | | | | |
| | AbM | QQDSWGLWT | 89-97 | 9 | 875 |
| | Kabat | QQDSWGLWT | 89-97 | 9 | 876 |
| | Contact | QQDSWGLW- | 89-96 | 8 | 877 |
| | IMGT | QQDSWGLWT | 89-97 | 9 | 878 |

P9-39

| | | | | | |
|---|---|---|---|---|---|
| CDR- | Chothia | GFTFSFY--- | 26-32 | 7 | 879 |
| H1 | | | | | |
| | AbM | GFTFSFYGIH | 26-35 | 10 | 880 |
| | Kabat | -----FYGIH | 31-35 | 5 | 881 |
| | Contact | ----SFYGIH | 30-35 | 6 | 882 |
| | IMGT | GFTFSFYG-- | 26-33 | 8 | 883 |
| CDR- | Chothia | -----YPYGGF------ | 52-57 | 6 | 884 |
| H2 | | --- | | | |
| | AbM | ---WIYPYGGFTD---- | 50-59 | 10 | 885 |
| | | --- | | | |
| | Kabat | --- | 50-66 | 17 | 886 |
| | | WIYPYGGFTDYADSVKG | | | |
| | Contact | WVAWIYPYGGFTD---- | 47-59 | 13 | 887 |
| | | --- | | | |
| | IMGT | ----IYPYGGFT----- | 51-58 | 8 | 888 |
| | | --- | | | |
| CDR- | Chothia | --SGFFAFDY | 99-106 | 8 | 889 |
| H3 | | | | | |
| | AbM | --SGFFAFDY | 99-106 | 8 | 890 |
| | Kabat | --SGFFAFDY | 99-106 | 8 | 891 |
| | Contact | ARSGFFAFD- | 97-105 | 9 | 892 |
| | IMGT | ARSGFFAFDY | 97-106 | 10 | 893 |
| CDR- | Chothia | RASQSVSSAVA-- | 24-34 | 11 | 894 |
| L1 | | | | | |
| | AbM | RASQSVSSAVA-- | 24-34 | 11 | 895 |
| | Kabat | RASQSVSSAVA-- | 24-34 | 11 | 896 |
| | Contact | ------SSAVAWY | 30-36 | 7 | 897 |
| | IMGT | ---QSVSSA---- | 27-32 | 6 | 898 |
| CDR- | Chothia | ----SASSLYS | 50-56 | 7 | 899 |
| L2 | | | | | |
| | AbM | ----SASSLYS | 50-56 | 7 | 900 |
| | Kabat | ----SASSLYS | 50-56 | 7 | 901 |
| | Contact | LLIYSASSLY- | 46-55 | 10 | 902 |
| | IMGT | ----SA----- | 50-51 | 2 | 903 |
| CDR- | Chothia | QQVQTSLAT | 89-97 | 9 | 904 |
| L3 | | | | | |
| | AbM | QQVQTSLAT | 89-97 | 9 | 905 |
| | Kabat | QQVQTSLAT | 89-97 | 9 | 906 |
| | Contact | QQVQTSLA- | 89-96 | 8 | 907 |
| | IMGT | QQVQTSLAT | 89-97 | 9 | 908 |

P9-49

| | | | | | |
|---|---|---|---|---|---|
| CDR- | Chothia | GFTFSWY--- | 26-32 | 7 | 939 |
| H1 | | | | | |
| | AbM | GFTFSWYEIH | 26-35 | 10 | 940 |
| | Kabat | -----WYEIH | 31-35 | 5 | 941 |
| | Contact | ----SWYEIH | 30-35 | 6 | 942 |
| | IMGT | GFTFSWYE-- | 26-33 | 8 | 943 |
| CDR- | Chothia | -----GPYSSY------ | 52-57 | 6 | 944 |
| H2 | | --- | | | |
| | AbM | ---RIGPYSSYTY---- | 50-59 | 10 | 945 |
| | | --- | | | |
| | Kabat | --- | 50-66 | 17 | 946 |
| | | RIGPYSSYTYYADSVKG | | | |
| | Contact | WVARIGPYSSYTY---- | 47-59 | 13 | 947 |
| | | --- | | | |
| | IMGT | ----IGPYSSYT----- | 51-58 | 8 | 948 |
| | | --- | | | |
| CDR- | Chothia | --TYYPSYGMDY | 99-108 | 10 | 949 |
| H3 | | | | | |
| | AbM | --TYYPSYGMDY | 99-108 | 10 | 950 |
| | Kabat | --TYYPSYGMDY | 99-108 | 10 | 951 |
| | Contact | ARTYYPSYGMD- | 97-107 | 11 | 952 |
| | IMGT | ARTYYPSYGMDY | 97-108 | 12 | 953 |
| CDR- | Chothia | RASQSVSSAVA-- | 24-34 | 11 | 954 |
| L1 | | | | | |
| | AbM | RASQSVSSAVA-- | 24-34 | 11 | 955 |
| | Kabat | RASQSVSSAVA-- | 24-34 | 11 | 956 |
| | Contact | ------SSAVAWY | 30-36 | 7 | 957 |
| | IMGT | ---QSVSSA---- | 27-32 | 6 | 958 |
| CDR- | Chothia | ----SASSLYS | 50-56 | 7 | 959 |
| L2 | | | | | |
| | AbM | ----SASSLYS | 50-56 | 7 | 960 |
| | Kabat | ----SASSLYS | 50-56 | 7 | 961 |
| | Contact | LLIYSASSLY- | 46-55 | 10 | 962 |
| | IMGT | ----SA----- | 50-51 | 2 | 963 |
| CDR- | Chothia | QQSFSSPVT | 89-97 | 9 | 964 |
| L3 | | | | | |
| | AbM | QQSFSSPVT | 89-97 | 9 | 965 |
| | Kabat | QQSFSSPVT | 89-97 | 9 | 966 |
| | Contact | QQSFSSPV- | 89-96 | 8 | 967 |
| | IMGT | QQSFSSPVT | 89-97 | 9 | 968 |

P9-54

| | | | | | |
|---|---|---|---|---|---|
| CDR- | Chothia | GFTFSTY--- | 26-32 | 7 | 969 |
| H1 | | | | | |
| | AbM | GFTFSTYFIH | 26-35 | 10 | 970 |
| | Kabat | -----TYFIH | 31-35 | 5 | 971 |
| | Contact | ----STYFIH | 30-35 | 6 | 972 |
| | IMGT | GFTFSTYF-- | 26-33 | 8 | 973 |
| CDR- | Chothia | -----SPSGSH------ | 52-57 | 6 | 974 |
| H2 | | --- | | | |
| | AbM | ---WISPSGSHTG---- | 50-59 | 10 | 975 |
| | | --- | | | |
| | Kabat | --- | 50-66 | 17 | 976 |
| | | WISPSGSHTGYADSVKG | | | |
| | Contact | WVAWISPSGSHTG---- | 47-59 | 13 | 977 |
| | | --- | | | |
| | IMGT | ----ISPSGSHT----- | 51-58 | 8 | 978 |
| | | --- | | | |
| CDR- | Chothia | --VRYPGVMDY | 99-107 | 9 | 979 |
| H3 | | | | | |
| | AbM | --VRYPGVMDY | 99-107 | 9 | 980 |
| | Kabat | --VRYPGVMDY | 99-107 | 9 | 981 |
| | Contact | ARVRYPGVMD- | 97-106 | 10 | 982 |
| | IMGT | ARVRYPGVMDY | 97-107 | 11 | 983 |
| CDR- | Chothia | RASQSVSSAVA-- | 24-34 | 11 | 984 |
| L1 | | | | | |
| | AbM | RASQSVSSAVA-- | 24-34 | 11 | 985 |
| | Kabat | RASQSVSSAVA-- | 24-34 | 11 | 986 |
| | Contact | ------SSAVAWY | 30-36 | 7 | 987 |
| | IMGT | ---QSVSSA---- | 27-32 | 6 | 988 |
| CDR- | Chothia | ----SASSLYS | 50-56 | 7 | 989 |
| L2 | | | | | |
| | AbM | ----SASSLYS | 50-56 | 7 | 990 |
| | Kabat | ----SASSLYS | 50-56 | 7 | 991 |
| | Contact | LLIYSASSLY- | 46-55 | 10 | 992 |
| | IMGT | ----SA----- | 50-51 | 2 | 993 |
| CDR- | Chothia | QQWYPSLIT | 89-97 | 9 | 994 |
| L3 | | | | | |
| | AbM | QQWYPSLIT | 89-97 | 9 | 995 |
| | Kabat | QQWYPSLIT | 89-97 | 9 | 996 |
| | Contact | QQWYPSLI- | 89-96 | 8 | 997 |
| | IMGT | QQWYPSLIT | 89-97 | 9 | 998 |

P9-58

| | | | | | |
|---|---|---|---|---|---|
| CDR- | Chothia | GFTFSRY--- | 26-32 | 7 | 999 |
| H1 | | | | | |
| | AbM | GFTFSRYYIH | 26-35 | 10 | 1000 |
| | Kabat | -----RYYIH | 31-35 | 5 | 1001 |
| | Contact | ----SRYYIH | 30-35 | 6 | 1002 |
| | IMGT | GFTFSRYY-- | 26-33 | 8 | 1003 |
| CDR- | Chothia | -----SSDSGY------ | 52-57 | 6 | 1004 |
| H2 | | --- | | | |
| | AbM | ---FISSDSGYTQ---- | 50-59 | 10 | 1005 |
| | | --- | | | |
| | Kabat | --- | 50-66 | 17 | 1006 |
| | | FISSDSGYTQYADSVKG | | | |
| | Contact | WVAFISSDSGYTQ---- | 47-59 | 13 | 1007 |
| | | --- | | | |
| | IMGT | ----ISSDSGYT----- | 51-58 | 8 | 1008 |
| | | --- | | | |
| CDR- | Chothia | --TMSYSALDY | 99-107 | 9 | 1009 |
| H3 | | | | | |
| | AbM | --TMSYSALDY | 99-107 | 9 | 1010 |
| | Kabat | --TMSYSALDY | 99-107 | 9 | 1011 |
| | Contact | ARTMSYSALD- | 97-106 | 10 | 1012 |
| | IMGT | ARTMSYSALDY | 97-107 | 11 | 1013 |
| CDR- | Chothia | RASQSVSSAVA-- | 24-34 | 11 | 1014 |
| L1 | | | | | |
| | AbM | RASQSVSSAVA-- | 24-34 | 11 | 1015 |
| | Kabat | RASQSVSSAVA-- | 24-34 | 11 | 1016 |
| | Contact | ------SSAVAWY | 30-36 | 7 | 1017 |
| | IMGT | ---QSVSSA---- | 27-32 | 6 | 1018 |
| CDR- | Chothia | ----SASSLYS | 50-56 | 7 | 1019 |
| L2 | | | | | |
| | AbM | ----SASSLYS | 50-56 | 7 | 1020 |
| | Kabat | ----SASSLYS | 50-56 | 7 | 1021 |
| | Contact | LLIYSASSLY- | 46-55 | 10 | 1022 |
| | IMGT | ----SA----- | 50-51 | 2 | 1023 |
| CDR- | Chothia | QQGFGFLVT | 89-97 | 9 | 1024 |
| L3 | | | | | |
| | AbM | QQGFGFLVT | 89-97 | 9 | 1025 |
| | Kabat | QQGFGFLVT | 89-97 | 9 | 1026 |
| | Contact | QQGFGFLV- | 89-96 | 8 | 1027 |
| | IMGT | QQGFGFLVT | 89-97 | 9 | 1028 |

Table 6 presents full immunoglobulin heavy chain and full immunoglobulin light chain sequences, and the VH and VL. sequences, of various ABS candidates formatted into a bivalent monospecific human full-length IgG1 architecture.

TABLE 6
full chain sequences and VH/VL sequences of candidate GAL9 ABS clones and
IgG formatted antibodies comprising GAL9 ABSs

| ABS | Full IgG | Full IgG | VH | VL |
|---|---|---|---|---|
| clone | Heavy Chain | Light Chain | sequence | sequence |
| P9-02B | EVQLVESGGGLVQPGG | DIQMTQSPSSLSASVGDRV | EVQLVESGGGL | DIQMTQSPSS |
| | SLRLSCAASGFTFSGYFI | TITCRASQSVSSAVAWYQQ | VQPGGSLRLSC | LSASVGDRV |
| | HWVRQAPGKGLEWVA | KPGKAPKLLIYSASSLYSG | AASGFTFSGYFI | TITCRASQSV |
| | RISSYGGHTDYADSVK | VPSRFSGSRSGTDFTLTISS | HWVRQAPGKG | SSAVAWYQQ |
| | GRFTISADTSKNTAYLQ | LQPEDFATYYCQQSYPTLG | LEWVARISSYG | KPGKAPKLLI |
| | MNSLRAEDTAVYYCAR | TFGQGTKVEIKRTVAAPSV | GHTDYADSVK | YSASSLYSGV |
| | AQYVPGLDYWGQGTL | FIFPPSDSQLKSGTASVVCL | GRFTISADTSKN | PSRFSGSRSG |
| | VTVSSASTKGPSVFPLA | LNNFYPREAKVQWKVDN | TAYLQMNSLRA | TDFTLTISSLQ |
| | PSSKSTSGGTAALGCLV | ALQSGNSQESVTEQDSKDS | EDTAVYYCAR | PEDFATYYC |
| | KDYFPEPVTVSWNSGA | TYSLSSTLTLSKADYEKHK | AQYVPGLDYW | QQSYPTLGTF |
| | LTSGVHTFPAVLQSSGL | VYACEVTHQGLSSPVTKSF | GQGTLVTVSS | GQGTKVEIKR |
| | YSLSSVVTVPSSSLGTQ | NRGEC | (SEQ ID | TV |
| | TYICNVNHKPSNTKVD | (SEQ ID NO: 1030) | NO: 1031) | (SEQ ID |
| | KKVEPKSCDKTHTCPP | | | NO: 1032) |
| | CPAPELLGGPSVFLFPP | | | |
| | KPKDTLMISRTPEVTCV | | | |
| | VVDVSHEDPEVKFNWY | | | |
| | VDGVEVHNAKTKPREE | | | |
| | QYNSTYRVVSVLTVLH | | | |
| | QDWLNGKEYKCKVSN | | | |
| | KALPAPIEKTISKAKGQ | | | |
| | PREPQVYTLPPSRDELT | | | |
| | KNQVSLTCLVKGFYPS | | | |
| | DIAVEWESNGQPENNY | | | |
| | KTTPPVLDSDGSFFLYS | | | |
| | KLTVDKSRWQQGNVFS | | | |
| | CSVMHEALHNHYTQKS | | | |
| | LSLSPGK | | | |
| | (SEQ ID NO: 1029) | | | |
| P9-04 | EVQLVESGGGLVQPGG | DIQMTQSPSSLSASVGDRV | EVQLVESGGGL | DIQMTQSPSS |
| | SLRLSCAASGFTFGSYFI | TITCRASQSVSSAVAWYQQ | VQPGGSLRLSC | LSASVGDRV |
| | HWVRQAPGKGLEWVA | KPGKAPKLLIYSASSLYSG | AASGFTFGSYFI | TITCRASQSV |
| | YISPTWGYTYYADSVK | VPSRFSGSRSGTDFTLTISS | HWVRQAPGKG | SSAVAWYQQ |
| | GRFTISADTSKNTAYLQ | LQPEDFATYYCQQVYSSPL | LEWVAYISPTW | KPGKAPKLLI |
| | MNSLRAEDTAVYYCAR | TFGQGTKVEIKRTVAAPSV | GYTYYADSVK | YSASSLYSGV |
| | AWFGFAFDYWGQGTL | FIFPPSDSQLKSGTASVVCL | GRFTISADTSKN | PSRFSGSRSG |
| | VTVSSASTKGPSVFPLA | LNNFYPREAKVQWKVDN | TAYLQMNSLRA | TDFTLTISSLQ |
| | PSSKSTSGGTAALGCLV | ALQSGNSQESVTEQDSKDS | EDTAVYYCAR | PEDFATYYC |
| | KDYFPEPVTVSWNSGA | TYSLSSTLTLSKADYEKHK | AWFGFAFDYW | QQVYSSPLTF |
| | LTSGVHTFPAVLQSSGL | VYACEVTHQGLSSPVTKSF | GQGTLVTVSS | GQGTKVEIKR |
| | YSLSSVVTVPSSSLGTQ | NRGEC | (SEQ ID | TV |
| | TYICNVNHKPSNTKVD | (SEQ ID NO: 1034) | NO: 1035) | (SEQ ID |
| | KKVEPKSCDKTHTCPP | | | NO: 1036) |
| | CPAPELLGGPSVFLFPP | | | |
| | KPKDTLMISRTPEVTCV | | | |
| | VVDVSHEDPEVKFNWY | | | |
| | VDGVEVHNAKTKPREE | | | |
| | QYNSTYRVVSVLTVLH | | | |
| | QDWLNGKEYKCKVSN | | | |
| | KALPAPIEKTISKAKGQ | | | |
| | PREPQVYTLPPSRDELT | | | |
| | KNQVSLTCLVKGFYPS | | | |
| | DIAVEWESNGQPENNY | | | |
| | KTTPPVLDSDGSFFLYS | | | |
| | KLTVDKSRWQQGNVFS | | | |
| | CSVMHEALHNHYTQKS | | | |
| | LSLSPGK | | | |
| | (SEQ ID NO: 1033) | | | |
| P9-05 | EVQLVESGGGLVQPGG | DIQMTQSPSSLSASVGDRV | EVQLVESGGGL | DIQMTQSPSS |
| | SLRLSCAASGFTFTSYYI | TITCRASQSVSSAVAWYQQ | VQPGGSLRLSC | LSASVGDRV |
| | HWVRQAPGKGLEWVA | KPGKAPKLLIYSASSLYSG | AASGFTFTSYYI | TITCRASQSV |
| | WIWPIGGYTYYADSVK | VPSRFSGSRSGTDFTLTISS | HWVRQAPGKG | SSAVAWYQQ |
| | GRFTISADTSKNTAYLQ | LQPEDFATYYCQQYYYSL | LEWVAWIWPIG | KPGKAPKLLI |
| | MNSLRAEDTAVYYCAR | LTFGQGTKVEIKRTVAAPS | GYTYYADSVK | YSASSLYSGV |
| | DAGSGFDYWGQGTLV | VFIFPPSDSQLKSGTASVVC | GRFTISADTSKN | PSRFSGSRSG |
| | TVSSASTKGPSVFPLAP | LLNNFYPREAKVQWKVDN | TAYLQMNSLRA | TDFTLTISSLQ |
| | SSKSTSGGTAALGCLV | ALQSGNSQESVTEQDSKDS | EDTAVYYCAR | PEDFATYYC |
| | KDYFPEPVTVSWNSGA | TYSLSSTLTLSKADYEKHK | DAGSGFDYWG | QQYYYSLLT |
| | LTSGVHTFPAVLQSSGL | VYACEVTHQGLSSPVTKSF | QGTLVTVSS | FGQGTKVEIK |
| | YSLSSVVTVPSSSLGTQ | NRGEC | (SEQ ID | RTV |
| | TYICNVNHKPSNTKVD | (SEQ ID NO: 1038) | NO: 1039) | (SEQ ID |
| | KKVEPKSCDKTHTCPP | | | NO: 1040) |
| | CPAPELLGGPSVFLFPP | | | |
| | KPKDTLMISRTPEVTCV | | | |
| | VVDVSHEDPEVKFNWY | | | |
| | VDGVEVHNAKTKPREE | | | |
| | QYNSTYRVVSVLTVLH | | | |
| | QDWLNGKEYKCKVSN | | | |
| | KALPAPIEKTISKAKGQ | | | |
| | PREPQVYTLPPSRDELT | | | |
| | KNQVSLTCLVKGFYPS | | | |
| | DIAVEWESNGQPENNY | | | |
| | KTTPPVLDSDGSFFLYS | | | |
| | KLTVDKSRWQQGNVFS | | | |
| | CSVMHEALHNHYTQKS | | | |
| | LSLSPGK | | | |
| | (SEQ ID NO: 1037) | | | |
| P9-08 | EVQLVESGGGLVQPGG | DIQMTQSPSSLSASVGDRV | EVQLVESGGGL | DIQMTQSPSS |
| | SLRLSCAASGFTFSRYA | TITCRASQSVSSAVAWYQQ | VQPGGSLRLSC | LSASVGDRV |
| | IHWVRQAPGKGLEWV | KPGKAPKLLIYSASSLYSG | AASGFTFSRYAI | TITCRASQSV |
| | AAIYSPTGYTDYADSV | VPSRFSGSRSGTDFTLTISS | HWVRQAPGKG | SSAVAWYQQ |
| | KGRFTISADTSKNTAYL | LQPEDFATYYCQQSYSALY | LEWVAAIYSPT | KPGKAPKLLI |
| | QMNSLRAEDTAVYYC | TFGQGTKVEIKRTVAAPSV | GYTDYADSVK | YSASSLYSGV |
| | AREGYIGMDYWGQGT | FIFPPSDSQLKSGTASVVCL | GRFTISADTSKN | PSRFSGSRSG |
| | LVTVSSASTKGPSVFPL | LNNFYPREAKVQWKVDN | TAYLQMNSLRA | TDFTLTISSLQ |
| | APSSKSTSGGTAALGCL | ALQSGNSQESVTEQDSKDS | EDTAVYYCARE | PEDFATYYC |
| | VKDYFPEPVTVSWNSG | TYSLSSTLTLSKADYEKHK | GYIGMDYWGQ | QQSYSALYTF |
| | ALTSGVHTFPAVLQSSG | VYACEVTHQGLSSPVTKSF | GTLVTVSS | GQGTKVEIKR |
| | LYSLSSVVTVPSSSLGT | NRGEC | (SEQ ID | TV |
| | QTYICNVNHKPSNTKV | (SEQ ID NO: 1042) | NO: 1043) | (SEQ ID |
| | DKKVEPKSCDKTHTCP | | | NO: 1044) |
| | PCPAPELLGGPSVFLFPP | | | |
| | KPKDTLMISRTPEVTCV | | | |
| | VVDVSHEDPEVKFNWY | | | |
| | VDGVEVHNAKTKPREE | | | |
| | QYNSTYRVVSVLTVLH | | | |
| | QDWLNGKEYKCKVSN | | | |
| | KALPAPIEKTISKAKGQ | | | |
| | PREPQVYTLPPSRDELT | | | |
| | KNQVSLTCLVKGFYPS | | | |
| | DIAVEWESNGQPENNY | | | |
| | KTTPPVLDSDGSFFLYS | | | |
| | KLTVDKSRWQQGNVFS | | | |
| | CSVMHEALHNHYTQKS | | | |
| | LSLSPGK | | | |
| | (SEQ ID NO: 1041) | | | |
| P9-09 | EVQLVESGGGLVQPGG | DIQMTQSPSSLSASVGDRV | EVQLVESGGGL | DIQMTQSPSS |
| | SLRLSCAASGFTFSSYFI | TITCRASQSVSSAVAWYQQ | VQPGGSLRLSC | LSASVGDRV |
| | HWVRQAPGKGLEWVA | KPGKAPKLLIYSASSLYSG | AASGFTFSSYFI | TITCRASQSV |
| | WIYSSGGYTYYADSVK | VPSRFSGSRSGTDFTLTISS | HWVRQAPGKG | SSAVAWYQQ |
| | GRFTISADTSKNTAYLQ | LQPEDFATYYCQQADPSLV | LEWVAWIYSSG | KPGKAPKLLI |
| | MNSLRAEDTAVYYCAR | TFGQGTKVEIKRTVAAPSV | GYTYYADSVK | YSASSLYSGV |
| | FTPSDLGYGLDYWGQG | FIFPPSDSQLKSGTASVVCL | GRFTISADTSKN | PSRFSGSRSG |
| | TLVTVSSASTKGPSVFP | LNNFYPREAKVQWKVDN | TAYLQMNSLRA | TDFTLTISSLQ |
| | LAPSSKSTSGGTAALGC | ALQSGNSQESVTEQDSKDS | EDTAVYYCARF | PEDFATYYC |
| | LVKDYFPEPVTVSWNS | TYSLSSTLTLSKADYEKHK | TPSDLGYGLDY | QQADPSLVTF |
| | GALTSGVHTFPAVLQSS | VYACEVTHQGLSSPVTKSF | WGQGTLVTVSS | GQGTKVEIKR |
| | GLYSLSSVVTVPSSSLG | NRGEC | (SEQ ID | TV |
| | TQTYICNVNHKPSNTK | (SEQ ID NO: 1046) | NO: 1047) | (SEQ ID |
| | VDKKVEPKSCDKTHTC | | | NO: 1048) |
| | PPCPAPELLGGPSVFLFP | | | |
| | PKPKDTLMISRTPEVTC | | | |
| | VVVDVSHEDPEVKFNW | | | |
| | YVDGVEVHNAKTKPRE | | | |
| | EQYNSTYRVVSVLTVL | | | |
| | HQDWLNGKEYKCKVS | | | |
| | NKALPAPIEKTISKAKG | | | |
| | QPREPQVYTLPPSRDEL | | | |
| | TKNQVSLTCLVKGFYP | | | |
| | SDIAVEWESNGQPENN | | | |
| | YKTTPPVLDSDGSFFLY | | | |
| | SKLTVDKSRWQQGNVF | | | |
| | SCSVMHEALHNHYTQK | | | |
| | SLSLSPGK | | | |
| | (SEQ ID NO: 1045) | | | |
| P9-10 | EVQLVESGGGLVQPGG | DIQMTQSPSSLSASVGDRV | EVQLVESGGGL | DIQMTQSPSS |
| | SLRLSCAASGFTFDYYV | TITCRASQSVSSAVAWYQQ | VQPGGSLRLSC | LSASVGDRV |
| | IHWVRQAPGKGLEWV | KPGKAPKLLIYSASSLYSG | AASGFTFDYYV | TITCRASQSV |
| | AAIDSYWGDTYYADSV | VPSRFSGSRSGTDFTLTISS | IHWVRQAPGKG | SSAVAWYQQ |
| | KGRFTISADTSKNTAYL | LQPEDFATYYCQQYSWSL | LEWVAAIDSY | KPGKAPKLLI |
| | QMNSLRAEDTAVYYC | WTFGQGTKVEIKRTVAAPS | WGDTYYADSV | YSASSLYSGV |
| | ARFYFYGFDYWGQGTL | VFIFPPSDSQLKSGTASVVC | KGRFTISADTSK | PSRFSGSRSG |
| | VTVSSASTKGPSVFPLA | LLNNFYPREAKVQWKVDN | NTAYLQMNSLR | TDFTLTISSLQ |
| | PSSKSTSGGTAALGCLV | ALQSGNSQESVTEQDSKDS | AEDTAVYYCA | PEDFATYYC |
| | KDYFPEPVTVSWNSGA | TYSLSSTLTLSKADYEKHK | RFYFYGFDYW | QQYSWSLWT |
| | LTSGVHTFPAVLQSSGL | VYACEVTHQGLSSPVTKSF | GQGTLVTVSS | FGQGTKVEIK |
| | YSLSSVVTVPSSSLGTQ | NRGEC | (SEQ ID | RTV |
| | TYICNVNHKPSNTKVD | (SEQ ID NO: 1050) | NO: 1051) | (SEQ ID |
| | KKVEPKSCDKTHTCPP | | | NO: 1052) |
| | CPAPELLGGPSVFLFPP | | | |
| | KPKDTLMISRTPEVTCV | | | |
| | VVDVSHEDPEVKFNWY | | | |
| | VDGVEVHNAKTKPREE | | | |
| | QYNSTYRVVSVLTVLH | | | |
| | QDWLNGKEYKCKVSN | | | |
| | KALPAPIEKTISKAKGQ | | | |
| | PREPQVYTLPPSRDELT | | | |
| | KNQVSLTCLVKGFYPS | | | |
| | DIAVEWESNGQPENNY | | | |
| | KTTPPVLDSDGSFFLYS | | | |
| | KLTVDKSRWQQGNVFS | | | |
| | CSVMHEALHNHYTQKS | | | |
| | LSLSPGK | | | |
| | (SEQ ID NO: 1049) | | | |
| P9-15 | EVQLVESGGGLVQPGG | DIQMTQSPSSLSASVGDRV | EVQLVESGGGL | DIQMTQSPSS |
| | SLRLSCAASGFTFRYY | TITCRASQSVSSAVAWYQQ | VQPGGSLRLSC | LSASVGDRV |
| | WIHWVRQAPGKGLEW | KPGKAPKLLIYSASSLYSG | AASGFTFRYYW | TITCRASQSV |
| | VAAIFPSGGITTYADSV | VPSRFSGSRSGTDFTLTISS | IHWVRQAPGKG | SSAVAWYQQ |
| | KGRFTISADTSKNTAYL | LQPEDFATYYCQQRDRSPY | LEWVAAIFPSG | KPGKAPKLLI |
| | QMNSLRAEDTAVYYC | TFGQGTKVEIKRTVAAPSV | GITTYADSVKG | YSASSLYSGV |
| | ARGWPWGLDYWGQGT | FIFPPSDSQLKSGTASVVCL | RFTISADTSKNT | PSRFSGSRSG |
| | LVTVSSASTKGPSVFPL | LNNFYPREAKVQWKVDN | AYLQMNSLRAE | TDFTLTISSLQ |
| | APSSKSTSGGTAALGCL | ALQSGNSQESVTEQDSKDS | DTAVYYCARG | PEDFATYYC |
| | VKDYFPEPVTVSWNSG | TYSLSSTLTLSKADYEKHK | WPWGLDYWG | QQRDRSPYTF |
| | ALTSGVHTFPAVLQSSG | VYACEVTHQGLSSPVTKSF | QGTLVTVSS | GQGTKVEIKR |
| | LYSLSSVVTVPSSSLGT | NRGEC | (SEQ ID | TV |
| | QTYICNVNHKPSNTKV | (SEQ ID NO: 1054) | NO: 1055) | (SEQ ID |
| | DKKVEPKSCDKTHTCP | | | NO: 1056) |
| | PCPAPELLGGPSVFLFPP | | | |
| | KPKDTLMISRTPEVTCV | | | |
| | VVDVSHEDPEVKFNWY | | | |
| | VDGVEVHNAKTKPREE | | | |
| | QYNSTYRVVSVLTVLH | | | |
| | QDWLNGKEYKCKVSN | | | |
| | KALPAPIEKTISKAKGQ | | | |
| | PREPQVYTLPPSRDELT | | | |
| | KNQVSLTCLVKGFYPS | | | |
| | DIAVEWESNGQPENNY | | | |
| | KTTPPVLDSDGSFFLYS | | | |
| | KLTVDKSRWQQGNVFS | | | |
| | CSVMHEALHNHYTQKS | | | |
| | LSLSPGK | | | |
| | (SEQ ID NO: 1053) | | | |
| P9-16 | EVQLVESGGGLVQPGG | DIQMTQSPSSLSASVGDRV | EVQLVESGGGL | DIQMTQSPSS |
| | SLRLSCAASGFTFSYYW | TITCRASQSVSSAVAWYQQ | VQPGGSLRLSC | LSASVGDRV |
| | IHWVRQAPGKGLEWV | KPGKAPKLLIYSASSLYSG | AASGFTFSYYW | TITCRASQSV |
| | ADIYPSSGYTYYADSV | VPSRFSGSRSGTDFTLTISS | IHWVRQAPGKG | SSAVAWYQQ |
| | KGRFTISADTSKNTAYL | LQPEDFATYYCQQYALRPL | LEWVADIYPSS | KPGKAPKLLI |
| | QMNSLRAEDTAVYYC | TFGQGTKVEIKRTVAAPSV | GYTYYADSVK | YSASSLYSGV |
| | ARGWYAAYGMDYWG | FIFPPSDSQLKSGTASVVCL | GRFTISADTSKN | PSRFSGSRSG |
| | QGTLVTVSSASTKGPSV | LNNFYPREAKVQWKVDN | TAYLQMNSLRA | TDFTLTISSLQ |
| | FPLAPSSKSTSGGTAAL | ALQSGNSQESVTEQDSKDS | EDTAVYYCAR | PEDFATYYC |
| | GCLVKDYFPEPVTVSW | TYSLSSTLTLSKADYEKHK | GWYAAYGMD | QQYALRPLTF |
| | NSGALTSGVHTFPAVL | VYACEVTHQGLSSPVTKSF | YWGQGTLVTV | GQGTKVEIKR |
| | QSSGLYSLSSVVTVPSS | NRGEC | SS | TV |
| | SLGTQTYICNVNHKPSN | (SEQ ID NO: 1058) | (SEQ ID | (SEQ ID |
| | TKVDKKVEPKSCDKTH | | NO: 1059) | NO: 1060) |
| | TCPPCPAPELLGGPSVF | | | |
| | LFPPKPKDTLMISRTPE | | | |
| | VTCVVVDVSHEDPEVK | | | |
| | FNWYVDGVEVHNAKT | | | |
| | KPREEQYNSTYRVVSV | | | |
| | LTVLHQDWLNGKEYK | | | |
| | CKVSNKALPAPIEKTIS | | | |
| | KAKGQPREPQVYTLPPS | | | |
| | RDELTKNQVSLTCLVK | | | |
| | GFYPSDIAVEWESNGQP | | | |
| | ENNYKTTPPVLDSDGSF | | | |
| | FLYSKLTVDKSRWQQG | | | |
| | NVFSCSVMHEALHNHY | | | |
| | TQKSLSLSPGK | | | |
| | (SEQ ID NO: 1057) | | | |
| P9-18 | EVQLVESGGGLVQPGG | DIQMTQSPSSLSASVGDRV | EVQLVESGGGL | DIQMTQSPSS |
| | SLRLSCAASGFTFSWYV | TITCRASQSVSSAVAWYQQ | VQPGGSLRLSC | LSASVGDRV |
| | IHWVRQAPGKGLEWV | KPGKAPKLLIYSASSLYSG | AASGFTFSWYV | TITCRASQSV |
| | AAIYPYHSKTYYADSV | VPSRFSGSRSGTDFTLTISS | IHWVRQAPGKG | SSAVAWYQQ |
| | KGRFTISADTSKNTAYL | LQPEDFATYYCQQYTDAP | LEWVAAIYPYH | KPGKAPKLLI |
| | QMNSLRAEDTAVYYC | WTFGQGTKVEIKRTVAAPS | SKTYYADSVKG | YSASSLYSGV |
| | ARGYWYGMDYWGQG | VFIFPPSDSQLKSGTASVVC | RFTISADTSKNT | PSRFSGSRSG |
| | TLVTVSSASTKGPSVFP | LLNNFYPREAKVQWKVDN | AYLQMNSLRAE | TDFTLTISSLQ |
| | LAPSSKSTSGGTAALGC | ALQSGNSQESVTEQDSKDS | DTAVYYCARG | PEDFATYYC |
| | LVKDYFPEPVTVSWNS | TYSLSSTLTLSKADYEKHK | YWYGMDYWG | QQYTDAPWT |
| | GALTSGVHTFPAVLQSS | VYACEVTHQGLSSPVTKSF | QGTLVTVSS | FGQGTKVEIK |
| | GLYSLSSVVTVPSSSLG | NRGEC | (SEQ ID | RTV |
| | TQTYICNVNHKPSNTK | (SEQ ID NO: 1062) | NO: 1063) | (SEQ ID |
| | VDKKVEPKSCDKTHTC | | | NO: 1064) |
| | PPCPAPELLGGPSVFLFP | | | |
| | PKPKDTLMISRTPEVTC | | | |
| | VVVDVSHEDPEVKFNW | | | |
| | YVDGVEVHNAKTKPRE | | | |
| | EQYNSTYRVVSVLTVL | | | |
| | HQDWLNGKEYKCKVS | | | |
| | NKALPAPIEKTISKAKG | | | |
| | QPREPQVYTLPPSRDEL | | | |
| | TKNQVSLTCLVKGFYP | | | |
| | SDIAVEWESNGQPENN | | | |
| | YKTTPPVLDSDGSFFLY | | | |
| | SKLTVDKSRWQQGNVF | | | |
| | SCSVMHEALHNHYTQK | | | |
| | SLSLSPGK | | | |
| | (SEQ ID NO: 1061) | | | |
| P9-19 | EVQLVESGGGLVQPGG | DIQMTQSPSSLSASVGDRV | EVQLVESGGGL | DIQMTQSPSS |
| | SLRLSCAASGFTFGYYY | TITCRASQSVSSAVAWYQQ | VQPGGSLRLSC | LSASVGDRV |
| | IHWVRQAPGKGLEWV | KPGKAPKLLIYSASSLYSG | AASGFTFGYYY | TITCRASQSV |
| | AWISPSGSVTAYADSV | VPSRFSGSRSGTDFTLTISS | IHWVRQAPGKG | SSAVAWYQQ |
| | KGRFTISADTSKNTAYL | LQPEDFATYYCQQYDTSPY | LEWVAWISPSG | KPGKAPKLLI |
| | QMNSLRAEDTAVYYC | TFGQGTKVEIKRTVAAPSV | SVTAYADSVKG | YSASSLYSGV |
| | ARGYYGGWGMDYWG | FIFPPSDSQLKSGTASVVCL | RFTISADTSKNT | PSRFSGSRSG |
| | QGTLVTVSSASTKGPSV | LNNFYPREAKVQWKVDN | AYLQMNSLRAE | TDFTLTISSLQ |
| | FPLAPSSKSTSGGTAAL | ALQSGNSQESVTEQDSKDS | DTAVYYCARG | PEDFATYYC |
| | GCLVKDYFPEPVTVSW | TYSLSSTLTLSKADYEKHK | YYGGWGMDY | QQYDTSPYTF |
| | NSGALTSGVHTFPAVL | VYACEVTHQGLSSPVTKSF | WGQGTLVTVSS | GQGTKVEIKR |
| | QSSGLYSLSSVVTVPSS | NRGEC | (SEQ ID | TV |
| | SLGTQTYICNVNHKPSN | (SEQ ID NO: 1066) | NO: 1067) | (SEQ ID |
| | TKVDKKVEPKSCDKTH | | | NO: 1068) |
| | TCPPCPAPELLGGPSVF | | | |
| | LFPPKPKDTLMISRTPE | | | |
| | VTCVVVDVSHEDPEVK | | | |
| | FNWYVDGVEVHNAKT | | | |
| | KPREEQYNSTYRVVSV | | | |
| | LTVLHQDWLNGKEYK | | | |
| | CKVSNKALPAPIEKTIS | | | |
| | KAKGQPREPQVYTLPPS | | | |
| | RDELTKNQVSLTCLVK | | | |
| | GFYPSDIAVEWESNGQP | | | |
| | ENNYKTTPPVLDSDGSF | | | |
| | FLYSKLTVDKSRWQQG | | | |
| | NVFSCSVMHEALHNHY | | | |
| | TQKSLSLSPGK | | | |
| | (SEQ ID NO: 1065) | | | |
| P9-20 | EVQLVESGGGLVQPGG | DIQMTQSPSSLSASVGDRV | EVQLVESGGGL | DIQMTQSPSS |
| | SLRLSCAASGFTFRYYY | TITCRASQSVSSAVAWYQQ | VQPGGSLRLSC | LSASVGDRV |
| | IHWVRQAPGKGLEWV | KPGKAPKLLIYSASSLYSG | AASGFTFRYYY | TITCRASQSV |
| | AGIYPYGGYTSYADSV | VPSRFSGSRSGTDFTLTISS | IHWVRQAPGKG | SSAVAWYQQ |
| | KGRFTISADTSKNTAYL | LQPEDFATYYCQQYYGSL | LEWVAGIYPYG | KPGKAPKLLI |
| | QMNSLRAEDTAVYYC | ATFGQGTKVEIKRTVAAPS | GYTSYADSVKG | YSASSLYSGV |
| | ARGYYVEGVLDYWGQ | VFIFPPSDSQLKSGTASVVC | RFTISADTSKNT | PSRFSGSRSG |
| | GTLVTVSSASTKGPSVF | LLNNFYPREAKVQWKVDN | AYLQMNSLRAE | TDFTLTISSLQ |
| | PLAPSSKSTSGGTAALG | ALQSGNSQESVTEQDSKDS | DTAVYYCARG | PEDFATYYC |
| | CLVKDYFPEPVTVSWN | TYSLSSTLTLSKADYEKHK | YYVEGVLDYW | QQYYGSLAT |
| | SGALTSGVHTFPAVLQS | VYACEVTHQGLSSPVTKSF | GQGTLVTVSS | FGQGTKVEIK |
| | SGLYSLSSVVTVPSSSL | NRGEC | (SEQ ID | RTV |
| | GTQTYICNVNHKPSNT | (SEQ ID NO: 1070) | NO: 1071) | (SEQ ID |
| | KVDKKVEPKSCDKTHT | | | NO: 1072) |
| | CPPCPAPELLGGPSVFL | | | |
| | FPXKPKDTLMISRTPEV | | | |
| | TCFVVDVSHEDPEVKF | | | |
| | NWYVDGVEVHNAKTK | | | |
| | PREEQYNSTYRVVSVL | | | |
| | TVLHQDWLNGKEYKC | | | |
| | KVSNKALPAPIEKTISK | | | |
| | AKGQPREPQVYTLPPSR | | | |
| | DELTKNQVSLTCLVKG | | | |
| | FYPSDIAVEWESNGQPE | | | |
| | NNYKTTPPVLDSDGSFF | | | |
| | LYSKLTVDKSRWQQGN | | | |
| | VFSCSVMHEALHNHYT | | | |
| | QKSLSLSPGK | | | |
| | (SEQ ID NO: 1069) | | | |
| P9-21 | EVQLVESGGGLVQPGG | DIQMTQSPSSLSASVGDRV | EVQLVESGGGL | DIQMTQSPSS |
| | SLRLSCAASGFTFSYYY | TITCRASQSVSSAVAWYQQ | VQPGGSLRLSC | LSASVGDRV |
| | IHWVRQAPGKGLEWV | KPGKAPKLLIYSASSLYSG | AASGFTFSYYYI | TITCRASQSV |
| | ARIHPPSGYTDYADSVK | VPSRFSGSRSGTDFTLTISS | HWVRQAPGKG | SSAVAWYQQ |
| | GRFTISADTSKNTAYLQ | LQPEDFATYYCQQSHWYP | LEWVARIHPPS | KPGKAPKLLI |
| | MNSLRAEDTAVYYCAR | FTFGQGTKVEIKRTVAAPS | GYTDYADSVK | YSASSLYSGV |
| | GYYVFGVMDYWGQGT | VFIFPPSDSQLKSGTASVVC | GRFTISADTSKN | PSRFSGSRSG |
| | LVTVSSASTKGPSVFPL | LLNNFYPREAKVQWKVDN | TAYLQMNSLRA | TDFTLTISSLQ |
| | APSSKSTSGGTAALGCL | ALQSGNSQESVTEQDSKDS | EDTAVYYCAR | PEDFATYYC |
| | VKDYFPEPVTVSWNSG | TYSLSSTLTLSKADYEKHK | GYYVFGVMDY | QQSHWYPFT |
| | ALTSGVHTFPAVLQSSG | VYACEVTHQGLSSPVTKSF | WGQGTLVTVSS | FGQGTKVEIK |
| | LYSLSSVVTVPSSSLGT | NRGEC | (SEQ ID | RTV |
| | QTYICNVNHKPSNTKV | (SEQ ID NO: 1074) | NO: 1075) | (SEQ ID |
| | DKKVEPKSCDKTHTCP | | | NO: 1076) |
| | PCPAPELLGGPSVFLFPP | | | |
| | KPKDTLMISRTPEVTCV | | | |
| | VVDVSHEDPEVKFNWY | | | |
| | VDGVEVHNAKTKPREE | | | |
| | QYNSTYRVVSVLTVLH | | | |
| | QDWLNGKEYKCKVSN | | | |
| | KALPAPIEKTISKAKGQ | | | |
| | PREPQVYTLPPSRDELT | | | |
| | KNQVSLTCLVKGFYPS | | | |
| | DIAVEWESNGQPENNY | | | |
| | KTTPPVLDSDGSFFLYS | | | |
| | KLTVDKSRWQQGNVFS | | | |
| | CSVMHEALHNHYTQKS | | | |
| | LSLSPGK | | | |
| | (SEQ ID NO: 1073) | | | |
| P9-22 | EVQLVESGGGLVQPGG | DIQMTQSPSSLSASVGDRV | EVQLVESGGGL | DIQMTQSPSS |
| | SLRLSCAASGFTFSSYYI | TITCRASQSVSSAVAWYQQ | VQPGGSLRLSC | LSASVGDRV |
| | HWVRQAPGKGLEWVA | KPGKAPKLLIYSASSLYSG | AASGFTFSSYYI | TITCRASQSV |
| | AIYPFSGGTYYADSVK | VPSRFSGSRSGTDFTLTISS | HWVRQAPGKG | SSAVAWYQQ |
| | GRFTISADTSKNTAYLQ | LQPEDFATYYCQQYKSSP | LEWVAAIYPFS | KPGKAPKLLI |
| | MNSLRAEDTAVYYCAR | WTFGQGTKVEIKRTVAAPS | GGTYYADSVK | YSASSLYSGV |
| | GYYVYVVMDYWGQG | VFIFPPSDSQLKSGTASVVC | GRFTISADTSKN | PSRFSGSRSG |
| | TLVTVSSASTKGPSVFP | LLNNFYPREAKVQWKVDN | TAYLQMNSLRA | TDFTLTISSLQ |
| | LAPSSKSTSGGTAALGC | ALQSGNSQESVTEQDSKDS | EDTAVYYCAR | PEDFATYYC |
| | LVKDYFPEPVTVSWNS | TYSLSSTLTLSKADYEKHK | GYYVYVVMDY | QQYKSSPWT |
| | GALTSGVHTFPAVLQSS | VYACEVTHQGLSSPVTKSF | WGQGTLVTVSS | FGQGTKVEIK |
| | GLYSLSSVVTVPSSSLG | NRGEC | (SEQ ID | RTV |
| | TQTYICNVNHKPSNTK | (SEQ ID NO: 1078) | NO: 1079) | (SEQ ID |
| | VDKKVEPKSCDKTHTC | | | NO: 1080) |
| | PPCPAPELLGGPSVFLFP | | | |
| | PKPKDTLMISRTPEVTC | | | |
| | VVVDVSHEDPEVKFNW | | | |
| | YVDGVEVHNAKTKPRE | | | |
| | EQYNSTYRVVSVLTVL | | | |
| | HQDWLNGKEYKCKVS | | | |
| | NKALPAPIEKTISKAKG | | | |
| | QPREPQVYTLPPSRDEL | | | |
| | TKNQVSLTCLVKGFYP | | | |
| | SDIAVEWESNGQPENN | | | |
| | YKTTPPVLDSDGSFFLY | | | |
| | SKLTVDKSRWQQGNVF | | | |
| | SCSVMHEALHNHYTQK | | | |
| | SLSLSPGK | | | |
| | (SEQ ID NO: 1077) | | | |
| P9-27 | EVQLVESGGGLVQPGG | DIQMTQSPSSLSASVGDRV | EVQLVESGGGL | DIQMTQSPSS |
| | SLRLSCAASGFTFWGY | TITCRASQSVSSAVAWYQQ | VQPGGSLRLSC | LSASVGDRV |
| | GIHWVRQAPGKGLEW | KPGKAPKLLIYSASSLYSG | AASGFTFWGYG | TITCRASQSV |
| | VAAIYPYGGSTYYADS | VPSRFSGSRSGTDFTLTISS | IHWVRQAPGKG | SSAVAWYQQ |
| | VKGRFTISADTSKNTAY | LQPEDFATYYCQQRYSTPV | LEWVAAIYPYG | KPGKAPKLLI |
| | LQMNSLRAEDTAVYYC | TFGQGTKVEIKRTVAAPSV | GSTYYADSVKG | YSASSLYSGV |
| | ARLSDIYHSFSGMDYW | FIFPPSDSQLKSGTASVVCL | RFTISADTSKNT | PSRFSGSRSG |
| | GQGTLVTVSSASTKGPS | LNNFYPREAKVQWKVDN | AYLQMNSLRAE | TDFTLTISSLQ |
| | VFPLAPSSKSTSGGTAA | ALQSGNSQESVTEQDSKDS | DTAVYYCARLS | PEDFATYYC |
| | LGCLVKDYFPEPVTVS | TYSLSSTLTLSKADYEKHK | DIYHSFSGMDY | QQRYSTPVTF |
| | WNSGALTSGVHTFPAV | VYACEVTHQGLSSPVTKSF | WGQGTLVTVSS | GQGTKVEIKR |
| | LQSSGLYSLSSVVTVPS | NRGEC | (SEQ ID | TV |
| | SSLGTQTYICNVNHKPS | (SEQ ID NO: 1082) | NO: 1083) | (SEQ ID |
| | NTKVDKKVEPKSCDKT | | | NO: 1084) |
| | HTCPPCPAPELLGGPSV | | | |
| | FLFPPKPKDTLMISRTPE | | | |
| | VTCVVVDVSHEDPEVK | | | |
| | FNWYVDGVEVHNAKT | | | |
| | KPREEQYNSTYRVVSV | | | |
| | LTVLHQDWLNGKEYK | | | |
| | CKVSNKALPAPIEKTIS | | | |
| | KAKGQPREPQVYTLPPS | | | |
| | RDELTKNQVSLTCLVK | | | |
| | GFYPSDIAVEWESNGQP | | | |
| | ENNYKTTPPVLDSDGSF | | | |
| | FLYSKLTVDKSRWQQG | | | |
| | NVFSCSVMHEALHNHY | | | |
| | TQKSLSLSPGK | | | |
| | (SEQ ID NO: 1081) | | | |
| P9-28 | EVQLVESGGGLVQPGG | DIQMTQSPSSLSASVGDRV | EVQLVESGGGL | DIQMTQSPSS |
| | SLRLSCAASGFTFGFYY | TITCRASQSVSSAVAWYQQ | VQPGGSLRLSC | LSASVGDRV |
| | IHWVRQAPGKGLEWV | KPGKAPKLLIYSASSLYSG | AASGFTFGFYYI | TITCRASQSV |
| | AFIDPHGGSTYYADSV | VPSRFSGSRSGTDFTLTISS | HWVRQAPGKG | SSAVAWYQQ |
| | KGRFTISADTSKNTAYL | LQPEDFATYYCQQGYSTL | LEWVAFIDPHG | KPGKAPKLLI |
| | QMNSLRAEDTAVYYC | VTFGQGTKVEIKRTVAAPS | GSTYYADSVKG | YSASSLYSGV |
| | ARLSYPGVLDYWGQGT | VFIFPPSDSQLKSGTASVVC | RFTISADTSKNT | PSRFSGSRSG |
| | LVTVSSASTKGPSVFPL | LLNNFYPREAKVQWKVDN | AYLQMNSLRAE | TDFTLTISSLQ |
| | APSSKSTSGGTAALGCL | ALQSGNSQESVTEQDSKDS | DTAVYYCARLS | PEDFATYYC |
| | VKDYFPEPVTVSWNSG | TYSLSSTLTLSKADYEKHK | YPGVLDYWGQ | QQGYSTLVT |
| | ALTSGVHTFPAVLQSSG | VYACEVTHQGLSSPVTKSF | GTLVTVSS | FGQGTKVEIK |
| | LYSLSSVVTVPSSSLGT | NRGEC | (SEQ ID | RTV |
| | QYICNVNHKPSNTKVD | (SEQ ID NO: 1086) | NO: 1087) | (SEQ ID |
| | KKVEPKSCDKTHTCPP | | | NO: 1088) |
| | CPAPELLGGPSVFLFPP | | | |
| | KPKDTLMISRTPEVTCV | | | |
| | VVDVSHEDPEVKFNWY | | | |
| | VDGVEVHNAKTKPREE | | | |
| | QYNSTYRVVSVLTVLH | | | |
| | QDWLNGKEYKCKVSN | | | |
| | KALPAPIEKTISKAKGQ | | | |
| | PREPQVYTLPPSRDELT | | | |
| | KNQVSLTCLVKGFYPS | | | |
| | DIAVEWESNGQPENNY | | | |
| | KTTPPVLDSDGSFFLYS | | | |
| | KLTVDKSRWQQGNVFS | | | |
| | CSVMHEALHNHYTQKS | | | |
| | LSLSPGK | | | |
| | (SEQ ID NO: 1085) | | | |
| P9-31 | EVQLVESGGGLVQPGG | DIQMTQSPSSLSASVGDRV | EVQLVESGGGL | DIQMTQSPSS |
| | SLRLSCAASGFTFSQYA | TITCRASQSVSRAVAWYQ | VQPGGSLRLSC | LSASVGDRV |
| | IHWVRQAPGKGLEWV | QKPGKAPKLLIYSASSLYS | AASGFTFSQYAI | TITCRASQSV |
| | ARIYPDSGYTYYADSV | GVPSRFSGSRSGTDFTLTIS | HWVRQAPGKG | SRAVAWYQQ |
| | KGRFTISADTSKNTAYL | SLQPEDFATYYCQQYYSPL | LEWVARIYPDS | KPGKAPKLLI |
| | QMNSLRAEDTAVYYC | LTFGQGTKVEIKRTVAAPS | GYTYYADSVK | YSASSLYSGV |
| | ARPYHQYAEGMDYWG | VFIFPPSDSQLKSGTASVVC | GRFTISADTSKN | PSRFSGSRSG |
| | QGTLVTVSSASTKGPSV | LLNNFYPREAKVQWKVDN | TAYLQMNSLRA | TDFTLTISSLQ |
| | FPLAPSSKSTSGGTAAL | ALQSGNSQESVTEQDSKDS | EDTAVYYCARP | PEDFATYYC |
| | GCLVKDYFPEPVTVSW | TYSLSSTLTLSKADYEKHK | YHQYAEGMDY | QQYYSPLLTF |
| | NSGALTSGVHTFPAVL | VYACEVTHQGLSSPVTKSF | WGQGTLVTVSS | GQGTKVEIKR |
| | QSSGLYSLSSVVTVPSS | NRGEC | (SEQ ID | TV |
| | SLGTQTYICNVNHKPSN | (SEQ ID NO: 1090) | NO: 1091) | (SEQ ID |
| | TKVDKKVEPKSCDKTH | | | NO: 1092) |
| | TCPPCPAPELLGGPSVF | | | |
| | LFPPKPKDTLMISRTPE | | | |
| | VTCVVVDVSHEDPEVK | | | |
| | FNWYVDGVEVHNAKT | | | |
| | KPREEQYNSTYRVVSV | | | |
| | LTVLHQDWLNGKEYK | | | |
| | CKVSNKALPAPIEKTIS | | | |
| | KAKGQPREPQVYTLPPS | | | |
| | RDELTKNQVSLTCLVK | | | |
| | GFYPSDIAVEWESNGQP | | | |
| | ENNYKTTPPVLDSDGSF | | | |
| | FLYSKLTVDKSRWQQG | | | |
| | NVFSCSVMHEALHNHY | | | |
| | TQKSLSLSPGK | | | |
| | (SEQ ID NO: 1089) | | | |
| P9-32 | EVQLVESGGGLVQPGG | DIQMTQSPSSLSASVGDRV | EVQLVESGGGL | DIQMTQSPSS |
| | SLRLSCAASGFTFSAYW | TITCRASQSVSSAVAWYQQ | VQPGGSLRLSC | LSASVGDRV |
| | IHWVRQAPGKGLEWV | KPGKAPKLLIYSASSLYSG | AASGFTFSAYW | TITCRASQSV |
| | ALIGPDGGYTYYADSV | VPSRFSGSRSGTDFTLTISS | IHWVRQAPGKG | SSAVAWYQQ |
| | KGRFTISADTSKNTAYL | LQPEDFATYYCQQWSSPL | LEWVALIGPDG | KPGKAPKLLI |
| | QMNSLRAEDTAVYYC | HTFGQGTKVEIKRTVAAPS | GYTYYADSVK | YSASSLYSGV |
| | ARQASRGLDYWGQGT | VFIFPPSDSQLKSGTASVVC | GRFTISADTSKN | PSRFSGSRSG |
| | LVTVSSASTKGPSVFPL | LLNNFYPREAKVQWKVDN | TAYLQMNSLRA | TDFTLTISSLQ |
| | APSSKSTSGGTAALGCL | ALQSGNSQESVTEQDSKDS | EDTAVYYCAR | PEDFATYYC |
| | VKDYFPEPVTVSWNSG | TYSLSSTLTLSKADYEKHK | QASRGLDYWG | QQWSSPLHT |
| | ALTSGVHTFPAVLQSSG | VYACEVTHQGLSSPVTKSF | QGTLVTVSS | FGQGTKVEIK |
| | LYSLSSVVTVPSSSLGT | NRGEC | (SEQ ID | RTV |
| | QTYICNVNHKPSNTKV | (SEQ ID NO: 1094) | NO: 1095) | (SEQ ID |
| | DKKVEPKSCDKTHTCP | | | NO: 1096) |
| | PCPAPELLGGPSVFLFPP | | | |
| | KPKDTLMISRTPEVTCV | | | |
| | VVDVSHEDPEVKFNWY | | | |
| | VDGVEVHNAKTKPREE | | | |
| | QYNSTYRVVSVLTVLH | | | |
| | QDWLNGKEYKCKVSN | | | |
| | KALPAPIEKTISKAKGQ | | | |
| | PREPQVYTLPPSRDELT | | | |
| | KNQVSLTCLVKGFYPS | | | |
| | DIAVEWESNGQPENNY | | | |
| | KTTPPVLDSDGSFFLYS | | | |
| | KLTVDKSRWQQGNVFS | | | |
| | CSVMHEALHNHYTQKS | | | |
| | LSLSPGK | | | |
| | (SEQ ID NO: 1093) | | | |
| P9-36 | EVQLVESGGGLVQPGG | DIQMTQSPSSLSASVGDRV | EVQLVESGGGL | DIQMTQSPSS |
| | SLRLSCAASGFTFGTYY | TITCRASQSVSSAVAWYQQ | VQPGGSLRLSC | LSASVGDRV |
| | IHWVRQAPGKGLEWV | KPGKAPKLLIYSASSLYSG | AASGFTFGTYYI | TITCRASQSV |
| | ASILSGGGYTVYADSV | VPSRFSGSRSGTDFTLTISS | HWVRQAPGKG | SSAVAWYQQ |
| | KGRFTISADTSKNTAYL | LQPEDFATYYCQQDSWGL | LEWVASILSGG | KPGKAPKLLI |
| | QMNSLRAEDTAVYYC | WTFGQGTKVEIKRTVAAPS | GYTVYADSVK | YSASSLYSGV |
| | ARRVYPGFDYWGQGT | VFIFPPSDSQLKSGTASVVC | GRFTISADTSKN | PSRFSGSRSG |
| | LVTVSSASTKGPSVFPL | LLNNFYPREAKVQWKVDN | TAYLQMNSLRA | TDFTLTISSLQ |
| | APSSKSTSGGTAALGCL | ALQSGNSQESVTEQDSKDS | EDTAVYYCARR | PEDFATYYC |
| | VKDYFPEPVTVSWNSG | TYSLSSTLTLSKADYEKHK | VYPGFDYWGQ | QQDSWGLWT |
| | ALTSGVHTFPAVLQSSG | VYACEVTHQGLSSPVTKSF | GTLVTVSS | FGQGTKVEIK |
| | LYSLSSVVTVPSSSLGT | NRGEC | (SEQ ID | RTV |
| | QTYICNVNHKPSNTKV | (SEQ ID NO: 1098) | NO: 1099) | (SEQ ID |
| | DKKVEPKSCDKTHTCP | | | NO: 1100) |
| | PCPAPELLGGPSVFLFPP | | | |
| | KPKDTLMISRTPEVTCV | | | |
| | VVDVSHEDPEVKFNWY | | | |
| | VDGVEVHNAKTKPREE | | | |
| | QYNSTYRVVSVLTVLH | | | |
| | QDWLNGKEYKCKVSN | | | |
| | KALPAPIEKTISKAKGQ | | | |
| | PREPQVYTLPPSRDELT | | | |
| | KNQVSLTCLVKGFYPS | | | |
| | DIAVEWESNGQPENNY | | | |
| | KTTPPVLDSDGSFFLYS | | | |
| | KLTVDKSRWQQGNVFS | | | |
| | CSVMHEALHNHYTQKS | | | |
| | LSLSPGK | | | |
| | (SEQ ID NO: 1097) | | | |
| P9-39 | EVQLVESGGGLVQPGG | DIQMTQSPSSLSASVGDRV | EVQLVESGGGL | DIQMTQSPSS |
| | SLRLSCAASGFTFSFYGI | TITCRASQSVSSAVAWYQQ | VQPGGSLRLSC | LSASVGDRV |
| | HWVRQAPGKGLEWVA | KPGKAPKLLIYSASSLYSG | AASGFTFSFYGI | TITCRASQSV |
| | WIYPYGGFTDYADSVK | VPSRFSGSRSGTDFTLTISS | HWVRQAPGKG | SSAVAWYQQ |
| | GRFTISADTSKNTAYLQ | LQPEDFATYYCQQVQTSL | LEWVAWIYPY | KPGKAPKLLI |
| | MNSLRAEDTAVYYCAR | ATFGQGTKVEIKRTVAAPS | GGFTDYADSVK | YSASSLYSGV |
| | SGFFAFDYWGQGTLVT | VFIFPPSDSQLKSGTASVVC | GRFTISADTSKN | PSRFSGSRSG |
| | VSSASTKGPSVFPLAPS | LLNNFYPREAKVQWKVDN | TAYLQMNSLRA | TDFTLTISSLQ |
| | SKSTSGGTAALGCLVK | ALQSGNSQESVTEQDSKDS | EDTAVYYCARS | PEDFATYYC |
| | DYFPEPVTVSWNSGAL | TYSLSSTLTLSKADYEKHK | GFFAFDYWGQ | QQVQTSLAT |
| | TSGVHTFPAVLQSSGLY | VYACEVTHQGLSSPVTKSF | GTLVTVSS | FGQGTKVEIK |
| | SLSSVVTVPSSSLGTQT | NRGEC | (SEQ ID | RTV |
| | YICNVNHKPSNTKVDK | (SEQ ID NO: 1102) | NO: 1103) | (SEQ ID |
| | KVEPKSCDKTHTCPPCP | | | NO: 1104) |
| | APELLGGPSVFLFPPKP | | | |
| | KDTLMISRTPEVTCVVV | | | |
| | DVSHEDPEVKFNWYVD | | | |
| | GVEVHNAKTKPREEQY | | | |
| | NSTYRVVSVLTVLHQD | | | |
| | WLNGKEYKCKVSNKA | | | |
| | LPAPIEKTISKAKGQPRE | | | |
| | PQVYTLPPSRDELTKNQ | | | |
| | VSLTCLVKGFYPSDIAV | | | |
| | EWESNGQPENNYKTTP | | | |
| | PVLDSDGSFFLYSKLTV | | | |
| | DKSRWQQGNVFSCSV | | | |
| | MHEALHNHYTQKSLSL | | | |
| | SPGK | | | |
| | (SEQ ID NO: 1101) | | | |
| P9-49 | EVQLVESGGGLVQPGG | DIQMTQSPSSLSASVGDRV | EVQLVESGGGL | DIQMTQSPSS |
| | SLRLSCAASGFTFSWYE | TITCRASQSVSSAVAWYQQ | VQPGGSLRLSC | LSASVGDRV |
| | IHWVRQAPGKGLEWV | KPGKAPKLLIYSASSLYSG | AASGFTFSWYE | TITCRASQSV |
| | ARIGPYSSYTYYADSVK | VPSRFSGSRSGTDFTLTISS | IHWVRQAPGKG | SSAVAWYQQ |
| | GRFTISADTSKNTAYLQ | LQPEDFATYYCQQSFSSPV | LEWVARIGPYS | KPGKAPKLLI |
| | MNSLRAEDTAVYYCAR | TFGQGTKVEIKRTVAAPSV | SYTYYADSVKG | YSASSLYSGV |
| | TYYPSYGMDYWGQGT | FIFPPSDSQLKSGTASVVCL | RFTISADTSKNT | PSRFSGSRSG |
| | LVTVSSASTKGPSVFPL | LNNFYPREAKVQWKVDN | AYLQMNSLRAE | TDFTLTISSLQ |
| | APSSKSTSGGTAALGCL | ALQSGNSQESVTEQDSKDS | DTAVYYCART | PEDFATYYC |
| | VKDYFPEPVTVSWNSG | TYSLSSTLTLSKADYEKHK | YYPSYGMDYW | QQSFSSPVTF |
| | ALTSGVHTFPAVLQSSG | VYACEVTHQGLSSPVTKSF | GQGTLVTVSS | GQGTKVEIKR |
| | LYSLSSVVTVPSSSLGT | NRGEC | (SEQ ID | TV |
| | QTYICNVNHKPSNTKV | (SEQ ID NO: 1106) | NO: 1107) | (SEQ ID |
| | DKKVEPKSCDKTHTCP | | | NO: 1108) |
| | PCPAPELLGGPSVFLFPP | | | |
| | KPKDTLMISRTPEVTCV | | | |
| | VVDVSHEDPEVKFNWY | | | |
| | VDGVEVHNAKTKPREE | | | |
| | QYNSTYRVVSVLTVLH | | | |
| | QDWLNGKEYKCKVSN | | | |
| | KALPAPIEKTISKAKGQ | | | |
| | PREPQVYTLPPSRDELT | | | |
| | KNQVSLTCLVKGFYPS | | | |
| | DIAVEWESNGQPENNY | | | |
| | KTTPPVLDSDGSFFLYS | | | |
| | KLTVDKSRWQQGNVFS | | | |
| | CSVMHEALHNHYTQKS | | | |
| | LSLSPGK | | | |
| | (SEQ ID NO: 1105) | | | |
| P9-54 | EVQLVESGGGLVQPGG | DIQMTQSPSSLSASVGDRV | EVQLVESGGGL | DIQMTQSPSS |
| | SLRLSCAASGFTFSTYFI | TITCRASQSVSSAVAWYQQ | VQPGGSLRLSC | LSASVGDRV |
| | HWVRQAPGKGLEWVA | KPGKAPKLLIYSASSLYSG | AASGFTFSTYFI | TITCRASQSV |
| | WISPSGSHTGYADSVK | VPSRFSGSRSGTDFTLTISS | HWVRQAPGKG | SSAVAWYQQ |
| | GRFTISADTSKNTAYLQ | LQPEDFATYYCQQWYPSLI | LEWVAWISPSG | KPGKAPKLLI |
| | MNSLRAEDTAVYYCAR | TFGQGTKVEIKRTVAAPSV | SHTGYADSVKG | YSASSLYSGV |
| | VRYPGVMDYWGQGTL | FIFPPSDSQLKSGTASVVCL | RFTISADTSKNT | PSRFSGSRSG |
| | VTVSSASTKGPSVFPLA | LNNFYPREAKVQWKVDN | AYLQMNSLRAE | TDFTLTISSLQ |
| | PSSKSTSGGTAALGCLV | ALQSGNSQESVTEQDSKDS | DTAVYYCARV | PEDFATYYC |
| | KDYFPEPVTVSWNSGA | TYSLSSTLTLSKADYEKHK | RYPGVMDYWG | QQWYPSLITF |
| | LTSGVHTFPAVLQSSGL | VYACEVTHQGLSSPVTKSF | QGTLVTVSS | GQGTKVEIKR |
| | YSLSSVVTVPSSSLGTQ | NRGEC | (SEQ ID | TV |
| | TYICNVNHKPSNTKVD | (SEQ ID NO: 1110) | NO: 1111) | (SEQ ID |
| | KKVEPKSCDKTHTCPP | | | NO: 1112) |
| | CPAPELLGGPSVFLFPP | | | |
| | KPKDTLMISRTPEVTCV | | | |
| | VVDVSHEDPEVKFNWY | | | |
| | VDGVEVHNAKTKPREE | | | |
| | QYNSTYRVVSVLTVLH | | | |
| | QDWLNGKEYKCKVSN | | | |
| | KALPAPIEKTISKAKGQ | | | |
| | PREPQVYTLPPSRDELT | | | |
| | KNQVSLTCLVKGFYPS | | | |
| | DIAVEWESNGQPENNY | | | |
| | KTTPPVLDSDGSFFLYS | | | |
| | KLTVDKSRWQQGNVFS | | | |
| | CSVMHEALHNHYTQKS | | | |
| | LSLSPGK | | | |
| | (SEQ ID NO: 1109) | | | |
| P9-55 | EVQLVESGGGLVQPGG | DIQMTQSPSSLSASVGDRV | EVQLVESGGGL | DIQMTQSPSS |
| NEG. | SLRLSCAASGFTFATYY | TITCRASQSVSSAVAWYQQ | VQPGGSLRLSC | LSASVGDRV |
| CON. | IHWVRQAPGKGLEWV | KPGKAPKLLIYSASSLYSG | AASGFTFATYYI | TITCRASQSV |
| | AYIDSESGYTYYADSV | VPSRFSGSRSGTDFTLTISS | HWVRQAPGKG | SSAVAWYQQ |
| | KGRFTISADTSKNTAYL | LQPEDFATYYCQQRYSSLL | LEWVAYIDSES | KPGKAPKLLI |
| | QMNSLRAEDTAVYYC | TFGQGTKVEIKRTVAAPSV | GYTYYADSVK | YSASSLYSGV |
| | ARVSRGSSGTHVMDY | FIFPPSDSQLKSGTASVVCL | GRFTISADTSKN | PSRFSGSRSG |
| | WGQGTLVTVSSASTKG | LNNFYPREAKVQWKVDN | TAYLQMNSLRA | TDFTLTISSLQ |
| | PSVFPLAPSSKSTSGGT | ALQSGNSQESVTEQDSKDS | EDTAVYYCAR | PEDFATYYC |
| | AALGCLVKDYFPEPVT | TYSLSSTLTLSKADYEKHK | VSRGSSGTHVM | QQRYSSLLTF |
| | VSWNSGALTSGVHTFP | VYACEVTHQGLSSPVTKSF | DYWGQGTLVT | GQGTKVEIKR |
| | AVLQSSGLYSLSSVVTV | NRGEC | VSS | TV |
| | PSSSLGTQTYICNVNHK | (SEQ ID NO: 1114) | (SEQ ID | (SEQ ID |
| | PSNTKVDKKVEPKSCD | | NO: 1115) | NO: 1116) |
| | KTHTCPPCPAPELLGGP | | | |
| | SVFLFPPKPKDTLMISR | | | |
| | TPEVTCVVVDVSHEDP | | | |
| | EVKFNWYVDGVEVHN | | | |
| | AKTKPREEQYNSTYRV | | | |
| | VSVLTVLHQDWLNGKE | | | |
| | YKCKVSNKALPAPIEKT | | | |
| | ISKAKGQPREPQVYTLP | | | |
| | PSRDELTKNQVSLTCLV | | | |
| | KGFYPSDIAVEWESNG | | | |
| | QPENNYKTTPPVLDSD | | | |
| | GSFFLYSKLTVDKSRW | | | |
| | QQGNVFSCSVMHEALH | | | |
| | NHYTQKSLSLSPGK | | | |
| | (SEQ ID NO: 1113) | | | |
| P9-58 | EVQLVESGGGLVQPGG | DIQMTQSPSSLSASVGDRV | EVQLVESGGGL | DIQMTQSPSS |
| | SLRLSCAASGFTFSRYY | TITCRASQSVSSAVAWYQQ | VQPGGSLRLSC | LSASVGDRV |
| | IHWVRQAPGKGLEWV | KPGKAPKLLIYSASSLYSG | AASGFTFSRYYI | TITCRASQSV |
| | AFISSDSGYTQYADSVK | VPSRFSGSRSGTDFTLTISS | HWVRQAPGKG | SSAVAWYQQ |
| | GRFTISADTSKNTAYLQ | LQPEDFATYYCQQGFGFLV | LEWVAFISSDS | KPGKAPKLLI |
| | MNSLRAEDTAVYYCAR | TFGQGTKVEIKRTVAAPSV | GYTQYADSVK | YSASSLYSGV |
| | TMSYSALDYWGQGTL | FIFPPSDSQLKSGTASVVCL | GRFTISADTSKN | PSRFSGSRSG |
| | VTVSSASTKGPSVFPLA | LNNFYPREAKVQWKVDN | TAYLQMNSLRA | TDFTLTISSLQ |
| | PSSKSTSGGTAALGCLV | ALQSGNSQESVTEQDSKDS | EDTAVYYCART | PEDFATYYC |
| | KDYFPEPVTVSWNSGA | TYSLSSTLTLSKADYEKHK | MSYSALDYWG | QQGFGFLVTF |
| | LTSGVHTFPAVLQSSGL | VYACEVTHQGLSSPVTKSF | QGTLVTVSS | GQGTKVEIKR |
| | YSLSSVVTVPSSSLGTQ | NRGEC | (SEQ ID | TV |
| | TYICNVNHKPSNTKVD | (SEQ ID NO: 1118) | NO: 1119) | (SEQ ID |
| | KKVEPKSCDKTHTCPP | | | NO: 1120) |
| | CPAPELLGGPSVFLFPP | | | |
| | KPKDTLMISRTPEVTCV | | | |
| | VVDVSHEDPEVKFNWY | | | |
| | VDGVEVHNAKTKPREE | | | |
| | QYNSTYRVVSVLTVLH | | | |
| | QDWLNGKEYKCKVSN | | | |
| | KALPAPIEKTISKAKGQ | | | |
| | PREPQVYTLPPSRDELT | | | |
| | KNQVSLTCLVKGFYPS | | | |
| | DIAVEWESNGQPENNY | | | |
| | KTTPPVLDSDGSFFLYS | | | |
| | KLTVDKSRWQQGNVFS | | | |
| | CSVMHEALHNHYTQKS | | | |
| | LSLSPGK | | | |
| | (SEQ ID NO: 1117) | | | |

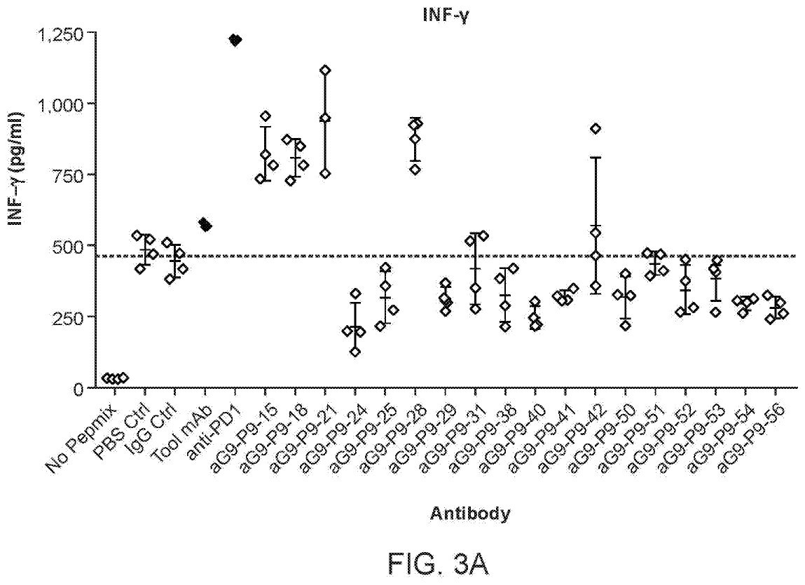

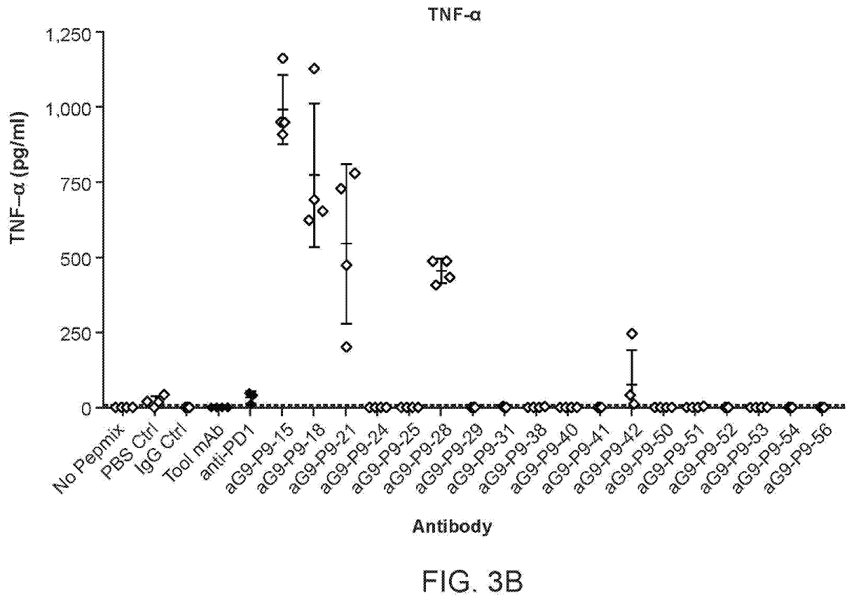

Select GAL9 binding candidates were analyzed for binding properties: cross-reactive binding with murine GAL9, qualitative binding, epitope binning (Bin 2—candidates bin with Commercial antibody Clone ECA8 from LS Bio [LS-C179448], Bin 3—candidates bin with Commercial antibody Clone ECA42 from LS Bio [LS-C179449], which is the “tool antibody” referenced in FIG. 3), and monovalent affinity binding. Analysis results are presented in Table 7.

TABLE 7
Candidate hGAL9 Binding Properties

| | Mouse | | | |
|---|---|---|---|---|
| | Cross- | Binding Off-Rate | | Calculated |
| ABS | reactivity | (++ = moderate, +++ = slow) | Bin | K_{D} (M) |
| P9-02B | Y | +++ | 1 | |
| P9-04 | | +++ | 3 | |
| P9-05 | Y | +++ | 2 | |
| P9-08 | | ++ | 3 | |
| P9-09 | Y | +++ | 1 | |
| P9-10 | | ++ | 2 | 5.77 × 10⁻⁹ |
| P9-15 | | ++ | 1 | 2.208 × 10⁻⁹ |
| P9-16 | Y | +++ | 1 | 2.87 × 10⁻⁹ |
| P9-18 | Y | +++ | 3 | 6.243 × 10⁻⁹ |
| P9-19 | | ++ | 1 | |
| P9-20 | Y | +++ | 1 | |
| P9-21 | Y | +++ | 3 | 2.749 × 10⁻⁹ |
| P9-22 | Y | +++ | 1 | 4.85 × 10⁻⁹ |
| P9-27 | | +++ | 2 | |
| P9-28 | | +++ | 1 | 3.358 × 10⁻⁹ |
| P9-31 | | +++ | 3 | |
| P9-32 | Y | +++ | 1 | 1.083 × 10⁻⁹ |
| P9-36 | | ++ | 1 | |
| P9-39 | | ++ | 1 | |
| P9-49 | | +++ | 1 | |
| P9-54 | | +++ | 1 | |
| P9-55 | | Negative | | |
| | | Control (NEG) | | |
| P9-58 | | ++ | 1 | |

Select GAL9 binding candidates were further analyzed for sequence motifs that could adversely affect antibody properties that are relevant to clinical development, such as stability, mutability, and immunogenicity. Computational analysis was performed according to Kumar and Singh (Developability of biotherapeutics: computational approaches. Boca Raton: CRC Press, Taylor & Francis Group, 2016). Analysis results are presented in Table 8, and demonstrate a limited number of adverse sequence motifs are present in the listed clones, indicating the potential for further clinical development.

TABLE 8
Candidate anti-human GAL9 Antibody Properties

| | | | | | | | | Number | | | |
|---|---|---|---|---|---|---|---|---|---|---|---|
| | CDR3 | | | | Number | Number | Number | N-linked | | Number | Number |
| | Loop | Yield | Mol Weight | Isoelectric | Deamidation | Isomerization | Fragmentation | Glycosylation | Cys in | Other | T-cell |
| ABS | Length | (ug/mL) | (kDa) | Point | Sites¹ | Sites² | Sites³ | Sites⁴ | CDR | Sites⁵ | Epitopes⁶ |
| P9-05 | 11 | 10 | 1.444 × 10⁵ | 8.32 | 0 | 1 | 1 | 0 | No | 0 | 0 |
| P9-10 | 11 | 33 | 1.450 × 10⁵ | 8.08 | 0 | 2 | 2 | 0 | No | 0 | 0 |
| P9-15 | 11 | 100 | 1.444 × 10⁵ | 8.59 | 0 | 1 | 1 | 0 | No | 0 | 1 |
| P9-16 | 13 | 180.9 | 1.450 × 10⁵ | 8.42 | 0 | 1 | 1 | 0 | No | 0 | 1 |
| P9-18 | 11 | 189.5 | 1.448 × 10⁵ | 8.42 | 0 | 1 | 3 | 0 | No | 0 | 0 |
| P9-21 | 13 | 162.5 | 1.451 × 10⁵ | 8.42 | 0 | 1 | 2 | 0 | No | 0 | 2 |
| P9-22 | 13 | 53.7 | 1.448 × 10⁵ | 8.50 | 0 | 1 | 1 | 0 | No | 0 | 1 |
| P9-28 | 12 | 85 | 1.440 × 10⁵ | 8.33 | 0 | 2 | 1 | 0 | No | 0 | 1 |
| P9-32 | 11 | 322.5 | 1.438 × 10⁵ | 8.43 | 0 | 2 | 1 | 0 | No | 0 | 1 |
| P9-55 | | | 1.452 × 10⁵ | 8.42 | 0 | 2 | 1 | 0 | No | 0 | 0 |

¹(NG, NS, NA, NH, ND)
²(DG, DP, DS)
³(DP, DY, HS, KT, HXS, SXH)
⁴(NXS/T)
⁵(LLQG (SEQ ID NO: 1121), HPQ, FHENSP (SEQ ID NO: 1122), LPRWG (SEQ ID NO: 1123), HHH)
⁶3% in at least 2 of DRB1_0101, DRB1_0301, DRB1_0401, DRB1_0701, DRB_1101, DRB1_1301, DRB1_1501, DRB1_0801

### 6.11.3. Example 2: Treatment with Anti-GAL9 Candidates Increases Cytokine Production by Human PBMCs

Candidate GAL9 ABSs were formatted into a bivalent monospecific native human full-length IgG1 heavy chain and light chain architectures (SEQ ID NO:5 and SEQ ID NO: 3, respectively) and were tested for their effect on cytokine production by PBMCs following peptide stimulation. PBMCs were stimulated essentially as described in Section 6.11.1 above. Briefly, PBMCs were harvested from human donors known to be responsive to human CMV virus (HCWV), placed in culture, and stimulated with HCMV PepMix to prime an antigen specific response, and treated with one of: control IgG, a comparator tool activating mAb (clone ECA42), α-PD1 (Nivolumab), or candidate anti-GAL9 antibodies. Cytokine secretion was measured at 24 and 72 hrs post-treatment by bead cytokine array. Results for INF-γ and TNF-α are depicted in FIGS. 3A and 3B, respectively. The data shown in FIGS. 3A-3B is described in more detail in the Tables 9 and 10 provided below.

TABLE 9
INF-γ 72 hr

| | | Average/donor | | | | |
|---|---|---|---|---|---|---|
| | | Donor 5 | Donor 19 | Donor 20 | Average | as % |
| IgG | pg/ml | 446 | 607 | 760 | | |
| P9-15 | pg/ml | 822 | 922 | 1114 | 1.61 | 61% |
| | Fold change | 1.85 | 1.52 | 1.47 | | |
| P9-18 | pg/ml | 808 | 795 | 845 | 1.41 | 41% |
| | Fold change | 1.81 | 1.31 | 1.11 | | |
| P9-21 | pg/ml | 938 | 1006 | 1089 | 1.73 | 73% |
| | Fold change | 2.10 | 1.66 | 1.43 | | |
| P9-28 | pg/ml | 873 | 951 | 1054 | 1.64 | 64% |
| | Fold change | 1.96 | 1.57 | 1.39 | | |

TABLE 10
TNF-α 72 hr

| | | Average/donor | | | | |
|---|---|---|---|---|---|---|
| | | Donor 5 | Donor 19 | Donor 20 | Average | as % |
| IgG | pg/ml | 2 | 111 | 189 | | |
| P9-15 | pg/ml | 992 | 987 | 950 | 193.58 | 19258% |
| | Fold change | 566.81 | 8.91 | 5.02 | | |
| P9-18 | pg/ml | 774 | 693 | 747 | 150.83 | 14983% |
| | Fold change | 442.30 | 6.26 | 3.95 | | |
| P9-21 | pg/ml | 546 | 520 | 612 | 106.63 | 10563% |
| | Fold change | 311.97 | 4.70 | 3.24 | | |
| P9-28 | pg/ml | 455 | 570 | 673 | 89.48 | 8848% |
| | Fold change | 259.74 | 5.14 | 3.56 | | |

Notably, PBMCs treated with candidates P9-15, P9-18, P9-21, and P9-28 demonstrated improved IFN-γ and TNF-α secretion following stimulation relative to both IgG control and the GAL9 comparator Tool antibody (clone ECA42). In addition, PBMCs treated with candidates P9-15, P9-18, P9-21, and P9-28 notably also demonstrated improved TNF-α production following stimulation relative to treatment with a commercial α-PD1 antibody. Thus, treatment of PBMCs with select anti-GAL9 candidates was able to improve cytokine secretion following peptide stimulation. Treatment with P9-54 resulted in a neutral response, with no significant difference in TNF-α and IFN-γ secretion (data not shown).

### 6.11.4. Example 3: Treatment with Anti-GAL9 Candidates Increases TNF-α Production by Natural Killer (NK) Cells

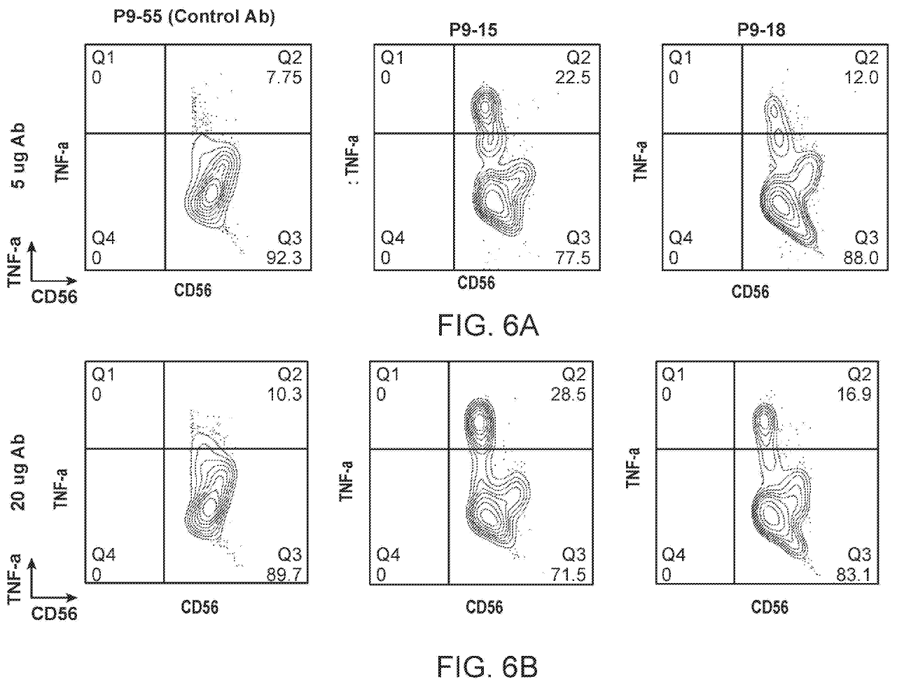

Candidate GAL9 ABSs were formatted into a bivalent monospecific native human full-length IgG1 heavy chain and light chains architectures (SEQ ID NO:5 and SEQ ID NO:3, respectively) and were tested for their effect on TNF-α secretion by NK Cells (lineage, CD56⁺) following 72 hours of peptide stimulation. NK Cells were treated with Control Antibody Clone 55, GAL9 antibody candidate P9-15 (Clone 15), or GAL9 antibody candidate P9-18 (Clone 18), at a dosage of 5 μg or 20 μg. After treatment, cells were assessed for levels of TNF-α secretion by flow cytometry. Representative data for the percentage of NK cells (CD56⁺) that secreted TNF-α are presented in FIG. 6.

Treatment with either GAL9 antibody candidate P9-18 or candidate P9-15 increased the percentages of NK cells that stained positive for TNF-α following stimulation, relative to the Clone P9-55, a negative control. In a population of NK cells treated with 5 μg of control antibody, 7.75% of such NK cells (CD56+) were TNF-α positive. By contrast, in a population of NK cells treated with 5 μg of P9-18, 12.0% of such NK cells were TNF-α positive. And NK cells treated with 5 μg of P9-15, 22.5% of such NK cells were TNF-α positive. See FIG. 6.

In a population of NK cells treated with 20 μg of control antibody, 10.3% of such NK cells (CD56+) were TNF-α positive. By contrast, in a population of NK cells treated with 20 μg of P9-18, 16.9% of such NK cells were TNF-α positive. And NK cells treated with 20 μg of P9-15, 28.5% of such NK cells were TNF-α positive. See FIG. 6.

Thus, treatment with select anti-GAL9 candidates was able to increase TNF-α production by NK cells following stimulation. See FIG. 6.

### 6.11.5. Example 4: Treatment with Anti-GAL9 Candidates Increases IL-12 Production by Dendritic Cells

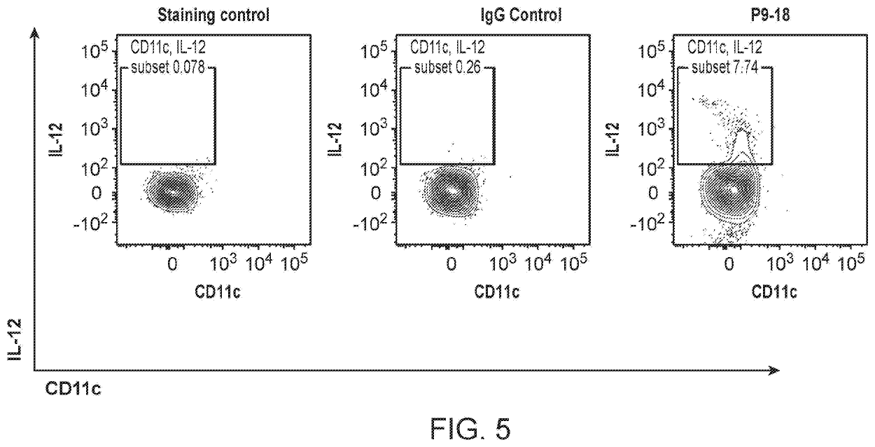

Candidate GAL9 ABSs were formatted into a bivalent monospecific native human full-length IgG1 heavy chain and light chains architectures (SEQ ID NO:5 and SEQ ID NO:3, respectively) and were tested for their effect on IL-12 secretion by dendritic cells (lineage negative, class II⁺, CD11c⁺) following peptide stimulation. PBMCs, which include the population of dendritic cells (DCs), were treated as described in Example 2 then assessed for levels of IL-12 secretion using an IL-12 Secretion Assay-Detection Kit (PE), Human (Cat. No. 130-092-124, Miltenyi Biotec) as per the manufacturers protocol. Representative data for the percentage of DCs that secreted IL-12 are presented in FIG. 5.

Notably, treatment with the GAL9 antibody candidate P9-18 increased the percentages of DCs that stained positive for IL-12 following stimulation, relative to the IgG control. In a population of DCs treated with control IgG, 0.26% of such DCs were IL-12 positive. By contrast, in a population of DCs treated with P9-18, 7.74% of such DCs were IL-12 positive, a 28-fold increase in IL-12 positive DCs relative to the IgG control-treated population. Thus, treatment of PBMCs with select anti-GAL9 candidates was able to increase IL-12 production by DCs following stimulation.

### 6.11.6. Example 5: Treatment with Anti-GAL9 Candidates Increases Surface Expression of Co-Stimulatory Molecules on CD8+ T Cells

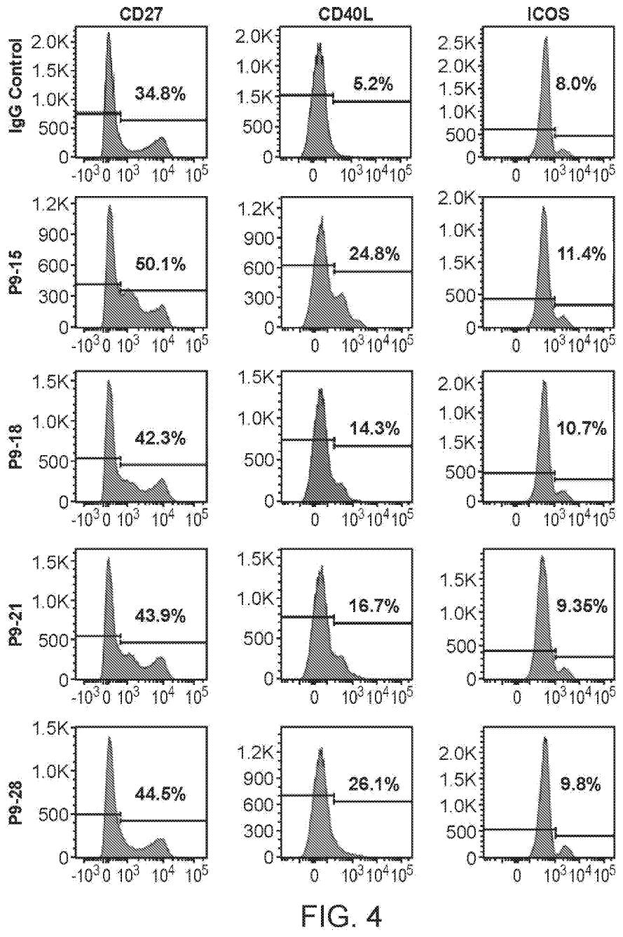

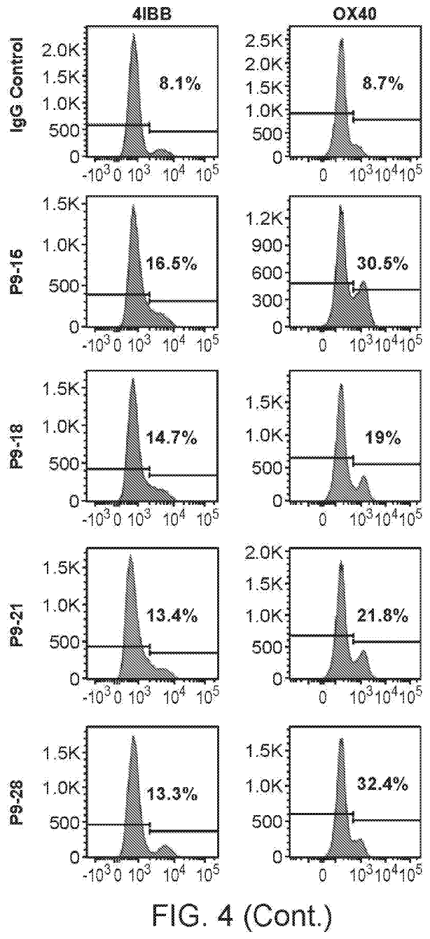

Candidate GAL9 ABSs that had been formatted into a bivalent monospecific native human full-length IgG1 heavy chain and light chain architectures (SEQ ID NO:5 and SEQ ID NO:3, respectively) were tested for their effect on immune stimulatory surface marker expression by CD8⁺ T-cells following peptide stimulation. PBMCs, which include the population of CD8+ T-cells, were treated as described in Example 2, stained with marker antibodies as described herein, then harvested for flow cytometry. Levels of the immune stimulatory surface markers CD27, CD42L, ICOS, 4-1BB, and X40 were assessed on CD8 T-cells. Data are shown in FIG. 4. “% value” represents the 00 of CD8+ T cells with detectable levels of the relevant marker. FIG. 4 indicates that treatment with the αGAL9 antibody candidates P9-15, P9-18, P9-21, and P9-28 increased the immune stimulatory surface markers CD27, CD40L, ICOS, 4-1BB, and OX40 in CD8+ T cells, as compared to an Ig control antibody clone ECA42.

Representative data for the percentage of CD8⁺ T-cells that stained positive for immune stimulatory surface marker are presented in Table 11 below.

TABLE 11
Percent CD8⁺ cells positive for selected costimulatory molecules

| Antibody | 4-1BB | CD27 | CD40L | ICOS | OX40 |
|---|---|---|---|---|---|
| IgG control | 8.13 | 34.8 | 5.21 | 8.03 | 8.68 |
| Comparator Tool mAb | 11.2 | 35.2 | 5.28 | 11.3 | 7.77 |
| (clone ECA42) | | | | | |
| α-PD-1 (Nivolumab) | 8.46 | 34.6 | 5 | 8.45 | 7.81 |
| α-GAL9 (P9-15) | 16.5 (2.1x) | 50 (1.4x) | 24.8 (4.8x) | 11.4 (1.4x) | 30.5 (3.5x) |
| α-GAL9 (P9-18) | 14.7 (1.8x) | 42.3 (1.2x) | 14.7 (2.7x) | 10.7 (1.3x) | 19 (2.2x) |
| α-GAL9 (P9-21) | 13.4 (1.6x) | 43.9 (1.3x) | 16.7 (3.2x) | 9.35 (1.2x) | 21.7 (2.5x) |
| α-GAL9 (P9-22) | 12.1 (1.5x) | 37.2 (1.1x) | 10.3 (2.0x) | 11.1 (1.4x) | 15.3 (1.8x) |
| α-GAL9 (P9-28) | 13.3 (1.6x) | 44.5 (1.3x) | 26.1 (5x) | 9.8 (1.2x) | 22.4 (2.6x) |

Notably, PBMCs treated with candidates P9-18 or P9-21 demonstrated increased percentages of CD8⁺ T-cells that stained positive for the various immune stimulatory surface markers following stimulation relative to the IgG control, the GAL9 comparator Tool antibody (clone ECA42), and α-PD1, including a greater than 2-fold increase in the percentage of CD8+ T-cells that stained positive for CD40L and OX40. Thus, treatment of PBMCs with select anti-GAL9 candidates was able to improve immune stimulatory surface marker expression by CD8⁺ T cells following stimulation. The same immune stimulatory response was observed with low responder PBMC cells, donor 5 (data not shown).

### 6.11.7. Example 6: Treatment with Anti-GAL9 Candidates Alters PD-L1 and PD-L2 Cell Surface Expression on Dendritic Cells (DCs)

Candidate GAL9 ABSs were formatted into a bivalent monospecific native human full-length IgG1 heavy chain and light chains architecture (SEQ ID NO:5 and SEQ ID NO:3, respectively) and were tested for their effect on PD-L1 and PD-L2 cell surface expression on dendritic cells (lineage negative, class II, CD11c⁺) following peptide stimulation. PBMCs, which include the population of dendritic cells (DCs), were treated as described in Example 2 then harvested for flow cytometry and the levels of PD-L1 and PD-L2 were assessed on DCs. Representative data for the percentage of DCs that stained positive for PD-L1 and PD-L2, as well as the geometric mean fluorescent intensity (GMI), are presented in Table 12 below.

TABLE 12
Percent DCs positive for surface PD-L1
and PD-L2, amount of PD-L1/PD-L2 (GMI)

| | % of cells with marker | | GMI | |
|---|---|---|---|---|
| Antibody | PD-L1 | PD-L2 | PD-L1 | PD-L2 |
| IgG control | 84.7 | 1.15 | 37,215 | 3,273 |
| Comparator Tool mAb | 88.2 | 1.58 | 50,395 | 3,616 |
| (clone ECA42) | | | | |
| α-GAL9 (P9-18) | 75.2 | 7.35 | 27,122 | 3,345 |
| α-GAL (P9-21) | 69.8 | 1.38 | 20,090 | 2,551 |

Notably, PBMCs treated with candidate P9-18 demonstrated increased percentages of DCs that stained positive for PD-L2 following stimulation relative to the IgG control and the GAL9 comparator Tool antibody (ECA42). Both P9-18 and P9-21 also demonstrated a decreased percentage of PD-L1, as well as decreased Geometric Mean Fluorescence (GMI) of PD-L1 on DCs. Thus, treatment of PBMCs with select anti-GAL9 candidates was able to alter PD-L1 and PD-L2 surface expression by DCs following stimulation.

### 6.11.8. Example 7: Treatment with Anti-GAL9 Candidates Leads to Clustering of GAL9 and PD-L2 on the Cell Surface of Dendritic Cells

Candidate GAL9 ABSs were formatted into a bivalent monospecific native human full-length IgG1 heavy chain and light chains architecture (SEQ ID NO:5 and SEQ ID NO:3, respectively) and were tested for their effect on clustering of GAL9, PD-L1, and PD-L2 on the cell-surface of dendritic cells (“DCs”).

PBMCs, which include the population of dendritic cells (DCs), were treated as described in Example 2 then fixed for confocal imaging analysis to assess GAL9, CD11c, and PD-L2 distribution on dendritic cells.

Results/Conclusion

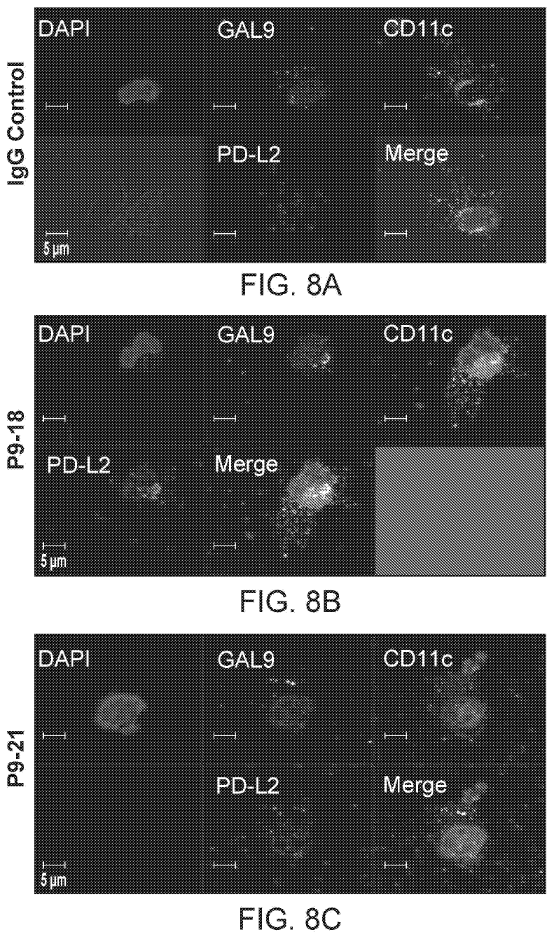

Confocal images of dendritic cells treated with IgG control (FIG. 8A), P9-18 (FIG. 8B), and P9-21 (FIG. 8C) are shown. The blue staining shows DNA (DAPI), the red staining shows PD-L2, the green staining shows CD11c, and the yellow staining shows GAL9. Non-labeled images are bright field; rendered in gray scale in the attached figures.

Treatment with candidate P9-18 or P9-21 demonstrated co-localization and clustering of GAL9 and PD-L2 on DCs (FIGS. 8B-8C), as compared to IgG control. Thus, treatment with P9-18 or P9-21 can induce co-localization and clustering of GAL9 and PD-L2 on the cell surface of DCs following stimulation.

### 6.11.9. Example 8: Treatment with Anti-GAL9 P9-18 Retains PD-L2 and PD-L1 Expression on Tumor Cells

Anti-GAL9 candidate P9-18 was tested for its effect on cellular retention and distribution of PD-L2 and PD-L1 in tumor cells.

Antibodies

Candidate GAL9 ABSs were formatted into a bivalent monospecific native human full-length IgG1 heavy chain and light chain architecture (SEQ ID NO:5 and SEQ ID NO:3, respectively). Anti-PD-L2 clone TY25 and anti-PD-L1 clone 10F.9G2 were obtained from BioXcell (Lebanon, N.H.).

Cell Culture and Immunostaining

CT26 tumor cells were cultured and treated with either anti-GAL9 candidate P9-18 or IgG control. Cells were fixed and stained with DAPI, anti-PD-L2, and anti-PD-L1 for confocal imaging analysis.

Results/Conclusion

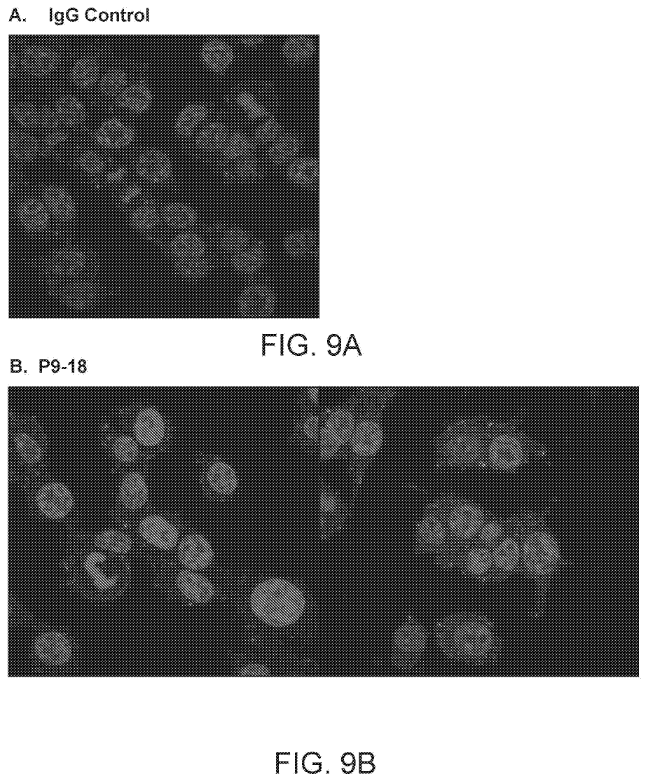

FIGS. 9A and 9B show representative confocal images of CT26 tumor cells after treatment with P9-18 or IgG control. The blue staining shows DNA (DAPI), red shows PD-L2, and green shows PD-L1; rendered in gray scale in the attached figures. The imaging demonstrated that PD-L2 and PD-L1 are retained on the surface of CT26 tumor cells after treatment with P9-18 compared to IgG control. See FIGS. 9A and 9B. The speckles in FIG. 9B highlight increased expression of PD-L2 and PD-L1 proteins.

### 6.11.10. Example 9: Treatment with Anti-GAL9 P9-18 or P9-21 Inhibits Tumor Growth in Colon and Melanoma Tumor Models

This study was conducted to determine if anti-GAL9 candidates P9-18 and P9-21 can inhibit tumor growth in a colon and melanoma tumor models.

Antibodies

Candidate GAL9 ABSs were formatted into a bivalent monospecific formatted on a mouse IgG2a backbone.

Animals and Treatment

BALB/c mice were implanted subcutaneously with CT26 tumor line and treated with anti-GAL9 candidates P9-18, P9-21, or IgG control. Treatments were intraperitoneal (I.P.), 200 μg, on days 7, 11, 15, and 19 with ten mice per treatment group. Tumor growth was assessed by measuring tumor volume. Mice were euthanized if tumors reached a volume of ˜1000 mm³.

C57BL/6 mice were implanted intradermally with a B16.F0 tumor line and treated with anti-GAL9 candidates P9-18, P9-21, or IgG control. Treatments were administered I.P., at 200 μg, on days 3, 7, 11, and 15, with ten mice per treatment group.

Results/Conclusion

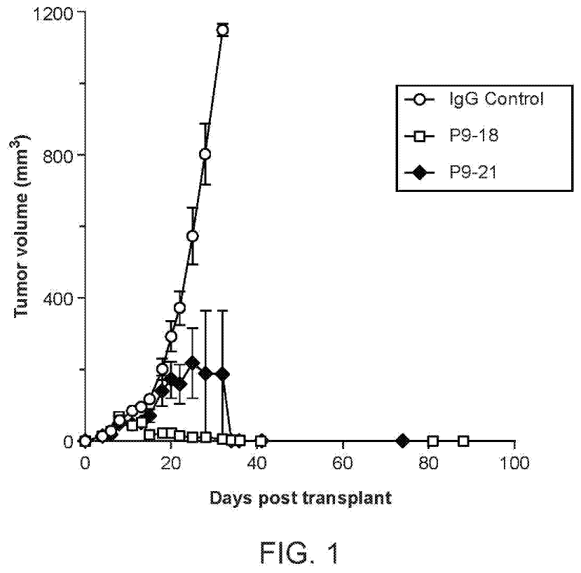

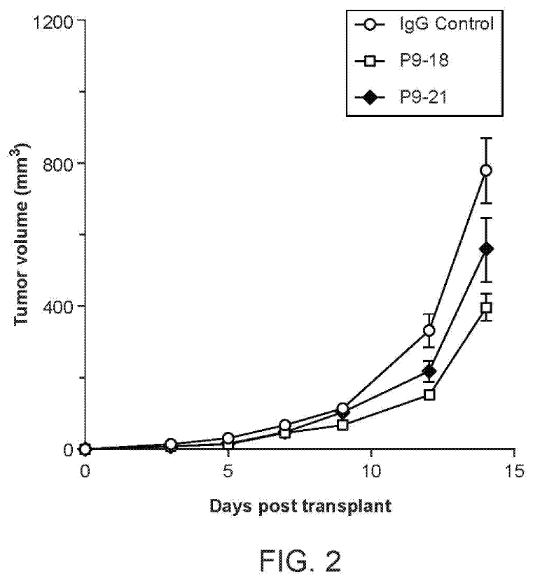

Mice treated with P9-18 or P9-21 demonstrated a complete regression of CT26 tumors, while mice treated with the IgG control demonstrated continued tumor growth. See FIG. 1. Mice treated with P9-18 or P9-21 demonstrated reduced B16.F0 tumor growth compared to mice treated with IgG control. See FIG. 2. Thus, P9-18 or P9-21 can inhibit tumor growth in colon and melanoma tumor models, including complete regression in some cases.

### 6.11.11. Example 10: Treatment with Anti-GAL9 P9-15 Results in Fewer Epstein-Barr Virus (EBV)-Induced Tumors and Reduces Viral Load

This study was conducted to determine the effect of anti-GAL9 P9-15 candidate on Epstein-Barr virus (EBV)-induced tumors in a humanized mouse model.

Epstein-Barr Virus (EBV) is a γ-herpes virus that infects human B cells. However, many human viruses do not infect mice. Therefore, to test the effect of anti-GAL9 P9-15 on EBV-induced tumor, we a used a humanized mouse engrafted with human CD34⁺ hematopoietic stem cells to make a mouse model reconstituted with human immune system cells.

Infection of Humanized Mice and Treatment

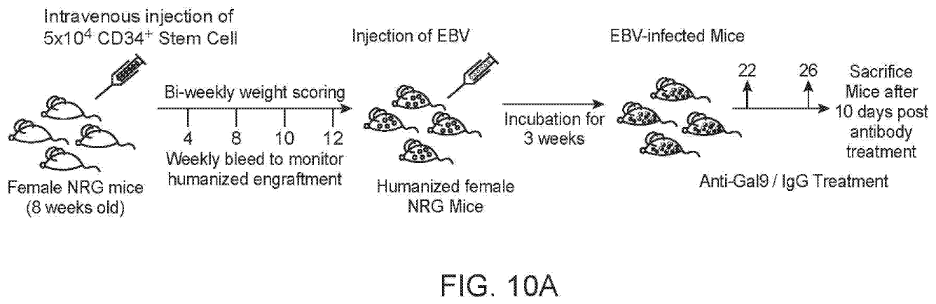

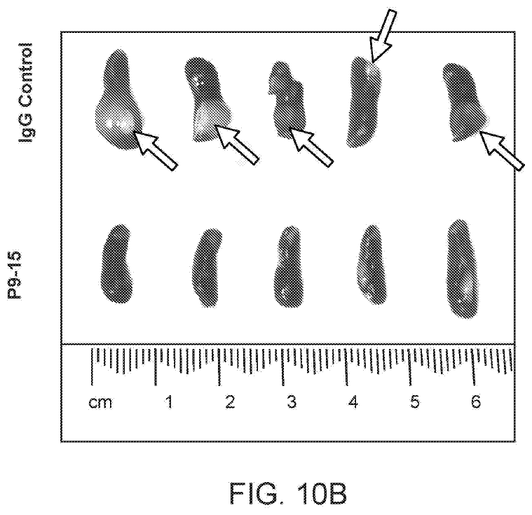

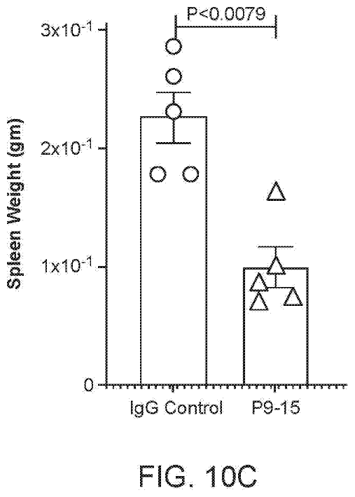

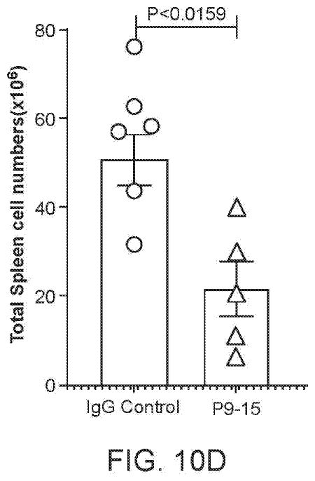

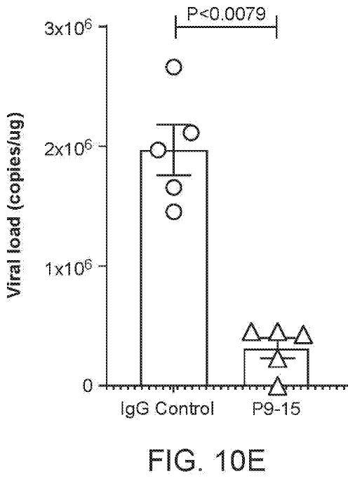

FIG. 10A shows a schematic of the overall treatment schedule used for the study. Briefly, immunodeficient mice were intravenously injected with CD34⁺ human stem cells and allowed to graft over the next 12 weeks. Humanized mice were then infected with EBV and incubated for 3 weeks to allow infection to occur. At the end of the infection period, the mice were treated with two dosages of anti-GAL9 P9-15 or IgG control on day 22 and day 26. Ten days post-treatment, living mice were euthanized and analyzed.

Generation of Humanized NRG Mice (hu-NRG)

Five female NRG (NOD-Rag1^{null} IL2rg^{null}, NOD rag gamma) were used for each treatment group. The Rag1^{null} mutation renders the mice B and T cell deficient and the IL2rg^{null} mutation prevents cytokine signaling through multiple receptors, leading to a deficiency in functional NK cells. NRG mice are therefore extremely immunodeficient, allowing for engraftment of human CD34⁺ hematopoietic stem cells.

The mice were irradiated twice, 3-4 hours apart, with 275cGy per dose (total of 550cGy), injected intravenously with 5×10⁴ CD34⁺ human stem cells, and then allowed to engraft for three weeks to produce the humanized NRG (“hu-NRG”) mice. The hu-NRG mice were weighed bi-weekly for 12 weeks to assess their health. In addition, tail bleeds were performed on week 4, week 8, and week 12 after administration of human CD34+ stem cells to monitor and confirm stable engraftment in the mice by flow cytometric analysis for detection of human CD45⁺ cells including total mononuclear cells (CD45⁺), T cells (CD3⁺) and B cells (CD19⁺).

Spleen Tumors

Following euthanasia, the spleens were excised and examined to determine the number of macroscopically visible tumors, cell number, and weight, except where the mice died or were euthanized for ethical reasons.

Assessment of EBV Viral Load

EBV loads in the spleen and blood were measured using real-time PCR.

Statistical Analyses

Mann-Whitney U test based on 2-sided tail was conducted using GraphPad Prism 7 Software (San Diego, Calif.).

Results/Conclusion

The spleens from mice treated with anti-GAL9 P9-15 mice showed fewer macroscopically visible tumors than spleens from mice treated with IgG control. See FIG. 10B. P9-15 treated mice had lighter spleen weight (average 0.100 g per spleen) compared to the IgG control treated mice (average 0.224 g per spleen, p-value<0.0079), as well as significantly fewer spleen cells (22.14×10⁶ in P9-15 treated mice compared to 51.04×10⁶ in IgG treated control, p-value<0.0159). See FIGS. 10C-10D. Data are shown as the mean; error bars are SEM.

In addition, treatment with P9-15 controlled the viral load by 88%. P9-15 treated mice had an average of 0.32×10⁶ copies/μg of EBV, while the IgG treated mice had an average of 2×10⁶ copies/μg of EBV (p-value<0.0079). See FIG. 10E. Data are shown as the mean; error bars are ±SEM. These results demonstrate that treatment with P9-15 can reduce the development of EBV-induced tumors as well as control viral load.

### 6.11.12. Example 11: Treatment with Anti-GAL9 P9-28 Results in Fewer Epstein-Barr Virus (EBV)-Induced Tumors

This study was conducted to determine the effect of GAL9 P9-28 candidate on Epstein-Barr virus (EBV)-induced tumors in a humanized mouse model.

Animals, Infection of Humanized Mice and Treatment

This study was conducted as described in Example 10 above.

Results/Conclusion

Anti-GAL9 P9-28 treated mice showed no visible macroscopic tumors on the spleens compared to IgG control. See FIG. 11. The anti-GAL9 P9-23 treated mice were unlikely to have tumors within the spleen, as inferred from the small spleen size with low cell numbers. These results demonstrate that treatment with P9-28 can reduce EBV-induced tumor development.

### 6.11.13. Example 12: Anti-GAL9 Silent Fc P9-18 (sFcP9-18) has an Antitumor Effect; sFcP9-18 and P9-18 can Establish Antitumor Immune Memory

This study was conducted to test the contribution of the Fc region to the antitumor effect of the immune-activating anti-Gal9 antibodies. In addition, a re-challenge study was conducted to determine if P9-18 or sFcP9-18 can establish antitumor immune memory.

Antibodies

P9-18 antigen-binding sites were formatted on either a murine IgG1 backbone, murine IgG2a backbone, or on a murine IgG2a backbone with Fc receptor-binding null mutations (sFc). The silent Fc (sFc) P9-18 antibody was made by making key point mutations that abrogate binding of the Fc to Fc receptors.

CT26 Cells

CT26 tumor cells were cultured in RPMI medium in a humidified incubator at 37° C., in an atmosphere of 5% CO₂ and 95% air.

Mice and Treatment Schedule

Seven to ten mice were implanted subcutaneously with 1×10⁵ CT26 tumor cells, and then treated with either control IgG (mouse IgG2a), P9-18-IgG1 (murine IgG1 backbone), FcR-silent sFcP9-18 (murine IgG2a backbone with Fc-receptor binding null mutations), or P9-18 (murine IgG2a backbone) I.P., at 200 μg on days 7, 11, 15, and 19.

Tumor Volume Growth

Mice were monitored for up to 143 days and tumors measured every 1-3 days by calipers. Tumor volume (mm³) was calculated according to the formula: tumor length×tumor width×2/2.

Complete Regression Response (CR)

Complete regression for the study was defined as a tumor volume 0 mm³ for 20 consecutive measurements during the study. Animals were scored every 1-3 days during the study for a complete regression (CR) event.

Re-Challenge of CT26 Tumors

Tumor-free mice surviving the original initial tumor clearance study were allowed to rest for 65-70 days after tumors cleared. On day 107, the animals were re-implanted with 1×10⁵ CT26 tumor cells with no additional treatment. New control mice were given a treatment with IgG2a control on day 113. Tumors were then allowed to grow for an additional 36 days. Tumor volume was determined as described above for days 107-143.

Results/Conclusion

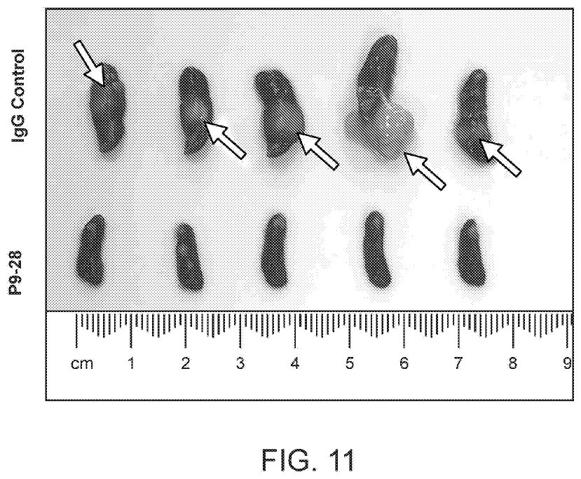

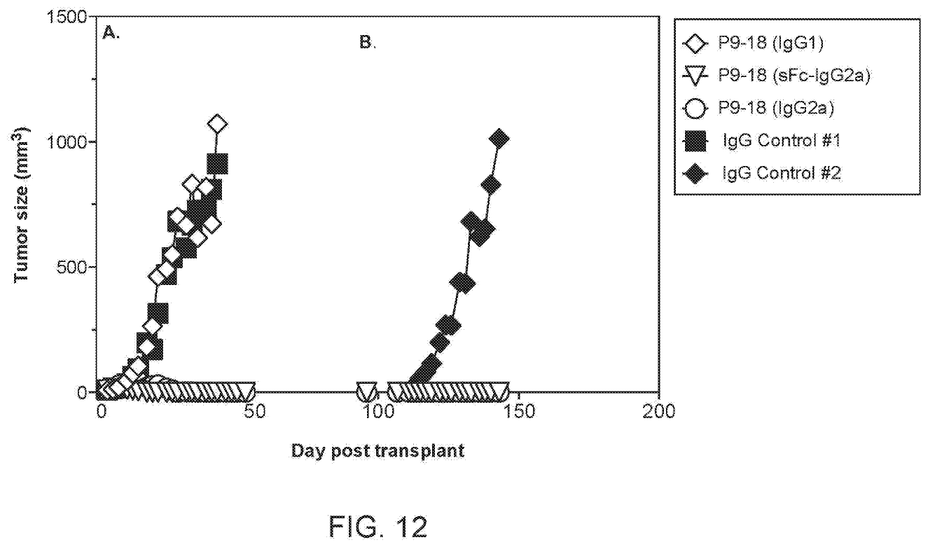

The results from the tumor growth study are shown in FIG. 12A. In mice administered the IgG (IgG2a) control antibody ( [symbol] ), tumors reached 900-1000 mm³ over the initial 50-day period. In contrast, treatment with P9-18-IgG2a ( [symbol] ) had 77% (7/9) CR, and treatment with sFcP9-18-IgG2a ( [symbol] ) had 70% (7/10) CR. These results demonstrate that the Fc region of the P9-18 antibody is not required for its antitumor effect.

The P9-18 ABS reformatted into an IgG1 backbone ( [symbol] ) did not inhibit tumor growth, showing similar tumor growth to the control.

The results from the re-challenge study are shown in FIG. 12B. Mice originally treated with P9-18-IgG2a had 100% (7/7) CR to new tumors, without additional treatment. Likewise, mice originally treated with sFcP9-18-IgG2a had 100% (7/7) CR to new tumors, without additional treatment. Treatment with the control IgG (IgG2a) antibody ( [symbol] ) showed similar tumor growth as in the initial tumor clearance study. These data demonstrate that mice treated with P9-18 or sFcP9-18 have established anti-tumor immune memory to CT26 tumor cells following initial treatment with P9-18-IgG2a or sFc9-18-IgG2a.

### 6.11.14. Example 13: Treatment with Anti-GAL9 P9-18 Increases PD-L2 Expression on Tumor-Associated Dendritic Cells and Tumor Cells

Anti-GAL9 P9-18 was tested for its effect on PD-L1 and PD-L2 cell surface expression on tumor-associated dendritic cells and tumor cells.

Animals and Treatment

Three to five BALB/c mice were implanted subcutaneously with CT26 tumor cells and treated with P9-18 ABS formatted on a mouse IgG2a backbone or with mouse IgG2a control. All treatments were administered (I.P.), at 200 μg, on days 7 and 11.

Flow Cytometry

Tumors were dissected on day 13, digested, and dissociated. Next, a CD45.1⁺ cell population, which includes immune and tumor cells, was isolated using anti-CD45.1 magnetic beads (Miltenyi Biotec, Germany). The CD45.1⁺ cell population was labelled and analyzed by flow cytometry for PD-L1 and PD-L2 cell surface expression on tumor-associated dendritic cells (CD11c+) and tumor cells. The reagents used are shown in Table 13 below.

TABLE 13
Reagents

| Reagents | Fluorophore | Clone | Supplier |
|---|---|---|---|
| CD45.1 | APC | A20 | eBioscience |
| CD11c | BV421 | N418 | BD Horizon |
| PD-L1 | BV605 | 10F.9G2 | Bioledgend |
| PD-L2* | AF488 | TY25 | BioXcell |
| CD45.1 Micro-Beads | | | Milteny Biotec |
| Lightning-Link Rapid | | | Bionovus Life |
| DyLight 488 Labelling | | | Sciences |

*labelled using Lightning-Link Rapid DyLight 488 labelling kit.

Statistical Analyses

Unpaired t test with Welch's correction was conducted using GraphPad Prism 7 Software (San Diego, Calif.).

Results/Conclusion

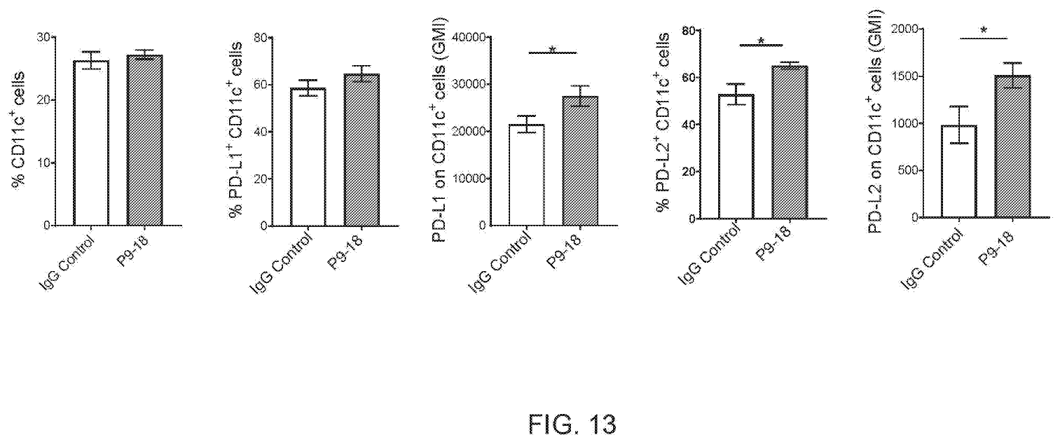

FIG. 13 shows the mean percentage of PD-L1⁺ or PD-L2⁺ tumor-associated dendritic cells (CD11c⁺) and the mean cell surface expression level (GMI) of PD-L1 or PD-L2 on tumor-associated dendritic cells (CD11c⁺) after treatment with P9-18 (murine IgG2a backbone) or control. Treatment with P9-18 significantly increased the percentage of PDL2⁺ tumor-associated dendritic cells. The amount of PD-L1 and PD-L2 expression (GMI) was also significantly increased on tumor-associated dendritic cells compared to control. See FIG. 13. Data are shown as the mean; error bars are ±SEM.

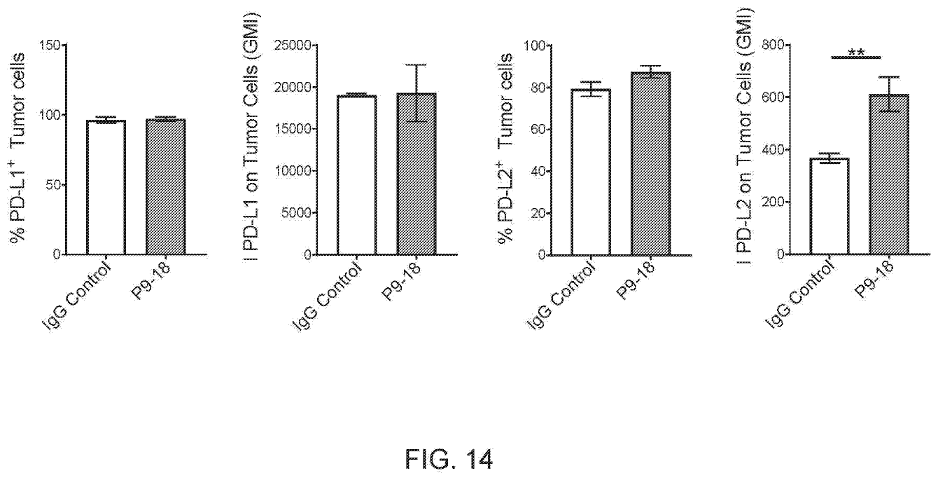

FIG. 14 shows the mean percentage of PD-L1⁺ or PD-L2⁺ tumor cells and the mean cell surface expression level (GMI) of PD-L1 or PD-L2 on tumor cells after treatment with P9-18 (murine IgG2a backbone) or IgG control. Treatment with P9-18 significantly increased the amount (GMI) of PD-L2 cell surface expression on tumor cells but not PD-L1 cell surface expression. See FIG. 14. Data are shown as the mean; error bars are ±SEM. Without wishing to be bound by any theory, we hypothesize that PD-L2⁺ tumor cells may inhibit PD-L1 binding to PD-1 on tumors.

### 7. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

1. A Galectin-9 (GAL9) antigen binding molecule, comprising: a first antigen binding site (ABS) specific for a first epitope of a first GAL9 antigen, wherein the first antigen binding site comprises all three VH CDRs and/or all three VL CDRs from any one of the ABS clones selected from P9-28, P9-02B, P9-04, P9-05, P9-08, P9-09, P9-10, P9-15, P9-16, P9-18, P9-19, P9-20, P9-21, P9-22, P9-27, P9-31, P9-32, P9-36, P9-39, P9-49, P9-54, and P9-58.

2.-3. (canceled)

4. The Galectin-9 (GAL9) antigen binding molecule of claim 1, wherein the first antigen binding site comprises the VL sequence and the VH sequence from any one of the ABS clones selected from P9-28, P9-02B, P9-04, P9-05, P9-08, P9-09, P9-10, P9-15, P9-16, P9-18, P9-19, P9-20, P9-21, P9-22, P9-27, P9-31, P9-32, P9-36, P9-39, P9-49, P9-54, and P9-58.

5. The GAL9 antigen binding molecule of claim 4, wherein the first antigen binding site (ABS) further comprises a first IgG heavy chain polypeptide and a first IgG light chain polypeptide.

6. The GAL9 antigen binding molecule of claim 1, wherein the GAL9 antigen is a human GAL9 antigen.

7. The GAL9 antigen binding molecule of claim 1, wherein the GAL9 antigen binding molecule further comprises a second antigen binding site (ABS).

8. The GAL9 antigen binding molecule of claim 7, wherein the second ABS is specific for: a GAL9 antigen; a second epitope of the first GAL9 antigen; the first epitope of the first GAL9 antigen and is identical to the first ABS; or an antigen other than the first GAL9 antigen.

9.-10. (canceled)

11. The GAL9 antigen binding molecule of claim 7, wherein the second ABS comprises: all three VH CDRs, all three VL CDRs, or all three VH CDRs and all three VL CDRs from another ABS clone; the VL sequence and the VH sequence from another ABS clone; or a full immunoglobulin heavy chain sequence comprising the VH sequence and a full immunoglobulin light chain sequence comprising the VL sequence from another ABS clone: wherein the another ABS clone is selected from P9-02B, P9-04, P9-05, P9-08, P9-09, P9-10, P9-15, P9-16, P9-18, P9-19, P9-20, P9-21, P9-22, P9-27, P9-28, P9-31, P9-32, P9-36, P9-39, P9-49, P9-54, and P9-58.

12.-14. (canceled)

15. The GAL9 antigen binding molecule of claim 1, wherein the first antigen binding site comprises all three VH CDRs, all three VL CDRs, or all three VH CDRs and all three VL CDRs from any one of the ABS clones selected from: P9-18, P9-15, P9-21, P9-22, P9-28, and P9-32; or selected from: P9-18, P9-15, P9-21, and P9-28.

16.-20. (canceled)

21. The GAL9 antigen binding molecule of claim 1, wherein the GAL9 antigen binding molecule comprises an antibody format selected from the group consisting of: full-length antibodies, Fab fragments, Fvs, scFvs, tandem scFvs, Diabodies, scDiabodies, DARTs, tandAbs, minibodies, and B-bodies.

22. The GAL9 antigen binding molecule of claim 1, wherein the GAL9 antigen binding molecule increases TNF-a secretion by activated immune cells upon contact, wherein the increase is greater than an 80-fold increase relative to activated immune cells treated with a control agent.

23. The GAL9 antigen binding molecule of claim 1, wherein the GAL9 antigen binding molecule increases IFN-g secretion by activated immune cells upon contact, wherein the increase is greater than a 1.2-fold increase relative to activated immune cells treated with a control agent.

24. The GAL9 antigen binding molecule of claim 1, wherein the GAL9 antigen binding molecule increases CD40L surface expression of activated CD8+ T-cells upon contact, wherein the increase is greater than a 2-fold increase relative to activated CD8+ T-cells treated with a control agent.

25. The GAL9 antigen binding molecule of claim 1, wherein the GAL9 antigen binding molecule increases OX40 surface expression of activated CD8+ T-cells upon contact, wherein the increase is greater than a 2-fold increase relative to activated CD8+ T-cells treated with a control agent.

26. The GAL9 antigen binding molecule of claim 1, wherein the GAL9 antigen binding molecule increases IL-12 production of activated dendritic cells (DCs) upon contact, wherein the increase is greater than a 20-fold increase relative to activated DCs treated with a control agent.

27. The GAL9 antigen binding molecule of claim 1, wherein the GAL9 antigen binding molecule increases PD-L2 surface expression on activated dendritic cells (DCs) upon contact, wherein the increase is greater than a 4-fold increase relative to activated DCs treated with a control agent.

28. The GAL9 antigen binding molecule of claim 22, wherein the control agent is a negative control agent or positive control agent or a control antibody.

29. (canceled)

30. The GAL9 antigen binding molecule of claim 28, wherein the control antibody is selected from the group consisting of: an ECA42 clone anti-GAL9 antibody, an RG9.1 clone anti-GAL9 antibody, an RG9.35 clone anti-GAL9 antibody, an anti-PD1 antibody, and a non-GAL9 binding isotype control antibody.

31. The GAL9 antigen binding molecule of claim 22, wherein the activated immune cells are activated by stimulation by a peptide or plurality of peptides known to induce an immune response.

32.-64. (canceled)

65. A pharmaceutical composition comprising the GAL9 antigen binding molecule of claim 1 and a pharmaceutically acceptable diluent.

66. A method for treating a subject with cancer, the method comprising administering a therapeutically effective amount of the pharmaceutical composition of claim 65 to the subject.

67. (canceled)
